(19) United States
(12) Patent Application Publication
(10) Pub. No.: US 2025/0213489 A1
(43) Pub. Date: Jul. 3, 2025

(54) OSMOTIC TABLETS OF DEXPRAMIPEXOLE AND METHODS OF MANUFACTURING AND USE THEREOF

(71) Applicant: Areteia Therapeutics, Inc., Chapel Hill, NC (US)

(72) Inventors: Samir Thakorbhai DESAI, Suwanee, GA (US); Gregory C. WILLIAMS, Mendham, NJ (US)

(73) Assignee: Areteia Therapeutics, Inc., Chapel Hill, NC (US)

(21) Appl. No.: 18/990,646

(22) Filed: Dec. 20, 2024

Related U.S. Application Data
(60) Provisional application No. 63/706,524, filed on Oct. 11, 2024.
(60) Provisional application No. 63/612,893, filed on Dec. 20, 2023.

(51) Int. Cl. A61K 9/28; A61K 9/00; A61K 9/20; A61K 31/428

(52) U.S. Cl. CPC A61K 9/2866; A61K 9/0004; A61K 9/0053; A61K 9/2009; A61K 9/2013; A61K 9/2027; A61K 9/2054; A61K 9/2853; A61K 31/428

(57) ABSTRACT

The present disclosure relates to pharmaceutical compositions of dexpramipexole, or a pharmaceutically acceptable salt thereof. In particular, the present disclosure relates to pharmaceutical compositions in the form of orally deliverable tablets. The pharmaceutical compositions of the present disclosure provide for a sustained release of dexpramipexole, or a pharmaceutically acceptable salt thereof. In certain embodiments, the pharmaceutical compositions of the present disclosure additionally provide for an immediate release of dexpramipexole, or a pharmaceutically acceptable salt thereof. The present disclosure further relates to methods of manufacturing the pharmaceutical compositions, and methods of using the pharmaceutical compositions to treat and prevent certain diseases, such as eosinophilic disorders, in a human subject.

### RELATED APPLICATIONS

This application claims priority to U.S. Provisional Application No. 63/706,524 filed Oct. 11, 2024, which is incorporated hereby by reference in its entirety. This application claims priority to U.S. Provisional Application No. 63/612,893 filed Dec. 20, 2023, which is incorporated hereby for reference in its entirety.

### FIELD OF THE DISCLOSURE

The present disclosure relates to pharmaceutical compositions of dexpramipexole, or a pharmaceutically acceptable salt thereof. In particular, the present disclosure relates to pharmaceutical compositions in the form of orally deliverable tablets providing for a sustained release of dexpramipexole, or a pharmaceutically acceptable salt thereof. In some aspects, the pharmaceutical compositions further comprise an immediate release layer comprising dexpramipexole, or a pharmaceutically acceptable salt thereof, and therefore are capable of providing for an immediate and a sustained release of the drug. The present disclosure further relates to methods of manufacturing the pharmaceutical compositions, as well as uses of the pharmaceutical compositions to treat or prevent certain diseases or conditions in human subjects, in particular such diseases or conditions that are related to elevated eosinophil levels.

### BACKGROUND OF THE DISCLOSURE

Dexpramipexole ((6R)-2-amino-4,5,6,7-tetrahydro-6-(propylamino)benzothiazole; (formula I)) is the enantiomer of pramipexole ((6S)-2-amino-4,5,6,7-tetrahydro-6-(propylamino)benzothiazole; (formula II)).

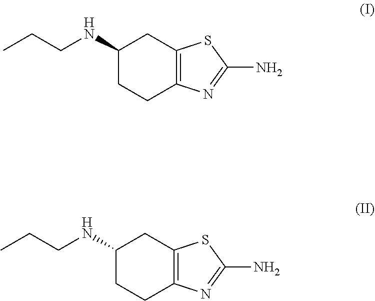

Pramipexole is a dopamine D2 receptor agonist used in the treatment of Parkinson's disease. Pramipexole is sold in the form of orally deliverable tablets under the brand Mirapex®, among others. Mirapex® tablets are available as immediate release (IR) and extended release (ER) formulations, and contain pramipexole as the dihydrochloride monohydrate salt. Mirapex® ER tablets for oral administration are available at a dose of 0.375 mg, 0.75 mg, 1.5 mg, 2.25 mg, 3 mg, 3.75 mg, or 4.5 mg of pramipexole dihydrochloride monohydrate per tablet given once daily (QD; quaque die). Inactive ingredients are, according to the manufacturer, hypromellose (hydroxypropyl methylcellulose), cornstarch, carbomer homopolymer, colloidal silicon dioxide, and magnesium stearate. The term “extended release” or “ER” is used herein synonymously with “sustained release.”

In contrast, the affinity of dexpramipexole for dopamine receptors was found to be greatly reduced when compared to pramipexole, which renders dexpramipexole unsuitable as a dopamine agonist. Instead, dexpramipexole was shown to selectively and significantly lower eosinophil counts in human blood and tissues. The drug is currently under clinical development for eosinophil-associated diseases, including eosinophilic asthma and chronic obstructive pulmonary disease (COPD).

In literature reporting testing of dexpramipexole compositions, the dexpramipexole compositions were immediate release (IR) tablets that are given twice a day (BID; bis in die). Doses evaluated include 37.5 mg, 75 mg and 150 mg of dexpramipexole dihydrochloride equivalent per tablet (corresponding to a daily dose of 75 mg, 150 mg, and 300 mg, respectively, which is up to 800-fold higher than the lowest daily dose of 0.375 mg pramipexole salt provided by Mirapex® ER tablets).

Because of the relatively high amount of dexpramipexole that is typically used per day (e.g., about 75 mg, about 150 mg, or about 300 mg dexpramipexole dihydrochloride equivalent per day), it could not have been reasonably expected that formulating the drug as a composition suitable for once daily oral administration to a human would be possible. A pharmaceutical composition that is intended for oral use, such as a tablet, must be swallowable by a human. This means, a certain maximum composition weight (typically about 1500 mg) should not be exceeded. If, however, the amount of the active ingredient is relatively high (such as in the event of dexpramipexole, or a pharmaceutically acceptable salt thereof, at doses typically used), the amount of additional ingredients that can be added to the pharmaceutical composition for formulation is limited and formulation possibilities are thus restricted. This can be problematic because, in general, a higher amount of active ingredient also requires a higher amount of additional ingredients to allow a pharmaceutical composition (such as a tablet) to be manufactured and sufficiently slow down the release of the active ingredient for a once daily formulation. Such additional ingredients can be one or more of a diluent, binder, glidant, and lubricant. In the case of osmotic tablets, for example, additional formulation components that contribute to the release of the drug and at the same time add to the composition weight are a semipermeable membrane coating and optionally also an osmotic agent such as sodium chloride. In contrast, as outlined above, for pramipexole, significantly lower amounts of drug are used per day (the highest dose in Mirapex® ER once daily formulations being 4.5 mg pramipexole dihydrochloride monohydrate). See, for example, Encyclopedia of Pharmaceutical Technology, Volume 1, Third Edition, edited by James Swarbrick, Chapter “Drug Delivery: Controlled Release” by Chien and Lin.

Surprisingly, the present disclosure provides dexpramipexole, or a pharmaceutically acceptable salt thereof, in the form of an orally deliverable tablet that is suitable for once daily administration to a human, which would not have been expected in view of constraints on tablet size coupled with the high amount of dexpramipexole, or a pharmaceutically acceptable salt thereof, needed for once daily administration.

### BRIEF SUMMARY OF THE DISCLOSURE

Although, as outlined above, it could not have been reasonably expected that formulating dexpramipexole, or a pharmaceutically acceptable salt thereof, as a composition suitable for once daily oral administration to a human would be possible, the present disclosure inter alia provides such a pharmaceutical composition. The pharmaceutical compositions of the present disclosure allow simplification of a patient's administration scheme by reducing the number of recommended daily intakes compared to immediate release dexpramipexole formulations known in the art, which improves patient's compliance and attenuates potential adverse events that are related, e.g., to high plasma concentration peaks.

In some aspects, the present disclosure provides a pharmaceutical composition in the form of an orally deliverable tablet comprising dexpramipexole, or a pharmaceutically acceptable salt thereof, wherein the pharmaceutical composition provides for a sustained release of dexpramipexole, or a pharmaceutically acceptable salt thereof that makes the tablet suitable for once daily oral administration to a human. In some aspects, the orally deliverable tablet provides for both an immediate release and a sustained release of dexpramipexole, or a pharmaceutically acceptable salt thereof.

In some aspects, the present disclosure provides a pharmaceutical composition in the form of an orally deliverable tablet comprising a tablet core, a semipermeable membrane coating surrounding the tablet core, and dexpramipexole, or a pharmaceutically acceptable salt thereof, in an amount of about 50 mg to about 400 mg of dexpramipexole dihydrochloride equivalent, wherein
at least about 70% of the amount of dexpramipexole, or a pharmaceutically acceptable salt thereof, is in the tablet core;
the tablet core comprises a homogeneous mixture of an inorganic osmotic agent and
the amount of dexpramipexole, or a pharmaceutically acceptable salt thereof, that is in the tablet core, wherein the inorganic osmotic agent constitutes about 10% to about 40% by weight of the tablet core;
the semipermeable membrane coating comprises about 5% to about 25% plasticizer by weight of the semipermeable membrane coating;
the weight ratio of the semipermeable membrane coating to the tablet core is about 0.03:1 to about 0.11:1; and
the weight of the tablet is about 1500 mg or less.

In some aspects, the tablet core further comprises additional excipients.

In some embodiments, the pre-blend comprises an antioxidant. In some embodiments, the antioxidant is capable of acting as a nitrite scavenger. Non-limiting examples of nitrite scavengers include, but are not limitd to, ascorbic acid, L-cysteine, caffeic acid, cysteine HCL, methionine, tartaric acid, gallic acid, uric acid, and sodium sulphite. In some embodiments, the pre-blend comprises one nitrite scavenger. In another embodiment, the pre-blend comprises multiple nitrite scavengers. In some embodiments, the nitrite scavengers constitute about 0.1% to about 1.5% by weight of the tablet core. In some embodiments, the nitrite scavenger constitutes about 0.1% to about 1.0% by weight of the tablet core. In some embodiments, the nitrite scavenger constitutes about 0.1% to about 0.5% by weight of the tablet core. In some embodiments, the nitrite scavenger constitutes about 0.5% to about 1.5% by weight of the tablet core. In some embodiments, the nitrite scavenger constitutes about 1% to about 1.5% by weight of the tablet core. In some embodiments, the nitrite scavenger constitutes about 0.1%, about 0.2%, about 0.3%, about 0.4%, about 0.5%, about 0.6%, about 0.7%, about 0.8%, about 0.9%, about 1%, about 1.1%, about 1.2%, about 1.3%, about 1.4%, or about 1.5% by weight of the tablet core.

In some aspects, the present disclosure provides a pharmaceutical composition in the form of an orally deliverable tablet comprising a tablet core, a semipermeable membrane coating surrounding the tablet core, and dexpramipexole, or a pharmaceutically acceptable salt thereof, in an amount of about 50 mg to about 400 mg of dexpramipexole dihydrochloride equivalent, wherein
at least about 70% of the amount of dexpramipexole, or a pharmaceutically acceptable salt thereof, is in the tablet core;
the tablet core comprises a homogeneous mixture of an inorganic osmotic agent and the amount of dexpramipexole, or a pharmaceutically acceptable salt thereof, that is in the tablet core, wherein the inorganic osmotic agent constitutes about 10% to about 40% by weight of the tablet core;
the semipermeable membrane coating comprises about 5% to about 40% plasticizer by weight of the semipermeable membrane coating;
the weight ratio of the semipermeable membrane coating to the tablet core is about 0.03:1 to about 0.11:1; and
the weight of the tablet is about 1500 mg or less.

In some aspects, the tablet core further comprises additional excipients.

Osmotic drug delivery systems providing sustained release of active ingredient(s) are generally also referred to as “osmotic-controlled release oral delivery system (OROS),” or briefly “osmotic pumps.” In addition, the orally deliverable tablets provided herein that comprise an osmotic agent are also referred to herein as “osmotic tablets.” The mechanism of osmosis is the spontaneous net movement of water molecules from a solution of lower solute concentration to another of higher solute concentration through a semipermeable membrane, which is generally designed to allow only the permeation of the water molecules, but not permeation of solute. An osmotic tablet generally comprises a tablet core comprising an active agent and optionally further an osmotic agent, wherein the tablet core is surrounded by a semipermeable membrane coating providing one or more delivery orifices. As an osmotic tablet passes through the body, water is absorbed through the semipermeable membrane coating by the mechanism of osmosis, and the resulting osmotic pressure in the tablet core pushes the active agent through the delivery orifice(s) in the semipermeable membrane coating into the gastrointestinal tract.

As shown in the Example section (see, in particular, Examples 1, 3, and 4), osmotic tablets comprising a mixture of dexpramipexole, or a pharmaceutically acceptable salt thereof, and an inorganic osmotic agent at the amounts specified herein (such as at about 20% inorganic osmotic agent by weight of the tablet core) in the tablet core, a plasticizer in the semipermeable membrane coating at the amounts specified herein (such as at about 15% by weight of the semipermeable membrane coating), and a weight ratio of the semipermeable membrane coating to the tablet core at the levels specified herein (such as about 0.06:1 to about 0.08:1) provide for in vitro dissolution profiles of dexpramipexole, or a pharmaceutically acceptable salt thereof, that render the osmotic tablets suitable for once daily administration to a human, while at the same time yielding sufficiently strong tablets for manufacturing (e.g., suitable tablet hardness). In contrast, the in vitro dissolution profiles were inferior (e.g., terminal release at 24 hours was lower) if, for example, dexpramipexole, or a pharmaceutically acceptable salt thereof, was not mixed with inorganic osmotic agent in the tablet core (see, for example, Example 1).

In some embodiments, the inorganic osmotic agent constitutes about 15% to about 35% by weight of the tablet core. In some embodiments, the inorganic osmotic agent constitutes about 15% to about 25% by weight of the tablet core. In some embodiments, the inorganic osmotic agent constitutes about 18% to about 22% by weight of the tablet core. In some embodiments, the inorganic osmotic agent constitutes about 20% by weight of the tablet core.

In some embodiments, the inorganic osmotic agent is sodium chloride, potassium chloride, magnesium chloride, sodium hydrogen phosphate, potassium hydrogen phosphate, or any combination thereof. In some embodiments, the inorganic osmotic agent is sodium chloride.

In some embodiments, the homogeneous mixture in the tablet core does not comprise polyethylene oxide. In some embodiments, the homogeneous mixture in the tablet core does not comprise a swellable polymeric osmotic agent.

In some embodiments, the semipermeable membrane coating further comprises cellulose acetate. In some embodiments, cellulose acetate has an acetyl content of about 35% to about 45%. In some embodiments, cellulose acetate has an acetyl content of about 38% to about 42%. In some embodiments, cellulose acetate has an acetyl content of about 40%.

In some embodiments, the semipermeable membrane coating comprises about 60% to about 95% cellulose acetate by weight of the semipermeable membrane coating. In some embodiments, the semipermeable membrane coating comprises about 75% to about 95% cellulose acetate by weight of the semipermeable membrane coating.

In some embodiments, the semipermeable membrane coating comprises about 70% to about 90% cellulose acetate by weight of the semipermeable membrane coating and about 30% to about 10% plasticizer by weight of the semipermeable membrane coating. In some embodiments, the semipermeable membrane coating comprises about 70% cellulose acetate by weight of the semipermeable membrane coating and about 30% plasticizer by weight of the semipermeable membrane coating. In some embodiments, the semipermeable membrane coating comprises about 75% cellulose acetate by weight of the semipermeable membrane coating and about 25% plasticizer by weight of the semipermeable membrane coating. In some embodiments, the semipermeable membrane coating comprises about 80% to about 90% cellulose acetate by weight of the semipermeable membrane coating and about 20% to about 10% plasticizer by weight of the semipermeable membrane coating. In some embodiments, the semipermeable membrane coating comprises about 83% to about 87% cellulose acetate by weight of the semipermeable membrane coating and about 17% to about 13% plasticizer by weight of the semipermeable membrane coating. In some embodiments, the semipermeable membrane coating comprises about 83% cellulose acetate by weight of the semipermeable membrane coating and about 17% plasticizer by weight of the semipermeable membrane coating. In some embodiments, the semipermeable membrane coating comprises about 85% cellulose acetate by weight of the semipermeable membrane coating and about 15% plasticizer by weight of the semipermeable membrane coating.

In some embodiments, the plasticizer constitutes about 10% to about 35% by weight of the semipermeable membrane coating. In some embodiments, the plasticizer constitutes about 15% to about 30% by weight of the semipermeable membrane coating. In some embodiments, the plasticizer constitutes about 30% by weight of the semipermeable membrane coating. In some embodiments, the plasticizer constitutes about 25% by weight of the semipermeable membrane coating. In some embodiments, the plasticizer constitutes about 10% to about 20% by weight of the semipermeable membrane coating. In some embodiments, the plasticizer constitutes about 13% to about 17% by weight of the semipermeable membrane coating. In some embodiments, the plasticizer constitutes about 17% by weight of the semipermeable membrane coating. In some embodiments, the plasticizer constitutes about 15% by weight of the semipermeable membrane coating.

In some embodiments, the plasticizer is polyethylene glycol, triethyl citrate, triacetin, diethyl phthalate, poloxamer, or any combination thereof. In some embodiments, the plasticizer is polyethylene glycol.

In some embodiments, the weight ratio of the semipermeable membrane coating to the tablet core is about 0.04:1 to about 0.1:1. In some embodiments, the weight ratio of the semipermeable membrane coating to the tablet core is about 0.05:1 to about 0.09:1. In some embodiments, the weight ratio of the semipermeable membrane coating to the tablet core is about 0.05:1. In some embodiments, the weight ratio of the semipermeable membrane coating to the tablet core is about 0.06:1 to about 0.08:1. In some embodiments, the weight ratio of the semipermeable membrane coating to the tablet core is about 0.06:1. In some embodiments, the weight ratio of the semipermeable membrane coating to the tablet core is about 0.07:1. In some embodiments, the weight ratio of semipermeable membrane coating to the tablet core is about 0.08:1.

In some embodiments, the homogeneous mixture in the tablet core further comprises additional excipients. In some embodiments, the homogeneous mixture in the tablet core further comprises microcrystalline cellulose, polyvinylpyrrolidone-vinyl acetate copolymer, magnesium stearate, or any combination thereof. In some embodiments, the homogeneous mixture in the tablet core further comprises microcrystalline cellulose, polyvinylpyrrolidone-vinyl acetate copolymer, and magnesium stearate. In some embodiments, microcrystalline cellulose constitutes about 28% to about 32% by weight of the tablet core. In some embodiments, polyvinylpyrrolidone-vinyl acetate copolymer constitutes about 5% to about 9% by weight of the tablet core. In some embodiments, magnesium stearate constitutes about 0.25% to about 0.75% by weight of the tablet core.

In some embodiments, the amount of dexpramipexole, or a pharmaceutically acceptable salt thereof, is about 75 mg to about 400 mg of dexpramipexole dihydrochloride equivalent. In some embodiments, the amount of dexpramipexole, or a pharmaceutically acceptable salt thereof, is about 75 mg to about 300 mg of dexpramipexole dihydrochloride equivalent. In some embodiments, the amount of dexpramipexole, or a pharmaceutically acceptable salt thereof, is about 50 mg to about 100 mg of dexpramipexole dihydrochloride equivalent. In some embodiments, the amount of dexpramipexole, or a pharmaceutically acceptable salt thereof, is about 75 mg of dexpramipexole dihydrochloride equivalent. In some embodiments, the amount of dexpramipexole, or a pharmaceutically acceptable salt thereof, is about 100 mg to about 400 mg of dexpramipexole dihydrochloride equivalent. In some embodiments, the amount of dexpramipexole, or a pharmaceutically acceptable salt thereof, is about 100 mg to about 350 mg of dexpramipexole dihydrochloride equivalent. In some embodiments, the amount of dexpramipexole, or a pharmaceutically acceptable salt thereof, is about 325 mg to about 400 mg of dexpramipexole dihydrochloride equivalent. In some embodiments, the amount of dexpramipexole, or a pharmaceutically acceptable salt thereof, is about 376 mg of dexpramipexole dihydrochloride equivalent. In some embodiments, the amount of dexpramipexole, or a pharmaceutically acceptable salt thereof, is about 275 mg to about 325 mg of dexpramipexole dihydrochloride equivalent. In some embodiments, the amount of dexpramipexole, or a pharmaceutically acceptable salt thereof, is about 300 mg of dexpramipexole dihydrochloride equivalent. In some embodiments, the amount of dexpramipexole, or a pharmaceutically acceptable salt thereof, is about 125 mg to about 175 mg of dexpramipexole dihydrochloride equivalent. In some embodiments, the amount of dexpramipexole, or a pharmaceutically acceptable salt thereof, is about 150 mg of dexpramipexole dihydrochloride equivalent.

In some embodiments, the pharmaceutically acceptable salt of dexpramipexole is a methane sulfonic acid salt, sulfuric acid salt, tartaric acid salt, p-toluene sulfonic acid salt, phosphoric acid salt, maleic acid salt, fumaric acid salt, malic acid salt, citric acid salt, succinic acid salt, or any combination thereof. In some embodiments, the pharmaceutically acceptable salt of dexpramipexole is dexpramipexole dihydrochloride or a hydrate thereof. In some embodiments, the pharmaceutically acceptable salt of dexpramipexole is as dexpramipexole dihydrochloride monohydrate.

In some embodiments, the weight of the tablet is about 1300 mg or less. In some embodiments, the weight of the tablet is about 900 mg to about 1300 mg or about 1000 mg to about 1200 mg. In some embodiments, the weight of the tablet is about 1000 mg or less. In some embodiments, the weight of the tablet is about 800 mg to about 1000 mg or about 820 mg to about 950 mg. In some embodiments, the weight of the tablet is about 825 mg to about 835 mg or about 930 mg to about 940 mg.

In some embodiments, at least about 80% of the amount of dexpramipexole, or a pharmaceutically acceptable salt thereof, is in the tablet core. In some embodiments, at least 99% or 100% of the amount of dexpramipexole, or a pharmaceutically acceptable salt thereof, is in the tablet core.

In some embodiments, at least 99% or 100% of the amount of dexpramipexole, or a pharmaceutically acceptable salt thereof, is in the tablet core and the homogeneous mixture in the tablet core consists essentially of.
(a) about 315 mg to about 325 mg dexpramipexole dihydrochloride monohydrate;
(b) about 227 mg to about 237 mg microcrystalline cellulose;
(c) about 48 mg to about 58 mg polyvinylpyrrolidone-vinyl acetate copolymer;
(d) about 147 mg to about 157 mg sodium chloride; and
(e) about 2 mg to about 6 mg magnesium stearate;
   wherein the tablet comprises about 42 mg to about 52 mg semipermeable membrane coating, and wherein the semipermeable membrane coating comprises about 83% to about 87% cellulose acetate by weight of the semipermeable membrane coating and about 17% to about 13% plasticizer by weight of the semipermeable membrane coating.

In some embodiments, at least 99% or 100% of the amount of dexpramipexole, or a pharmaceutically acceptable salt thereof, is in the tablet core and the homogeneous mixture in the tablet core consists essentially of:
(a) about 319 mg dexpramipexole dihydrochloride monohydrate;
(b) about 232 mg microcrystalline cellulose;
(c) about 53 mg polyvinylpyrrolidone-vinyl acetate copolymer;
(d) about 152 mg sodium chloride; and
(e) about 4 mg magnesium stearate;
   wherein the tablet comprises about 47 mg semipermeable membrane coating, and wherein the semipermeable membrane coating comprises about 85% cellulose acetate by weight of the semipermeable membrane coating and about 15% plasticizer by weight of the semipermeable membrane coating.

In some embodiments, the plasticizer is polyethylene glycol, triethyl citrate, triacetin, diethyl phthalate, poloxamer, or any combination thereof. In some embodiments, the plasticizer is polyethylene glycol. In some embodiments, cellulose acetate has an acetyl content of about 35% to about 45%. In some embodiments, cellulose acetate has an acetyl content of about 40%.

In some embodiments, the pharmaceutical composition further comprising a film coating surrounding the semipermeable membrane coating. In some embodiments, the film coating comprises polyvinyl alcohol. In some embodiments, the film coating further comprises titanium dioxide, polyethylene glycol, and talc.

In some embodiments,
about 80% to about 95% of the amount of dexpramipexole, or a pharmaceutically acceptable salt thereof, is in the tablet core;
the tablet further comprises an immediate release drug coating surrounding the semipermeable membrane coating; and
about 20% to about 5% of the amount of dexpramipexole, or a pharmaceutically acceptable salt thereof, is in the drug coating.

In some embodiments, about 90% of the amount of dexpramipexole, or a pharmaceutically acceptable salt thereof, is in the tablet core and about 10% of the amount of dexpramipexole, or a pharmaceutically acceptable salt thereof, is in the drug coating.

In some embodiments, about 82% to about 88% of the amount of dexpramipexole, or a pharmaceutically acceptable salt thereof, is in the tablet core and about 18% to about 12% of the amount of dexpramipexole, or a pharmaceutically acceptable salt thereof, is in the drug coating. In some embodiments, about 85% of the amount of dexpramipexole, or a pharmaceutically acceptable salt thereof, is in the tablet core and about 15% of the amount of dexpramipexole, or a pharmaceutically acceptable salt thereof, is in the drug coating.

In some embodiments, the drug coating further comprises a binder. In some embodiments, the weight ratio of dexpramipexole dihydrochloride monohydrate equivalent to binder in the drug coating is about 1:1 to about 1:4. In some embodiments, the weight ratio of dexpramipexole dihydrochloride monohydrate equivalent to binder in the drug coating is about 1:3 or about 1:2. In some embodiments, the binder comprises hydroxypropyl methylcellulose and polyethylene glycol.

In some embodiments, the tablet further comprises a seal coating between the semipermeable membrane coating and the drug coating. In some embodiments, the seal coating comprises hydroxypropyl methylcellulose and polyethylene glycol.

In some embodiments, the homogeneous mixture in the tablet core consists essentially of:
(a) about 265 mg to about 275 mg dexpramipexole dihydrochloride monohydrate;
(b) about 192 mg to about 202 mg microcrystalline cellulose;
(c) about 40 mg to about 50 mg polyvinylpyrrolidone-vinyl acetate copolymer;
(d) about 125 mg to about 135 mg sodium chloride; and
(e) about 1 mg to about 5 mg magnesium stearate;
   wherein the tablet comprises about 47 mg to about 57 mg semipermeable membrane coating, wherein the semipermeable membrane coating comprises about 83% to about 87% cellulose acetate by weight of the semipermeable membrane coating and about 17% to about 13% plasticizer by weight of the semipermeable membrane coating;
   wherein the tablet further comprises about 15 mg to about 25 mg of a seal coating surrounding the semipermeable membrane coating, wherein the seal coating comprises hydroxypropyl methylcellulose and polyethylene glycol; and
   wherein the tablet further comprises about 185 mg to about 195 mg of an immediate release drug coating surrounding the seal coating, wherein the drug coating comprises about 45 mg to about 50 mg dexpramipexole dihydrochloride monohydrate, and about 140 mg to about 150 mg of a binder, wherein the binder comprises hydroxypropyl methylcellulose and polyethylene glycol.

In some embodiments, the homogeneous mixture in the tablet core consists essentially of:
(a) about 271 mg dexpramipexole dihydrochloride monohydrate;
(b) about 197 mg microcrystalline cellulose;
(c) about 45 mg polyvinylpyrrolidone-vinyl acetate copolymer;
(d) about 129 mg sodium chloride; and
(e) about 3 mg magnesium stearate;
   wherein the tablet comprises about 52 mg semipermeable membrane coating, wherein the semipermeable membrane coating comprises about 85% cellulose acetate by weight of the semipermeable membrane coating and about 15% plasticizer by weight of the semipermeable membrane coating;
   wherein the tablet comprises about 21 mg seal coating; and
   wherein the tablet comprises about 191.4 mg drug coating, wherein the drug coating comprises about 47.85 mg dexpramipexole dihydrochloride monohydrate, and about 143.55 mg of a binder, wherein the binder comprises hydroxypropyl methylcellulose and polyethylene glycol.

In some embodiments, the plasticizer is polyethylene glycol, triethyl citrate, triacetin, diethyl phthalate, poloxamer, or any combination thereof. In some embodiments, the plasticizer is polyethylene glycol. In some embodiments, cellulose acetate in the semipermeable membrane coating has an acetyl content of about 35% to about 45%. In some embodiments, cellulose acetate in the semipermeable membrane coating has an acetyl content of about 40%.

In some embodiments, the pharmaceutical composition further comprises a film coating surrounding the drug coating. In some embodiments, the film coating comprises polyvinyl alcohol. In some embodiments, the film coating further comprises titanium dioxide, polyethylene glycol, and talc.

In some embodiments, at least 99% or 100% of the amount of dexpramipexole, or a pharmaceutically acceptable salt thereof, is in the tablet core and the homogeneous mixture in the tablet core consists essentially of:
(a) about 390 mg to about 410 mg dexpramipexole dihydrochloride monohydrate;
(b) about 280 mg to about 300 mg microcrystalline cellulose;
(c) about 57 mg to about 77 mg polyvinylpyrrolidone-vinyl acetate copolymer;
(d) about 180 mg to about 200 mg sodium chloride; and
(e) about 3 mg to about 7 mg magnesium stearate;
   wherein the tablet comprises about 54 mg to about 74 mg semipermeable membrane coating, and wherein the semipermeable membrane coating comprises about 81% to about 85% cellulose acetate by weight of the semipermeable membrane coating and about 15% to about 19% plasticizer by weight of the semipermeable membrane coating.

In some embodiments, at least 99% or 100% of the amount of dexpramipexole, or a pharmaceutically acceptable salt thereof, is in the tablet core and the homogeneous mixture in the tablet core consists essentially of:
(a) about 400 mg dexpramipexole dihydrochloride monohydrate;
(b) about 290 mg microcrystalline cellulose;
(c) about 67 mg polyvinylpyrrolidone-vinyl acetate copolymer;
(d) about 190 mg sodium chloride; and
(e) about 5 mg magnesium stearate;
   wherein the tablet comprises about 64 mg semipermeable membrane coating, and wherein the semipermeable membrane coating comprises about 83% cellulose acetate by weight of the semipermeable membrane coating and about 17% plasticizer by weight of the semipermeable membrane coating.

In some embodiments, the microcrystalline cellulose is silicified microcrystalline cellulose. In some embodiments, the plasticizer is polyethylene glycol, triethyl citrate, triacetin, diethyl phthalate, poloxamer, or any combination thereof. In some embodiments, the plasticizer is polyethylene glycol. In some embodiments, cellulose acetate has an acetyl content of about 35% to about 45%. In some embodiments, cellulose acetate has an acetyl content of about 40%.

In some embodiments, the pharmaceutical composition further comprising a film coating surrounding the semipermeable membrane coating. In some embodiments, the film coating comprises polyvinyl alcohol. In some embodiments, the film coating further comprises titanium dioxide, polyethylene glycol, and talc.

In some embodiments, at least 99% or 100% of the amount of dexpramipexole, or a pharmaceutically acceptable salt thereof, is in the tablet core and the homogeneous mixture in the tablet core consists essentially of:
(a) about 310 mg to about 330 mg dexpramipexole dihydrochloride monohydrate;
(b) about 222 mg to about 242 mg microcrystalline cellulose;
(c) about 43 mg to about 63 mg polyvinylpyrrolidone-vinyl acetate copolymer;
(d) about 142 mg to about 162 mg sodium chloride; and
(e) about 2 mg to about 6 mg magnesium stearate;
   wherein the tablet comprises about 24 mg to about 44 mg semipermeable membrane coating, and wherein the semipermeable membrane coating comprises about 73% to about 77% cellulose acetate by weight of the semipermeable membrane coating and about 23% to about 27% plasticizer by weight of the semipermeable membrane coating.

In some embodiments, at least 99% or 100% of the amount of dexpramipexole, or a pharmaceutically acceptable salt thereof, is in the tablet core and the homogeneous mixture in the tablet core consists essentially of:
(a) about 319 mg dexpramipexole dihydrochloride monohydrate;
(b) about 232 mg microcrystalline cellulose;
(c) about 53 mg polyvinylpyrrolidone-vinyl acetate copolymer;
(d) about 152 mg sodium chloride; and
(e) about 4 mg magnesium stearate;
   wherein the tablet comprises about 34 mg semipermeable membrane coating, and wherein the semipermeable membrane coating comprises about 75% cellulose acetate by weight of the semipermeable membrane coating and about 25% plasticizer by weight of the semipermeable membrane coating.

In some embodiments, the microcrystalline cellulose is silicified microcrystalline cellulose. In some embodiments, the plasticizer is polyethylene glycol, triethyl citrate, triacetin, diethyl phthalate, poloxamer, or any combination thereof. In some embodiments, the plasticizer is polyethylene glycol. In some embodiments, cellulose acetate has an acetyl content of about 35% to about 45%. In some embodiments, cellulose acetate has an acetyl content of about 40%.

In some embodiments, the pharmaceutical composition further comprising a film coating surrounding the semipermeable membrane coating. In some embodiments, the film coating comprises polyvinyl alcohol. In some embodiments, the film coating further comprises titanium dioxide, polyethylene glycol, and talc.

In some embodiments, at least 99% or 100% of the amount of dexpramipexole, or a pharmaceutically acceptable salt thereof, is in the tablet core and the homogeneous mixture in the tablet core consists essentially of:
(a) about 310 mg to about 330 mg dexpramipexole dihydrochloride monohydrate;
(b) about 222 mg to about 242 mg microcrystalline cellulose;
(c) about 43 mg to about 63 mg polyvinylpyrrolidone-vinyl acetate copolymer;
(d) about 142 mg to about 162 mg sodium chloride; and
(e) about 2 mg to about 6 mg magnesium stearate;
   wherein the tablet comprises about 43 mg to about 63 mg semipermeable membrane coating, and wherein the semipermeable membrane coating comprises about 68% to about 72% cellulose acetate by weight of the semipermeable membrane coating and about 28% to about 32% plasticizer by weight of the semipermeable membrane coating.

In some embodiments, at least 99% or 100% of the amount of dexpramipexole, or a pharmaceutically acceptable salt thereof, is in the tablet core and the homogeneous mixture in the tablet core consists essentially of:
(a) about 319 mg dexpramipexole dihydrochloride monohydrate;
(b) about 232 mg microcrystalline cellulose;
(c) about 53 mg polyvinylpyrrolidone-vinyl acetate copolymer;
(d) about 152 mg sodium chloride; and
(e) about 4 mg magnesium stearate;
   wherein the tablet comprises about 53 mg semipermeable membrane coating, and wherein the semipermeable membrane coating comprises about 70% cellulose acetate by weight of the semipermeable membrane coating and about 30% plasticizer by weight of the semipermeable membrane coating.

In some embodiments, the microcrystalline cellulose is silicified microcrystalline cellulose. In some embodiments, the plasticizer is polyethylene glycol, triethyl citrate, triacetin, diethyl phthalate, poloxamer, or any combination thereof. In some embodiments, the plasticizer is polyethylene glycol. In some embodiments, cellulose acetate has an acetyl content of about 35% to about 45%. In some embodiments, cellulose acetate has an acetyl content of about 40%.

In some embodiments, the pharmaceutical composition further comprising a film coating surrounding the semipermeable membrane coating. In some embodiments, the film coating comprises polyvinyl alcohol. In some embodiments, the film coating further comprises titanium dioxide, polyethylene glycol, and talc.

In some embodiments, the semipermeable membrane coating comprises one or more delivery orifices. In some embodiments, the one or more delivery orifices have a diameter of about 0.35 mm to about 2.0 mm. In some embodiments, the one or more delivery orifices have a diameter of about 0.4 mm to about 0.6 mm. In some embodiments, the one or more delivery orifices have a diameter of about 0.5 mm. In some embodiments, the semipermeable membrane coating comprises one to four delivery orifices. In some embodiments, the semipermeable membrane coating comprises one delivery orifice.

In some embodiments, the tablet is an oval shaped tablet.

In some embodiments, the length of the short axis of the tablet is about 8.5 mm to about 10.5 mm, the length of the long axis of the tablet is about 16 mm to about 18 mm, and the thickness of the tablet is about 5 mm to about 8 mm.

In some embodiments, about 55% to about 85% of dexpramipexole, or the pharmaceutically acceptable salt thereof, is released at about 12 hours of incubation of the tablet in about 900 mL of 50 mM monobasic potassium phosphate buffer, pH 6.8, at a temperature of 37±0.5° C. as measured using an USP type I apparatus operated at a spindle rotation speed of about 100 rpm.

In some embodiments, the tablet core is a single-layer tablet core.

In some embodiments, dexpramipexole, or the pharmaceutically acceptable salt thereof, in the pharmaceutical composition has a chiral purity for dexpramipexole, or the pharmaceutically salt thereof, of 99.96% or more.

In some embodiments, the pharmaceutical composition comprises 0.02% or less pramipexole, or a pharmaceutically acceptable salt thereof, by weight of the tablet. In some embodiments, the pharmaceutical composition comprises 0.015% or less pramipexole, or a pharmaceutically acceptable salt thereof, by weight of the tablet.

Furthermore, in some aspects, the present disclosure provides a method of manufacturing a pharmaceutical composition in the form of an orally deliverable tablet comprising dexpramipexole, or a pharmaceutically acceptable salt thereof, wherein the pharmaceutical composition provides for a sustained release of dexpramipexole, or a pharmaceutically acceptable salt thereof, that makes the tablet suitable for once daily oral administration to a human. In particular, in some aspects, the present disclosure provides a method of manufacturing a pharmaceutical composition in the form of an orally deliverable tablet comprising dexpramipexole, or a pharmaceutically acceptable salt thereof, the method comprising:
preparing a pre-blend comprising dexpramipexole, or a pharmaceutically acceptable salt thereof, and an inorganic osmotic agent;
preparing a blend comprising the pre-blend and a lubricant;
compressing the blend to form a tablet core; and
coating the tablet core with a semipermeable membrane coating comprising a plasticizer;
   wherein dexpramipexole, or the pharmaceutically acceptable salt thereof, constitutes about 37% to about 47% of dexpramipexole dihydrochloride monohydrate equivalent by weight of the blend, wherein the inorganic osmotic agent constitutes about 10% to about 40% by weight of the blend, wherein the semipermeable membrane coating comprises about 5% to about 25% plasticizer by weight of the semipermeable membrane coating, and wherein the weight ratio of the semipermeable membrane coating to the tablet core is about 0.03:1 to about 0.11:1.

In some aspects, the pre-blend further comprises additional excipients. In some aspects, the blend further comprises additional excipients.

In some aspects, the present disclosure provides a method of manufacturing a pharmaceutical composition in the form of an orally deliverable tablet comprising dexpramipexole, or a pharmaceutically acceptable salt thereof, wherein the pharmaceutical composition provides for a sustained release of dexpramipexole, or a pharmaceutically acceptable salt thereof, that makes the tablet suitable for once daily oral administration to a human. In particular, in some aspects, the present disclosure provides a method of manufacturing a pharmaceutical composition in the form of an orally deliverable tablet comprising dexpramipexole, or a pharmaceutically acceptable salt thereof, the method comprising:
preparing a pre-blend comprising dexpramipexole, or a pharmaceutically acceptable salt thereof, and an inorganic osmotic agent;
preparing a blend comprising the pre-blend and a lubricant;
compressing the blend to form a tablet core; and
coating the tablet core with a semipermeable membrane coating comprising a plasticizer;
   wherein dexpramipexole, or the pharmaceutically acceptable salt thereof, constitutes about 37% to about 47% of dexpramipexole dihydrochloride monohydrate equivalent by weight of the blend, wherein the inorganic osmotic agent constitutes about 10% to about 40% by weight of the blend, wherein the semipermeable membrane coating comprises about 5% to about 40% plasticizer by weight of the semipermeable membrane coating, and wherein the weight ratio of the semipermeable membrane coating to the tablet core is about 0.03:1 to about 0.11:1.

In some aspects, the pre-blend further comprises additional excipients. In some aspects, the blend further comprises additional excipients.

Although the osmotic tablets described herein can in principle be prepared using any suitable manufacturing process known to a person skilled in the art, the manufacturing process provided herein was shown to be particularly robust (for example, independent of a manufacturing scale) and reproducible (for example, resulting in a particularly low tablet weight variation; see, in particular, Example 7).

In some embodiments, dexpramipexole, or the pharmaceutically acceptable salt thereof, constitutes about 40% to about 44% of dexpramipexole dihydrochloride monohydrate equivalent by weight of the blend. In some embodiments, dexpramipexole, or the pharmaceutically acceptable salt thereof, constitutes about 42% of dexpramipexole dihydrochloride monohydrate equivalent by weight of the blend.

In some embodiments, the inorganic osmotic agent constitutes about 15% to about 35% by weight of the blend. In some embodiments, the inorganic osmotic agent constitutes about 15% to about 25% by weight of the blend. In some embodiments, the inorganic osmotic agent constitutes about 18% to about 22% by weight of the blend. In some embodiments, the inorganic osmotic agent constitutes about 20% by weight of the blend.

In some embodiments, the inorganic osmotic agent is sodium chloride, potassium chloride, magnesium chloride, sodium hydrogen phosphate, potassium hydrogen phosphate, or any combination thereof. In some embodiments, the inorganic osmotic agent is sodium chloride.

In some embodiments, the pre-blend further comprises microcrystalline cellulose, polyvinylpyrrolidone-vinyl acetate copolymer, or both. In some embodiments, the pre-blend further comprises microcrystalline cellulose and polyvinylpyrrolidone-vinyl acetate copolymer. In some embodiments, microcrystalline cellulose constitutes about 25% to about 35% by weight of the blend. In some embodiments, polyvinylpyrrolidone-vinyl acetate copolymer constitutes about 5% to about 9% by weight of the blend.

In some embodiments, the lubricant is magnesium stearate. In some embodiments, the lubricant constitutes about 0.25% to about 0.75% by weight of the blend.

In some embodiments, the blend does not comprise polyethylene oxide. In some embodiments, the blend does not comprise a swellable polymeric osmotic agent.

In some embodiments, the pharmaceutically acceptable salt of dexpramipexole in the pre-blend is dexpramipexole dihydrochloride or a hydrate thereof. In some embodiments, the pharmaceutically acceptable salt of dexpramipexole in the pre-blend is dexpramipexole dihydrochloride monohydrate.

In some embodiments, the blend consists essentially of:
(a) about 40% to 44% dexpramipexole dihydrochloride monohydrate by weight of the blend;
(b) about 28% to about 32% microcrystalline cellulose by weight of the blend;
(c) about 5% to about 9% polyvinylpyrrolidone-vinyl acetate copolymer by weight of the blend;
(d) about 18% to about 22% sodium chloride by weight of the blend; and
(e) about 0.25% to about 0.75% magnesium stearate by weight of the blend,
   wherein sodium chloride is the inorganic osmotic agent and magnesium stearate is the lubricant.

In some embodiments, the blend consists essentially of:
(a) about 42% dexpramipexole dihydrochloride monohydrate by weight of the blend;
(b) about 30.5% microcrystalline cellulose by weight of the blend;
(c) about 7% polyvinylpyrrolidone-vinyl acetate copolymer by weight of the blend;
(d) about 20% sodium chloride by weight of the blend; and
(e) about 0.5% magnesium stearate by weight of the blend,
   wherein sodium chloride is the inorganic osmotic agent and magnesium stearate is the lubricant.

In some embodiments, coating the tablet core with the semipermeable membrane coating comprises spraying a semipermeable membrane coating solution comprising the plasticizer on the tablet core, optionally wherein the semipermeable membrane coating solution further comprises cellulose acetate. In some embodiments, cellulose acetate has an acetyl content of about 35% to about 45%. In some embodiments, cellulose acetate has an acetyl content of about 38% to about 42%. In some embodiments, cellulose acetate has an acetyl content of about 40%. In some embodiments, the plasticizer and cellulose acetate constitute about 3% to about 7% by weight of the semipermeable membrane coating solution. In some embodiments, the plasticizer and cellulose acetate constitute about 4% by weight of the semipermeable membrane coating solution. In some embodiments, the plasticizer and cellulose acetate constitute about 5% by weight of the semipermeable membrane coating solution. In some embodiments, the plasticizer and cellulose acetate constitute about 6% by weight of the semipermeable membrane coating solution.

In some embodiments, the plasticizer constitutes about 8% to about 40% plasticizer by weight of the semipermeable membrane coating. In some embodiments, the plasticizer constitutes about 10% to about 35% plasticizer by weight of the semipermeable membrane coating. In some embodiments, the plasticizer constitutes about 30% plasticizer by weight of the semipermeable membrane coating. In some embodiments, the plasticizer constitutes about 25% plasticizer by weight of the semipermeable membrane coating. In some embodiments, the plasticizer constitutes about 8% to about 22% plasticizer by weight of the semipermeable membrane coating. In some embodiments, the plasticizer constitutes about 10% to about 20% by weight of the semipermeable membrane coating. In some embodiments, the plasticizer constitutes about 13% to about 20% by weight of the semipermeable membrane coating. In some embodiments, the plasticizer constitutes about 18% by weight of the semipermeable membrane coating. In some embodiments, the plasticizer constitutes about 13% to about 17% by weight of the semipermeable membrane coating. In some embodiments, the plasticizer constitutes about 15% by weight of the semipermeable membrane coating.

In some embodiments, the semipermeable membrane coating further comprises cellulose acetate. In some embodiments, the semipermeable membrane coating further comprises about 60% to about 95% cellulose acetate by weight of the semipermeable membrane coating. In some embodiments, the semipermeable membrane coating further comprises about 75% to about 95% cellulose acetate by weight of the semipermeable membrane coating.

In some embodiments, the semipermeable membrane coating comprises about 65% to about 90% cellulose acetate by weight of the semipermeable membrane coating and about 10% to about 35% plasticizer by weight of the semipermeable membrane coating. In some embodiments, the semipermeable membrane coating comprises about 70% cellulose acetate by weight of the semipermeable membrane coating and about 30% plasticizer by weight of the semipermeable membrane coating. In some embodiments, the semipermeable membrane coating comprises about 75% cellulose acetate by weight of the semipermeable membrane coating and about 25% plasticizer by weight of the semipermeable membrane coating. In some embodiments, the semipermeable membrane coating comprises about 80% to about 90% cellulose acetate by weight of the semipermeable membrane coating and about 20% to about 10% plasticizer by weight of the semipermeable membrane coating. In some embodiments, the semipermeable membrane coating comprises about 83% to about 87% cellulose acetate by weight of the semipermeable membrane coating and about 17% to about 13% plasticizer by weight of the semipermeable membrane coating. In some embodiments, the semipermeable membrane coating comprises about 83% cellulose acetate by weight of the semipermeable membrane coating and about 17% plasticizer by weight of the semipermeable membrane coating. In some embodiments, the semipermeable membrane coating comprises about 85% cellulose acetate by weight of the semipermeable membrane coating and about 15% plasticizer by weight of the semipermeable membrane coating.

In some embodiments, cellulose acetate has an acetyl content of about 35% to about 45%. In some embodiments, cellulose acetate has an acetyl content of about 40%.

In some embodiments, the plasticizer is polyethylene glycol, triethyl citrate, triacetin, diethyl phthalate, poloxamer, or any combination thereof. In some embodiments, the plasticizer is polyethylene glycol.

In some embodiments, the weight ratio of the semipermeable membrane coating to the tablet core is about 0.04:1 to about 0.1:1. In some embodiments, the weight ratio of the semipermeable membrane coating to the tablet core is about 0.05:1 to about 0.09:1. In some embodiments, the weight ratio of the semipermeable membrane coating to the tablet core is about 0.05:1. In some embodiments, the weight ratio of the semipermeable membrane coating to the tablet core is about 0.06:1 to about 0.08:1. In some embodiments, the weight ratio of the semipermeable membrane coating to the tablet core is about 0.06:1. In some embodiments, the weight ratio of the semipermeable membrane coating to the tablet core is about 0.07:1. In some embodiments, the weight ratio of the semipermeable membrane coating to the tablet core is about 0.08:1.

In some embodiments, the method further comprises:
drilling one or more delivery orifices in the semipermeable membrane coating.

In some embodiments, the one or more delivery orifices are drilled in the semipermeable membrane coating by laser drilling. In some embodiments, the one or more delivery orifices are drilled in the semipermeable membrane coating by mechanical drilling. In some embodiments, the one or more delivery orifices have a diameter of about 0.35 mm to about 2.0 mm. In some embodiments, the one or more delivery orifices have a diameter of about 0.4 mm to about 0.6 mm. In some embodiments, the one or more delivery orifices have a diameter of about 0.5 mm. In some embodiments, the number of delivery orifices is one to four. In some embodiments, the number of delivery orifices is one.

In some embodiments, the method further comprises:
coating the semipermeable membrane coating with a film coating.

In some embodiments, the film coating comprises polyvinyl alcohol. In some embodiments, the film coating further comprises titanium dioxide, polyethylene glycol, and talc.

In some embodiments, the method further comprises:
coating the semipermeable membrane coating with a seal coating.

In some embodiments, the seal coating comprises hydroxypropyl methylcellulose and polyethylene glycol.

In some embodiments, the method further comprises:
coating the seal coating with an immediate release drug coating comprising dexpramipexole, or a pharmaceutically acceptable salt thereof.

In some embodiments, the method further comprises:
coating the semipermeable membrane coating with an immediate release drug coating comprising dexpramipexole, or a pharmaceutically acceptable salt thereof.

In some embodiments, the drug coating further comprises a binder. In some embodiments, the weight ratio of dexpramipexole dihydrochloride monohydrate equivalent to binder in the drug coating is about 1:1 to about 1:4. In some embodiments, the weight ratio of dexpramipexole dihydrochloride monohydrate equivalent to binder in the drug coating is about 1:3 or about 1:2. In some embodiments, the binder comprises hydroxypropylmethylcellulose and polyethylene glycol.

In some embodiments, the pharmaceutically acceptable salt of dexpramipexole in the drug coating is dexpramipexole dihydrochloride or a hydrate thereof. In some embodiments, the pharmaceutically acceptable salt of dexpramipexole in the drug coating is dexpramipexole dihydrochloride monohydrate.

In some embodiments, the method further comprises:
coating the drug coating with a film coating.

In some embodiments, the film coating comprises polyvinyl alcohol. In some embodiments, the film coating further comprises titanium dioxide, polyethylene glycol, and talc.

Further, the present disclosure provides methods of treating or preventing certain diseases in a human subject in need thereof, the methods comprising orally administering to the human subject the pharmaceutical compositions as described herein. As dexpramipexole lowers eosinophil counts in vivo, the pharmaceutical compositions as described herein can generally be applied for treating or preventing eosinophilic disorders in humans. Non-limiting examples of diseases that can be treated or prevented with the pharmaceutical compositions as described herein are asthma and chronic obstructive pulmonary disease (COPD).

As shown in Example 7.5, different osmotic tablets (some providing for a comparably faster and others providing for a comparably slower release of drug) were prepared for evaluation in clinical trials. The exemplary tablet for evaluation in clinical trials providing for faster release comprises the entire drug amount (i.e., 300 mg dexpramipexole dihydrochloride equivalent) in a sustained release form, whereas the exemplary tablet for evaluation in clinical trials providing for slower release provides a certain amount of the entire drug amount additionally in an immediate release form (i.e., 255 mg dexpramipexole dihydrochloride equivalent in sustained release form and 45 mg dexpramipexole dihydrochloride equivalent in immediate release form). A Phase I clinical trial for evaluation of the exemplary osmotic tablets is conducted (see Example 8). Additionally, Example 9 shows different osmotic tablets prepared and clinically evaluated.

In some aspects, the present disclosure is further directed to a method of treating or preventing asthma in a human subject in need thereof, the method comprising orally administering to the human subject the pharmaceutical composition as described herein. In some embodiments, the asthma is eosinophilic asthma.

In some aspects, the present disclosure is further directed to a method of treating or preventing chronic obstructive pulmonary disease in a human subject in need thereof, the method comprising orally administering to the human subject the pharmaceutical composition as described herein.

In some aspects, the present disclosure is further directed to a method of treating or preventing an eosinophilic disorder in a human subject in need thereof, the method comprising orally administering to the human subject the pharmaceutical composition as described herein. In some embodiments, the eosinophilic disorder is selected from the group consisting of hypereosinophilic syndrome, chronic rhinosinusitis with nasal polyps, nasal polyposis, atopic dermatitis, eosinophilic granulomatosis with polyangiitis, eosinophilic gastroenteritis, eosinophilic esophagitis, and any combination thereof.

In some embodiments, the pharmaceutical composition is administered once daily.

In some aspects, the present disclosure is further directed to the pharmaceutical composition as described herein for use as a medicament. In some aspects, the present disclosure is further directed to the pharmaceutical composition as described herein for use in a method of treating or preventing as described herein.

In some aspects, the present disclosure is further directed to the use of the pharmaceutical composition as described herein in the manufacture of a medicament for a method of treating or preventing as described herein.

### BRIEF DESCRIPTION OF THE DRAWINGS

FIG. 1 In vitro dissolution profiles of osmotic tablets comprising dexpramipexole dihydrochloride monohydrate with or without inorganic osmotic agent in the tablet core, in 900 mL monobasic potassium phosphate buffer, pH 6.8 (Baskets at 100 rpm).

FIG. 2 In vitro dissolution profiles of osmotic tablets comprising dexpramipexole dihydrochloride monohydrate with 20% w/w or 30% w/w inorganic osmotic agent in the tablet core, in 900 mL monobasic potassium phosphate buffer, pH 6.8 (Baskets at 100 rpm).

FIG. 3 In vitro dissolution profiles of osmotic tablets comprising dexpramipexole dihydrochloride monohydrate with Polyox WSR N80 or Polyox WSR 205 swellable polymeric osmotic agent in the tablet core, in 900 mL monobasic potassium phosphate buffer, pH 6.8 (Baskets at 100 rpm).

FIG. 4 In vitro dissolution profiles of osmotic tablets comprising dexpramipexole dihydrochloride monohydrate with 5% w/w PEG 3350 (of semipermeable membrane coating) or 15% w/w PEG 3350 (of semipermeable membrane coating) as plasticizer, in 900 mL monobasic potassium phosphate buffer, pH 6.8 (Baskets at 100 rpm).

FIG. 5 In vitro dissolution profiles of osmotic tablets comprising dexpramipexole dihydrochloride monohydrate with 4% w/w, 5.5% w/w or 7% w/w coating weight gain, in 900 mL monobasic potassium phosphate buffer, pH 6.8 (Baskets at 100 rpm).

FIG. 6 In vitro dissolution profiles of osmotic tablets comprising dexpramipexole dihydrochloride monohydrate with one delivery orifice or four delivery orifices, in 900 mL monobasic potassium phosphate buffer, pH 6.8 (Baskets at 100 rpm).

FIG. 7 In vitro dissolution profiles of osmotic tablets comprising dexpramipexole dihydrochloride monohydrate in 900 mL monobasic potassium phosphate buffer, pH 6.8 (Baskets at 100 rpm).

FIG. 8 In vitro dissolution profiles of osmotic tablets comprising dexpramipexole dihydrochloride monohydrate in 0.1 N HCl (Baskets at 100 rpm).

FIG. 9 In vitro dissolution profiles of osmotic tablets comprising dexpramipexole dihydrochloride monohydrate in McIlvaine Buffer, pH 4.5 (Baskets at 100 rpm).

FIG. 10 Tablet weight variation plot for batch AZF-1-FD-4-11A.

FIG. 11 Tablet weight variation plot for batch AZF-1-FD-4-11B.

FIG. 12 Reproducibility between smaller scale and engineering batches (slow and fast prototypes). In vitro dissolution profiles of osmotic tablets comprising dexpramipexole dihydrochloride monohydrate in 900 mL monobasic potassium phosphate buffer, pH 6.8 (Baskets at 100 rpm).

FIG. 13 Scanning electron microscopy of the surface of tablets of Lot No. AZF-1-FD-5-2B after immediate release drug coating.

FIG. 14 Reproducibility between smaller scale, engineering, and clinical trial batches (fast prototypes). In vitro dissolution profiles of osmotic tablets comprising dexpramipexole dihydrochloride monohydrate in 900 mL monobasic potassium phosphate buffer, pH 6.8 (Baskets at 100 rpm).

FIG. 15 Reproducibility between smaller scale, engineering, and clinical trial batches (slow prototypes). In vitro dissolution profiles of osmotic tablets comprising dexpramipexole dihydrochloride monohydrate in 900 mL monobasic potassium phosphate buffer, pH 6.8 (Baskets at 100 rpm).

FIG. 16 Schematic outline of Phase I Clinical Trial EXHALE-6.

FIG. 17 Dissolution of Dexpramipexole Dihydrochloride Controlled-Release Osmotic Tablets, 376 mg with 15% pore former in 900 mL pH 0.1 N HCl (Baskets at 100 rpm).

FIG. 18 Dissolution of Dexpramipexole Dihydrochloride Controlled-Release Osmotic Tablets, 376 mg with 20% pore former in 900 mL pH 0.1 N HCl (Baskets at 100 rpm).

FIG. 19 Dissolution of Dexpramipexole Dihydrochloride Controlled-Release Osmotic Tablets, 300 mg with 20% pore former in 900 mL pH 0.1 N HCl (Baskets at 100 rpm).

FIG. 20 Dissolution of Dexpramipexole Dihydrochloride Controlled-Release Osmotic Tablets, 300 mg with 25% pore former in 900 mL pH 0.1 N HCl (Baskets at 100 rpm).

FIG. 21 Dissolution of Dexpramipexole Dihydrochloride Controlled-Release Osmotic Tablets, 300 mg with 10% Sodium Chloride in 900 mL pH 0.1 N HCl (Baskets at 100 rpm).

FIG. 22 Process schematic chart of Common blend of Dexpramipexole ER Osmotic tablets of Lot No. AZF-1-FD-7-4.

FIG. 23 Weight variation plot for first Confirmatory batch of ER6, Lot No. AZF-1-FD-5-25A

FIG. 24 Coated tablets of Dexpramipexole ER Osmotic 280 mg tablets (376 mg diHCl) confirmation batch, Lot No. AZF-1-FD-5-25A.

FIG. 25 Dissolution of Dexpramipexole ER Osmotic 280 mg tablets (376 mg diHCl) first confirmatory batch (Lot No. AZF-1-FD-5-25A1/A2) in 900 mL pH 0.1 N HCl (Baskets at 100 rpm).

FIG. 26 Weight variation plot for first Confirmatory batch of ER8, Lot No. AZF-1-FD-5-25B.

FIG. 27 Coated tablets of Dexpramipexole ER Osmotic 223 mg tablets (300 mg diHCl) confirmation batch, Lot No. AZF-1-FD-5-25B.

FIG. 28 Dissolution of Dexpramipexole ER Osmotic 223 mg tablets (300 mg diHCl) first confirmatory batch (Lot No. AZF-1-FD-5-25B) in 900 mL pH 0.1 N HCl (Baskets at 100 rpm).

FIG. 29 Process schematic chart of Common blend of Dexpramipexole ER Osmotic tablets of Lot No. AZF-1-FD-7-4.

FIG. 30 Dissolution of Dexpramipexole ER Osmotic 280 mg tablets (376 mg diHCl) second confirmatory batch (Lot No. AZF-1-FD-7-4C) in 900 mL pH 0.1 N HCl (Baskets at 100 rpm).

FIG. 31 Dissolution of Dexpramipexole ER Osmotic 223 mg tablets (300 mg diHCl) second confirmatory batch (Lot No. AZF-1-FD-7-4A/4B) in 900 mL pH 0.1 N HCl (Baskets at 100 rpm).

FIG. 32 Dissolution between lab confirmatory batch, and clinical batch for Dexpramipexole Dihydrochloride CR Osmotic Tablets, 376 mg (ER6) in 0.1 N HCl (Baskets at 100 rpm).

FIG. 33 Dissolution between lab confirmatory batch, and clinical batch for Dexpramipexole Dihydrochloride CR Osmotic Tablets, 300 mg (ER8) in 0.1 N HCl (Baskets at 100 rpm).

FIGS. 34-36 Images of defective functional coated Dexpramipexole Dihydrochloride CR Osmotic Tablets, 300 mg of Batch No. 6045203 (ER8).

### DETAILED DESCRIPTION OF THE DISCLOSURE

Unless defined otherwise, all technical and scientific terms used herein have the same meaning as commonly understood by one of ordinary skill in the art to which this disclosure belongs. In case of conflict, the present application including the definitions will control. Unless otherwise required by context, singular terms shall include pluralities and plural terms shall include the singular. In general, the headings provided herein are not to be understood as limiting the various aspects and embodiments of the present disclosure. All publications, patents and other references mentioned herein are incorporated by reference in their entireties for all purposes as if each individual publication or patent application were specifically and individually indicated to be incorporated by reference.

Although methods and materials similar or equivalent to those described herein can be used in practice or testing of the present disclosure, suitable methods and materials are described below. The materials, methods and examples are illustrative only and are not intended to be limiting. Other features and advantages of the disclosure will be apparent from the detailed description and from the claims.

### I—Definitions

In the following, some definitions are provided to further define the present disclosure. Additional definitions may be found throughout the specification.

The singular forms “a”, “an”, and “the” include plural referents unless the context clearly dictates otherwise. The terms “a” (or “an”), as well as the terms “one or more” and “at least one” can be used interchangeably herein. In certain aspects, the term “a” or “an” means “single”. In other aspects, the term “a” or “an” includes “two or more” or “multiple”.

The term “about” is used herein to mean approximately, roughly, around, or in the regions of When the term “about” is used in conjunction with a numerical value or range, it modifies that value or range by extending the boundaries above and below the numerical value(s) set forth by a variance of 10 percent, up or down (higher or lower). For example, within the meaning of the present disclosure, “about 50%” means in the range of 45%-55%, and “about 50% to about 60%” means in the range of 45%-66%. In addition, when the term “about” is used in conjunction with a “%” value, the recited value or range cannot exceed 100%. For example, within the meaning of the present disclosure, “about 99%” means in the range of 89.1%-100%.

The term “and/or” where used herein is to be taken as specific disclosure of each of the specified features or components with or without the other. Thus, the term “and/or” as used in a phrase such as “A and/or B” herein is intended to include “A and B”, “A or B”, “A” (alone), and “B” (alone). Likewise, the term “and/or” as used in a phrase such as “A, B, and/or C” is intended to encompass each of the following aspects: “A, B, and C”, “A, B, or C”, “A or C”, “A or B”, “B or C”, “A and C”, “A and B”, “B and C”, “A” (alone), “B” (alone), and “C” (alone).

As used herein, the term “comprising” means “including, but not limited to”.

As used herein, the term “consisting essentially of” means the method or composition includes the steps or components specifically recited, and may also include those that do not materially affect the basic and novel characteristics of the method or composition. The basic and novel characteristics of the tablets of the present disclosure are that the tablets are suitable for oral administration to a human (i.e., the tablets described herein do not exceed a tablet weight of about 1500 mg) once daily and thereby provide for an amount of dexpramipexole, or a pharmaceutically acceptable salt thereof, as specified herein (i.e., for an amount of about 50 mg to about 400 mg dexpramipexole dihydrochloride equivalent). Accordingly, the basic and novel characteristics of the methods of manufacturing of the present disclosure are that the methods result in the tablets as described in this paragraph.

As used herein, the term “consisting of” means the method or composition includes only the steps or components specifically recited thereafter.

As used herein, the term “any combination thereof” or “a combination thereof” covers any combination of the components or steps recited before the term. For example, the expression “a composition further comprising A, B, C, D, or any combination thereof” includes inter alia “a composition further comprising A,” “a composition further comprising A and B,” “a composition further comprising B and D,” “a composition further comprising A, B and C,” and “a composition further comprising A, B, C, and D”.

The term “homogeneous mixture” as used herein refers to a mixture wherein all of the ingredients have been thoroughly mixed such that the composition of the mixture is substantially the same throughout different portions of the mixture.

As used herein, the terms “pre-blend” and “blend” refer to homogeneous mixtures of the corresponding ingredients in the pre-blend or blend, respectively. A “blend” refers to a homogeneous mixture that comprises additional ingredients compared to a corresponding “pre-blend.”

The term “osmotic agent,” as used herein, refers to a substance that creates osmotic pressure in the osmotic drug delivery system, i.e., that possesses osmogenic activity. In some aspects, the osmotic agent is an inorganic osmotic agent. In some aspects, the inorganic osmotic agent is an inorganic salt of an alkali metal or an alkaline earth metal. In some aspects, the inorganic osmotic agent is sodium chloride, potassium chloride, magnesium chloride, sodium hydrogen phosphate, potassium hydrogen phosphate, or any combination thereof. In some aspects, an osmotic agent is an organic osmotic agent. In some aspects, the organic osmotic agent is an organic amino compound, a polyol, or both. In some aspects, the organic osmotic agent is taurine, glycine, lactose, fructose, sorbitol, dextrose, sucrose, xylitol, mannitol, or any combination thereof. In some aspects, the osmotic agent is a swellable polymeric osmotic agent. In some aspects, the swellable polymeric osmotic agent is polyethylene oxide, vinyl acetate copolymer, polyethylene glycol, or any combination thereof. “Swellable” in this context means that the polymer increases in volume when being exposed to an aqueous environment (such as gastric juice). In some aspects, the tablets comprise a combination of one or more different osmotic agents (such as a combination of inorganic and organic osmotic agents).

The term “semipermeable membrane,” as used herein, refers to a membrane that is substantially rigid, non-stretchable and substantially permeable to water, but substantially impermeable to a solute (including dexpramipexole, or a pharmaceutically acceptable salt thereof, and an osmotic agent). A “semipermeable membrane coating,” as used herein, provides one or more delivery orifices (through which the active agent can be released from the tablet core; i.e., the one or more delivery orifices reach from the semipermeable membrane coating to the tablet core, or, in other words, the one or more delivery orifices pierce through the semipermeable membrane coating). In some embodiments, the semipermeable membrane coating comprises one or more delivery orifices (wherein “comprises one or more delivery orifices” means that the one or more delivery orifices are present in the semipermeable membrane coating already prior to administration to a human; the one or more delivery orifices can be obtained, e.g., by laser and/or mechanical drilling). In some embodiments, the semipermeable membrane coating comprises one or more pore-forming agents (that dissolve and leach out of the semipermeable membrane coating upon exposure to aqueous solution, thereby resulting in the formation of one or more delivery orifices in situ). An example of such a pore-forming agent is polyethylene glycol. In some embodiments, the semipermeable membrane coating comprises one or more delivery orifices and comprises one or more pore-forming agents, such as polyethylene glycol.

The term “orally deliverable,” as used herein, means that a corresponding pharmaceutical composition (such as a tablet) is suitable for oral administration to a human by swallowing the composition as a whole.

The term “core,” as used herein, refers to the part of the pharmaceutical composition described herein that is enclosed within the semipermeable membrane coating. If the pharmaceutical composition is in the form of an orally deliverable tablet, the term “tablet core,” as used herein, refers to the part of the tablet that is enclosed within the semipermeable membrane coating of the tablet. The tablet core may be, for example, a single-layer tablet core or a multi-layer tablet core (e.g., bi-layer, tri-layer, etc.), as described further below in the specification.

The term “dexpramipexole,” as used herein, refers to (6R)-2-amino-4,5,6,7-tetrahydro-6-(propylamino)benzothiazole. The chemical structure of dexpramipexole is given above in formula (I). The term “dexpramipexole” refers to the “free base” (i.e., the neutral form of dexpramipexole as shown above in formula (I)) unless the context clearly dictates otherwise.

The term “pharmaceutically acceptable salt” is meant to indicate those salts that are suitable for use in contact with the tissues of a human without undue toxicity, irritation, allergic response, and the like, and are commensurate with a reasonable benefit/risk ratio. For example, Remington's Pharmaceutical Sciences, 23^{th} ed. (2020) and Berge et al. (1977), J. Pharm. Sciences, Vol 6., 1-19 describe pharmaceutically acceptable salts in detail. Pharmaceutically acceptable salts can generally also be in the form of hydrates (such as in the form of a monohydrate). In some embodiments, the pharmaceutically acceptable salt of dexpramipexole is dexpramipexole dihydrochloride or a hydrate thereof (such as a monohydrate). Examples of suitable pharmaceutically acceptable salts of dexpramipexole within the scope of the present disclosure include, but are not limited to, dexpramipexole dihydrochloride and dexpramipexole dihydrochloride monohydrate. Further suitable “pharmaceutically acceptable salts” of dexpramipexole within the scope of the present disclosure are described throughout the specification.

The term “equivalent” is used herein to specify a certain amount of an ingredient with a reference to a more specific form (such as a specific salt) of the ingredient. For example, the term “dexpramipexole dihydrochloride equivalent” is used to specify the amount of dexpramipexole, or a pharmaceutically acceptable salt thereof, in a pharmaceutical composition that corresponds the same quantity as dexpramipexole dihydrochloride. For instance, an amount of 319 mg dexpramipexole dihydrochloride monohydrate corresponds to 300 mg dexpramipexole dihydrochloride equivalent, and an amount of 223 mg dexpramipexole corresponds to 300 mg dexpramipexole dihydrochloride equivalent. As another example, an amount of 160 mg dexpramipexole dihydrochloride monohydrate corresponds to 150 mg dexpramipexole dihydrochloride equivalent, and an amount of 112 mg dexpramipexole corresponds to 150 mg dexpramipexole dihydrochloride equivalent.

The term “weight of the tablet,” as used herein, refers to the weight of the entire tablet, including the weight of any coating present. For example, in embodiments wherein the tablet comprises a film coating, the term “weight of the tablet” also includes the weight of the film coating.

The term “immediate release” or “IR,” as used herein, means a release of an active agent (also referred to as “active ingredient” or “drug” herein) to an environment over a period of seconds to no more than about 60 minutes after administration. Typically, “immediate release” means that no less than 80% of the active agent in the immediate release formulation are released within 45 minutes after administration.

“Immediate release,” as used herein, means that no effort is taken either through formulation or processing to modify the release of the drug from the dosage form. Typically, greater than 80% of drug is dissolved within 1 hour when analyzed with a USP method.

“Sustained release,” as used herein, refers to products where the time period of release is extended through formulation and/or processing of the drug product. Sustained release of an active agent to an environment occurs over a period of about eight hours, about 12 hours, about 16 hours, about 18 hours, about 20 hours, about 24 hours, or more than about 24 hours. A sustained release can begin within a few minutes after administration (such as within about 5 min or about 10 min after administration); the release can also begin after an expiration of a delay period (lag time) after administration. In some aspects, the active agent is released to an environment over a period of about 5 min to about eight hours, about 5 min to about 12 hours, about 5 min to about 16 hours, about 5 min to about 18 hours, about 5 min to about 20 hours, about 5 min to about 24 hours, or about 5 min to more than about 24 hours after administration. In some aspects, the active agent is released to an environment over a period of about 10 min to about eight hours, about 10 min to about 12 hours, about 10 min to about 16 hours, about 10 min to about 18 hours, about 10 min to about 20 hours, about 10 min to about 24 hours, or about 10 min to more than about 24 hours after administration. In certain embodiments, the drug is released at a substantially constant rate or pulsatile rate over an extended period of time. In some aspects, the sustained release provides for a constant drug level in the blood or target tissue of a human to which the sustained release device (such as a tablet) is administered.

“Extended release,” as used herein, refers to products where either the rate of release is controlled/reduced or the duration of release is extended through formulation and/or processing of the drug product.

“Controlled release,” as used herein, refers to products where the rate of drug release has been intentionally modified through formulation and/or processing of the drug product.

The terms “controlled release,” “sustained release,” or “extended release” or “ER” are used interchangeably herein.

The term “treating” refers to alleviating of the signs or symptoms associated with a specific disorder, disease, or condition, and/or removing of the signs or symptoms associated with a specific disorder, disease, or condition, and/or preventing of the worsening of the signs or symptoms associated with a specific disorder, disease, or condition. In some aspects, a treatment alleviates signs or symptoms associated with a specific disorder, disease, or condition. In other aspects, the treatment removes signs or symptoms associated with a specific disorder, disease, or condition. In other aspects, the treatment prevents worsening of the signs or symptoms associated with a specific disorder, disease, or condition.

The term “preventing” refers to prophylaxis of a specific disorder, disease, or condition in a human. In certain aspects, the human may be predisposed to the specific disorder, disease, or condition but does not yet experience or display the pathology, signs, or symptoms of the specific disorder, disease, or condition.

The term “in need thereof,” as used herein, means that the human subject has a need for the particular treatment or prevention and that the treatment or prevention is being given to the subject for that particular purpose.

### II—Pharmaceutical Compositions

In some aspects, the present disclosure is directed to a pharmaceutical composition in the form of an orally deliverable tablet comprising a tablet core, a semipermeable membrane coating surrounding the tablet core, and dexpramipexole, or a pharmaceutically acceptable salt thereof.

In some aspects, the amount of dexpramipexole, or a pharmaceutically acceptable salt thereof, is about 50 mg to about 400 mg of dexpramipexole dihydrochloride equivalent. In some aspects, at least about 70% of the amount of dexpramipexole, or a pharmaceutically acceptable salt thereof, in the pharmaceutical composition is in the tablet core. In some aspects, the tablet core comprises a homogeneous mixture of an inorganic osmotic agent and the amount of dexpramipexole, or a pharmaceutically acceptable salt thereof, that is in the tablet core, wherein the inorganic osmotic agent constitutes about 10% to about 40% by weight of the tablet core. In some aspects, the semipermeable membrane coating comprises about 5% to about 25% plasticizer by weight of the semipermeable membrane coating. In some aspects, the weight ratio of the semipermeable membrane coating to the tablet core is about 0.03:1 to about 0.11:1. In some aspects, the weight of the tablet is about 1500 mg or less.

In some aspects, the amount of dexpramipexole, or a pharmaceutically acceptable salt thereof, is about 50 mg to about 400 mg of dexpramipexole dihydrochloride equivalent. In some aspects, at least about 70% of the amount of dexpramipexole, or a pharmaceutically acceptable salt thereof, in the pharmaceutical composition is in the tablet core. In some aspects, the tablet core comprises a homogeneous mixture of an inorganic osmotic agent and the amount of dexpramipexole, or a pharmaceutically acceptable salt thereof, that is in the tablet core, wherein the inorganic osmotic agent constitutes about 10% to about 40% by weight of the tablet core. In some aspects, the semipermeable membrane coating comprises about 5% to about 40% plasticizer by weight of the semipermeable membrane coating. In some aspects, the weight ratio of the semipermeable membrane coating to the tablet core is about 0.03:1 to about 0.11:1. In some aspects, the weight of the tablet is about 1500 mg or less.

In some aspects, the present disclosure is directed to a pharmaceutical composition in the form of an orally deliverable tablet comprising a tablet core, a semipermeable membrane coating surrounding the tablet core, and dexpramipexole, or a pharmaceutically acceptable salt thereof, in an amount of about 50 mg to about 400 mg of dexpramipexole dihydrochloride equivalent, wherein
at least about 70% of the amount of dexpramipexole, or a pharmaceutically acceptable salt thereof, is in the tablet core;
the tablet core comprises a homogeneous mixture of an inorganic osmotic agent and the amount of dexpramipexole, or a pharmaceutically acceptable salt thereof, that is in the tablet core, wherein the inorganic osmotic agent constitutes about 10% to about 40% by weight of the tablet core;
the semipermeable membrane coating comprises about 5% to about 25% plasticizer by weight of the semipermeable membrane coating;
the weight ratio of the semipermeable membrane coating to the tablet core is about 0.03:1 to about 0.11:1; and
the weight of the tablet is about 1500 mg or less.

In some aspects, the tablet core further comprises additional excipients.

In some aspects, the present disclosure is directed to a pharmaceutical composition in the form of an orally deliverable tablet comprising a tablet core, a semipermeable membrane coating surrounding the tablet core, and dexpramipexole, or a pharmaceutically acceptable salt thereof, in an amount of about 50 mg to about 400 mg of dexpramipexole dihydrochloride equivalent, wherein
at least about 70% of the amount of dexpramipexole, or a pharmaceutically acceptable salt thereof, is in the tablet core;
the tablet core comprises a homogeneous mixture of an inorganic osmotic agent and the amount of dexpramipexole, or a pharmaceutically acceptable salt thereof, that is in the tablet core, wherein the inorganic osmotic agent constitutes about 10% to about 40% by weight of the tablet core;
the semipermeable membrane coating comprises about 5% to about 40% plasticizer by weight of the semipermeable membrane coating;
the weight ratio of the semipermeable membrane coating to the tablet core is about 0.03:1 to about 0.11:1; and
the weight of the tablet is about 1500 mg or less.

In some aspects, the tablet core further comprises additional excipients.

The pharmaceutical composition according to the present disclosure is further described in the sections below within this chapter (“II—Pharmaceutical Composition”). As acknowledged by a person of ordinary skill in the art, the embodiments described below may be combined to further embodiments. For example, one or more embodiments described under section “3. Tablet Core” may be combined with one or more embodiments described under section “4. Semipermeable membrane coating” to a further embodiment.

### 1. Pharmaceutically Acceptable Salts of Dexpramipexole

In some embodiments, the pharmaceutically acceptable salt of dexpramipexole is an inorganic acid salt, an organic acid salt, or an amino acid salt.

In some embodiments, the pharmaceutically acceptable salt of dexpramipexole is a halogenic acid salt.

In some embodiments, the halogenic acid salt is a hydrobromic, hydrochloric, hydrofluoric, or hydroiodic acid salt.

In some embodiments, the inorganic acid salt is a nitric, perchloric, sulfuric, or phosphoric acid salt.

In some embodiments, the organic acid salt is a sulfonic, tartaric acid, acetic, malic, fumaric, succinic, citric, benzoic, gluconic, lactic, mandelic, mucic, pamoic, pantothenic, exalic or maleic acid salt. In some embodiments, the sulfonic acid salt is a methane sulfonic, trifluoromethane sulfonic, ethane sulfonic, benzene sulfonic orp-toluene sulfonic acid salt.

In some embodiments, the amino acid salt is aspartic or glutamic acid salt.

In some embodiments, the pharmaceutically acceptable salt is a methane sulfonic acid salt (“mesylate salt”), sulfuric acid salt (“sulfate salt”), tartaric acid salt (“tartrate salt”), p-toluene sulfonic acid salt (“tosylate salt”), phosphoric acid salt (“phosphate salt”), maleic acid salt (“maleate salt”), fumaric acid salt (“fumarate salt”), malic acid salt (“malate salt”), citric acid salt (“citrate salt”), succinic acid salt (“succinate salt”), or any combination thereof.

In some specific embodiments, the pharmaceutically acceptable salt is a methane sulfonic acid salt, phosphoric acid salt, fumaric acid salt, or any combination thereof.

The acid addition salt may be a mono- or di-acid addition salt, such as dihydrobromic, dihydrochloric, dihydrofluoric, dihydroiodic, disulfuric, diphosphoric, or diorganic acid salt. The acid addition salt may additionally be in the form of a hydrate, such as a dihydrochloride monohydrate.

In some embodiments, the pharmaceutically acceptable salt of dexpramipexole is dexpramipexole dihydrochloride or a hydrate thereof (such as a monohydrate).

In some specific embodiments, the pharmaceutically acceptable salt of dexpramipexole is dexpramipexole dihydrochloride. In some specific embodiments, the pharmaceutically acceptable salt of dexpramipexole is dexpramipexole dihydrochloride monohydrate.

### 2. Amount of Dexpramipexole, or a Pharmaceutically Acceptable Salt Thereof

As outlined above, in some aspects, the pharmaceutical composition in the form of an orally deliverable tablet comprises dexpramipexole, or a pharmaceutically acceptable salt thereof, in an amount of about 50 mg to about 400 mg of dexpramipexole dihydrochloride equivalent.

In some embodiments, the amount of dexpramipexole, or a pharmaceutically acceptable salt thereof, in the pharmaceutical composition is about 50 mg to about 350 mg of dexpramipexole dihydrochloride equivalent. In some embodiments, the amount of dexpramipexole, or a pharmaceutically acceptable salt thereof, in the pharmaceutical composition is about 55 mg to about 340 mg, about 60 mg to about 330 mg, about 65 mg to about 320 mg, or about 70 mg to about 310 mg of dexpramipexole dihydrochloride equivalent. In some embodiments, the amount of dexpramipexole, or a pharmaceutically acceptable salt thereof, in the pharmaceutical composition is about 75 mg to about 300 mg (such as about 75 mg, about 150 mg, or about 300 mg) of dexpramipexole dihydrochloride equivalent. In some embodiments, the amount of dexpramipexole, or a pharmaceutically acceptable salt thereof, in the pharmaceutical composition is about 75 mg to about 400 mg (such as about 75 mg, about 150 mg, about 300 mg, or about 376 mg) of dexpramipexole dihydrochloride equivalent.

In specific embodiments, the amount of dexpramipexole, or a pharmaceutically acceptable salt thereof, in the pharmaceutical composition is about 75 mg of dexpramipexole dihydrochloride equivalent. In other specific embodiments, the amount of dexpramipexole, or a pharmaceutically acceptable salt thereof, in the pharmaceutical composition is about 150 mg of dexpramipexole dihydrochloride equivalent. In yet other specific embodiments, the amount of dexpramipexole, or a pharmaceutically acceptable salt thereof, in the pharmaceutical composition is about 300 mg of dexpramipexole dihydrochloride equivalent. In yet other specific embodiments, the amount of dexpramipexole, or a pharmaceutically acceptable salt thereof, in the pharmaceutical composition is about 376 mg of dexpramipexole dihydrochloride equivalent.

In some embodiments, the amount of dexpramipexole, or a pharmaceutically acceptable salt thereof, in the pharmaceutical composition is about 50 mg, about 55 mg, about 60 mg, about 65 mg, about 70 mg, about 75 mg, about 80 mg, about 85 mg, about 90 mg, about 95 mg, about 100 mg, about 105 mg, about 110 mg, about 115 mg, about 120 mg, about 125 mg, about 130 mg, about 135 mg, about 140 mg, about 145 mg, about 150 mg, about 155 mg, about 160 mg, about 165 mg, about 170 mg, about 175 mg, about 180 mg, about 185 mg, about 190 mg, about 195 mg, about 200 mg, about 205 mg, about 210 mg, about 215 mg, about 220 mg, about 225 mg, about 230 mg, about 235 mg, about 240 mg, about 245 mg, about 250 mg, about 255 mg, about 260 mg, about 265 mg, about 270 mg, about 275 mg, about 280 mg, about 285 mg, about 290 mg, about 295 mg, about 300 mg, about 305 mg, about 310 mg, about 315 mg, about 320 mg, about 325 mg, about 330 mg, about 335 mg, about 340 mg, about 345 mg, about 350 mg, about 355 mg, about 360 mg, about 365 mg, about 370 mg, about 375 mg, about 380 mg, about 385 mg, about 390 mg, about 395 mg, or about 400 mg of dexpramipexole dihydrochloride equivalent. In some embodiments, the amount of dexpramipexole, or a pharmaceutically acceptable salt thereof, in the pharmaceutical composition is about 75 mg, about 150 mg, about 300 mg, or about 376 mg of dexpramipexole dihydrochloride equivalent.

In some embodiments, the amount of dexpramipexole, or a pharmaceutically acceptable salt thereof, in the pharmaceutical composition is about 50 mg to about 100 mg of dexpramipexole dihydrochloride equivalent. In some embodiments, the amount of dexpramipexole, or a pharmaceutically acceptable salt thereof, in the pharmaceutical composition is about 55 mg to about 95 mg of dexpramipexole dihydrochloride equivalent. In some embodiments, the amount of dexpramipexole, or a pharmaceutically acceptable salt thereof, in the pharmaceutical composition is about 60 mg to about 90 mg of dexpramipexole dihydrochloride equivalent. In some embodiments, the amount of dexpramipexole, or a pharmaceutically acceptable salt thereof, in the pharmaceutical composition is about 65 mg to about 85 mg of dexpramipexole dihydrochloride equivalent. In some embodiments, the amount of dexpramipexole, or a pharmaceutically acceptable salt thereof, in the pharmaceutical composition is about 70 mg to about 80 mg of dexpramipexole dihydrochloride equivalent. In specific embodiments, the amount of dexpramipexole, or a pharmaceutically acceptable salt thereof, in the pharmaceutical composition is about 75 mg of dexpramipexole dihydrochloride equivalent.

In some embodiments, the amount of dexpramipexole, or a pharmaceutically acceptable salt thereof, in the pharmaceutical composition is about 100 mg to about 350 mg of dexpramipexole dihydrochloride equivalent. In some embodiments, the amount of dexpramipexole is about 105 mg to about 345 mg of dexpramipexole dihydrochloride equivalent. In some embodiments, the amount of dexpramipexole is about 110 mg to about 340 mg of dexpramipexole dihydrochloride equivalent. In some embodiments, the amount of dexpramipexole is about 115 mg to about 335 mg of dexpramipexole dihydrochloride equivalent. In some embodiments, the amount of dexpramipexole is about 120 mg to about 335 mg of dexpramipexole dihydrochloride equivalent. In some embodiments, the amount of dexpramipexole is about 120 mg to about 330 mg of dexpramipexole dihydrochloride equivalent. In some embodiments, the amount of dexpramipexole, or a pharmaceutically acceptable salt thereof, in the pharmaceutical composition is about 125 mg to about 325 mg of dexpramipexole dihydrochloride equivalent. In some embodiments, the amount of dexpramipexole, or a pharmaceutically acceptable salt thereof, in the pharmaceutical composition is about 130 mg to about 320 mg of dexpramipexole dihydrochloride equivalent. In some embodiments, the amount of dexpramipexole, or a pharmaceutically acceptable salt thereof, in the pharmaceutical composition is about 135 mg to about 315 mg of dexpramipexole dihydrochloride equivalent. In some embodiments, the amount of dexpramipexole, or a pharmaceutically acceptable salt thereof, in the pharmaceutical composition is about 140 mg to about 310 mg of dexpramipexole dihydrochloride equivalent. In some embodiments, the amount of dexpramipexole, or a pharmaceutically acceptable salt thereof, in the pharmaceutical composition is about 145 mg to about 305 mg of dexpramipexole dihydrochloride equivalent. In some embodiments, the amount of dexpramipexole, or a pharmaceutically acceptable salt thereof, in the pharmaceutical composition is about 150 mg to about 300 mg of dexpramipexole dihydrochloride equivalent.

In some embodiments, the amount of dexpramipexole, or a pharmaceutically acceptable salt thereof, in the pharmaceutical composition is about 200 mg to about 400 mg, about 205 mg to about 395 mg, about 210 mg to about 390 mg, about 215 mg to about 385 mg, about 220 mg to about 380 mg, about 225 mg to about 375 mg, about 230 mg to about 370 mg, about 235 mg to about 365 mg, about 240 mg to about 360 mg, or about 245 mg to about 355 mg of dexpramipexole dihydrochloride equivalent. In some embodiments, the amount of dexpramipexole, or a pharmaceutically acceptable salt thereof, is about 250 mg to about 350 mg, about 255 mg to about 345 mg, about 260 mg to about 340 mg, about 265 mg to about 335 mg, or about 270 mg to about 330 mg of dexpramipexole dihydrochloride equivalent. In some embodiments, the amount of dexpramipexole, or a pharmaceutically acceptable salt thereof, in the pharmaceutical composition is about 275 mg to about 325 mg of dexpramipexole dihydrochloride equivalent. In some embodiments, the amount of dexpramipexole, or a pharmaceutically acceptable salt thereof, in the pharmaceutical composition is about 280 mg to about 320 mg of dexpramipexole dihydrochloride equivalent. In some embodiments, the amount of dexpramipexole, or a pharmaceutically acceptable salt thereof, in the pharmaceutical composition is about 285 mg to about 315 mg of dexpramipexole dihydrochloride equivalent. In some embodiments, the amount of dexpramipexole, or a pharmaceutically acceptable salt thereof, in the pharmaceutical composition is about 290 mg to about 310 mg of dexpramipexole dihydrochloride equivalent. In some embodiments, the amount of dexpramipexole, or a pharmaceutically acceptable salt thereof, in the pharmaceutical composition is about 295 mg to about 305 mg of dexpramipexole dihydrochloride equivalent. In specific embodiments, the amount of dexpramipexole, or a pharmaceutically acceptable salt thereof, in the pharmaceutical composition is about 300 mg of dexpramipexole dihydrochloride equivalent.

In some embodiments, the amount of dexpramipexole, or a pharmaceutically acceptable salt thereof, in the pharmaceutical composition is about 200 mg to about 400 mg, about 225 mg to about 395 mg, about 250 mg to about 390 mg, about 275 mg to about 385 mg, or about 300 mg to about 380 mg of dexpramipexole dihydrochloride equivalent. In some embodiments, the amount of dexpramipexole, or a pharmaceutically acceptable salt thereof, in the pharmaceutical composition is about 300 mg to about 400 mg of dexpramipexole dihydrochloride equivalent. In some embodiments, the amount of dexpramipexole, or a pharmaceutically acceptable salt thereof, in the pharmaceutical composition is about 350 mg to about 390 mg of dexpramipexole dihydrochloride equivalent. In some embodiments, the amount of dexpramipexole, or a pharmaceutically acceptable salt thereof, in the pharmaceutical composition is about 360 mg to about 380 mg of dexpramipexole dihydrochloride equivalent. In specific embodiments, the amount of dexpramipexole, or a pharmaceutically acceptable salt thereof, in the pharmaceutical composition is about 376 mg of dexpramipexole dihydrochloride equivalent.

In some embodiments, the amount of dexpramipexole, or a pharmaceutically acceptable salt thereof, in the pharmaceutical composition is about 50 mg to about 250 mg, about 55 mg to about 245 mg, about 60 mg to about 240 mg, about 65 mg to about 235 mg, about 70 mg to about 230 mg about 75 mg to about 225 mg, about 80 mg to about 220 mg, about 85 mg to about 215 mg, about 90 mg to about 210 mg, about 95 mg to about 205 mg, about 100 mg to about 200 mg, about 105 mg to about 195 mg, about 110 mg to about 190 mg, about 115 mg to about 185 mg, or about 120 mg to about 180 mg of dexpramipexole dihydrochloride equivalent. In some embodiments, the amount of dexpramipexole, or a pharmaceutically acceptable salt thereof, in the pharmaceutical composition is about 125 mg to about 175 mg of dexpramipexole dihydrochloride equivalent. In some embodiments, the amount of dexpramipexole, or a pharmaceutically acceptable salt thereof, in the pharmaceutical composition is about 130 mg to about 170 mg of dexpramipexole dihydrochloride equivalent. In some embodiments, the amount of dexpramipexole, or a pharmaceutically acceptable salt thereof, in the pharmaceutical composition is about 135 mg to about 165 mg of dexpramipexole dihydrochloride equivalent. In some embodiments, the amount of dexpramipexole, or a pharmaceutically acceptable salt thereof, in the pharmaceutical composition is about 140 mg to about 160 mg of dexpramipexole dihydrochloride equivalent. In some embodiments, the amount of dexpramipexole, or a pharmaceutically acceptable salt thereof, in the pharmaceutical composition is about 145 mg to about 165 mg of dexpramipexole dihydrochloride equivalent. In specific embodiments, the amount of dexpramipexole, or a pharmaceutically acceptable salt thereof, in the pharmaceutical composition is about 150 mg of dexpramipexole dihydrochloride equivalent.

In some embodiments, the pharmaceutical composition comprises about 50 mg to about 400 mg dexpramipexole dihydrochloride monohydrate.

In some embodiments, the pharmaceutical composition comprises about 60 mg to about 340 mg dexpramipexole dihydrochloride monohydrate.

In some embodiments, the pharmaceutical composition comprises about 60 mg to about 100 mg dexpramipexole dihydrochloride monohydrate. In some embodiments, the pharmaceutical composition comprises about 65 mg to about 95 mg dexpramipexole dihydrochloride monohydrate. In some embodiments, the pharmaceutical composition comprises about 70 mg to about 90 mg dexpramipexole dihydrochloride monohydrate. In some embodiments, the pharmaceutical composition comprises about 75 mg to about 85 mg dexpramipexole dihydrochloride monohydrate. In specific embodiments, the pharmaceutical composition comprises about 80 mg dexpramipexole dihydrochloride monohydrate.

In some embodiments, the pharmaceutical composition comprises about 140 mg to about 180 mg dexpramipexole dihydrochloride monohydrate. In some embodiments, the pharmaceutical composition comprises about 145 mg to about 175 mg dexpramipexole dihydrochloride monohydrate. In some embodiments, the pharmaceutical composition comprises about 150 mg to about 170 mg dexpramipexole dihydrochloride monohydrate. In some embodiments, the pharmaceutical composition comprises about 155 mg to about 165 mg dexpramipexole dihydrochloride monohydrate. In specific embodiments, the pharmaceutical composition comprises about 160 mg dexpramipexole dihydrochloride monohydrate.

In some embodiments, the pharmaceutical composition comprises about 300 mg to about 340 mg dexpramipexole dihydrochloride monohydrate. In some embodiments, the pharmaceutical composition comprises about 305 mg to about 335 mg dexpramipexole dihydrochloride monohydrate. In some embodiments, the pharmaceutical composition comprises about 310 mg to about 330 mg dexpramipexole dihydrochloride monohydrate. In some embodiments, the pharmaceutical composition comprises about 315 mg to about 325 mg dexpramipexole dihydrochloride monohydrate. In specific embodiments, the pharmaceutical composition comprises about 319 mg dexpramipexole dihydrochloride monohydrate.

In some embodiments, the pharmaceutical composition comprises about 300 mg to about 400 mg dexpramipexole dihydrochloride monohydrate. In some embodiments, the pharmaceutical composition comprises about 325 mg to about 400 mg dexpramipexole dihydrochloride monohydrate. In some embodiments, the pharmaceutical composition comprises about 350 mg to about 400 mg dexpramipexole dihydrochloride monohydrate. In some embodiments, the pharmaceutical composition comprises about 375 mg to about 400 mg dexpramipexole dihydrochloride monohydrate. In specific embodiments, the pharmaceutical composition comprises about 400 mg dexpramipexole dihydrochloride monohydrate.

In some embodiments, the pharmaceutical composition comprises about 60 mg, about 65 mg, about 70 mg, about 75 mg, about 80 mg, about 85 mg, about 90 mg, about 95 mg, about 100 mg, about 105 mg, about 110 mg, about 115 mg, about 120 mg, about 125 mg, about 130 mg, about 135 mg, about 140 mg, about 145 mg, about 150 mg, about 155 mg, about 160 mg, about 165 mg, about 170 mg, about 175 mg, about 180 mg, about 185 mg, about 190 mg, about 195 mg, about 200 mg, about 205 mg, about 210 mg, about 215 mg, about 220 mg, about 225 mg, about 230 mg, about 235 mg, about 240 mg, about 245 mg, about 250 mg, about 255 mg, about 260 mg, about 265 mg, about 270 mg, about 275 mg, about 280 mg, about 285 mg, about 290 mg, about 295 mg, about 300 mg, about 305 mg, about 310 mg, about 315 mg, about 319 mg, about 320 mg, about 325 mg, about 330 mg, about 335 mg, about 340 mg, about 345 mg, about 350 mg, about 355 mg, about 360 mg, about 365 mg, about 370 mg, about 375 mg, about 380 mg, about 385 mg, about 390 mg, about 395 mg, or about 400 mg dexpramipexole dihydrochloride monohydrate.

### 3. Tablet Core

As outlined above, the pharmaceutical composition in the form of an orally deliverable tablet according to the present disclosure comprises a tablet core. In some aspects, at least about 70% of the amount of dexpramipexole, or a pharmaceutically acceptable salt thereof, of the pharmaceutical composition is in the tablet core. In some aspects, the tablet core comprises a homogeneous mixture of an inorganic osmotic agent and the amount of dexpramipexole, or a pharmaceutically acceptable salt thereof, that is in the tablet core, wherein the inorganic osmotic agent constitutes about 10% to about 40% by weight of the tablet core. In some aspects, the homogenous mixture in the tablet core further comprises additional excipients.

In some embodiments, the pre-blend comprises an antioxidant. In some embodiments, the antioxidant is capable of acting as a nitrite scavenger. Non-limiting examples of nitrite scavengers include, but are not limitd to, ascorbic acid, L-cysteine, caffeic acid, cysteine HCL, methionine, tartaric acid, gallic acid, uric acid, and sodium sulphite. In some embodiments, the pre-blend comprises one nitrite scavenger. In another embodiment, the pre-blend comprises multiple nitrite scavengers. In some embodiments, the nitrite scavengers constitute about 0.1% to about 1.5% by weight of the tablet core. In some embodiments, the nitrite scavenger constitutes about 0.1% to about 1.0% by weight of the tablet core. In some embodiments, the nitrite scavenger constitutes about 0.1% to about 0.5% by weight of the tablet core. In some embodiments, the nitrite scavenger constitutes about 0.5% to about 1.5% by weight of the tablet core. In some embodiments, the nitrite scavenger constitutes about 1% to about 1.5% by weight of the tablet core. In some embodiments, the nitrite scavenger constitutes about 0.1%, about 0.2%, about 0.3%, about 0.4%, about 0.5%, about 0.6%, about 0.7%, about 0.8%, about 0.9%, about 1%, about 1.1%, about 1.2%, about 1.3%, about 1.4%, or about 1.5% by weight of the tablet core.

In some embodiments, the inorganic osmotic agent constitutes about 11% to about 39%, about 12% to about 38%, about 13% to about 37%, or about 14% to about 36% by weight of the tablet core. In some embodiments, the inorganic osmotic agent constitutes about 15% to about 35%, about 16% to about 34%, about 17% to about 33%, about 18% to about 32%, or about 19% to about 31% by weight of the tablet core. In some embodiments, the inorganic osmotic agent constitutes about 20% to about 30% by weight of the tablet core. In specific embodiments, the inorganic osmotic agent constitutes about 20% by weight of the tablet core. In other specific embodiments, the inorganic osmotic agent constitutes about 30% by weight of the tablet core.

In some embodiments, the inorganic osmotic agent constitutes about 10% to about 30%, about 11% to about 29%, about 12% to about 28%, about 13% to about 27%, about 14% to about 26%, about 15% to about 25%, about 16% to about 24%, about 17% to about 23%, about 18% to about 22%, about 19% to about 21%, or about 20% by weight of the tablet core.

In some embodiments, the inorganic osmotic agent constitutes about 20% to about 30% by weight of the tablet core. In some embodiments, the inorganic osmotic agent constitutes about 15% to about 25% by weight of the tablet core. In some embodiments, the inorganic osmotic agent constitutes about 18% to about 22% by weight of the tablet core. In specific embodiments, the inorganic osmotic agent constitutes about 20% by weight of the tablet core.

In some embodiments, the inorganic osmotic agent constitutes about 10%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, about 30%, about 31%, about 32%, about 33%, about 34%, about 35%, about 36%, about 37%, about 38%, about 39%, or about 40% by weight of the tablet core.

In some embodiments, the tablet core comprises about 80 mg to about 200 mg, about 85 mg to about 195 mg, about 90 mg to about 190 mg, about 95 mg to about 185 mg, about 100 mg to about 180 mg, about 105 mg to about 175 mg, about 110 mg to about 170 mg, about 115 mg to about 165 mg, about 120 mg to about 160 mg, or about 125 mg to about 155 mg inorganic osmotic agent. In some embodiments, the tablet core comprises about 125 mg to about 155 mg inorganic osmotic agent.

In some embodiments, the tablet core comprises about 100 mg to about 200 mg, about 105 mg to about 195 mg, about 110 mg to about 190 mg, about 115 mg to about 185 mg, about 120 mg to about 180 mg, about 125 mg to about 175 mg, about 130 mg to about 170 mg, about 135 mg to about 165 mg, about 140 mg to about 160 mg, or about 145 mg to about 155 mg inorganic osmotic agent. In some embodiments, the tablet core comprises about 145 mg to about 155 mg inorganic osmotic agent. In some embodiments, the tablet core comprises about 152 mg inorganic osmotic agent.

In some embodiments, the tablet core comprises about 80 mg to about 180 mg, about 85 mg to about 175 mg, about 90 mg to about 170 mg, about 95 mg to about 165 mg, about 100 mg to about 160 mg, about 105 mg to about 155 mg, about 110 mg to about 150 mg, about 115 mg to about 145 mg, about 120 mg to about 140 mg, about 125 mg to about 135 mg inorganic osmotic agent. In some embodiments, the tablet core comprises about 125 mg to about 135 mg inorganic osmotic agent. In some embodiments, the tablet core comprises about 129 mg inorganic osmotic agent.

In some embodiments, the tablet core comprises about 80 mg, about 85 mg, about 90 mg, about 95 mg, about 100 mg, about 105 mg, about 110 mg, about 115 mg, about 120 mg, about 125 mg, about 129 mg, about 130 mg, about 135 mg, about 140 mg, about 145 mg, about 150 mg, about 152 mg, about 155 mg, about 160 mg, about 165 mg, about 170 mg, about 175 mg, about 180 mg, about 185 mg, about 190 mg, about 195 mg, or about 200 mg inorganic osmotic agent.

In some embodiments, the inorganic osmotic agent is sodium chloride, potassium chloride, magnesium chloride, sodium hydrogen phosphate, potassium hydrogen phosphate, or any combination thereof.

In some embodiments, the inorganic osmotic salt is a metal halide. In some embodiments, the inorganic osmotic salt is an alkali metal halide or an earth metal halide.

In specific embodiments, the inorganic osmotic agent is sodium chloride.

In some embodiments, sodium chloride constitutes about 11% to about 39%, about 12% to about 38%, about 13% to about 37%, or about 14% to about 36% by weight of the tablet core. In some embodiments, sodium chloride constitutes about 15% to about 35%, about 16% to about 34%, about 17% to about 33%, about 18% to about 32%, or about 19% to about 31% by weight of the tablet core. In some embodiments, sodium chloride constitutes about 20% to about 30% by weight of the tablet core. In specific embodiments, sodium chloride constitutes about 20% by weight of the tablet core. In other specific embodiments, sodium chloride constitutes about 30% by weight of the tablet core.

In some embodiments, sodium chloride constitutes about 10% to about 30%, about 11% to about 29%, about 12% to about 28%, about 13% to about 27%, about 14% to about 26%, about 15% to about 25%, about 16% to about 24%, about 17% to about 23%, about 18% to about 22%, about 19% to about 21%, or about 20% by weight of the tablet core.

In some embodiments, sodium chloride constitutes about 20% to about 30% by weight of the tablet core. In some embodiments, sodium chloride constitutes about 15% to about 25% by weight of the tablet core. In some embodiments, sodium chloride constitutes about 18% to about 22% by weight of the tablet core. In specific embodiments, sodium chloride constitutes about 20% by weight of the tablet core.

In some embodiments, sodium chloride constitutes about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, about 30%, about 31%, about 32%, about 33%, about 34%, about 35%, about 36%, about 37%, about 38%, about 39%, or about 40% by weight of the tablet core.

In some embodiments, the tablet core comprises about 80 mg to about 200 mg, about 85 mg to about 195 mg, about 90 mg to about 190 mg, about 95 mg to about 185 mg, about 100 mg to about 180 mg, about 105 mg to about 175 mg, about 110 mg to about 170 mg, about 115 mg to about 165 mg, about 120 mg to about 160 mg, or about 125 mg to about 155 mg sodium chloride. In some embodiments, the tablet core comprises about 125 mg to about 155 mg sodium chloride.

In some embodiments, the tablet core comprises about 100 mg to about 200 mg, about 105 mg to about 195 mg, about 110 mg to about 190 mg, about 115 mg to about 185 mg, about 120 mg to about 180 mg, about 125 mg to about 175 mg, about 130 mg to about 170 mg, about 135 mg to about 165 mg, about 140 mg to about 160 mg, or about 145 mg to about 155 mg sodium chloride. In some embodiments, the tablet core comprises about 145 mg to about 155 mg sodium chloride. In some embodiments, the tablet core comprises about 152 mg sodium chloride.

In some embodiments, the tablet core comprises about 80 mg to about 180 mg, about 85 mg to about 175 mg, about 90 mg to about 170 mg, about 95 mg to about 165 mg, about 100 mg to about 160 mg, about 105 mg to about 155 mg, about 110 mg to about 150 mg, about 115 mg to about 145 mg, about 120 mg to about 140 mg, about 125 mg to about 135 mg sodium chloride. In some embodiments, the tablet core comprises about 125 mg to about 135 mg sodium chloride. In some embodiments, the tablet core comprises about 129 mg sodium chloride.

In some embodiments, the tablet core comprises about 80 mg, about 85 mg, about 90 mg, about 95 mg, about 100 mg, about 105 mg, about 110 mg, about 115 mg, about 120 mg, about 125 mg, about 129 mg, about 130 mg, about 135 mg, about 140 mg, about 145 mg, about 150 mg, about 152 mg, about 155 mg, about 160 mg, about 165 mg, about 170 mg, about 175 mg, about 180 mg, about 185 mg, about 190 mg, about 195 mg, or about 200 mg sodium chloride.

In some embodiments, the tablet core further comprises other osmotic agents, such as organic osmotic agents.

In some specific embodiments, the homogeneous mixture in the tablet core does not comprise polyethylene oxide. In some specific embodiments, the homogeneous mixture in the tablet core does not comprise a swellable polymeric osmotic agent.

In some embodiments, the homogeneous mixture in the tablet core further comprises a lubricant. As used herein, the term “lubricant” is intended to mean substances used in tablet formulations to reduce friction during tablet compression. Examples of suitable lubricants are stearic acid and salts thereof such as magnesium stearate, calcium stearate, zinc stearate; glyceryl behenate, sodium stearyl fumarate; polyethylene glycols; silicone dioxide (such as colloidal silicon dioxide); talc; and the like.

In some embodiments, the lubricant constitutes about 0.1% to about 0.9%, about 0.15% to about 0.85%, about 0.2% to about 0.8%, or about 0.25% to about 0.75% by weight of the tablet core. In some embodiments, the lubricant constitutes about 0.3% to about 0.7% or about 0.35% to about 0.65% by weight of the tablet core. In some embodiments, the lubricant constitutes about 0.4% to about 0.6% by weight of the tablet core. In some embodiments, the lubricant constitutes about 0.45% to about 0.55% by weight of the tablet core. In specific embodiments, the lubricant constitutes about 0.5% by weight of the tablet core.

In some embodiments, the lubricant constitutes about 0.1%, about 0.15%, about 0.2%, about 0.25%, about 0.3%, about 0.35%, about 0.4%, about 0.45%, about 0.5%, about 0.55%, about 0.6%, about 0.65%, about 0.7%, about 0.75%, about 0.8%, about 0.85%, or about 0.9% by weight of the tablet core.

In some embodiments, the tablet core comprises about 1 mg to about 7 mg, about 1.5 mg to about 6.5 mg, about 2 mg to about 6 mg, or about 2.5 mg to about 5.5 mg lubricant. In some embodiments, the tablet core comprises about 3 mg to about 5 mg lubricant. In some embodiments, the tablet core comprises about 3.5 mg to about 4.5 mg lubricant. In some embodiments, the tablet core comprises about 2.5 mg to about 3.5 mg lubricant. In some embodiments, the tablet core comprises about 3 mg to about 4 mg lubricant. In some embodiments, the tablet core comprises about 3 mg lubricant. In some embodiments, the tablet core comprises about 4 mg lubricant.

In some embodiments, the tablet core comprises about 1 mg, about 1.5 mg, about 2 mg, about 2.5 mg, about 3 mg, about 3.5 mg, about 4 mg, about 4.5 mg, about 5 mg, about 5.5 mg, about 6 mg, about 6.5 mg, or about 7 mg lubricant.

In specific embodiments, the lubricant is magnesium stearate.

In some embodiments, magnesium stearate constitutes about 0.1% to about 0.9%, about 0.15% to about 0.85%, about 0.2% to about 0.8%, or about 0.25% to about 0.75% by weight of the tablet core. In some embodiments, magnesium stearate constitutes about 0.3% to about 0.7% or about 0.35% to about 0.65% by weight of the tablet core. In some embodiments, magnesium stearate constitutes about 0.4% to about 0.6% by weight of the tablet core. In some embodiments, magnesium stearate constitutes about 0.45% to about 0.55% by weight of the tablet core. In specific embodiments, magnesium stearate constitutes about 0.5% by weight of the tablet core.

In some embodiments, magnesium stearate constitutes about 0.1%, about 0.15%, about 0.2%, about 0.25%, about 0.3%, about 0.35%, about 0.4%, about 0.45%, about 0.5%, about 0.55%, about 0.6%, about 0.65%, about 0.7%, about 0.75%, about 0.8%, about 0.85%, or about 0.9% by weight of the tablet core.

In some embodiments, the tablet core comprises about 1 mg to about 7 mg, about 1.5 mg to about 6.5 mg, about 2 mg to about 6 mg, or about 2.5 mg to about 5.5 mg magnesium stearate. In some embodiments, the tablet core comprises about 3 mg to about 5 mg magnesium stearate. In some embodiments, the tablet core comprises about 3.5 mg to about 4.5 mg magnesium stearate. In some embodiments, the tablet core comprises about 2.5 mg to about 3.5 mg magnesium stearate. In some embodiments, the tablet core comprises about 3 mg to about 4 mg magnesium stearate. In some embodiments, the tablet core comprises about 3 mg magnesium stearate. In some embodiments, the tablet core comprises about 4 mg magnesium stearate.

In some embodiments, the tablet core comprises about 1 mg, about 1.5 mg, about 2 mg, about 2.5 mg, about 3 mg, about 3.5 mg, about 4 mg, about 4.5 mg, about 5 mg, about 5.5 mg, about 6 mg, about 6.5 mg, or about 7 mg magnesium stearate.

In some embodiments, the homogenous mixture in the tablet core further comprises a glidant. As used herein, the term “glidant” is intended to mean substances used in tablet formulations to improve flowability during tablet manufacturing. Examples of suitable glidants are silicon dioxide, colloidal silicon dioxide, ascorbyl palmitate, calcium palmitate, starch, talc, and the like. In some embodiments, the glidant is silicon dioxide and/or talc.

In some embodiments, the homogeneous mixture in the tablet core further comprises a diluent. As used herein, the term “diluent” is intended to mean an inert substance used as filler to create the desired bulk, flow properties, or compression characteristics in the preparation of tablets. Examples of suitable diluents are microcrystalline cellulose; powdered cellulose; precipitated calcium carbonate; sorbitol; starch; anhydrous lactose; lactose monohydrate; sugar alcohols such as sorbitol, xylitol and mannitol; and the like. In some embodiments, the diluent is microcrystalline cellulose, and the microcrystalline cellulose is silicified microcrystalline cellulose.

In some embodiments, the diluent constitutes about 20% to about 40%, about 21% to about 39%, about 22% to about 38%, about 23% to about 37%, about 24% to about 36%, or about 25% to about 35% by weight of the tablet core. In some embodiments, the diluent constitutes about 26% to about 34% by weight of the tablet core. In some embodiments, the diluent constitutes about 27% to about 33% by weight of the tablet core. In some embodiments, the diluent constitutes about 28% to about 32% by weight of the tablet core. In some embodiments, the diluent constitutes about 29% to about 31% by weight of the tablet core. In some embodiments, the diluent constitutes about 29% to about 33% by weight of the tablet core. In some embodiments, the diluent constitutes about 30% to about 32% by weight of the tablet core. In some embodiments, the diluent constitutes about 31% by weight of the tablet core. In some embodiments, the diluent constitutes about 30% to about 31% by weight of the tablet core.

In some embodiments, the diluent constitutes about 20%, about 21%, about 22%, about 23%, about 24%, about 25%, about 26%, about 27%, about 28%, about 29%, about 30%, about 30.5%, about 31%, about 32%, about 33%, about 34%, about 35%, about 36%, about 37%, about 38%, about 39%, or about 40% by weight of the tablet core. In specific embodiments, the diluent constitutes about 30.5% by weight of the tablet core.

In some embodiments, the tablet core comprises about 150 mg to about 300 mg diluent. In some embodiments, the tablet core comprises about 150 mg to about 280 mg, about 155 mg to about 275 mg, about 160 mg to about 270 mg, about 165 mg to about 265 mg, about 170 mg to about 260 mg, about 175 mg to about 255 mg, about 180 mg to about 250 mg, or about 185 mg to about 245 mg diluent. In some embodiments, the tablet core comprises about 190 mg to about 240 mg or about 195 mg to about 235 mg diluent. In some embodiments, the tablet core comprises about 180 mg to about 220 mg, about 185 mg to about 215 mg, about 190 mg to about 210 mg, or about 195 mg to about 205 mg diluent. In specific embodiments, the tablet core comprises about 197 mg diluent. In some embodiments, the tablet core comprises about 210 mg to about 250 mg, about 215 mg to about 245 mg, about 220 mg to about 240 mg, about 225 mg to about 235 mg, or about 230 mg to about 235 mg diluent. In specific embodiments, the tablet core comprises about 232 mg diluent.

In some embodiments, the tablet core comprises about 150 mg, about 155 mg, about 160 mg, about 165 mg, about 170 mg, about 175 mg, about 180 mg, about 185 mg, about 190 mg, about 195 mg, about 197 mg, about 200 mg, about 205 mg, about 210 mg, about 215 mg, about 220 mg, about 225 mg, about 230 mg, about 235 mg, about 240 mg, about 245 mg, about 250 mg, about 255 mg, about 260 mg, about 265 mg, about 270 mg, about 275 mg, about 280 mg, about 285 mg, about 290 mg, about 295 mg, or about 300 mg diluent.

In specific embodiments, the diluent is microcrystalline cellulose.

In some embodiments, microcrystalline cellulose constitutes about 20% to about 40%, about 21% to about 39%, about 22% to about 38%, about 23% to about 37%, about 24% to about 36%, or about 25% to about 35% by weight of the tablet core. In some embodiments, microcrystalline cellulose constitutes about 26% to about 34% by weight of the tablet core. In some embodiments, microcrystalline cellulose constitutes about 27% to about 33% by weight of the tablet core. In some embodiments, microcrystalline cellulose constitutes about 28% to about 32% by weight of the tablet core. In some embodiments, microcrystalline cellulose constitutes about 29% to about 31% by weight of the tablet core. In some embodiments, microcrystalline cellulose constitutes about 29% to about 33% by weight of the tablet core. In some embodiments, microcrystalline cellulose constitutes about 30% to about 32% by weight of the tablet core. In some embodiments, microcrystalline cellulose constitutes about 31% by weight of the tablet core. In some embodiments, microcrystalline cellulose constitutes about 30% to about 31% by weight of the tablet core.

In some embodiments, microcrystalline cellulose constitutes about 20%, about 21%, about 22%, about 23%, about 24%, about 25%, about 26%, about 27%, about 28%, about 29%, about 30%, about 30.5%, about 31%, about 32%, about 33%, about 34%, about 35%, about 36%, about 37%, about 38%, about 39%, or about 40% by weight of the tablet core. In specific embodiments, microcrystalline cellulose constitutes about 30.5% by weight of the tablet core.

In some embodiments, the tablet core comprises about 150 mg to about 300 mg microcrystalline cellulose. In some embodiments, the tablet core comprises about 150 mg to about 280 mg, about 155 mg to about 275 mg, about 160 mg to about 270 mg, about 165 mg to about 265 mg, about 170 mg to about 260 mg, about 175 mg to about 255 mg, about 180 mg to about 250 mg, or about 185 mg to about 245 mg microcrystalline cellulose. In some embodiments, the tablet core comprises about 190 mg to about 240 mg or about 195 mg to about 235 mg microcrystalline cellulose. In some embodiments, the tablet core comprises about 180 mg to about 220 mg, about 185 mg to about 215 mg, about 190 mg to about 210 mg, or about 195 mg to about 205 mg microcrystalline cellulose. In specific embodiments, the tablet core comprises about 197 mg microcrystalline cellulose. In some embodiments, the tablet core comprises about 210 mg to about 250 mg, about 215 mg to about 245 mg, about 220 mg to about 240 mg, about 225 mg to about 235 mg, or about 230 mg to about 235 mg microcrystalline cellulose. In specific embodiments, the tablet core comprises about 232 mg microcrystalline cellulose.

In some embodiments, the tablet core comprises about 150 mg, about 155 mg, about 160 mg, about 165 mg, about 170 mg, about 175 mg, about 180 mg, about 185 mg, about 190 mg, about 195 mg, about 197 mg, about 200 mg, about 205 mg, about 210 mg, about 215 mg, about 220 mg, about 225 mg, about 230 mg, about 235 mg, about 240 mg, about 245 mg, about 250 mg, about 255 mg, about 260 mg, about 265 mg, about 270 mg, about 275 mg, about 280 mg, about 285 mg, about 290 mg, about 295 mg, or about 300 mg microcrystalline cellulose.

In specific embodiments, the diluent is silicified microcrystalline cellulose.

In some embodiments, silicified microcrystalline cellulose constitutes about 20% to about 40%, about 21% to about 39%, about 22% to about 38%, about 23% to about 37%, about 24% to about 36%, or about 25% to about 35% by weight of the tablet core. In some embodiments, silicified microcrystalline cellulose constitutes about 26% to about 34% by weight of the tablet core. In some embodiments, silicified microcrystalline cellulose constitutes about 27% to about 33% by weight of the tablet core. In some embodiments, silicified microcrystalline cellulose constitutes about 28% to about 32% by weight of the tablet core. In some embodiments, silicified microcrystalline cellulose constitutes about 29% to about 31% by weight of the tablet core. In some embodiments, silicified microcrystalline cellulose constitutes about 29% to about 33% by weight of the tablet core. In some embodiments, silicified microcrystalline cellulose constitutes about 30% to about 32% by weight of the tablet core. In some embodiments, silicified microcrystalline cellulose constitutes about 31% by weight of the tablet core. In some embodiments, silicified microcrystalline cellulose constitutes about 30% to about 31% by weight of the tablet core.

In some embodiments, silicified microcrystalline cellulose constitutes about 20%, about 21%, about 22%, about 23%, about 24%, about 25%, about 26%, about 27%, about 28%, about 29%, about 30%, about 30.5%, about 31%, about 32%, about 33%, about 34%, about 35%, about 36%, about 37%, about 38%, about 39%, or about 40% by weight of the tablet core. In specific embodiments, silicified microcrystalline cellulose constitutes about 30.5% by weight of the tablet core.

In some embodiments, the tablet core comprises about 150 mg to about 300 mg silicified microcrystalline cellulose. In some embodiments, the tablet core comprises about 150 mg to about 280 mg, about 155 mg to about 275 mg, about 160 mg to about 270 mg, about 165 mg to about 265 mg, about 170 mg to about 260 mg, about 175 mg to about 255 mg, about 180 mg to about 250 mg, or about 185 mg to about 245 mg silicified microcrystalline cellulose. In some embodiments, the tablet core comprises about 190 mg to about 240 mg or about 195 mg to about 235 mg silicified microcrystalline cellulose. In some embodiments, the tablet core comprises about 180 mg to about 220 mg, about 185 mg to about 215 mg, about 190 mg to about 210 mg, or about 195 mg to about 205 mg silicified microcrystalline cellulose. In specific embodiments, the tablet core comprises about 197 mg silicified microcrystalline cellulose. In some embodiments, the tablet core comprises about 210 mg to about 250 mg, about 215 mg to about 245 mg, about 220 mg to about 240 mg, about 225 mg to about 235 mg, or about 230 mg to about 235 mg silicified microcrystalline cellulose. In specific embodiments, the tablet core comprises about 232 mg silicified microcrystalline cellulose.

In some embodiments, the tablet core comprises about 150 mg, about 155 mg, about 160 mg, about 165 mg, about 170 mg, about 175 mg, about 180 mg, about 185 mg, about 190 mg, about 195 mg, about 197 mg, about 200 mg, about 205 mg, about 210 mg, about 215 mg, about 220 mg, about 225 mg, about 230 mg, about 235 mg, about 240 mg, about 245 mg, about 250 mg, about 255 mg, about 260 mg, about 265 mg, about 270 mg, about 275 mg, about 280 mg, about 285 mg, about 290 mg, about 295 mg, or about 300 mg silicified microcrystalline cellulose.

In some embodiments, the homogeneous mixture in the tablet core further comprises a binder. As used herein, the term “binder” is intended to mean an inert substance used to aid in particle cohesion. Examples of suitable binders are polyvinylpyrrolidone-vinyl acetate copolymer (also referred to as “copovidone”); gelatin; cellulose; cellulose derivatives; polyvinylpyrrolidone; starch; sucrose; polyethylene glycol; and the like.

In some embodiments, the binder constitutes about 2% to about 12%, about 2% to about 8%, about 3% to about 11%, or about 4% to about 10% by weight of the tablet core. In some embodiments, the binder constitutes about 5% to about 9% by weight of the tablet core. In some embodiments, the binder constitutes about 6% to about 8% by weight of the tablet core. In some embodiments, the binder constitutes about 6.5% to about 7.5% by weight of the tablet core. In specific embodiments, the binder constitutes about 7% by weight of the tablet core.

In some embodiments, the binder constitutes about 2%, about 3%, about 4%, about 5%, about 6%, about 6.5%, about 7%, about 7.5%, about 8%, about 9%, about 10%, about 11%, or about 12% by weight of the tablet core.

In some embodiments, the tablet core comprises about 30 mg to about 70 mg or about 35 mg to about 65 mg binder. In some embodiments, the tablet core comprises about 40 mg to about 60 mg or about 45 mg to about 55 mg binder. In some embodiments, the tablet core comprises about 40 mg to about 50 mg or about 43 mg to about 47 mg binder. In some embodiments, the tablet core comprises about 50 mg to about 60 mg or about 50 mg to about 50 mg binder. In specific embodiments, the tablet core comprises about 45 mg or about 53 mg binder.

In some embodiments, the tablet core comprises about 30 mg, about 35 mg, about 40 mg, about 45 mg, about 50 mg, about 53 mg, about 55 mg, about 60 mg, about 65 mg, or about 75 mg binder.

In specific embodiments, the binder is polyvinylpyrrolidone-vinyl acetate copolymer.

In some embodiments, polyvinylpyrrolidone-vinyl acetate copolymer constitutes about 2% to about 12%, 2% to about 8%, about 3% to about 11%, or about 4% to about 10% by weight of the tablet core. In some embodiments, polyvinylpyrrolidone-vinyl acetate copolymer constitutes about 5% to about 9% by weight of the tablet core. In some embodiments, polyvinylpyrrolidone-vinyl acetate copolymer constitutes about 6% to about 8% by weight of the tablet core. In some embodiments, polyvinylpyrrolidone-vinyl acetate copolymer constitutes about 6.5% to about 7.5% by weight of the tablet core. In specific embodiments, polyvinylpyrrolidone-vinyl acetate copolymer constitutes about 7% by weight of the tablet core.

In some embodiments, the binder constitutes about 2%, about 3%, about 4%, about 5%, about 6%, about 6.5%, about 7%, about 7.5%, about 8%, about 9%, about 10%, about 11%, or about 12% by weight of the tablet core.

In some embodiments, the tablet core comprises about 30 mg to about 70 mg or about 35 mg to about 65 mg polyvinylpyrrolidone-vinyl acetate copolymer. In some embodiments, the tablet core comprises about 40 mg to about 60 mg or about 45 mg to about 55 mg polyvinylpyrrolidone-vinyl acetate copolymer. In some embodiments, the tablet core comprises about 40 mg to about 50 mg or about 43 mg to about 47 mg polyvinylpyrrolidone-vinyl acetate copolymer. In some embodiments, the tablet core comprises about 50 mg to about 60 mg or about 50 mg to about 50 mg polyvinylpyrrolidone-vinyl acetate copolymer. In specific embodiments, the tablet core comprises about 45 mg or about 53 mg polyvinylpyrrolidone-vinyl acetate copolymer.

In some embodiments, the tablet core comprises about 30 mg, about 35 mg, about 40 mg, about 45 mg, about 50 mg, about 53 mg, about 55 mg, about 60 mg, about 65 mg, or about 75 mg polyvinylpyrrolidone-vinyl acetate copolymer.

In specific embodiments, the homogeneous mixture in the tablet core further comprises microcrystalline cellulose, polyvinylpyrrolidone-vinyl acetate copolymer, magnesium stearate, or any combination thereof. In yet more specific embodiments, the homogeneous mixture in the tablet core further comprises microcrystalline cellulose, polyvinylpyrrolidone-vinyl acetate copolymer, and magnesium stearate.

In some embodiments, the homogeneous mixture in the tablet core further comprises about 28% to about 34% microcrystalline cellulose by weight of the tablet core, about 5% to about 9% polyvinylpyrrolidone-vinyl acetate copolymer by weight of the tablet core, and about 0.25% to about 0.75% magnesium stearate by weight of the tablet core. In some embodiments, the homogeneous mixture in the tablet core further comprises about 29% to about 33% microcrystalline cellulose by weight of the tablet core, about 5.5% to about 8.5% polyvinylpyrrolidone-vinyl acetate copolymer by weight of the tablet core, and about 0.4% to about 0.6% magnesium stearate by weight of the tablet core. In some embodiments, the homogeneous mixture in the tablet core further comprises about 30% to about 32% microcrystalline cellulose by weight of the tablet core, about 6% to about 8% polyvinylpyrrolidone-vinyl acetate copolymer by weight of the tablet core, and about 0.45% to about 0.55% magnesium stearate by weight of the tablet core. In some embodiments, the homogeneous mixture in the tablet core further comprises about 31% (such as about 30.5%) microcrystalline cellulose by weight of the tablet core, about 7% polyvinylpyrrolidone-vinyl acetate copolymer by weight of the tablet core, and about 0.5% magnesium stearate by weight of the tablet core.

In some embodiments, the homogeneous mixture in the tablet core further comprises about 180 mg to about 250 mg microcrystalline cellulose, about 30 mg to about 60 mg polyvinylpyrrolidone-vinyl acetate copolymer, and about 1 mg to about 6 mg magnesium stearate. In some embodiments, the homogeneous mixture in the tablet core further comprises about 195 mg to about 235 mg microcrystalline cellulose, about 40 mg to about 55 mg polyvinylpyrrolidone-vinyl acetate copolymer, and about 2 mg to about 5 mg magnesium stearate. In some embodiments, the homogeneous mixture in the tablet core further comprises about 225 mg to about 235 mg microcrystalline cellulose, about 50 mg to about 55 mg polyvinylpyrrolidone-vinyl acetate copolymer, and about 3 mg to about 4 mg magnesium stearate. In some embodiments, the homogeneous mixture in the tablet core further comprises about 190 mg to about 200 mg microcrystalline cellulose, about 40 mg to about 50 mg polyvinylpyrrolidone-vinyl acetate copolymer, and about 3 mg to about 4 mg magnesium stearate. In specific embodiments, the homogeneous mixture in the tablet core further comprises about 232 mg microcrystalline cellulose, about 53 mg polyvinylpyrrolidone-vinyl acetate copolymer, and about 4 mg magnesium stearate. In specific embodiments, the homogeneous mixture in the tablet core further comprises about 197 mg microcrystalline cellulose, about 45 mg polyvinylpyrrolidone-vinyl acetate copolymer, and about 3 mg magnesium stearate. In specific embodiments, the homogeneous mixture in the tablet core further comprises about 290 mg microcrystalline cellulose, about 67 mg polyvinylpyrrolidone-vinyl acetate copolymer, and about 5 mg magnesium stearate.

In some embodiments, the amount of dexpramipexole, or a pharmaceutically acceptable salt thereof, that is in the tablet core constitutes about 30% to about 50% of dexpramipexole dihydrochloride monohydrate equivalent by weight of the tablet core. In some embodiments, the amount of dexpramipexole, or a pharmaceutically acceptable salt thereof, that is in the tablet core constitutes about 32% to about 52%, about 33% to about 51%, about 34% to about 50%, about 35% to about 49%, about 36% to about 48%, about 37% to about 47%, about 38% to about 46%, about 39% to about 45%, about 40% to about 44%, about 41% to about 43%, or about 42% of dexpramipexole dihydrochloride monohydrate equivalent by weight of the tablet core. In some embodiments, the amount of dexpramipexole, or a pharmaceutically acceptable salt thereof, that is in the tablet core constitutes about 35% to about 45% of dexpramipexole dihydrochloride monohydrate equivalent by weight of the tablet core. In some embodiments, the amount of dexpramipexole, or a pharmaceutically acceptable salt thereof, that is in the tablet core constitutes about 37% to about 45% of dexpramipexole dihydrochloride monohydrate equivalent by weight of the tablet core. In some embodiments, the amount of dexpramipexole, or a pharmaceutically acceptable salt thereof, that is in the tablet core constitutes about 40% to about 44% of dexpramipexole dihydrochloride monohydrate equivalent by weight of the tablet core. In specific embodiments, the amount of dexpramipexole, or a pharmaceutically acceptable salt thereof, that is in the tablet core constitutes about 42% of dexpramipexole dihydrochloride monohydrate equivalent by weight of the tablet core. In some embodiments, the pharmaceutically acceptable salt of dexpramipexole is dexpramipexole dihydrochloride monohydrate.

In specific embodiments, the amount of dexpramipexole, or a pharmaceutically acceptable salt thereof, that is in the tablet core constitutes about 35% to about 45% of dexpramipexole dihydrochloride monohydrate equivalent by weight of the tablet core.

In some embodiments, the amount of dexpramipexole, or a pharmaceutically acceptable salt thereof, that is in the tablet core constitutes about 30%, about 31%, about 32%, about 33%, about 34%, about 35%, about 36%, about 37%, about 38%, about 39%, about 40%, about 41%, about 42%, about 43%, about 44%, about 45%, about 46%, about 47%, about 48%, about 49%, about 50%, about 51%, or about 52% of dexpramipexole dihydrochloride monohydrate equivalent by weight of the tablet core. In some embodiments, the pharmaceutically acceptable salt of dexpramipexole is dexpramipexole dihydrochloride monohydrate.

In some embodiments, the amount of dexpramipexole, or a pharmaceutically acceptable salt thereof, that is in the tablet core is about 200 mg to about 400 mg, about 205 mg to about 395 mg, about 210 mg to about 390 mg, about 215 mg to about 385 mg, about 220 mg to about 380 mg, about 225 mg to about 375 mg, about 230 mg to about 370 mg, about 235 mg to about 365 mg, about 240 mg to about 360 mg, about 245 mg to about 355 mg, about 250 mg to about 350 mg, about 255 mg to about 345 mg, about 260 mg to about 340 mg, about 265 mg to about 335 mg, about 270 mg to about 330 mg, about 275 mg to about 325 mg, about 280 mg to about 320 mg, about 285 mg to about 315 mg, about 290 mg to about 310 mg, about 295 mg to about 305 mg, or about 300 mg of dexpramipexole dihydrochloride equivalent. In specific embodiments, the amount of dexpramipexole, or a pharmaceutically acceptable salt thereof, that is in the tablet core is about 300 mg of dexpramipexole dihydrochloride equivalent. In some embodiments, the pharmaceutically acceptable salt of dexpramipexole is dexpramipexole dihydrochloride monohydrate.

In some embodiments, the amount of dexpramipexole, or a pharmaceutically acceptable salt thereof, that is in the tablet core is about 155 mg to about 355 mg, about 160 mg to about 350 mg, about 165 mg to about 345 mg, about 170 mg to about 340 mg, about 175 mg to about 335 mg, about 180 mg to about 330 mg, about 185 mg to about 325 mg, about 190 mg to about 320 mg, about 195 mg to about 315 mg, about 200 mg to about 310 mg, about 205 mg to about 305 mg, about 210 mg to about 300 mg, about 215 mg to about 295 mg, about 220 mg to about 290 mg, about 225 mg to about 285 mg, about 230 mg to about 280 mg, about 235 mg to about 275 mg, about 240 mg to about 270 mg, about 245 mg to about 265 mg, about 250 mg to about 260 mg, or about 255 mg of dexpramipexole dihydrochloride equivalent. In specific embodiments, the amount of dexpramipexole, or a pharmaceutically acceptable salt thereof, that is in the tablet core is about 255 mg of dexpramipexole dihydrochloride equivalent. In some embodiments, the pharmaceutically acceptable salt of dexpramipexole is dexpramipexole dihydrochloride monohydrate.

In some embodiments, the amount of dexpramipexole, or a pharmaceutically acceptable salt thereof, that is in the tablet core is about 150 mg to about 400 mg dexpramipexole dihydrochloride equivalent. In some embodiments, the amount of dexpramipexole, or a pharmaceutically acceptable salt thereof, that is in the tablet core is about 200 mg to about 400 mg dexpramipexole dihydrochloride equivalent. In some embodiments, the amount of dexpramipexole, or a pharmaceutically acceptable salt thereof, that is in the tablet core is about 250 mg to about 350 mg dexpramipexole dihydrochloride equivalent. In some embodiments, the amount of dexpramipexole, or a pharmaceutically acceptable salt thereof, that is in the tablet core is about 220 mg to about 280 mg dexpramipexole dihydrochloride equivalent. In some embodiments, the amount of dexpramipexole, or a pharmaceutically acceptable salt thereof, that is in the tablet core is about 270 mg to about 330 mg dexpramipexole dihydrochloride equivalent. In some embodiments, the amount of dexpramipexole, or a pharmaceutically acceptable salt thereof, that is in the tablet core is about 290 mg to about 310 mg dexpramipexole dihydrochloride equivalent. In specific embodiments, the amount of dexpramipexole, or a pharmaceutically acceptable salt thereof, that is in the tablet core is about 300 mg dexpramipexole dihydrochloride equivalent. In some embodiments, the amount of dexpramipexole, or a pharmaceutically acceptable salt thereof, that is in the tablet core is about 245 mg to about 265 mg dexpramipexole dihydrochloride equivalent. In specific embodiments, the amount of dexpramipexole, or a pharmaceutically acceptable salt thereof, that is in the tablet core is about 255 mg dexpramipexole dihydrochloride equivalent. In some embodiments, the pharmaceutically acceptable salt of dexpramipexole is dexpramipexole dihydrochloride monohydrate.

In some embodiments, the amount of dexpramipexole, or a pharmaceutically acceptable salt thereof, that is in the tablet core is about 300 mg to about 400 mg, about 310 mg to about 395 mg, about 320 mg to about 390 mg, about 330 mg to about 385 mg, or about 340 mg to about 380 mg of dexpramipexole dihydrochloride equivalent. In specific embodiments, the amount of dexpramipexole, or a pharmaceutically acceptable salt thereof, that is in the tablet core is about 376 mg of dexpramipexole dihydrochloride equivalent. In some embodiments, the pharmaceutically acceptable salt of dexpramipexole is dexpramipexole dihydrochloride monohydrate.

In some embodiments, the amount of dexpramipexole, or a pharmaceutically acceptable salt thereof, that is in the tablet core is about 150 mg to about 400 mg dexpramipexole dihydrochloride equivalent. In some embodiments, the amount of dexpramipexole, or a pharmaceutically acceptable salt thereof, that is in the tablet core is about 200 mg to about 400 mg dexpramipexole dihydrochloride equivalent. In some embodiments, the amount of dexpramipexole, or a pharmaceutically acceptable salt thereof, that is in the tablet core is about 250 mg to about 400 mg dexpramipexole dihydrochloride equivalent. In some embodiments, the amount of dexpramipexole, or a pharmaceutically acceptable salt thereof, that is in the tablet core is about 275 mg to about 400 mg dexpramipexole dihydrochloride equivalent. In some embodiments, the amount of dexpramipexole, or a pharmaceutically acceptable salt thereof, that is in the tablet core is about 300 mg to about 400 mg dexpramipexole dihydrochloride equivalent. In some embodiments, the amount of dexpramipexole, or a pharmaceutically acceptable salt thereof, that is in the tablet core is about 325 mg to about 400 mg dexpramipexole dihydrochloride equivalent. In some embodiments, the amount of dexpramipexole, or a pharmaceutically acceptable salt thereof, that is in the tablet core is about 350 mg to about 400 mg dexpramipexole dihydrochloride equivalent. In specific embodiments, the amount of dexpramipexole, or a pharmaceutically acceptable salt thereof, that is in the tablet core is about 376 mg dexpramipexole dihydrochloride equivalent. In some embodiments, the pharmaceutically acceptable salt of dexpramipexole is dexpramipexole dihydrochloride monohydrate.

In some embodiments, at least about 71%, at least about 72%, at least about 73%, at least about 74%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or about 100% of the amount of dexpramipexole, or a pharmaceutically acceptable salt thereof, is in the tablet core.

In some embodiments, at least about 75% of the amount of dexpramipexole, or a pharmaceutically acceptable salt thereof, is in the tablet core. In some embodiments, at least about 80% of the amount of dexpramipexole, or a pharmaceutically acceptable salt thereof, is in the tablet core. In some embodiments, at least about 85% of the amount of dexpramipexole, or a pharmaceutically acceptable salt thereof, is in the tablet core. In some embodiments, at least about 90% of the amount of dexpramipexole, or a pharmaceutically acceptable salt thereof, is in the tablet core. In some embodiments, at least about 95% of the amount of dexpramipexole, or a pharmaceutically acceptable salt thereof, is in the tablet core. In some embodiments, at least about 98% of the amount of dexpramipexole, or a pharmaceutically acceptable salt thereof, is in the tablet core. In some embodiments, at least about 99% of the amount of dexpramipexole, or a pharmaceutically acceptable salt thereof, is in the tablet core. In some embodiments, about 100% of the amount of dexpramipexole, or a pharmaceutically acceptable salt thereof, is in the tablet core. In some embodiments, at least 99% or 100% of the amount of dexpramipexole, or a pharmaceutically acceptable salt thereof, is in the tablet core.

In some embodiments, about 70%, about 71%, about 72%, about 73%, about 74%, about 75%, about 76%, about 77%, about 78%, about 79%, about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, or about 100% of the amount of dexpramipexole, or a pharmaceutically acceptable salt thereof, is in the tablet core. In some embodiments, 99% or 100% of the amount of dexpramipexole, or a pharmaceutically acceptable salt thereof, is in the tablet core.

In some embodiments, about 70% of the amount of dexpramipexole, or a pharmaceutically acceptable salt thereof, is in the tablet core. In some embodiments, about 75% of the amount of dexpramipexole, or a pharmaceutically acceptable salt thereof, is in the tablet core. In some embodiments, about 80% of the amount of dexpramipexole, or a pharmaceutically acceptable salt thereof, is in the tablet core. In specific embodiments, about 85% of the amount of dexpramipexole, or a pharmaceutically acceptable salt thereof, is in the tablet core. In some embodiments, about 90% of the amount of dexpramipexole, or a pharmaceutically acceptable salt thereof, is in the tablet core. In some embodiments, about 95% of the amount of dexpramipexole, or a pharmaceutically acceptable salt thereof, is in the tablet core. In some embodiments, about 98% of the amount of dexpramipexole, or a pharmaceutically acceptable salt thereof, is in the tablet core. In some embodiments, about 99% of the amount of dexpramipexole, or a pharmaceutically acceptable salt thereof, is in the tablet core. In specific embodiments, about 100% of the amount of dexpramipexole, or a pharmaceutically acceptable salt thereof, is in the tablet core.

In some embodiments, at least 99% or 100% of the amount of dexpramipexole, or a pharmaceutically acceptable salt thereof, is in the tablet core, and the homogeneous mixture in the tablet core consists essentially of:
(a) about 315 mg to about 325 mg dexpramipexole dihydrochloride monohydrate;
(b) about 227 mg to about 237 mg microcrystalline cellulose;
(c) about 48 mg to about 58 mg polyvinylpyrrolidone-vinyl acetate copolymer;
(d) about 147 mg to about 157 mg sodium chloride; and
(e) about 2 mg to about 6 mg magnesium stearate.

In some embodiments, at least 99% or 100% of the amount of dexpramipexole, or a pharmaceutically acceptable salt thereof, is in the tablet core, and the homogeneous mixture in the tablet core consists essentially of:
(a) about 319 mg dexpramipexole dihydrochloride monohydrate;
(b) about 232 mg microcrystalline cellulose;
(c) about 53 mg polyvinylpyrrolidone-vinyl acetate copolymer;
(d) about 152 mg sodium chloride; and
(e) about 4 mg magnesium stearate.

In some embodiments, at least 99% or 100% of the amount of dexpramipexole, or a pharmaceutically acceptable salt thereof, is in the tablet core, and the homogeneous mixture in the tablet core consists essentially of:
(a) about 390 mg to about 410 mg dexpramipexole dihydrochloride monohydrate;
(b) about 285 mg to about 295 mg microcrystalline cellulose;
(c) about 62 mg to about 72 mg polyvinylpyrrolidone-vinyl acetate copolymer;
(d) about 185 mg to about 195 mg sodium chloride; and
(e) about 3 mg to about 7 mg magnesium stearate.

In some embodiments, at least 99% or 100% of the amount of dexpramipexole, or a pharmaceutically acceptable salt thereof, is in the tablet core, and the homogeneous mixture in the tablet core consists essentially of:
(a) about 400 mg dexpramipexole dihydrochloride monohydrate;
(b) about 290 mg microcrystalline cellulose;
(c) about 67 mg polyvinylpyrrolidone-vinyl acetate copolymer;
(d) about 190 mg sodium chloride; and
(e) about 5 mg magnesium stearate.

In some embodiments, at least 99% or 100% of the amount of dexpramipexole, or a pharmaceutically acceptable salt thereof, is in the tablet core, and the homogeneous mixture in the tablet core consists essentially of:
(a) about 40% to about 45% dexpramipexole dihydrochloride monohydrate by weight of the tablet core;
(b) about 28% to about 33% microcrystalline cellulose by weight of the tablet core;
(c) about 4.5% to about 9.5% polyvinylpyrrolidone-vinyl acetate copolymer by weight of the tablet core;
(d) about 17.5% to about 22.5% of sodium chloride by weight of the tablet core; and
(e) about 0.25% to about 0.75% magnesium stearate by weight of the tablet core.

In some embodiments, at least 99% or 100% of the amount of dexpramipexole, or a pharmaceutically acceptable salt thereof, is in the tablet core, and the homogeneous mixture in the tablet core consists essentially of:
(a) about 42% dexpramipexole dihydrochloride monohydrate by weight of the tablet core;
(b) about 30.5% microcrystalline cellulose by weight of the tablet core;
(c) about 7% polyvinylpyrrolidone-vinyl acetate copolymer by weight of the tablet core;
(d) about 20% of sodium chloride by weight of the tablet core; and
(e) about 0.5% magnesium stearate by weight of the tablet core.

In some embodiments, at least 99% or 100% of the amount of dexpramipexole, or a pharmaceutically acceptable salt thereof, is in the tablet core, and the homogeneous mixture in the tablet core consists essentially of:
(a) about 315 mg to about 325 mg dexpramipexole dihydrochloride monohydrate;
(b) about 28% to about 33% microcrystalline cellulose by weight of the tablet core;
(c) about 4.5% to about 9.5% polyvinylpyrrolidone-vinyl acetate copolymer by weight of the tablet core;
(d) about 17.5% to about 22.5% of sodium chloride by weight of the tablet core; and
(e) about 0.25% to about 0.75% magnesium stearate by weight of the tablet core.

In some embodiments, at least 99% or 100% of the amount of dexpramipexole, or a pharmaceutically acceptable salt thereof, is in the tablet core, and the homogeneous mixture in the tablet core consists essentially of:
(a) about 319 mg dexpramipexole dihydrochloride monohydrate;
(b) about 30.5% microcrystalline cellulose by weight of the tablet core;
(c) about 7% polyvinylpyrrolidone-vinyl acetate copolymer by weight of the tablet core;
(d) about 20% of sodium chloride by weight of the tablet core; and
(e) about 0.5% magnesium stearate by weight of the tablet core.

In some embodiments, at least 99% or 100% of the amount of dexpramipexole, or a pharmaceutically acceptable salt thereof, is in the tablet core, and the homogeneous mixture in the tablet core consists essentially of:
(a) about 390 mg to about 410 mg dexpramipexole dihydrochloride monohydrate;
(b) about 28% to about 33% microcrystalline cellulose by weight of the tablet core;
(c) about 4.5% to about 9.5% polyvinylpyrrolidone-vinyl acetate copolymer by weight of the tablet core;
(d) about 17.5% to about 22.5% of sodium chloride by weight of the tablet core; and
(e) about 0.25% to about 0.75% magnesium stearate by weight of the tablet core.

In some embodiments, at least 99% or 100% of the amount of dexpramipexole, or a pharmaceutically acceptable salt thereof, is in the tablet core, and the homogeneous mixture in the tablet core consists essentially of:
(a) about 400 mg dexpramipexole dihydrochloride monohydrate;
(b) about 30.5% microcrystalline cellulose by weight of the tablet core;
(c) about 7% polyvinylpyrrolidone-vinyl acetate copolymer by weight of the tablet core;
(d) about 20% of sodium chloride by weight of the tablet core; and
(e) about 0.5% magnesium stearate by weight of the tablet core.

In some embodiments, the weight of the tablet core is about 400 mg to about 1000 mg, about 450 mg to about 950 mg, about 500 mg to about 900 mg, about 550 mg to about 850 mg, or about 600 mg to about 800 mg. In some embodiments, the weight of the tablet core is about 600 mg to about 700 mg. In some embodiments, the weight of the tablet core is about 600 mg to about 690 mg, about 610 mg to about 680 mg, about 620 mg to about 670 mg, about 630 mg to about 660 mg, or about 640 mg to about 650 mg. In specific embodiments, the weight of the tablet core is about 640 mg to about 650 mg, such as about 646 mg. In some embodiments, the weight of the tablet core is about 700 mg to about 800 mg. In some embodiments, the weight of the tablet core is about 710 mg to about 810 mg, about 720 mg to about 800 mg, about 730 mg to about 790 mg, about 740 mg to about 780 mg, or about 750 mg to about 770 mg. In specific embodiments, the weight of the tablet core is about 755 mg to about 765 mg, such as about 760 mg. In some embodiments, the weight of the tablet core is about 400 mg, about 450 mg, about 500 mg, about 550 mg, about 600 mg, about 650 mg, about 700 mg, about 750 mg, about 800 mg, about 850 mg, about 900 mg, about 950 mg, or about 1000 mg. In some embodiments, the weight of the tablet core is about 1000 mg or less. In some embodiments, the weight of the tablet core is about 950 mg or less. In some embodiments, the weight of the tablet core is about 900 mg or less. In some embodiments, the weight of the tablet core is about 850 mg or less. In some embodiments, the weight of the tablet core is about 800 mg or less. In some embodiments, the weight of the tablet core is about 750 mg or less. In some embodiments, the weight of the tablet core is about 700 mg or less. In some embodiments, the weight of the tablet core is about 650 mg or less. In some embodiments, the weight of the tablet core is at least about 500 mg, at least about 550 mg, at least about 600 mg, at least about 650 mg, at least about 700 mg, or at least about 750 mg.

In some embodiments, the hardness of the tablet core is about 5 to about 30 kilopond (kp). In some embodiments, the hardness of the tablet core is about 10 to about 25 kp. In some embodiments, the hardness of the tablet core is about 10 to about 20 kp. In some embodiments, the hardness of the tablet core is about 15 to about 25 kp. In some embodiments, the hardness of the tablet core is about 5 kp, about 10 kp, about 15 kp, about 20 kp, about 25 kp, or about 30 kp. In some embodiments, the hardness of the tablet core is about 20 kp. In some embodiments, the hardness of the tablet core is about 15 kp.

In some embodiments, the tablet core is a single-layer tablet core. A “single-layer” tablet core as used herein in the context of the pharmaceutical composition of the present disclosure refers to a tablet core consisting of a single layer of the homogeneous mixture described above, which comprises the inorganic osmotic agent and dexpramipexole, or a pharmaceutically acceptable salt thereof, in the amounts specified herein. An example of an osmotic tablet with a single-layer tablet core is an elementary osmotic pump. In some embodiments, the pharmaceutical composition is an elementary osmotic pump.

In other embodiments, the tablet core is a bi-layer tablet core. A “bi-layer” tablet core as used herein in the context of the pharmaceutical composition of the present disclosure refers to a tablet core consisting of a first and a second layer. More specifically, the “bi-layer” tablet core consists of a first layer providing the homogeneous mixture described herein, which comprises the inorganic osmotic agent and dexpramipexole, or the pharmaceutically acceptable salt thereof, in the amounts specified herein, and a second layer, the second layer comprising an additional osmotic agent, such as a swellable polymeric osmotic agent, but the second layer does not comprise dexpramipexole, or a pharmaceutically acceptable salt thereof. An example of an osmotic tablet with a bi-layer tablet core is a push-pull osmotic pump. In some embodiments, the pharmaceutical composition is a push-pull osmotic pump.

### 4. Semipermeable Membrane Coating

As outlined above, the pharmaceutical composition in the form of an orally deliverable tablet according to the present disclosure comprises a tablet core and a semipermeable membrane coating surrounding the tablet core. In general, the semipermeable membrane coating surrounding the tablet core is present in an amount sufficient to provide complete coverage of the tablet core.

The term “plasticizer” as used herein in the context of the semipermeable membrane refers to a substance that lowers the glass transition temperature, enhances flexibility and affects permeability of the semipermeable membrane, and subsequently release rate.

In some embodiments, the plasticizer constitutes about 5% to about 40%, 5% to about 35%, 5% to about 30%, 5% to about 25%, about 6% to about 24%, about 7% to about 23%, about 8% to about 22%, or about 9% to about 21% by weight of the semipermeable membrane coating. In some embodiments, the plasticizer constitutes about 5% by weight of the semipermeable membrane coating. In some embodiments, the plasticizer constitutes about 10% to about 20% by weight of the semipermeable membrane coating. In some embodiments, the plasticizer constitutes about 11% to about 19%, about 12% to about 18%, or about 13% to about 17% by weight of the semipermeable membrane coating. In some embodiments, the plasticizer constitutes about 14% to about 16% by weight of the semipermeable membrane coating. In some embodiments, the plasticizer constitutes about 14.5% to about 15.5% by weight of the semipermeable membrane coating. In specific embodiments, the plasticizer constitutes about 15% by weight of the semipermeable membrane coating.

In specific embodiments, the plasticizer constitutes about 18% by weight of the semipermeable membrane coating. In specific embodiments, the plasticizer constitutes about 25% by weight of the semipermeable membrane coating. In specific embodiments, the plasticizer constitutes about 30% by weight of the semipermeable membrane coating. In some embodiments, the plasticizer constitutes about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, about 30%, about 31%, about 32%, about 33%, about 34%, about 35%, about 36%, about 37%, about 38%, about 39%, or about 40% by weight of the semipermeable membrane coating.

In some embodiments, the semipermeable membrane coating in addition to a plasticizer further comprises a polymer that is substantially permeable to water but substantially impermeable to solutes including dexpramipexole, or a pharmaceutically acceptable salt thereof. In some embodiments, the semipermeable membrane comprises cellulose esters, like diacetate, propionate, acetate, triacetate, and acetate butyrate. In some embodiments, the semipermeable membrane comprises cellulose ethers, such as ethyl cellulose. In specific embodiments, the semipermeable membrane coating comprises cellulose acetate.

In some embodiments, the semipermeable membrane coating comprises about 65% to about 95% cellulose acetate by weight of the semipermeable membrane coating. In some embodiments, the semipermeable membrane coating comprises about 70% to about 95% cellulose acetate by weight of the semipermeable membrane coating. In some embodiments, the semipermeable membrane coating comprises about 75% to about 95% cellulose acetate by weight of the semipermeable membrane coating. In some embodiments, the semipermeable membrane coating comprises about 76% to about 94%, about 77% to about 93%, about 78% to about 92%, or about 79% to about 91% cellulose acetate by weight of the semipermeable membrane coating. In some embodiments, the semipermeable membrane coating comprises about 80% to about 90% cellulose acetate by weight of the semipermeable membrane coating. In some embodiments, the semipermeable membrane coating comprises about 81% to about 89% or about 82% to about 88% cellulose acetate by weight of the semipermeable membrane coating. In some embodiments, the semipermeable membrane coating comprises about 83% to about 87% cellulose acetate by weight of the semipermeable membrane coating. In some embodiments, the semipermeable membrane coating comprises about 84% to about 86% cellulose acetate by weight of the semipermeable membrane coating. In specific embodiments, the semipermeable membrane coating comprises about 85% cellulose acetate by weight of the semipermeable membrane coating. In specific embodiments, the semipermeable membrane coating comprises about 83% cellulose acetate by weight of the semipermeable membrane coating. In specific embodiments, the semipermeable membrane coating comprises about 75% cellulose acetate by weight of the semipermeable membrane coating. In specific embodiments, the semipermeable membrane coating comprises about 70% cellulose acetate by weight of the semipermeable membrane coating.

In some embodiments, the semipermeable membrane coating comprises about 65%, about 66%, 67%, 68%, 69%, 70%, 71%, 72%, 73%, 74%, 75%, about 76%, about 77%, about 78%, about 79%, about 80%, about 81%, about 82%, about 83%, about 84%, about 85%, about 86%, about 87%, about 88%, about 89%, about 90%, about 91%, about 92%, about 93%, about 94% or about 95% cellulose acetate by weight of the semipermeable membrane coating.

In some embodiments, the semipermeable membrane coating comprises about 65% to about 95% cellulose acetate by weight of the semipermeable membrane coating and about 35% to about 5% plasticizer by weight of the semipermeable membrane coating. In some embodiments, the semipermeable membrane coating comprises about 70% to about 95% cellulose acetate by weight of the semipermeable membrane coating and about 30% to about 5% plasticizer by weight of the semipermeable membrane coating. In some embodiments, the semipermeable membrane coating comprises about 75% to about 95% cellulose acetate by weight of the semipermeable membrane coating and about 25% to about 5% plasticizer by weight of the semipermeable membrane coating. In some embodiments, the semipermeable membrane coating comprises about 80% to about 90% cellulose acetate by weight of the semipermeable membrane coating and about 20% to about 10% plasticizer by weight of the semipermeable membrane coating. In some embodiments, the semipermeable membrane coating comprises about 83% to about 87% cellulose acetate by weight of the semipermeable membrane coating and about 17% to about 13% plasticizer by weight of the semipermeable membrane coating. In some embodiments, the semipermeable membrane coating comprises about 84% to about 86% cellulose acetate by weight of the semipermeable membrane coating and about 16% to about 14% plasticizer by weight of the semipermeable membrane coating. In specific embodiments, the semipermeable membrane coating comprises about 85% cellulose acetate by weight of the semipermeable membrane coating and about 15% plasticizer by weight of the semipermeable membrane coating. In specific embodiments, the semipermeable membrane coating comprises about 83% cellulose acetate by weight of the semipermeable membrane coating and about 17% plasticizer by weight of the semipermeable membrane coating. In specific embodiments, the semipermeable membrane coating comprises about 75% cellulose acetate by weight of the semipermeable membrane coating and about 25% plasticizer by weight of the semipermeable membrane coating. In specific embodiments, the semipermeable membrane coating comprises about 70% cellulose acetate by weight of the semipermeable membrane coating and about 30% plasticizer by weight of the semipermeable membrane coating.

In some embodiments, the semipermeable membrane coating comprises about 95% cellulose acetate by weight of the semipermeable membrane coating and about 5% plasticizer by weight of the semipermeable membrane coating.

In some embodiments, the plasticizer in the semipermeable membrane coating is polyethylene glycol, triethyl citrate, triacetin, diethyl phthalate, poloxamer, or any combination thereof.

In specific embodiments, the plasticizer is polyethylene glycol.

In some embodiments, the semipermeable membrane coating comprises about 65% to about 95% cellulose acetate by weight of the semipermeable membrane coating and about 35% to about 5% polyethylene glycol by weight of the semipermeable membrane coating. In some embodiments, the semipermeable membrane coating comprises about 70% to about 95% cellulose acetate by weight of the semipermeable membrane coating and about 30% to about 5% polyethylene glycol by weight of the semipermeable membrane coating. In some embodiments, the semipermeable membrane coating comprises about 75% to about 95% cellulose acetate by weight of the semipermeable membrane coating and about 25% to about 5% polyethylene glycol by weight of the semipermeable membrane coating. In specific embodiments, the semipermeable membrane coating comprises about 80% to about 90% cellulose acetate by weight of the semipermeable membrane coating and about 20% to about 10% polyethylene glycol by weight of the semipermeable membrane coating. In more specific embodiments, the semipermeable membrane coating comprises about 83% to about 87% cellulose acetate by weight of the semipermeable membrane coating and about 17% to about 13% polyethylene glycol by weight of the semipermeable membrane coating. In yet more specific embodiments, the semipermeable membrane coating comprises about 85% cellulose acetate by weight of the semipermeable membrane coating and about 15% polyethylene glycol by weight of the semipermeable membrane coating. In specific embodiments, the semipermeable membrane coating comprises about 83% cellulose acetate by weight of the semipermeable membrane coating and about 17% polyethylene glycol by weight of the semipermeable membrane coating. In specific embodiments, the semipermeable membrane coating comprises about 75% cellulose acetate by weight of the semipermeable membrane coating and about 25% polyethylene glycol by weight of the semipermeable membrane coating. In specific embodiments, the semipermeable membrane coating comprises about 70% cellulose acetate by weight of the semipermeable membrane coating and about 30% polyethylene glycol by weight of the semipermeable membrane coating.

In some embodiments, the cellulose acetate in the semipermeable membrane coating has an acetyl content of about 30% to about 50%. In some embodiments, the cellulose acetate in the semipermeable membrane coating has an acetyl content of about 31% to about 49%, about 32% to about 48%, about 33% to about 47%, or about 34% to about 46%. In some embodiments, the cellulose acetate in the semipermeable membrane coating has an acetyl content of about 35% to about 45%. In some embodiments, the cellulose acetate in the semipermeable membrane coating has an acetyl content of about 36% to about 44%, about 37% to about 41%, or about 38% to about 42%. In some embodiments, the cellulose acetate in the semipermeable membrane coating has an acetyl content of about 39% to about 41%. In specific embodiments, the cellulose acetate in the semipermeable membrane coating has an acetyl content of about 40%.

In some embodiments, the cellulose acetate in the semipermeable membrane coating has an acetyl content of about 30%, about 31%, about 32%, about 33%, about 34%, about 35%, about 36%, about 37%, about 38%, about 39%, about 40%, about 41%, about 42%, about 43%, about 44%, about 45%, about 46%, about 47%, about 48%, about 49%, or about 50%.

The weight ratio of the semipermeable membrane coating to the tablet core is also referred to herein (in particular in the Example section below) as “coating weight gain”. For example, a coating weight gain of about 5.5% w/w refers to a weight ratio of the semipermeable membrane coating to the tablet core of about 0.055:1.

In some embodiments, the weight ratio of the semipermeable membrane coating to the tablet core is about 0.04:1 to about 0.10:1. In some embodiments, the weight ratio of the semipermeable membrane coating to the tablet core is about 0.05:1 to about 0.09:1. In some embodiments, the weight ratio of the semipermeable membrane coating to the tablet core is about 0.06:1 to about 0.08:1.

In some embodiments, the weight ratio of the semipermeable membrane coating to the tablet core is about 0.05:1 to about 0.07:1. In some embodiments, the weight ratio of the semipermeable membrane coating to the tablet core is about 0.055:1 to about 0.065:1.

In specific embodiments, the weight ratio of the semipermeable membrane coating to the tablet core is about 0.045:1. In specific embodiments, the weight ratio of the semipermeable membrane coating to the tablet core is about 0.055:1. In other specific embodiments, the weight ratio of the semipermeable membrane coating to the tablet core is about 0.06:1. In yet other specific embodiments, the weight ratio of the semipermeable membrane coating to the tablet core is about 0.062:1. In yet other specific embodiments, the weight ratio of the semipermeable membrane coating to the tablet core is about 0.07:1.

In some embodiments, the weight ratio of the semipermeable membrane coating to the tablet core is about 0.07:1 to about 0.09:1. In some embodiments, the weight ratio of the semipermeable membrane coating to the tablet core is about 0.075:1 to about 0.085:1. In specific embodiments, the weight ratio of the semipermeable membrane coating to the tablet core is about 0.08:1.

In specific embodiments, the weight ratio of the semipermeable membrane coating to the tablet core is about 0.06:1 to about 0.08:1.

In some embodiments, the weight ratio of the semipermeable membrane coating to the tablet core is about 0.04:1, about 0.045:1, about 0.05:1, about 0.055:1, about 0.06:1, about 0.062:1, about 0.065:1, about 0.07:1, about 0.075:1, about 0.08:1, about 0.085:1, about 0.09:1, about 0.095:1, or about 0.10:1.

In some embodiments, the semipermeable membrane coating constitutes about 2% to about 10% by weight of the tablet. In some embodiments, the semipermeable membrane coating constitutes about 3% to about 9% by weight of the tablet. In some embodiments, the semipermeable membrane coating constitutes about 4% to about 8% by weight of the tablet. In some embodiments, the semipermeable membrane coating constitutes about 5% to about 7% by weight of the tablet. In some embodiments, the semipermeable membrane coating constitutes about 6% by weight of the tablet.

In some embodiments, the semipermeable membrane coating constitutes about 2%, about 3%, about 4%, about 5%, about 6%, about 7%, about 8%, about 9%, or about 10% by weight of the tablet.

In some embodiments, the tablet comprises about 30 mg to about 70 mg semipermeable membrane coating. In some embodiments, the tablet comprises about 40 mg to about 60 mg semipermeable membrane coating. In some embodiments, the tablet comprises about 45 mg to about 55 mg semipermeable membrane coating. In some embodiments, the tablet comprises about 47 mg to about 52 mg semipermeable membrane coating.

In some embodiments, the tablet comprises about 43 mg to about 51 mg or about 44 mg to about 50 mg semipermeable membrane coating. In some embodiments, the tablet comprises about 45 mg to about 49 mg semipermeable membrane coating. In some embodiments, the tablet comprises about 46 mg to about 48 mg, such as about 47 mg, semipermeable membrane coating.

In some embodiments, the tablet comprises about 48 mg to about 56 mg or about 49 mg to about 55 mg semipermeable membrane coating. In some embodiments, the tablet comprises about 50 mg to about 54 mg semipermeable membrane coating. In some embodiments, the tablet comprises about 51 mg to about 53 mg, such as about 52 mg, semipermeable membrane coating.

In some embodiments, the tablet comprises about 30 mg, about 31 mg, about 32 mg, about 33 mg, about 34 mg, about 35 mg, about 36 mg, about 37 mg, about 38 mg, about 39 mg, about 40 mg, about 41 mg, about 42 mg, about 43 mg, about 44 mg, about 45 mg, about 46 mg, about 47 mg, about 48 mg, about 49 mg, about 50 mg, about 51 mg, about 52 mg, about 53 mg, about 55 mg, about 56 mg, about 57 mg, about 58 mg, about 59 mg, about 60 mg, about 61 mg, about 62 mg, about 63 mg, about 64 mg, about 65 mg, about 66 mg, about 67 mg, about 68 mg, about 69 mg, or about 70 mg semipermeable membrane coating.

In some embodiments, the semipermeable membrane coating thickness is about 100 μm to about 700 μm.

In some embodiments, the semipermeable membrane coating thickness is about 100 μm to about 300 μm. In some embodiments, the semipermeable membrane coating thickness is about 150 μm to about 250 μm or about 200 μm to about 220 μm. In some embodiments, the semipermeable membrane coating thickness is about 210 μm. In some embodiments, the semipermeable membrane coating thickness is about 100 μm, about 110 μm, about 120 μm, about 130 μm, about 140 μm, about 150 μm, about 160 μm, about 170 μm, about 180 μm, about 190 μm, about 200 μm, about 210 μm, about 220 μm, about 230 μm, about 240 μm, about 250 μm, about 260 μm, about 270 μm, about 280 μm, about 290 μm, or about 300 μm.

In some embodiments, the semipermeable membrane coating thickness is about 500 μm to about 650 μm or about 580 μm to about 600 μm. In some embodiments, the semipermeable membrane coating thickness is about 592 μm. In some embodiments, the semipermeable membrane coating thickness is about 500 μm, about 510 μm, about 520 μm, about 530 μm, about 540 μm, about 550 μm, about 560 μm, about 570 μm, about 580 μm, about 590 μm, or about 600 μm.

In some embodiments, the semipermeable membrane coating comprises one or more drug delivery orifices (also simply referred to herein as “delivery orifices”). Dexpramipexole, or a pharmaceutically acceptable salt thereof, can be released from the tablet core through the one or more delivery orifices. The delivery orifices pierce through the semipermeable membrane coating.

In some embodiments, the one or more delivery orifices are obtained by drilling. In some embodiments, the one or more delivery orifices are obtained by mechanical drilling. In some embodiments, the one or more delivery orifices are obtained by laser drilling. The laser beam generally used for laser drilling is CO₂. In embodiments wherein the one or more delivery orifices are obtained by mechanical drilling and/or laser drilling, the delivery orifices are present in the semipermeable membrane coating already prior to administration to a human.

In some embodiments, the semipermeable membrane coating comprises one or more pore-forming agents, such as polyethylene glycol or sorbitol. The pore-forming agent dissolves and leaches out of the semipermeable membrane coating upon exposure to aqueous solution, thereby resulting in the formation of one or more delivery orifices in situ. The pore-forming agent may start to leach out of the semipermeable membrane coating after about four hours in aqueous solution. Leaching out of the pore-forming agent increases the permeability and porosity of the semipermeable membrane coating leading to release of drug from the pores generated in the membrane in situ.

In some embodiments, the semipermeable membrane coating provides for a combination of delivery orifices that are present in the semipermeable membrane coating prior to administration (obtained by drilling, such as mechanical drilling and/or laser drilling) and delivery orifices that are formed in situ in the membrane after administration (by leaching out of pore-forming agent in the semipermeable membrane coating, whereas the pore-forming agent may be polyethylene glycol).

In some embodiments, the semipermeable membrane coating comprises one or more delivery orifices having a diameter of about 0.35 mm to about 2.0 mm. In some embodiments, the one or more delivery orifices have a diameter of about 0.5 mm to about 1.5 mm, about 0.6 mm to about 1.4 mm, about 0.7 mm to about 1.3 mm, about 0.8 mm to about 1.2 mm, or about 1 mm to about 1.2 mm. In some embodiments, the one or more delivery orifices have a diameter of about 0.4 mm to about 1.0 mm, about 0.4 mm to about 0.9 mm, about 0.4 mm to about 0.8 mm, or about 0.4 mm to about 0.7 mm. In some embodiments, the one or more delivery orifices have a diameter of about 0.4 mm to about 0.6 mm. In some embodiments, the one or more delivery orifices have a diameter of about 0.45 mm to about 0.55 mm. In specific embodiments, the one or more delivery orifices have a diameter of about 0.5 mm. In some embodiments, the one or more delivery orifices have a diameter of about 0.5 to about 1.5 mm. In some embodiments, the one or more delivery orifices have a diameter of about 0.7 to about 1.3 mm. In some embodiments, the one or more delivery orifices have a diameter of about 0.8 to about 1.2 mm. In some embodiments, the one or more delivery orifices have a diameter of about 1 mm to about 1.2 mm. In some embodiments, the one or more delivery orifices have a diameter of about 1 mm. In some embodiments, the one or more delivery orifices have a diameter of about 1.1 mm. In some embodiments, the one or more delivery orifices have a diameter of about 1.2 mm.

In some embodiments, the one or more delivery orifices have a diameter of about 0.35 mm, about 0.4 mm, about 0.45 mm, about 0.5 mm, about 0.55 mm, about 0.6 mm, about 0.65 mm, about 0.7 mm, about 0.75 mm, about 0.8 mm, about 0.85 mm, about 0.9 mm, about 0.95 mm, about 1.0 mm, about 1.05 mm, about 1.1 mm, about 1.15 mm, about 1.2 mm, about 1.25 mm, about 1.3 mm, about 1.35 mm, about 1.4 mm, about 1.45 mm, about 1.5 mm, about 1.55 mm, about 1.6 mm, about 1.65 mm, about 1.7 mm, about 1.75 mm, about 1.8 mm, about 1.85 mm, about 1.9 mm, about 1.95 mm, or about 2.0 mm.

Within the meaning of this disclosure, the “diameter” of a delivery orifice, as used herein, is referring to the diameter of the delivery orifice as measured at the surface of the semipermeable membrane coating, which is not in contact with, for example, a seal coating or a film coating, but instead is in contact with the tablet core.

While mechanical drilling usually results in a cylindrical delivery orifice with a (substantially) constant diameter throughout the entire orifice, laser drilling usually results in a conical delivery orifice, for which the diameter is getting narrower towards the tablet core. A delivery orifice created by laser drilling thus usually provides the smallest diameter at the surface of the semipermeable membrane coating, which is in contact with the tablet core (said smallest diameter being referred to herein as “diameter”), and the largest diameter at the opposite entrance site of the delivery orifice (said largest diameter being referred to herein as “outer diameter” of a delivery orifice obtained by laser drilling). In some embodiments wherein the one or more delivery orifices are obtained by laser drilling, the one or more delivery orifices obtained by laser drilling have a diameter of about 0.4 mm to about 0.6 mm and an outer diameter of about 0.8 mm to about 1.4 mm. In specific embodiments, the one or more delivery orifices obtained by laser drilling have a diameter of about 0.5 mm and an outer diameter of about 1 mm to about 1.2 mm.

In some embodiments, the semipermeable membrane coating comprises one to ten delivery orifices. In some embodiments, the semipermeable membrane coating comprises one to eight delivery orifices. In some embodiments, the semipermeable membrane coating comprises one to six delivery orifices. In some embodiments, the semipermeable membrane coating comprises one to four delivery orifices. In some embodiments, the semipermeable membrane coating comprises four delivery orifices. In some embodiments, the semipermeable membrane coating comprises three delivery orifices. In some embodiments, the semipermeable membrane coating comprises two delivery orifices. In specific embodiments, the semipermeable membrane coating comprises one delivery orifice.

In some of the embodiments, wherein the semipermeable membrane coating comprises one or more drug delivery orifices obtained by drilling, the one or more delivery orifices are obtained by drilling after the semipermeable membrane coating is further coated with a film coating, optionally wherein the semipermeable membrane coating is also coated with a seal coating and/or immediate release drug coating (see also embodiments below). Thus, in such embodiments, also the film coating, and optionally also the seal coating and/or the immediate release drug coating if present, comprise the one or more delivery orifices (i.e., the one or more delivery orifices span not only through the semipermeable membrane coating, but also through the film coating and optionally also through the seal coating and/or the immediate release coating if present). In some of the embodiments described in this paragraph, the one or more delivery orifices obtained by laser drilling have a diameter of about 0.4 mm to about 0.6 mm and an outer diameter of about 0.8 mm to about 1.4 mm. In specific embodiments of the embodiments described in this paragraph, the one or more delivery orifices obtained by laser drilling have a diameter of about 0.5 mm and an outer diameter of about 1 mm to about 1.2 mm.

In some embodiments, at least 99% or 100% of the amount of dexpramipexole, or a pharmaceutically acceptable salt thereof, is in the tablet core and the homogeneous mixture in the tablet core consists essentially of:
(a) about 315 mg to about 325 mg dexpramipexole dihydrochloride monohydrate;
(b) about 227 mg to about 237 mg microcrystalline cellulose;
(c) about 48 mg to about 58 mg polyvinylpyrrolidone-vinyl acetate copolymer;
(d) about 147 mg to about 157 mg sodium chloride; and
(e) about 2 mg to about 6 mg magnesium stearate;
   wherein the tablet comprises about 42 mg to about 52 mg semipermeable membrane coating, and wherein the semipermeable membrane coating comprises about 83% to about 87% cellulose acetate by weight of the semipermeable membrane coating and about 17% to about 13% plasticizer by weight of the semipermeable membrane coating. In specific embodiments, the plasticizer is polyethylene glycol.

In some embodiments, at least 99% or 100% of the amount of dexpramipexole, or a pharmaceutically acceptable salt thereof, is in the tablet core and the homogeneous mixture in the tablet core consists essentially of:
(a) about 319 mg dexpramipexole dihydrochloride monohydrate;
(b) about 232 mg microcrystalline cellulose;
(c) about 53 mg polyvinylpyrrolidone-vinyl acetate copolymer;
(d) about 152 mg sodium chloride; and
(e) about 4 mg magnesium stearate;
   wherein the tablet comprises about 47 mg semipermeable membrane coating, and wherein the semipermeable membrane coating comprises about 85% cellulose acetate by weight of the semipermeable membrane coating and about 15% plasticizer by weight of the semipermeable membrane coating. In specific embodiments, the plasticizer is polyethylene glycol.

In some embodiments, at least 99% or 100% of the amount of dexpramipexole, or a pharmaceutically acceptable salt thereof, is in the tablet core and the homogeneous mixture in the tablet core consists essentially of:
(a) about 315 mg to about 325 mg dexpramipexole dihydrochloride monohydrate;
(b) about 227 mg to about 237 mg microcrystalline cellulose;
(c) about 48 mg to about 58 mg polyvinylpyrrolidone-vinyl acetate copolymer;
(d) about 147 mg to about 157 mg sodium chloride; and
(e) about 2 mg to about 6 mg magnesium stearate;
   wherein the tablet comprises about 29 mg to about 39 mg semipermeable membrane coating, and wherein the semipermeable membrane coating comprises about 73% to about 77% cellulose acetate by weight of the semipermeable membrane coating and about 27% to about 23% plasticizer by weight of the semipermeable membrane coating. In specific embodiments, the plasticizer is polyethylene glycol.

In some embodiments, at least 99% or 100% of the amount of dexpramipexole, or a pharmaceutically acceptable salt thereof, is in the tablet core and the homogeneous mixture in the tablet core consists essentially of:
(a) about 319 mg dexpramipexole dihydrochloride monohydrate;
(b) about 232 mg microcrystalline cellulose;
(c) about 53 mg polyvinylpyrrolidone-vinyl acetate copolymer;
(d) about 152 mg sodium chloride; and
(e) about 4 mg magnesium stearate;
   wherein the tablet comprises about 34 mg semipermeable membrane coating, and wherein the semipermeable membrane coating comprises about 75% cellulose acetate by weight of the semipermeable membrane coating and about 25% plasticizer by weight of the semipermeable membrane coating. In specific embodiments, the plasticizer is polyethylene glycol.

In some embodiments, at least 99% or 100% of the amount of dexpramipexole, or a pharmaceutically acceptable salt thereof, is in the tablet core and the homogeneous mixture in the tablet core consists essentially of:
(a) about 315 mg to about 325 mg dexpramipexole dihydrochloride monohydrate;
(b) about 227 mg to about 237 mg microcrystalline cellulose;
(c) about 48 mg to about 58 mg polyvinylpyrrolidone-vinyl acetate copolymer;
(d) about 147 mg to about 157 mg sodium chloride; and
(e) about 2 mg to about 6 mg magnesium stearate;
   wherein the tablet comprises about 48 mg to about 58 mg semipermeable membrane coating, and wherein the semipermeable membrane coating comprises about 67% to about 73% cellulose acetate by weight of the semipermeable membrane coating and about 33% to about 27% plasticizer by weight of the semipermeable membrane coating. In specific embodiments, the plasticizer is polyethylene glycol.

In some embodiments, at least 99% or 100% of the amount of dexpramipexole, or a pharmaceutically acceptable salt thereof, is in the tablet core and the homogeneous mixture in the tablet core consists essentially of:
(a) about 319 mg dexpramipexole dihydrochloride monohydrate;
(b) about 232 mg microcrystalline cellulose;
(c) about 53 mg polyvinylpyrrolidone-vinyl acetate copolymer;
(d) about 152 mg sodium chloride; and
(e) about 4 mg magnesium stearate;
   wherein the tablet comprises about 53 mg semipermeable membrane coating, and wherein the semipermeable membrane coating comprises about 70% cellulose acetate by weight of the semipermeable membrane coating and about 30% plasticizer by weight of the semipermeable membrane coating. In specific embodiments, the plasticizer is polyethylene glycol.

In some embodiments, at least 99% or 100% of the amount of dexpramipexole, or a pharmaceutically acceptable salt thereof, is in the tablet core and the homogeneous mixture in the tablet core consists essentially of:
(a) about 390 mg to about 410 mg dexpramipexole dihydrochloride monohydrate;
(b) about 285 mg to about 295 mg microcrystalline cellulose;
(c) about 62 mg to about 72 mg polyvinylpyrrolidone-vinyl acetate copolymer;
(d) about 185 mg to about 195 mg sodium chloride; and
(e) about 3 mg to about 7 mg magnesium stearate;
   wherein the tablet comprises about 59 mg to about 69 mg semipermeable membrane coating, and wherein the semipermeable membrane coating comprises about 81% to about 85% cellulose acetate by weight of the semipermeable membrane coating and about 19% to about 15% plasticizer by weight of the semipermeable membrane coating. In specific embodiments, the plasticizer is polyethylene glycol.

In some embodiments, at least 99% or 100% of the amount of dexpramipexole, or a pharmaceutically acceptable salt thereof, is in the tablet core and the homogeneous mixture in the tablet core consists essentially of:
(a) about 400 mg dexpramipexole dihydrochloride monohydrate;
(b) about 290 mg microcrystalline cellulose;
(c) about 67 mg polyvinylpyrrolidone-vinyl acetate copolymer;
(d) about 190 mg sodium chloride; and
(e) about 5 mg magnesium stearate;
   wherein the tablet comprises about 64 mg semipermeable membrane coating, and wherein the semipermeable membrane coating comprises about 83% cellulose acetate by weight of the semipermeable membrane coating and about 17% plasticizer by weight of the semipermeable membrane coating. In specific embodiments, the plasticizer is polyethylene glycol.

In some embodiments, at least 99% or 100% of the amount of dexpramipexole, or a pharmaceutically acceptable salt thereof, is in the tablet core and the homogeneous mixture in the tablet core consists essentially of:
(a) about 40% to about 45% dexpramipexole dihydrochloride monohydrate by weight of the tablet core;
(b) about 28% to about 33% microcrystalline cellulose by weight of the tablet core;
(c) about 4.5% to about 9.5% polyvinylpyrrolidone-vinyl acetate copolymer by weight of the tablet core;
(d) about 17.5% to about 22.5% of sodium chloride by weight of the tablet core; and
(e) about 0.25% to about 0.75% magnesium stearate by weight of the tablet core;
   wherein the semipermeable membrane coating comprises about 83% to about 87% cellulose acetate by weight of the semipermeable membrane coating and about 17% to about 13% plasticizer by weight of the semipermeable membrane coating, and wherein the weight ratio of the semipermeable membrane coating to the tablet core is about 0.06:1 to about 0.08:1. In some embodiments, the plasticizer is polyethylene glycol. In some embodiments, the weight ratio of the semipermeable membrane coating to the tablet core is about 0.06:1. In some embodiments, the weight ratio of the semipermeable membrane coating to the tablet core is about 0.062:1. In some embodiments, the weight ratio of the semipermeable membrane coating to the tablet core is about 0.08:1.

In some embodiments, at least 99% or 100% of the amount of dexpramipexole, or a pharmaceutically acceptable salt thereof, is in the tablet core and the homogeneous mixture in the tablet core consists essentially of:
(a) about 42% dexpramipexole dihydrochloride monohydrate by weight of the tablet core;
(b) about 31% microcrystalline cellulose by weight of the tablet core;
(c) about 7% polyvinylpyrrolidone-vinyl acetate copolymer by weight of the tablet core;
(d) about 20% of sodium chloride by weight of the tablet core; and
(e) about 0.5% magnesium stearate by weight of the tablet core;
   wherein the semipermeable membrane coating comprises about 85% cellulose acetate by weight of the semipermeable membrane coating and about 15% plasticizer by weight of the semipermeable membrane coating, and wherein the weight ratio of the semipermeable membrane coating to the tablet core is about 0.06:1 to about 0.08:1. In some embodiments, the plasticizer is polyethylene glycol. In some embodiments, the weight ratio of the semipermeable membrane coating to the tablet core is about 0.06:1. In some embodiments, the weight ratio of the semipermeable membrane coating to the tablet core is about 0.062:1. In some embodiments, the weight ratio of the semipermeable membrane coating to the tablet core is about 0.08:1.

In some embodiments, at least 99% or 100% of the amount of dexpramipexole, or a pharmaceutically acceptable salt thereof, is in the tablet core and the homogeneous mixture in the tablet core consists essentially of:
(a) about 40% to about 45% dexpramipexole dihydrochloride monohydrate by weight of the tablet core;
(b) about 28% to about 33% microcrystalline cellulose by weight of the tablet core;
(c) about 4.5% to about 9.5% polyvinylpyrrolidone-vinyl acetate copolymer by weight of the tablet core;
(d) about 17.5% to about 22.5% of sodium chloride by weight of the tablet core; and
(e) about 0.25% to about 0.75% magnesium stearate by weight of the tablet core;

wherein the semipermeable membrane coating comprises about 81% to about 85% cellulose acetate by weight of the semipermeable membrane coating and about 19% to about 15% plasticizer by weight of the semipermeable membrane coating, and wherein the weight ratio of the semipermeable membrane coating to the tablet core is about 0.06:1 to about 0.08:1. In some embodiments, the plasticizer is polyethylene glycol. In some embodiments, the weight ratio of the semipermeable membrane coating to the tablet core is about 0.07:1. In some embodiments, the weight ratio of the semipermeable membrane coating to the tablet core is about 0.067:1.

In some embodiments, at least 99% or 100% of the amount of dexpramipexole, or a pharmaceutically acceptable salt thereof, is in the tablet core and the homogeneous mixture in the tablet core consists essentially of:
(a) about 42% dexpramipexole dihydrochloride monohydrate by weight of the tablet core;
(b) about 31% microcrystalline cellulose by weight of the tablet core;
(c) about 7% polyvinylpyrrolidone-vinyl acetate copolymer by weight of the tablet core;
(d) about 20% of sodium chloride by weight of the tablet core; and
(e) about 0.5% magnesium stearate by weight of the tablet core;
wherein the semipermeable membrane coating comprises about 83% cellulose acetate by weight of the semipermeable membrane coating and about 17% plasticizer by weight of the semipermeable membrane coating, and wherein the weight ratio of the semipermeable membrane coating to the tablet core is about 0.06:1 to about 0.08:1. In some embodiments, the plasticizer is polyethylene glycol. In some embodiments, the weight ratio of the semipermeable membrane coating to the tablet core is about 0.07:1. In some embodiments, the weight ratio of the semipermeable membrane coating to the tablet core is about 0.067:1.

In some embodiments, at least 99% or 100% of the amount of dexpramipexole, or a pharmaceutically acceptable salt thereof, is in the tablet core and the homogeneous mixture in the tablet core consists essentially of:
(a) about 40% to about 45% dexpramipexole dihydrochloride monohydrate by weight of the tablet core;
(b) about 28% to about 33% microcrystalline cellulose by weight of the tablet core;
(c) about 4.5% to about 9.5% polyvinylpyrrolidone-vinyl acetate copolymer by weight of the tablet core;
(d) about 17.5% to about 22.5% of sodium chloride by weight of the tablet core; and
(e) about 0.25% to about 0.75% magnesium stearate by weight of the tablet core;
wherein the semipermeable membrane coating comprises about 73% to about 77% cellulose acetate by weight of the semipermeable membrane coating and about 27% to about 23% plasticizer by weight of the semipermeable membrane coating, and wherein the weight ratio of the semipermeable membrane coating to the tablet core is about 0.03:1 to about 0.07:1. In some embodiments, the plasticizer is polyethylene glycol. In some embodiments, the weight ratio of the semipermeable membrane coating to the tablet core is about 0.05:1. In some embodiments, the weight ratio of the semipermeable membrane coating to the tablet core is about 0.045:1.

In some embodiments, at least 99% or 100% of the amount of dexpramipexole, or a pharmaceutically acceptable salt thereof, is in the tablet core and the homogeneous mixture in the tablet core consists essentially of:
(a) about 42% dexpramipexole dihydrochloride monohydrate by weight of the tablet core;
(b) about 31% microcrystalline cellulose by weight of the tablet core;
(c) about 7% polyvinylpyrrolidone-vinyl acetate copolymer by weight of the tablet core;
(d) about 20% of sodium chloride by weight of the tablet core; and
(e) about 0.5% magnesium stearate by weight of the tablet core;
wherein the semipermeable membrane coating comprises about 75% cellulose acetate by weight of the semipermeable membrane coating and about 25% plasticizer by weight of the semipermeable membrane coating, and wherein the weight ratio of the semipermeable membrane coating to the tablet core is about 0.03:1 to about 0.07:1. In some embodiments, the plasticizer is polyethylene glycol. In some embodiments, the weight ratio of the semipermeable membrane coating to the tablet core is about 0.05:1. In some embodiments, the weight ratio of the semipermeable membrane coating to the tablet core is about 0.045:1.

In some embodiments, at least 99% or 100% of the amount of dexpramipexole, or a pharmaceutically acceptable salt thereof, is in the tablet core and the homogeneous mixture in the tablet core consists essentially of:
(a) about 40% to about 45% dexpramipexole dihydrochloride monohydrate by weight of the tablet core;
(b) about 28% to about 33% microcrystalline cellulose by weight of the tablet core;
(c) about 4.5% to about 9.5% polyvinylpyrrolidone-vinyl acetate copolymer by weight of the tablet core;
(d) about 17.5% to about 22.5% of sodium chloride by weight of the tablet core; and
(e) about 0.25% to about 0.75% magnesium stearate by weight of the tablet core;
wherein the semipermeable membrane coating comprises about 68% to about 72% cellulose acetate by weight of the semipermeable membrane coating and about 32% to about 28% plasticizer by weight of the semipermeable membrane coating, and wherein the weight ratio of the semipermeable membrane coating to the tablet core is about 0.05:1 to about 0.09:1. In some embodiments, the plasticizer is polyethylene glycol. In some embodiments, the weight ratio of the semipermeable membrane coating to the tablet core is about 0.07:1.

In some embodiments, at least 99% or 100% of the amount of dexpramipexole, or a pharmaceutically acceptable salt thereof, is in the tablet core and the homogeneous mixture in the tablet core consists essentially of:
(a) about 42% dexpramipexole dihydrochloride monohydrate by weight of the tablet core;
(b) about 31% microcrystalline cellulose by weight of the tablet core;
(c) about 7% polyvinylpyrrolidone-vinyl acetate copolymer by weight of the tablet core;
(d) about 20% of sodium chloride by weight of the tablet core; and
(e) about 0.5% magnesium stearate by weight of the tablet core;
wherein the semipermeable membrane coating comprises about 70% cellulose acetate by weight of the semipermeable membrane coating and about 30% plasticizer by weight of the semipermeable membrane coating, and wherein the weight ratio of the semipermeable membrane coating to the tablet core is about 0.05:1 to about 0.09:1. In some embodiments, the plasticizer is polyethylene glycol. In some embodiments, the weight ratio of the semipermeable membrane coating to the tablet core is about 0.07:1.

In some embodiments, at least 99% or 100% of the amount of dexpramipexole, or a pharmaceutically acceptable salt thereof, is in the tablet core and the homogeneous mixture in the tablet core consists essentially of:
(a) about 315 mg to about 325 mg dexpramipexole dihydrochloride monohydrate;
(b) about 28% to about 33% microcrystalline cellulose by weight of the tablet core;
(c) about 4.5% to about 9.5% polyvinylpyrrolidone-vinyl acetate copolymer by weight of the tablet core;
(d) about 17.5% to about 22.5% of sodium chloride by weight of the tablet core; and
(e) about 0.25% to about 0.75% magnesium stearate by weight of the tablet core;
   wherein the semipermeable membrane coating comprises about 83% to about 87% cellulose acetate by weight of the semipermeable membrane coating and about 17% to about 13% plasticizer by weight of the semipermeable membrane coating, and wherein the weight ratio of the semipermeable membrane coating to the tablet core is about 0.06:1 to about 0.08:1. In some embodiments, the plasticizer is polyethylene glycol. In some embodiments, the weight ratio of the semipermeable membrane coating to the tablet core is about 0.06:1. In some embodiments, the weight ratio of the semipermeable membrane coating to the tablet core is about 0.062:1. In some embodiments, the weight ratio of the semipermeable membrane coating to the tablet core is about 0.08:1.

In some embodiments, at least 99% or 100% of the amount of dexpramipexole, or a pharmaceutically acceptable salt thereof, is in the tablet core and the homogeneous mixture in the tablet core consists essentially of:
(a) about 319 mg dexpramipexole dihydrochloride monohydrate;
(b) about 31% microcrystalline cellulose by weight of the tablet core;
(c) about 7% polyvinylpyrrolidone-vinyl acetate copolymer by weight of the tablet core;
(d) about 20% of sodium chloride by weight of the tablet core; and
(e) about 0.5% magnesium stearate by weight of the tablet core;
   wherein the semipermeable membrane coating comprises about 85% cellulose acetate by weight of the semipermeable membrane coating and about 15% plasticizer by weight of the semipermeable membrane coating, and wherein the weight ratio of the semipermeable membrane coating to the tablet core is about 0.06:1 to about 0.08:1. In some embodiments, the plasticizer is polyethylene glycol. In some embodiments, the weight ratio of the semipermeable membrane coating to the tablet core is about 0.06:1. In some embodiments, the weight ratio of the semipermeable membrane coating to the tablet core is about 0.062:1. In some embodiments, the weight ratio of the semipermeable membrane coating to the tablet core is about 0.08:1.

In some embodiments, at least 99% or 100% of the amount of dexpramipexole, or a pharmaceutically acceptable salt thereof, is in the tablet core and the homogeneous mixture in the tablet core consists essentially of:
(a) about 390 mg to about 410 mg dexpramipexole dihydrochloride monohydrate;
(b) about 28% to about 33% microcrystalline cellulose by weight of the tablet core;
(c) about 4.5% to about 9.5% polyvinylpyrrolidone-vinyl acetate copolymer by weight of the tablet core;
(d) about 17.5% to about 22.5% of sodium chloride by weight of the tablet core; and
(e) about 0.25% to about 0.75% magnesium stearate by weight of the tablet core;
wherein the semipermeable membrane coating comprises about 81% to about 85% cellulose acetate by weight of the semipermeable membrane coating and about 19% to about 15% plasticizer by weight of the semipermeable membrane coating, and wherein the weight ratio of the semipermeable membrane coating to the tablet core is about 0.06:1 to about 0.08:1. In some embodiments, the plasticizer is polyethylene glycol. In some embodiments, the weight ratio of the semipermeable membrane coating to the tablet core is about 0.07:1. In some embodiments, the weight ratio of the semipermeable membrane coating to the tablet core is about 0.067:1.

In some embodiments, at least 99% or 100% of the amount of dexpramipexole, or a pharmaceutically acceptable salt thereof, is in the tablet core and the homogeneous mixture in the tablet core consists essentially of:
(a) about 400 mg dexpramipexole dihydrochloride monohydrate;
(b) about 31% microcrystalline cellulose by weight of the tablet core;
(c) about 7% polyvinylpyrrolidone-vinyl acetate copolymer by weight of the tablet core;
(d) about 20% of sodium chloride by weight of the tablet core; and
(e) about 0.5% magnesium stearate by weight of the tablet core;
wherein the semipermeable membrane coating comprises about 83% cellulose acetate by weight of the semipermeable membrane coating and about 17% plasticizer by weight of the semipermeable membrane coating, and wherein the weight ratio of the semipermeable membrane coating to the tablet core is about 0.06:1 to about 0.08:1. In some embodiments, the plasticizer is polyethylene glycol. In some embodiments, the weight ratio of the semipermeable membrane coating to the tablet core is about 0.07:1. In some embodiments, the weight ratio of the semipermeable membrane coating to the tablet core is about 0.067:1.

In some embodiments, at least 99% or 100% of the amount of dexpramipexole, or a pharmaceutically acceptable salt thereof, is in the tablet core and the homogeneous mixture in the tablet core consists essentially of:
(a) about 315 mg to about 325 mg dexpramipexole dihydrochloride monohydrate;
(b) about 28% to about 33% microcrystalline cellulose by weight of the tablet core;
(c) about 4.5% to about 9.5% polyvinylpyrrolidone-vinyl acetate copolymer by weight of the tablet core;
(d) about 17.5% to about 22.5% of sodium chloride by weight of the tablet core; and
(e) about 0.25% to about 0.75% magnesium stearate by weight of the tablet core;
wherein the semipermeable membrane coating comprises about 73% to about 77% cellulose acetate by weight of the semipermeable membrane coating and about 27% to about 23% plasticizer by weight of the semipermeable membrane coating, and wherein the weight ratio of the semipermeable membrane coating to the tablet core is about 0.03:1 to about 0.07:1. In some embodiments, the plasticizer is polyethylene glycol. In some embodiments, the weight ratio of the semipermeable membrane coating to the tablet core is about 0.05:1. In some embodiments, the weight ratio of the semipermeable membrane coating to the tablet core is about 0.045:1.

In some embodiments, at least 99% or 100% of the amount of dexpramipexole, or a pharmaceutically acceptable salt thereof, is in the tablet core and the homogeneous mixture in the tablet core consists essentially of:
(a) about 319 mg dexpramipexole dihydrochloride monohydrate;
(b) about 31% microcrystalline cellulose by weight of the tablet core;
(c) about 7% polyvinylpyrrolidone-vinyl acetate copolymer by weight of the tablet core;
(d) about 20% of sodium chloride by weight of the tablet core; and
(e) about 0.5% magnesium stearate by weight of the tablet core;
wherein the semipermeable membrane coating comprises about 75% cellulose acetate by weight of the semipermeable membrane coating and about 25% plasticizer by weight of the semipermeable membrane coating, and wherein the weight ratio of the semipermeable membrane coating to the tablet core is about 0.03:1 to about 0.07:1. In some embodiments, the plasticizer is polyethylene glycol. In some embodiments, the weight ratio of the semipermeable membrane coating to the tablet core is about 0.05:1. In some embodiments, the weight ratio of the semipermeable membrane coating to the tablet core is about 0.045:1.

In some embodiments, at least 99% or 100% of the amount of dexpramipexole, or a pharmaceutically acceptable salt thereof, is in the tablet core and the homogeneous mixture in the tablet core consists essentially of:
(a) about 315 mg to about 325 mg dexpramipexole dihydrochloride monohydrate;
(b) about 28% to about 33% microcrystalline cellulose by weight of the tablet core;
(c) about 4.5% to about 9.5% polyvinylpyrrolidone-vinyl acetate copolymer by weight of the tablet core;
(d) about 17.5% to about 22.5% of sodium chloride by weight of the tablet core; and
(e) about 0.25% to about 0.75% magnesium stearate by weight of the tablet core;

wherein the semipermeable membrane coating comprises about 68% to about 72% cellulose acetate by weight of the semipermeable membrane coating and about 32% to about 28% plasticizer by weight of the semipermeable membrane coating, and wherein the weight ratio of the semipermeable membrane coating to the tablet core is about 0.05:1 to about 0.09:1. In some embodiments, the plasticizer is polyethylene glycol. In some embodiments, the weight ratio of the semipermeable membrane coating to the tablet core is about 0.07:1.

In some embodiments, at least 99% or 100% of the amount of dexpramipexole, or a pharmaceutically acceptable salt thereof, is in the tablet core and the homogeneous mixture in the tablet core consists essentially of:
(a) about 319 mg dexpramipexole dihydrochloride monohydrate;
(b) about 31% microcrystalline cellulose by weight of the tablet core;
(c) about 7% polyvinylpyrrolidone-vinyl acetate copolymer by weight of the tablet core;
(d) about 20% of sodium chloride by weight of the tablet core; and
(e) about 0.5% magnesium stearate by weight of the tablet core;
wherein the semipermeable membrane coating comprises about 70% cellulose acetate by weight of the semipermeable membrane coating and about 30% plasticizer by weight of the semipermeable membrane coating, and wherein the weight ratio of the semipermeable membrane coating to the tablet core is about 0.05:1 to about 0.09:1. In some embodiments, the plasticizer is polyethylene glycol. In some embodiments, the weight ratio of the semipermeable membrane coating to the tablet core is about 0.07:1.

### 5. Immediate Release Drug Coating

In general, the tablet core and the semipermeable membrane coating surrounding the tablet core as described herein provide dexpramipexole, or a pharmaceutically acceptable salt thereof, in a sustained release form. In embodiments wherein about 70% or more than about 70%, but less than 100% of the amount of dexpramipexole, or a pharmaceutically acceptable salt thereof, in the pharmaceutical composition is in the tablet core, the remaining amount of dexpramipexole, or a pharmaceutically acceptable salt thereof, is provided in an immediate release form within the pharmaceutical composition. Thus, in some aspects, the present disclosure is directed to a pharmaceutical composition comprising dexpramipexole, or a pharmaceutically acceptable salt thereof, in both a sustained release and an immediate release form.

In certain such embodiments, wherein the pharmaceutical composition comprises dexpramipexole, or a pharmaceutically acceptable salt thereof, in both a sustained release and an immediate release form, the immediate release form of dexpramipexole, or a pharmaceutically acceptable salt thereof, is provided in form of an immediate release drug coating (briefly referred to herein also as “drug coating”) surrounding the semipermeable membrane coating. Within the meaning of the present disclosure, “an immediate release drug coating surrounding the semipermeable membrane coating” does not exclude that further coatings might be present between the immediate release drug coating and the semipermeable membrane coating, such as a seal coating (see section 6 below). In general, the drug coating is present in an amount sufficient to provide complete coverage of the tablet core and the semipermeable membrane coating and optionally also the seal coating if present.

In the embodiments described below in this section (“5. Immediate release drug coating”), the tablet further comprises an immediate release drug coating surrounding the semipermeable membrane coating.

In some embodiments, about 70% to about 99% of the amount of dexpramipexole, or a pharmaceutically acceptable salt thereof, is in the tablet core and about 30% to about 1% of the amount of dexpramipexole, or a pharmaceutically acceptable salt thereof, is in the drug coating. In some embodiments, about 75% to about 98% of the amount of dexpramipexole, or a pharmaceutically acceptable salt thereof, is in the tablet core and about 25% to about 2% of the amount of dexpramipexole, or a pharmaceutically acceptable salt thereof, is in the drug coating. In some embodiments, about 80% to about 95% of the amount of dexpramipexole, or a pharmaceutically acceptable salt thereof, is in the tablet core and about 20% to about 5% of the amount of dexpramipexole, or a pharmaceutically acceptable salt thereof, is in the drug coating. In some embodiments, about 80% to about 90% of the amount of dexpramipexole, or a pharmaceutically acceptable salt thereof, is in the tablet core and about 20% to about 10% of the amount of dexpramipexole, or a pharmaceutically acceptable salt thereof, is in the drug coating. In some embodiments, about 81% to about 89% of dexpramipexole, or a pharmaceutically acceptable salt thereof, is in the tablet core and about 19% to about 11% of the amount of dexpramipexole, or a pharmaceutically acceptable salt thereof, is in the drug coating. In some embodiments, about 82% to about 88% of the amount of dexpramipexole, or a pharmaceutically acceptable salt thereof, is in the tablet core and about 18% to about 12% of the amount of dexpramipexole, or a pharmaceutically acceptable salt thereof, is in the drug coating. In some embodiments, about 83% to about 87% of dexpramipexole, or a pharmaceutically acceptable salt thereof, is in the tablet core and about 17% to about 13% of the amount of dexpramipexole, or a pharmaceutically acceptable salt thereof, is in the drug coating. In some embodiments, about 84% to about 86% of the amount of dexpramipexole, or a pharmaceutically acceptable salt thereof, is in the tablet core and about 16% to about 14% of the amount of dexpramipexole, or a pharmaceutically acceptable salt thereof, is in the drug coating. In specific embodiments, about 85% of the amount of dexpramipexole, or a pharmaceutically acceptable salt thereof, is in the tablet core and about 15% of the amount of dexpramipexole, or a pharmaceutically acceptable salt thereof, is in the drug coating.

In some embodiments, about 70% of the amount of dexpramipexole, or a pharmaceutically acceptable salt thereof, is in the tablet core and about 30% of the amount of dexpramipexole, or a pharmaceutically acceptable salt thereof, is in the drug coating. In some embodiments, about 80% of the amount of dexpramipexole, or a pharmaceutically acceptable salt thereof, is in the tablet core and about 20% of the amount of dexpramipexole, or a pharmaceutically acceptable salt thereof, is in the drug coating. In some embodiments, about 85% of the amount of dexpramipexole, or a pharmaceutically acceptable salt thereof, is in the tablet core and about 15% of the amount of dexpramipexole, or a pharmaceutically acceptable salt thereof, is in the drug coating. In some embodiments, about 90% of the amount of dexpramipexole, or a pharmaceutically acceptable salt thereof, is in the tablet core and about 10% of the amount of dexpramipexole, or a pharmaceutically acceptable salt thereof, is in the drug coating.

In some embodiments, about 30%, about 29%, about 28%, about 27%, about 26%, about 25%, about 24%, about 23%, about 22%, about 21%, about 20%, about 19%, about 18%, about 17%, about 16%, about 15%, about 14%, about 13%, about 12%, about 11%, about 10%, about 9%, about 8%, about 7%, about 6%, about 5%, about 4%, about 3%, about 2%, or about 1% of the amount of dexpramipexole, or a pharmaceutically acceptable salt thereof, is in the drug coating.

In specific embodiments, about 85% of the amount of dexpramipexole, or a pharmaceutically acceptable salt thereof, is in the tablet core and about 15% of the amount of dexpramipexole, or a pharmaceutically acceptable salt thereof, is in the drug coating.

In some embodiments, the drug coating comprises about 30 mg to about 60 mg of dexpramipexole dihydrochloride equivalent. In some embodiments, the drug coating comprises about 35 mg to about 55 mg of dexpramipexole dihydrochloride equivalent. In some embodiments, the drug coating comprises about 40 mg to about 50 mg of dexpramipexole dihydrochloride equivalent. In some embodiments, the drug coating comprises about 41 mg to about 49 mg, about 42 mg to about 48 mg, about 43 mg to about 47 mg, or about 44 mg to about 46 mg of dexpramipexole dihydrochloride equivalent. In specific embodiments, the drug coating comprises about 45 mg of dexpramipexole dihydrochloride equivalent.

In some embodiments, the drug coating comprises about 30 mg, about 31 mg, about 32 mg, about 33 mg, about 34 mg, about 35 mg, about 36 mg, about 37 mg, about 38 mg, about 39 mg, about 40 mg, about 41 mg, about 42 mg, about 43 mg, about 44 mg, about 45 mg, about 46 mg, about 47 mg, about 48 mg, about 49 mg, about 50 mg, about 51 mg, about 52 mg, about 53 mg, about 54 mg, about 55 mg, about 56 mg, about 57 mg, about 58 mg, about 59 mg, or about 60 mg.

In some embodiments, the tablet core comprises about 240 mg to about 270 mg dexpramipexole dihydrochloride equivalent and the drug coating comprises about 30 mg to about 60 mg dexpramipexole dihydrochloride equivalent. In some embodiments, the tablet core comprises about 270 mg dexpramipexole dihydrochloride equivalent and the drug coating comprises about 30 mg dexpramipexole dihydrochloride equivalent. In some embodiments, the tablet core comprises about 250 mg to about 260 mg dexpramipexole dihydrochloride equivalent and the drug coating comprises about 40 mg to about 50 mg dexpramipexole dihydrochloride equivalent. In specific embodiments, the tablet core comprises about 255 mg dexpramipexole dihydrochloride equivalent and the drug coating comprises about 45 mg dexpramipexole dihydrochloride equivalent.

In some embodiments, the drug coating further comprises a binder. Providing a binder in a drug coating can enhance the adherence of the drug coating to other coatings in the tablet that are in contact with the drug coating (such as a seal coating or a semipermeable membrane coating; and/or a film coating). Exemplary suitable binders are hydroxypropyl methylcellulose, polyethylene glycol, polypropylene glycol, polyoxyethylene-polypropylene copolymer, polyethylene ester, polyethylene sorbitan ester, polyethylene oxide, polyvinyl alcohol, cellulose acetate, or any combination thereof. In some embodiments, the binder comprises hydroxypropyl methylcellulose, polyethylene glycol, or both. In specific embodiments, the binder comprises hydroxypropyl methylcellulose and polyethylene glycol. In other specific embodiments, the binder consists of hydroxypropyl methylcellulose and polyethylene glycol. In some embodiments, the drug coating consists of dexpramipexole, or a pharmaceutically acceptable salt thereof, and the binder.

In some embodiments, the weight ratio of dexpramipexole dihydrochloride monohydrate equivalent to binder in the drug coating is about 1:1 to about 1:4. In some embodiments, the weight ratio of dexpramipexole dihydrochloride monohydrate equivalent to binder in the drug coating is about 1:2 to about 1:4. In some embodiments, the weight ratio of dexpramipexole dihydrochloride monohydrate equivalent to binder in the drug coating is about 1:2.5 to about 1:3.5. In specific embodiments, the weight ratio of dexpramipexole dihydrochloride monohydrate equivalent to binder in the drug coating is about 1:3. In even more specific embodiments, the weight ratio of dexpramipexole dihydrochloride monohydrate equivalent to binder in the drug coating is about 1:3 and the binder comprises hydroxypropyl methylcellulose and polyethylene glycol. In other specific embodiments, the weight ratio of dexpramipexole dihydrochloride monohydrate equivalent to binder in the drug coating is about 1:2.

In some embodiments, the weight ratio of dexpramipexole dihydrochloride monohydrate equivalent to binder in the drug coating is about 1:1, about 1:1.5, about 1:2, about 1:2.5, about 1:3, about 1:3.5, or about 1:4.

In some embodiments, the drug coating comprises about 30 mg to about 200 mg of binder. In some embodiments, the drug coating comprises about 40 mg to about 150 mg of binder. In some embodiments, the drug coating comprises about 30 mg, about 35 mg, about 40 mg, about 45 mg, about 50 mg, about 55 mg, about 60 mg, about 65 mg, about 70 mg, about 75 mg, about 80 mg, about 85 mg, about 90 mg, about 95 mg, about 100 mg, about 105 mg, about 110 mg, about 115 mg, about 120 mg, about 125 mg, about 130 mg, about 135 mg, about 140 mg, about 145 mg, about 150 mg, about 155 mg, about 160 mg, about 165 mg, about 170 mg, about 175 mg, about 180 mg, about 185 mg, about 190 mg, about 195 mg, or about 200 mg of binder. In specific embodiments, the drug coating comprises about 45 mg of binder. In specific embodiments, the drug coating comprises about 50 mg of binder. In specific embodiments, the drug coating comprises about 48 mg of binder.

In some embodiments, the drug coating comprises about 130 mg to about 160 mg or about 135 mg to about 155 mg of binder. In some embodiments, the drug coating comprises about 140 mg to about 150 mg or about 140 mg to about 148 mg of binder. In some embodiments, the drug coating comprises about 142 mg to about 146 mg of binder. In specific embodiments, the drug coating comprises about 144 mg of binder. In some embodiments, the drug coating comprises about 130 mg, about 132 mg, about 134 mg, about 136 mg, about 138 mg, about 140 mg, about 142 mg, about 144 mg, about 146 mg, about 148 mg, about 150 mg, about 152 mg, about 154 mg, about 156 mg, about 158 mg, or about 160 mg of binder.

In some embodiments, the drug coating comprises about 80 mg to about 110 mg or about 85 mg to about 105 mg of binder. In some embodiments, the drug coating comprises about 90 mg to about 100 mg or about 94 mg to about 98 mg of binder. In some embodiments, the drug coating comprises about 95 mg to about 97 mg of binder. In specific embodiments, the drug coating comprises about 96 mg of binder. In some embodiments, the drug coating comprises about 80 mg, about 82 mg, about 84 mg, about 86 mg, about 88 mg, about 90 mg, about 92 mg, about 94 mg, about 96 mg, about 98 mg, about 100 mg, about 102 mg, about 104 mg, about 106 mg, about 108 mg, or about 110 mg of binder.

In some embodiments, the pharmaceutical composition comprises about 30 mg to about 200 mg of drug coating. In some embodiments, the pharmaceutical composition comprises about 40 mg to about 150 mg of drug coating. In some embodiments, the pharmaceutical composition comprises about 30 mg, about 35 mg, about 40 mg, about 45 mg, about 50 mg, about 55 mg, about 60 mg, about 65 mg, about 70 mg, about 75 mg, about 80 mg, about 85 mg, about 90 mg, about 95 mg, about 100 mg, about 105 mg, about 110 mg, about 115 mg, about 120 mg, about 125 mg, about 130 mg, about 135 mg, about 140 mg, about 145 mg, about 150 mg, about 155 mg, about 160 mg, about 165 mg, about 170 mg, about 175 mg, about 180 mg, about 185 mg, about 190 mg, about 195 mg, or about 200 mg of drug coating. In specific embodiments, the pharmaceutical composition comprises about 90 mg of drug coating. In specific embodiments, the pharmaceutical composition comprises about 95 mg of drug coating. In specific embodiments, the pharmaceutical composition comprises about 100 mg of drug coating. In specific embodiments, the pharmaceutical composition comprises about 96 mg of drug coating.

In some embodiments, the pharmaceutical composition comprises about 170 mg to about 210 mg drug coating. In some embodiments, the pharmaceutical composition comprises about 175 mg to about 205 mg drug coating. In some embodiments, the pharmaceutical composition comprises about 180 mg to about 200 mg drug coating. In some embodiments, the pharmaceutical composition comprises about 185 mg to about 195 mg drug coating. In some embodiments, the pharmaceutical composition comprises about 186 mg to about 194 mg, about 187 mg to about 193 mg, or about 188 mg to about 192 mg drug coating. In some embodiments, the pharmaceutical composition comprises about 189 mg to about 193 mg drug coating. In some embodiments, the pharmaceutical composition comprises about 190 mg to about 192 mg drug coating. In specific embodiments, the pharmaceutical composition comprises about 191 mg drug coating. In some embodiments, the pharmaceutical composition comprises about 170 mg, about 171 mg, about 172 mg, about 173 mg, about 174 mg, about 175 mg, about 176 mg, about 177 mg, about 178 mg, about 179 mg, about 180 mg, about 181 mg, about 182 mg, about 183 mg, about 184 mg, about 185 mg, about 186 mg, about 187 mg, about 188 mg, about 189 mg, about 190 mg, about 191 mg, about 192 mg, about 193 mg, about 194 mg, about 195 mg, about 196 mg, about 197 mg, about 198 mg, about 199 mg, about 200 mg, about 201 mg, about 202 mg, about 203 mg, about 204 mg, about 205 mg, about 206 mg, about 207 mg, about 208 mg, about 209 mg, or about 210 mg drug coating.

In some embodiments, the pharmaceutical composition comprises about 120 mg to about 160 mg drug coating. In some embodiments, the pharmaceutical composition comprises about 125 mg to about 155 mg drug coating. In some embodiments, the pharmaceutical composition comprises about 130 mg to about 150 mg drug coating. In some embodiments, the pharmaceutical composition comprises about 135 mg to about 145 mg drug coating. In some embodiments, the pharmaceutical composition comprises about 140 mg to about 145 mg drug coating, such as about 144 mg. In some embodiments, the pharmaceutical composition comprises about 120 mg, about 121 mg, about 122 mg, about 123 mg, about 124 mg, about 125 mg, about 126 mg, about 127 mg, about 128 mg, about 129 mg, about 130 mg, about 131 mg, about 132 mg, about 133 mg, about 134 mg, about 135 mg, about 136 mg, about 137 mg, about 138 mg, about 139 mg, about 140 mg, about 141 mg, about 142 mg, about 143 mg, about 144 mg, about 145 mg, about 146 mg, about 147 mg, about 148 mg, about 149 mg, about 150 mg, about 151 mg, about 152 mg, about 153 mg, about 154 mg, about 155 mg, about 156 mg, about 157 mg, about 158 mg, about 159 mg, or about 160 mg drug coating.

In some embodiments, the weight ratio of the drug coating to the tablet core is about 0.1:1 to about 0.5:1, about 0.11:1 to about 0.49:1, about 0.12:1 to about 0.48:1, about 0.13:1 to about 0.47:1, about 0.14:1 to about 0.46:1, about 0.15:1 to about 0.45:1, about 0.16:1 to about 0.44:1, about 0.17:1 to about 0.43:1, about 0.18:1 to about 0.42:1, about 0.19:1 to about 0.41:1, about 0.20:1 to about 0.40:1, about 0.21:1 to about 0.39:1, about 0.22:1 to about 0.38:1, about 0.23:1 to about 0.37:1, about 0.24:1 to about 0.36:1, about 0.25:1 to about 0.35:1, about 0.26:1 to about 0.34:1, about 0.27:1 to about 0.33:1, or about 0.28:1 to about 0.32:1. In some embodiments, the weight ratio of the drug coating to the tablet core is about 0.26:1 to about 0.28:1. In some embodiments, the weight ratio of the drug coating to the tablet core is about 0.27:1. In some embodiments, the weight ratio of the drug coating to the tablet core is about 0.24:1 to about 0.3:1. In some embodiments, the weight ratio of the drug coating to the tablet core is about 0.25:1 to about 0.29:1. In some embodiments, the weight ratio of the drug coating to the tablet core is about 0.26:1 to about 0.28:1. In some embodiments, the weight ratio of the drug coating to the tablet core is about 0.27:1. In some embodiments, the weight ratio of the drug coating to the tablet core is about 0.29:1 to about 0.31:1. In some embodiments, the weight ratio of the drug coating to the tablet core is about 0.30:1. In some embodiments, the weight ratio of the drug coating to the tablet core is about 0.296:1. In some embodiments, the weight ratio of the drug coating to the tablet core is about 0.3:1. In some embodiments, the weight ratio of the drug coating to the tablet core is about 0.26:1, about 0.27:1, about 0.28:1, about 0.29:1, about 0.30:1, about 0.31:1, about 0.32:1, about 0.33:1, or about 0.34:1.

In some embodiments, the weight ratio of the drug coating to the tablet core is about 0.1:1. In some embodiments, the weight ratio of the drug coating to the tablet core is about 0.11:1. In some embodiments, the weight ratio of the drug coating to the tablet core is about 0.12:1. In some embodiments, the weight ratio of the drug coating to the tablet core is about 0.13:1. In some embodiments, the weight ratio of the drug coating to the tablet core is about 0.14:1. In some embodiments, the weight ratio of the drug coating to the tablet core is about 0.15:1.

In some embodiments, the weight ratio of the drug coating to the tablet core is about 0.18:1 to about 0.26:1. In some embodiments, the weight ratio of the drug coating to the tablet core is about 0.19:1 to about 0.25:1. In some embodiments, the weight ratio of the drug coating to the tablet core is about 0.20:1 to about 0.24:1. In some embodiments, the weight ratio of the drug coating to the tablet core is about 0.21:1 to about 0.23:1. In some embodiments, the weight ratio of the drug coating to the tablet core is about 0.22:1. In some embodiments, the weight ratio of the drug coating to the tablet core is about 0.18:1, about 0.19:1, about 0.20:1, about 0.21:1, about 0.22:1, about 0.23:1, about 0.24:1, about 0.25:1, or about 0.26:1.

In some embodiments, the drug coating constitutes about 10% to about 30% by weight of the tablet. In some embodiments, the drug coating constitutes about 15% to about 25% by weight of the tablet. In some embodiments, the drug coating constitutes about 16% to about 24% or about 17% to about 23% by weight of the tablet. In some embodiments, the drug coating constitutes about 18% to about 22% by weight of the tablet. In some embodiments, the drug coating constitutes about 19% to about 21% by weight of the tablet. In some embodiments, the drug coating constitutes about 20% by weight of the tablet. In some embodiments, the drug coating constitutes about 10% to about 22% by weight of the tablet. In some embodiments, the drug coating constitutes about 12% to about 20% or about 13% to about 19% by weight of the tablet. In some embodiments, the drug coating constitutes about 14% to about 18% by weight of the tablet. In some embodiments, the drug coating constitutes about 15% to about 17% by weight of the tablet. In some embodiments, the drug coating constitutes about 16% by weight of the tablet. In some embodiments, the drug coating constitutes about 10%, about 11%, about 12%, about 13%, about 14%, about 15%, about 16%, about 17%, about 18%, about 19%, or about 20%, about 21%, about 22%, about 23%, about 24%, about 25%, about 26%, about 27%, about 28%, about 29%, or about 30% by weight of the tablet.

In some embodiments, the amount of dexpramipexole, or a pharmaceutically acceptable salt thereof, in the tablet core is in the form of dexpramipexole dihydrochloride or a hydrate thereof. In some embodiments, the amount of dexpramipexole, or a pharmaceutically acceptable salt thereof that is in the immediate release drug coating is in the form of dexpramipexole dihydrochloride or a hydrate thereof. In some embodiments, the amount of dexpramipexole, or a pharmaceutically acceptable salt thereof, in the tablet core and the amount of dexpramipexole, or a pharmaceutically acceptable salt thereof that is in the immediate release drug coating is in the form of dexpramipexole dihydrochloride or a hydrate thereof.

In some embodiments, the amount of dexpramipexole, or a pharmaceutically acceptable salt thereof, in the tablet core is in the form of dexpramipexole dihydrochloride monohydrate. In some embodiments, amount of dexpramipexole, or a pharmaceutically acceptable salt thereof, in the immediate release drug coating is in the form of dexpramipexole dihydrochloride monohydrate. In specific embodiments, the amount of dexpramipexole, or a pharmaceutically acceptable salt thereof, in the tablet core is in the form of dexpramipexole dihydrochloride monohydrate and the amount of dexpramipexole, or a pharmaceutically acceptable salt thereof, in the drug coating is in the form of dexpramipexole dihydrochloride monohydrate.

In some embodiments, the amount of dexpramipexole, or a pharmaceutically acceptable salt thereof, in the tablet core is in the form of dexpramipexole dihydrochloride. In some embodiments, amount of dexpramipexole, or a pharmaceutically acceptable salt thereof, in the immediate release drug coating is in the form of dexpramipexole dihydrochloride. In specific embodiments, the amount of dexpramipexole, or a pharmaceutically acceptable salt thereof, in the tablet core is in the form of dexpramipexole dihydrochloride and the amount of dexpramipexole, or a pharmaceutically acceptable salt thereof, in the drug coating is in the form of dexpramipexole dihydrochloride.

### 6. Seal Coating

In some embodiments described above wherein the pharmaceutical composition further comprises an immediate release drug coating surrounding the semipermeable membrane coating, the tablet further comprises a seal coating between the semipermeable membrane coating and the immediate release drug coating. In such embodiments, the seal coating surrounds the semipermeable membrane coating and the immediate release drug coating surrounds the seal coating.

In some embodiments, the seal coating is a nonfunctional coating. The term “nonfunctional” in this context means that the seal coating is water-soluble and dissolves quickly once it meets aqueous release media, thus not having substantial effect on drug release properties of the pharmaceutical composition. However, the term “nonfunctional” in this context does not imply that the seal coating serves no useful purpose. For example, the seal coating can protect the tablet core and semipermeable membrane coating from absorbing moisture. In general, the seal coating is present in an amount sufficient to provide complete coverage of the tablet core and the semipermeable membrane coating.

In some embodiments, the seal coating comprises polyvinyl alcohol, cellulose acetate, methacrylic acid, methyl acrylate, methyl methacrylate, dibutyl sebacate, hydroxypropyl methylcellulose, polyethylene glycol, or any combination thereof. In some embodiments, the seal coating comprises hydroxypropyl methylcellulose and/or polyethylene glycol.

In some embodiments, the seal coating consists of hydroxypropyl methylcellulose and polyethylene glycol.

In some embodiments, the pharmaceutical composition comprises about 10 mg to about 30 mg seal coating. In some embodiments, the pharmaceutical composition comprises about 15 mg to about 25 mg seal coating. In some embodiments, the pharmaceutical composition comprises about 20 mg to about 25 mg seal coating. In some embodiments, the pharmaceutical composition comprises about 16 mg to about 24 mg, about 17 mg to about 23 mg, or about 18 mg to about 22 mg seal coating. In some embodiments, the pharmaceutical composition comprises about 20 mg to about 22 mg seal coating. In some embodiments, the pharmaceutical composition comprises about 21 mg seal coating. In some embodiments, the pharmaceutical composition comprises about 10 mg, about 12 mg, about 14 mg, about 15 mg, about 16 mg, about 18 mg, about 20 mg, about 21 mg, about 22 mg, about 24 mg, about 26 mg, about 28 mg, or about 30 mg seal coating.

In some embodiments, the weight ratio of the seal coating to the tablet core is about 0.01:1 to about 0.05:1. In some embodiments, the weight ratio of the seal coating to the tablet core is about 0.02:1 to about 0.04:1. In some embodiments, the weight ratio of the seal coating to the tablet core is about 0.03:1. In some embodiments, the weight ratio of the seal coating to the tablet core is about 0.032:1. In some embodiments, the weight ratio of the seal coating to the tablet core is about 0.01:1, about 0.02:1, about 0.03:1, about 0.04:1, or about 0.05:1.

In some embodiments, the seal coating constitutes about 1% to about 4% by weight of the tablet. In some embodiments, the seal coating constitutes about 1.5% to about 3.5% or about 1.5% to about 3% or about 1.5% to about 2.5% or about 1.5% to about 2% by weight of the tablet. In some embodiments, the seal coating constitutes about 2% to about 3% or about 2% to about 2.5% by weight of the tablet. In some embodiments, the seal coating constitutes about 2% by weight of the tablet. In some embodiments, the seal coating constitutes about 2.2% by weight of the tablet.

In some embodiments, the homogeneous mixture in the tablet core consists essentially of:
(a) about 265 mg to about 275 mg dexpramipexole dihydrochloride monohydrate;
(b) about 192 mg to about 202 mg microcrystalline cellulose;
(c) about 40 mg to about 50 mg polyvinylpyrrolidone-vinyl acetate copolymer;
(d) about 125 mg to about 135 mg sodium chloride; and
(e) about 1 mg to about 5 mg magnesium stearate;
   wherein the tablet comprises about 47 mg to about 57 mg semipermeable membrane coating, wherein the semipermeable membrane coating comprises about 83% to about 87% cellulose acetate by weight of the semipermeable membrane coating and about 17% to about 13% plasticizer by weight of the semipermeable membrane coating;
   wherein the tablet further comprises about 15 mg to about 25 mg of a seal coating surrounding the semipermeable membrane coating, wherein the seal coating comprises hydroxypropyl methylcellulose and polyethylene glycol; and
   wherein the tablet further comprises about 185 mg to about 195 mg of an immediate release drug coating surrounding the seal coating, wherein the drug coating comprises about 45 mg to about 50 mg dexpramipexole dihydrochloride monohydrate and about 140 mg to about 150 mg of a binder, wherein the binder comprises hydroxypropyl methylcellulose and polyethylene glycol.

In some embodiments, the homogeneous mixture in the tablet core consists essentially of:
(a) about 271 mg dexpramipexole dihydrochloride monohydrate;
(b) about 197 mg microcrystalline cellulose;
(c) about 45 mg polyvinylpyrrolidone-vinyl acetate copolymer;
(d) about 129 mg sodium chloride; and
(e) about 3 mg magnesium stearate;
   wherein the tablet comprises about 52 mg semipermeable membrane coating, wherein the semipermeable membrane coating comprises about 85% cellulose acetate by weight of the semipermeable membrane coating and about 15% plasticizer by weight of the semipermeable membrane coating;
   wherein the tablet further comprises about 21 mg of a seal coating surrounding the semipermeable membrane coating, wherein the seal coating comprises hydroxypropyl methylcellulose and polyethylene glycol; and
   wherein the tablet further comprises about 191.4 mg of an immediate release drug coating surrounding the seal coating, wherein the drug coating comprises about 47.85 mg dexpramipexole dihydrochloride monohydrate and about 143.55 mg of a binder, wherein the binder comprises hydroxypropyl methylcellulose and polyethylene glycol.

In some embodiments, the homogeneous mixture in the tablet core consists essentially of:
(a) about 265 mg to about 275 mg dexpramipexole dihydrochloride monohydrate;
(b) about 192 mg to about 202 mg microcrystalline cellulose;
(c) about 40 mg to about 50 mg polyvinylpyrrolidone-vinyl acetate copolymer;
(d) about 125 mg to about 135 mg sodium chloride; and
(e) about 1 mg to about 5 mg magnesium stearate;
   wherein the tablet comprises about 47 mg to about 57 mg semipermeable membrane coating, wherein the semipermeable membrane coating comprises about 83% to about 87% cellulose acetate by weight of the semipermeable membrane coating and about 17% to about 13% plasticizer by weight of the semipermeable membrane coating;
   wherein the tablet further comprises about 15 mg to about 25 mg of a seal coating surrounding the semipermeable membrane coating, wherein the seal coating comprises hydroxypropyl methylcellulose and polyethylene glycol; and
   wherein the tablet further comprises about 91 mg to about 101 mg of an immediate release drug coating surrounding the seal coating, wherein the drug coating comprises about 45 mg to about 50 mg dexpramipexole dihydrochloride monohydrate and about 45 mg to about 50 mg of a binder, wherein the binder comprises hydroxypropyl methylcellulose and polyethylene glycol.

In some embodiments, the homogeneous mixture in the tablet core consists essentially of:
(a) about 271 mg dexpramipexole dihydrochloride monohydrate;
(b) about 197 mg microcrystalline cellulose;
(c) about 45 mg polyvinylpyrrolidone-vinyl acetate copolymer;
(d) about 129 mg sodium chloride; and
(e) about 3 mg magnesium stearate;
   wherein the tablet comprises about 52 mg semipermeable membrane coating, wherein the semipermeable membrane coating comprises about 85% cellulose acetate by weight of the semipermeable membrane coating and about 15% plasticizer by weight of the semipermeable membrane coating;
   wherein the tablet further comprises about 21 mg of a seal coating surrounding the semipermeable membrane coating, wherein the seal coating comprises hydroxypropyl methylcellulose and polyethylene glycol; and
   wherein the tablet further comprises about 96 mg of an immediate release drug coating surrounding the seal coating, wherein the drug coating comprises about 47.85 mg dexpramipexole dihydrochloride monohydrate and about 47.85 mg of a binder, wherein the binder comprises hydroxypropyl methylcellulose and polyethylene glycol.

In some embodiments, the homogeneous mixture in the tablet core consists essentially of:
(a) about 40% to about 45% dexpramipexole dihydrochloride monohydrate by weight of the tablet core;
(b) about 28% to about 33% microcrystalline cellulose by weight of the tablet core;
(c) about 4.5% to about 9.5% polyvinylpyrrolidone-vinyl acetate copolymer by weight of the tablet core;
(d) about 17.5% to about 22.5% of sodium chloride by weight of the tablet core; and
(e) about 0.25% to about 0.75% magnesium stearate by weight of the tablet core;
   wherein the semipermeable membrane coating comprises about 83% to about 87% cellulose acetate by weight of the semipermeable membrane coating and about 17% to about 13% plasticizer by weight of the semipermeable membrane coating and wherein the weight ratio of the semipermeable membrane coating to the tablet core is about 0.07:1 to about 0.09:1; wherein the tablet further comprises a seal coating surrounding the semipermeable membrane coating, wherein the seal coating comprises hydroxypropyl methylcellulose and polyethylene glycol, and wherein the weight ratio of the seal coating to the tablet core is about 0.02:1 to about 0.04:1; and
   wherein the tablet further an immediate release drug coating surrounding the seal coating, wherein the drug coating comprises about 45 mg to about 50 mg dexpramipexole dihydrochloride monohydrate and about 140 mg to about 150 mg of a binder, wherein the binder comprises hydroxypropyl methylcellulose and polyethylene glycol, and wherein the weight ratio of the drug coating to the tablet core is about 0.29:1 to about 0.31:1. In some embodiments, the weight ratio of the semipermeable membrane coating to the tablet core is about 0.08:1, the weight ratio of the seal coating to the tablet core is about 0.03:1, and the weight ratio of the drug coating to the tablet core is about 0.30:1.

In some embodiments, the homogeneous mixture in the tablet core consists essentially of:
(a) about 42% dexpramipexole dihydrochloride monohydrate by weight of the tablet core;
(b) about 31% microcrystalline cellulose by weight of the tablet core;
(c) about 7% polyvinylpyrrolidone-vinyl acetate copolymer by weight of the tablet core;
(d) about 20% of sodium chloride by weight of the tablet core; and
(e) about 0.5% magnesium stearate by weight of the tablet core;
   wherein the semipermeable membrane coating comprises about 85% cellulose acetate by weight of the semipermeable membrane coating and about 15% plasticizer by weight of the semipermeable membrane coating and wherein the weight ratio of the semipermeable membrane coating to the tablet core is about 0.07:1 to about 0.09:1;
   wherein the tablet further comprises a seal coating surrounding the semipermeable membrane coating, wherein the seal coating comprises hydroxypropyl methylcellulose and polyethylene glycol, and wherein the weight ratio of the seal coating to the tablet core is about 0.02:1 to about 0.04:1; and
   wherein the tablet further an immediate release drug coating surrounding the seal coating, wherein the drug coating comprises about 48 mg dexpramipexole dihydrochloride monohydrate and about 144 mg of a binder, wherein the binder comprises hydroxypropyl methylcellulose and polyethylene glycol, and wherein the weight ratio of the drug coating to the tablet core is about 0.29:1 to about 0.31:1. In some embodiments, the weight ratio of the semipermeable membrane coating to the tablet core is about 0.08:1, the weight ratio of the seal coating to the tablet core is about 0.03:1, and the weight ratio of the drug coating to the tablet core is about 0.30:1.

In some embodiments, the homogeneous mixture in the tablet core consists essentially of:
(a) about 40% to about 45% dexpramipexole dihydrochloride monohydrate by weight of the tablet core;
(b) about 28% to about 33% microcrystalline cellulose by weight of the tablet core;
(c) about 4.5% to about 9.5% polyvinylpyrrolidone-vinyl acetate copolymer by weight of the tablet core;
(d) about 17.5% to about 22.5% of sodium chloride by weight of the tablet core; and
(e) about 0.25% to about 0.75% magnesium stearate by weight of the tablet core;
   wherein the semipermeable membrane coating comprises about 83% to about 87% cellulose acetate by weight of the semipermeable membrane coating and about 17% to about 13% plasticizer by weight of the semipermeable membrane coating and wherein the weight ratio of the semipermeable membrane coating to the tablet core is about 0.07:1 to about 0.09:1;
   wherein the tablet further comprises a seal coating surrounding the semipermeable membrane coating, wherein the seal coating comprises hydroxypropyl methylcellulose and polyethylene glycol, and wherein the weight ratio of the seal coating to the tablet core is about 0.02:1 to about 0.04:1; and
   wherein the tablet further an immediate release drug coating surrounding the seal coating, wherein the drug coating comprises about 45 mg to about 50 mg dexpramipexole dihydrochloride monohydrate and about 45 mg to about 50 mg of a binder, wherein the binder comprises hydroxypropyl methylcellulose and polyethylene glycol, and wherein the weight ratio of the drug coating to the tablet core is about 0.1:1 to about 0.2:1. In some embodiments, the weight ratio of the semipermeable membrane coating to the tablet core is about 0.08:1, the weight ratio of the seal coating to the tablet core is about 0.03:1, and the weight ratio of the drug coating to the tablet core is about 0.15:1.

In some embodiments, the homogeneous mixture in the tablet core consists essentially of:
(a) about 42% dexpramipexole dihydrochloride monohydrate by weight of the tablet core;
(b) about 31% microcrystalline cellulose by weight of the tablet core;
(c) about 7% polyvinylpyrrolidone-vinyl acetate copolymer by weight of the tablet core;
(d) about 20% of sodium chloride by weight of the tablet core; and
(e) about 0.5% magnesium stearate by weight of the tablet core;
   wherein the semipermeable membrane coating comprises about 85% cellulose acetate by weight of the semipermeable membrane coating and about 15% plasticizer by weight of the semipermeable membrane coating and wherein the weight ratio of the semipermeable membrane coating to the tablet core is about 0.07:1 to about 0.09:1;
   wherein the tablet further comprises a seal coating surrounding the semipermeable membrane coating, wherein the seal coating comprises hydroxypropyl methylcellulose and polyethylene glycol, and wherein the weight ratio of the seal coating to the tablet core is about 0.02:1 to about 0.04:1; and
   wherein the tablet further an immediate release drug coating surrounding the seal coating, wherein the drug coating comprises about 48 mg dexpramipexole dihydrochloride monohydrate and about 48 mg of a binder, wherein the binder comprises hydroxypropyl methylcellulose and polyethylene glycol, and wherein the weight ratio of the drug coating to the tablet core is about 0.13:1 to about 0.17:1. In some embodiments, the weight ratio of the semipermeable membrane coating to the tablet core is about 0.08:1, the weight ratio of the seal coating to the tablet core is about 0.03:1, and the weight ratio of the drug coating to the tablet core is about 0.15:1.

In some embodiments, the homogeneous mixture in the tablet core consists essentially of:
(a) about 265 mg to about 275 mg dexpramipexole dihydrochloride monohydrate;
(b) about 28% to about 33% microcrystalline cellulose by weight of the tablet core;
(c) about 4.5% to about 9.5% polyvinylpyrrolidone-vinyl acetate copolymer by weight of the tablet core;
(d) about 17.5% to about 22.5% of sodium chloride by weight of the tablet core; and
(e) about 0.25% to about 0.75% magnesium stearate by weight of the tablet core;
   wherein the semipermeable membrane coating comprises about 83% to about 87% cellulose acetate by weight of the semipermeable membrane coating and about 17% to about 13% plasticizer by weight of the semipermeable membrane coating and wherein the weight ratio of the semipermeable membrane coating to the tablet core is about 0.07:1 to about 0.09:1; wherein the tablet further comprises a seal coating surrounding the semipermeable membrane coating, wherein the seal coating comprises hydroxypropyl methylcellulose and polyethylene glycol, and wherein the weight ratio of the seal coating to the tablet core is about 0.02:1 to about 0.04:1; and
   wherein the tablet further an immediate release drug coating surrounding the seal coating, wherein the drug coating comprises about 45 mg to about 50 mg dexpramipexole dihydrochloride monohydrate and about 140 mg to about 150 mg of a binder, wherein the binder comprises hydroxypropyl methylcellulose and polyethylene glycol, and wherein the weight ratio of the drug coating to the tablet core is about 0.29:1 to about 0.31:1. In some embodiments, the weight ratio of the semipermeable membrane coating to the tablet core is about 0.08:1, the weight ratio of the seal coating to the tablet core is about 0.03:1, and the weight ratio of the drug coating to the tablet core is about 0.30:1.

In some embodiments, the homogeneous mixture in the tablet core consists essentially of:
(a) about 271 mg dexpramipexole dihydrochloride monohydrate;
(b) about 31% microcrystalline cellulose by weight of the tablet core;
(c) about 7% polyvinylpyrrolidone-vinyl acetate copolymer by weight of the tablet core;
(d) about 20% of sodium chloride by weight of the tablet core; and
(e) about 0.5% magnesium stearate by weight of the tablet core;
   wherein the semipermeable membrane coating comprises about 85% cellulose acetate by weight of the semipermeable membrane coating and about 15% plasticizer by weight of the semipermeable membrane coating and wherein the weight ratio of the semipermeable membrane coating to the tablet core is about 0.07:1 to about 0.09:1;
   wherein the tablet further comprises a seal coating surrounding the semipermeable membrane coating, wherein the seal coating comprises hydroxypropyl methylcellulose and polyethylene glycol, and wherein the weight ratio of the seal coating to the tablet core is about 0.02:1 to about 0.04:1; and
   wherein the tablet further an immediate release drug coating surrounding the seal coating, wherein the drug coating comprises about 48 mg dexpramipexole dihydrochloride monohydrate and about 144 mg of a binder, wherein the binder comprises hydroxypropyl methylcellulose and polyethylene glycol, and wherein the weight ratio of the drug coating to the tablet core is about 0.29:1 to about 0.31:1. In some embodiments, the weight ratio of the semipermeable membrane coating to the tablet core is about 0.08:1, the weight ratio of the seal coating to the tablet core is about 0.03:1, and the weight ratio of the drug coating to the tablet core is about 0.30:1.

In some embodiments, the homogeneous mixture in the tablet core consists essentially of:
(a) about 265 mg to about 275 mg dexpramipexole dihydrochloride monohydrate;
(b) about 28% to about 33% microcrystalline cellulose by weight of the tablet core;
(c) about 4.5% to about 9.5% polyvinylpyrrolidone-vinyl acetate copolymer by weight of the tablet core;
(d) about 17.5% to about 22.5% of sodium chloride by weight of the tablet core; and
(e) about 0.25% to about 0.75% magnesium stearate by weight of the tablet core;
   wherein the semipermeable membrane coating comprises about 83% to about 87% cellulose acetate by weight of the semipermeable membrane coating and about 17% to about 13% plasticizer by weight of the semipermeable membrane coating and wherein the weight ratio of the semipermeable membrane coating to the tablet core is about 0.07:1 to about 0.09:1;
   wherein the tablet further comprises a seal coating surrounding the semipermeable membrane coating, wherein the seal coating comprises hydroxypropyl methylcellulose and polyethylene glycol, and wherein the weight ratio of the seal coating to the tablet core is about 0.02:1 to about 0.04:1; and
   wherein the tablet further an immediate release drug coating surrounding the seal coating, wherein the drug coating comprises about 45 mg to about 50 mg dexpramipexole dihydrochloride monohydrate and about 45 mg to about 50 mg of a binder, wherein the binder comprises hydroxypropyl methylcellulose and polyethylene glycol, and wherein the weight ratio of the drug coating to the tablet core is about 0.1:1 to about 0.2:1. In some embodiments, the weight ratio of the semipermeable membrane coating to the tablet core is about 0.08:1, the weight ratio of the seal coating to the tablet core is about 0.03:1, and the weight ratio of the drug coating to the tablet core is about 0.15:1.

In some embodiments, the homogeneous mixture in the tablet core consists essentially of:
(a) about 271 mg dexpramipexole dihydrochloride monohydrate;
(b) about 31% microcrystalline cellulose by weight of the tablet core;
(c) about 7% polyvinylpyrrolidone-vinyl acetate copolymer by weight of the tablet core;
(d) about 20% of sodium chloride by weight of the tablet core; and
(e) about 0.5% magnesium stearate by weight of the tablet core;
   wherein the semipermeable membrane coating comprises about 85% cellulose acetate by weight of the semipermeable membrane coating and about 15% plasticizer by weight of the semipermeable membrane coating and wherein the weight ratio of the semipermeable membrane coating to the tablet core is about 0.07:1 to about 0.09:1;
   wherein the tablet further comprises a seal coating surrounding the semipermeable membrane coating, wherein the seal coating comprises hydroxypropyl methylcellulose and polyethylene glycol, and wherein the weight ratio of the seal coating to the tablet core is about 0.02:1 to about 0.04:1; and
   wherein the tablet further an immediate release drug coating surrounding the seal coating, wherein the drug coating comprises about 48 mg dexpramipexole dihydrochloride monohydrate and about 48 mg of a binder, wherein the binder comprises hydroxypropyl methylcellulose and polyethylene glycol, and wherein the weight ratio of the drug coating to the tablet core is about 0.13:1 to about 0.17:1. In some embodiments, the weight ratio of the semipermeable membrane coating to the tablet core is about 0.08:1, the weight ratio of the seal coating to the tablet core is about 0.03:1, and the weight ratio of the drug coating to the tablet core is about 0.15:1.

### 7. Film Coating

In some embodiments described above, 100% of the amount of dexpramipexole, or a pharmaceutically acceptable salt thereof, is in the tablet core. In such embodiments, the entire amount of dexpramipexole, or a pharmaceutically acceptable salt thereof, is provided in a sustained release form in the pharmaceutical composition. The pharmaceutical composition of such embodiments comprises the tablet core as described above and the semipermeable membrane coating as described above. In certain such embodiments, the pharmaceutical composition further comprises a film coating as described in this section, the film coating surrounding the semipermeable membrane coating. In general, the film coating is applied in an amount sufficient to provide complete coverage of the tablet core and the semipermeable membrane coating.

In some embodiments described above, about 70% or more than about 70%, but less than 100% of the amount of dexpramipexole, or a pharmaceutically acceptable salt thereof, is in the tablet core. In such embodiments, the amount of dexpramipexole, or a pharmaceutically acceptable salt thereof, that is in the tablet core is provided in a sustained release form and the remaining amount of dexpramipexole, or a pharmaceutically acceptable salt thereof, is provided in an immediate release form in the pharmaceutical composition. In certain such embodiments, the pharmaceutical composition comprises the tablet core as described above, the semipermeable membrane coating as described above, the immediate release drug coating as described above, and optionally the seal coating between the semipermeable membrane coating and the immediate release coating as described above. In some of those embodiments, the pharmaceutical composition further comprises a film coating as described in this section, the film coating surrounding the immediate release drug coating. In general, the film coating is applied in an amount sufficient to provide complete coverage of the tablet core, the semipermeable membrane coating, the drug coating, and optionally also the seal coating if present.

In some embodiments, the film coating is a nonfunctional coating. The term “nonfunctional” in this context means having no substantial effect on drug release properties of the pharmaceutical composition, but does not imply that the film coating serves no useful purpose. For example, such film coating can impart a distinctive appearance to the tablet (such as providing for a specific color of the tablets), provide protection against attrition during packaging and transportation, and improve ease of swallowing. In some embodiments, the film coating provides a moisture-barrier.

In some embodiments, the film coating further comprises a color additive. A color additive, as defined by regulation, is any dye, pigment, or substance that can provide color to a food, drug, or cosmetic. Examples of suitable color additives include but are not limited to iron oxide (e.g., ferric oxide red), lead oxide, and copper sulfate.

In general, a tablet can comprise more than one film coating.

In some embodiments, the film coating comprises cellulose acetate, methacrylic acid, methyl acrylate, methyl methacrylate, dibutyl sebacate, polyethylene glycol, polyvinyl alcohol, hydroxypropyl methylcellulose (HPMC), hydroxypropyl cellulose (HPC), or any combination thereof.

In some embodiments, the film coating comprises polyvinyl alcohol. In some specific embodiments, the film coating comprises polyvinyl alcohol, titanium dioxide, polyethylene glycol, and talc. In other specific embodiments, the film coating consists of polyvinyl alcohol, titanium dioxide, polyethylene glycol, and talc.

In some embodiments, the pharmaceutical composition comprises about 10 mg to about 40 mg film coating. In some embodiments, the pharmaceutical composition comprises about 15 mg to about 35 mg film coating. In some embodiments, the pharmaceutical composition comprises about 20 mg to about 30 mg film coating. In some embodiments, the pharmaceutical composition comprises about 22 mg to about 26 mg film coating. In some embodiments, the pharmaceutical composition comprises about 24 mg film coating. In some embodiments, the pharmaceutical composition comprises about 25 mg to about 29 mg film coating. In some embodiments, the pharmaceutical composition comprises about 27 mg film coating. In some embodiments, the pharmaceutical composition comprises about 10 mg, about 12 mg, about 14 mg, about 15 mg, about 16 mg, about 18 mg, about 20 mg, about 21 mg, about 22 mg, about 23 mg, about 24 mg, about 25 mg, about 26 mg, about 27 mg, about 28 mg, about 29 mg, about 30 mg, about 32 mg, about 34 mg, about 35 mg, about 36 mg, about 38 mg, or about 40 mg film coating.

In some embodiments, the weight ratio of the film coating to the tablet core is about 0.01:1 to about 0.06:1. In some embodiments, the weight ratio of the film coating to the tablet core is about 0.02:1 to about 0.05:1. In some embodiments, the weight ratio of the film coating to the tablet core is about 0.03:1 to about 0.04:1. In some embodiments, the weight ratio of the film coating to the tablet core is about 0.03:1. In some embodiments, the weight ratio of the film coating to the tablet core is about 0.04:1. In some embodiments, the weight ratio of the film coating to the tablet core is about 0.032:1. In some embodiments, the weight ratio of the film coating to the tablet core is about 0.042:1. In some embodiments, the weight ratio of the film coating to the tablet core is about 0.01:1, about 0.015:1, about 0.02:1, about 0.025:1, about 0.03:1, about 0.035:1, about 0.04:1, about 0.045:1, about 0.05:1, about 0.055:1, or about 0.06:1.

In some embodiments, the film coating constitutes about 1% to about 5% by weight of the tablet. In some embodiments, the film coating constitutes about 2% to about 4% by weight of the tablet. In some embodiments, the film coating constitutes about 3% by weight of the tablet. In some embodiments, the film coating constitutes about 1%, about 1.5%, about 2%, about 2.5%, about 3%, about 3.5%, about 4%, about 4.5%, or about 5% by weight of the tablet.

### 8. Further Embodiments of the Pharmaceutical Composition

In some embodiments, the weight of the tablet is about 1400 mg or less. In some embodiments, the weight of the tablet is about 1300 mg or less. In some embodiments, the weight of the tablet is about 1200 mg or less. In some embodiments, the weight of the tablet is about 1100 mg or less. In some embodiments, the weight of the tablet is about 1000 mg or less. In some embodiments, the weight of the tablet is about 950 mg or less. In some embodiments, the weight of the tablet is about 900 mg or less.

In some embodiments, the weight of the tablet is at least about 100 mg, at least about 150 mg, at least about 200 mg, at least about 250 mg, at least about 300 mg, at least about 350 mg, at least about 400 mg, at least about 450 mg, at least about 500 mg, at least about 550 mg, at least about 600 mg, at least about 650 mg, at least about 700 mg, at least about 750 mg, at least about 800 mg, at least about 850 mg, or at least about 900 mg. In specific embodiments, the weight of the tablet is at least 700 mg. In other specific embodiments, the weight of the tablet is at least about 800 mg. In yet other specific embodiments, the weight of the tablet is at least about 900 mg.

In some embodiments, the weight of the tablet is about 100 mg to about 1500 mg, about 150 mg to about 1500 mg, about 200 mg to about 1500 mg, about 250 mg to about 1500 mg, about 300 mg to about 1500 mg, about 350 mg to about 1500 mg, about 400 mg to about 1500 mg, about 450 mg to about 1500 mg, or about 500 mg to about 1500 mg. In some embodiments, the weight of the tablet is about 550 mg to about 1500 mg, about 600 mg to about 1500 mg, about 650 mg to about 1500 mg, about 700 mg to about 1500 mg, about 750 mg to about 1500 mg, about 800 mg to about 1500 mg, about 850 mg to about 1500 mg, or about 900 mg to about 1500 mg.

In some embodiments, the weight of the tablet is about 100 mg to about 1300 mg, about 150 mg to about 1300 mg, about 200 mg to about 1300 mg, about 250 mg to about 1300 mg, about 300 mg to about 1300 mg, about 350 mg to about 1300 mg, about 400 mg to about 1300 mg, about 450 mg to about 1300 mg, or about 500 mg to about 1300 mg. In some embodiments, the weight of the tablet is about 550 mg to about 1300 mg, about 600 mg to about 1300 mg, about 650 mg to about 1300 mg, about 700 mg to about 1300 mg, about 750 mg to about 1300 mg, about 800 mg to about 1300 mg, about 850 mg to about 1300 mg, or about 900 mg to about 1300 mg.

In some embodiments, the weight of the tablet is about 100 mg to about 1000 mg, about 150 mg to about 1000 mg, about 200 mg to about 1000 mg, about 250 mg to about 1000 mg, about 300 mg to about 1000 mg, about 350 mg to about 1000 mg, about 400 mg to about 1000 mg, about 450 mg to about 1000 mg, or about 500 mg to about 1000 mg. In some embodiments, the weight of the tablet is about 550 mg to about 1000 mg, about 600 mg to about 1000 mg, about 650 mg to about 1000 mg, about 700 mg to about 1000 mg, about 750 mg to about 1000 mg, about 800 mg to about 1000 mg, about 850 mg to about 1000 mg, or about 900 mg to about 1000 mg.

In some embodiments, the weight of the tablet is about 700 mg to about 1000 mg. In some embodiments, the weight of the tablet is about 800 mg to about 1000 mg. In some embodiments, the weight of the tablet is about 900 mg to about 1000 mg. In some embodiments, the weight of the tablet is about 800 mg to about 950 mg. In some embodiments, the weight of the tablet is about 820 mg to about 950 mg.

In some embodiments, the weight of the tablet is about 820 mg to about 840 mg. In some embodiments, the weight of the tablet is about 825 mg to about 835 mg. In some embodiments, the weight of the tablet is about 829 mg to about 833 mg. In some embodiments, the weight of the tablet is about 831 mg.

In some embodiments, the weight of the tablet is about 925 mg to about 945 mg. In some embodiments, the weight of the tablet is about 930 mg to about 940 mg. In some embodiments, the weight of the tablet is about 935 mg to about 940 mg. In some embodiments, the weight of the tablet is about 937 mg.

In specific embodiments, the weight of the tablet is about 1000 mg or less. In other specific embodiments, the weight of the tablet is less than about 1000 mg. In some embodiments, the weight of the tablet is at least about 800 mg.

In some embodiments, the weight of the tablet is about 800 mg, about 805 mg, about 810 mg, about 815 mg, about 820 mg, about 825 mg, about 830 mg, about 831 mg, about 835 mg, about 840 mg, about 845 mg, about 850 mg, about 855 mg, about 860 mg, about 865 mg, about 870 mg, about 875 mg, about 880 mg, about 885 mg, about 890 mg, about 895 mg, about 900 mg, about 905 mg, about 910 mg, about 915 mg, about 920 mg, about 925 mg, about 930 mg, about 935 mg, about 937 mg, about 940 mg, about 945 mg, about 950 mg, about 955 mg, about 960 mg, about 965 mg, about 970 mg, about 975 mg, about 980 mg, about 985 mg, about 990 mg, about 995 mg, about 1000 mg, about 1010 mg, about 1020 mg, about 1030 mg, about 1040 mg, about 1050 mg, about 1060 mg, about 1070 mg, about 1080 mg, about 1090 mg, or about 1100 mg.

Tablets can be of any suitable shape, for example round, oval, elliptic, capsule, cylindrical, square, rectangle, or triangle. In some embodiments, the tablet is an oval shaped tablet. An “oval shaped” tablet has a long axis and a short axis with no flat edged sides. The ratio of the long and short axis can vary, thereby resulting in different shaped oval tablets.

In some embodiments, the length of the short axis of the tablet is about 8.5 mm to about 10.5 mm, the length of the long axis of the tablet is about 16 mm to about 18 mm, and the thickness of the tablet is about 5 mm to about 8 mm. In some embodiments, the length of the short axis of the tablet is about 9 mm to about 10 mm, the length of the long axis of the tablet is about 16.5 mm to about 17.5 mm, and the thickness of the tablet is about 5.5 mm to about 7.5 mm. In specific embodiments, the length of the short axis of the tablet is about 9.5 mm and/or the length of the long axis of the tablet is about 17.2 mm.

In some embodiments, about 10% to about 40%, or about 20% to about 30%, or about 25% of dexpramipexole, or the pharmaceutically acceptable salt thereof, is released at about 4 hours of incubation of the tablet in about 900 mL of 50 mM monobasic potassium phosphate buffer, pH 6.8, at a temperature of 37±0.5° C. as measured using an USP type I apparatus operated at a spindle rotation speed of about 100 rpm. In some embodiments, about 40% to about 60%, or about 40% to about 50%, or about 50% to about 60% of dexpramipexole, or the pharmaceutically acceptable salt thereof, is released at about 8 hours of incubation of the tablet in about 900 mL of 50 mM monobasic potassium phosphate buffer, pH 6.8, at a temperature of 37±0.5° C. as measured using an USP type I apparatus operated at a spindle rotation speed of about 100 rpm. In some embodiments, about 55% to about 85%, or about 60% to about 70%, or about 70% to about 80% of dexpramipexole, or the pharmaceutically acceptable salt thereof, is released at about 12 hours of incubation of the tablet in about 900 mL of 50 mM monobasic potassium phosphate buffer, pH 6.8, at a temperature of 37±0.5° C. as measured using an USP type I apparatus operated at a spindle rotation speed of about 100 rpm. In some embodiments, about 70% to about 95%, or about 75% to about 85%, or about 80% to about 90% of dexpramipexole, or the pharmaceutically acceptable salt thereof, is released at about 16 hours of incubation of the tablet in about 900 mL of 50 mM monobasic potassium phosphate buffer, pH 6.8, at a temperature of 37±0.5° C. as measured using an USP type I apparatus operated at a spindle rotation speed of about 100 rpm. In some embodiments, about 75% to about 100%, or about 80% to about 90%, or about 85% to about 95% of dexpramipexole, or the pharmaceutically acceptable salt thereof, is released at about 20 hours of incubation of the tablet in about 900 mL of 50 mM monobasic potassium phosphate buffer, pH 6.8, at a temperature of 37±0.5° C. as measured using an USP type I apparatus operated at a spindle rotation speed of about 100 rpm. In some embodiments, about 80% to about 100%, or about 80% to about 90%, or about 90% to about 100% of dexpramipexole, or the pharmaceutically acceptable salt thereof, is released at about 24 hours of incubation of the tablet in about 900 mL of 50 mM monobasic potassium phosphate buffer, pH 6.8, at a temperature of 37±0.5° C. as measured using an USP type I apparatus operated at a spindle rotation speed of about 100 rpm.

In some embodiments, at least about 70% but no more than about 80% of dexpramipexole, or the pharmaceutically acceptable salt thereof, is released at about 12 hours of incubation of the tablet in about 900 mL of 50 mM monobasic potassium phosphate buffer, pH 6.8, at a temperature of 37±0.5° C. as measured using an USP type I apparatus operated at a spindle rotation speed of about 100 rpm. In some embodiments, at least about 60% but no more than about 70% of dexpramipexole, or the pharmaceutically acceptable salt thereof, is released at about 12 hours of incubation of the tablet in about 900 mL of 50 mM monobasic potassium phosphate buffer, pH 6.8, at a temperature of 37±0.5° C. as measured using an USP type I apparatus operated at a spindle rotation speed of about 100 rpm. In some embodiments, at least about 75% but no more than about 85% of dexpramipexole, or the pharmaceutically acceptable salt thereof, is released at about 16 hours of incubation of the tablet in about 900 mL of 50 mM monobasic potassium phosphate buffer, pH 6.8, at a temperature of 37±0.5° C. as measured using an USP type I apparatus operated at a spindle rotation speed of about 100 rpm. In some embodiments, at least about 80% but no more than about 90% of dexpramipexole, or the pharmaceutically acceptable salt thereof, is released at about 16 hours of incubation of the tablet in about 900 mL of 50 mM monobasic potassium phosphate buffer, pH 6.8, at a temperature of 37±0.5° C. as measured using an USP type I apparatus operated at a spindle rotation speed of about 100 rpm. In some embodiments, at least about 85% but no more than about 95% of dexpramipexole, or the pharmaceutically acceptable salt thereof, is released at about 24 hours of incubation of the tablet in about 900 mL of 50 mM monobasic potassium phosphate buffer, pH 6.8, at a temperature of 37±0.5° C. as measured using an USP type I apparatus operated at a spindle rotation speed of about 100 rpm. In some embodiments, at least about 90% of dexpramipexole, or the pharmaceutically acceptable salt thereof, is released at about 24 hours of incubation of the tablet in about 900 mL of 50 mM monobasic potassium phosphate buffer, pH 6.8, at a temperature of 37±0.5° C. as measured using an USP type I apparatus operated at a spindle rotation speed of about 100 rpm.

In some embodiments, about 10% to about 30%, or about 15% to about 25% of dexpramipexole, or the pharmaceutically acceptable salt thereof, is released at about 4 hours of incubation of the tablet in about 900 mL of McIlvaine buffer, pH 4.5, at a temperature of 37±0.5° C. as measured using an USP type I apparatus operated at a spindle rotation speed of about 100 rpm. In some embodiments, about 30% to about 50%, or about 35% to about 45% of dexpramipexole, or the pharmaceutically acceptable salt thereof, is released at about 8 hours of incubation of the tablet in about 900 mL of McIlvaine buffer, pH 4.5, at a temperature of 37±0.5° C. as measured using an USP type I apparatus operated at a spindle rotation speed of about 100 rpm. In some embodiments, about 50% to about 70%, or about 50% to about 60%, or about 60% to about 70% of dexpramipexole, or the pharmaceutically acceptable salt thereof, is released at about 12 hours of incubation of the tablet in about 900 mL of McIlvaine buffer, pH 4.5, at a temperature of 37±0.5° C. as measured using an USP type I apparatus operated at a spindle rotation speed of about 100 rpm. In some embodiments, about 65% to about 85%, or about 65% to about 75%, or about 75% to about 85% of dexpramipexole, or the pharmaceutically acceptable salt thereof, is released at about 16 hours of incubation of the tablet in about 900 mL of McIlvaine buffer, pH 4.5, at a temperature of 37±0.5° C. as measured using an USP type I apparatus operated at a spindle rotation speed of about 100 rpm. In some embodiments, about 75% to about 95%, or about 75% to about 80%, or about 85% to about 90% of dexpramipexole, or the pharmaceutically acceptable salt thereof, is released at about 20 hours of incubation of the tablet in about 900 mL of McIlvaine buffer, pH 4.5, at a temperature of 37±0.5° C. as measured using an USP type I apparatus operated at a spindle rotation speed of about 100 rpm. In some embodiments, about 75% to about 95%, or about 75% to about 85%, or about 85% to about 95% of dexpramipexole, or the pharmaceutically acceptable salt thereof, is released at about 24 hours of incubation of the tablet in about 900 mL of McIlvaine buffer, pH 4.5, at a temperature of 37±0.5° C. as measured using an USP type I apparatus operated at a spindle rotation speed of about 100 rpm.

In some embodiments, at least about 50% but no more than about 60% of dexpramipexole, or the pharmaceutically acceptable salt thereof, is released at about 12 hours of incubation of the tablet in about 900 mL of McIlvaine buffer, pH 4.5, at a temperature of 37±0.5° C. as measured using an USP type I apparatus operated at a spindle rotation speed of about 100 rpm. In some embodiments, at least about 60% but no more than about 70% of dexpramipexole, or the pharmaceutically acceptable salt thereof, is released at about 12 hours of incubation of the tablet in about 900 mL of McIlvaine buffer, pH 4.5, at a temperature of 37±0.5° C. as measured using an USP type I apparatus operated at a spindle rotation speed of about 100 rpm. In some embodiments, at least about 65% but no more than about 75% of dexpramipexole, or the pharmaceutically acceptable salt thereof, is released at about 16 hours of incubation of the tablet in about 900 mL of McIlvaine buffer, pH 4.5, at a temperature of 37±0.5° C. as measured using an USP type I apparatus operated at a spindle rotation speed of about 100 rpm. In some embodiments, at least about 75% but no more than about 85% of dexpramipexole, or the pharmaceutically acceptable salt thereof, is released at about 16 hours of incubation of the tablet in about 900 mL of McIlvaine buffer, pH 4.5, at a temperature of 37±0.5° C. as measured using an USP type I apparatus operated at a spindle rotation speed of about 100 rpm. In some embodiments, at least about 75% but no more than about 85% of dexpramipexole, or the pharmaceutically acceptable salt thereof, is released at about 24 hours of incubation of the tablet in about 900 mL of McIlvaine buffer, pH 4.5, at a temperature of 37±0.5° C. as measured using an USP type I apparatus operated at a spindle rotation speed of about 100 rpm. In some embodiments, at least about 85% but no more than about 95% of dexpramipexole, or the pharmaceutically acceptable salt thereof, is released at about 24 hours of incubation of the tablet in about 900 mL of McIlvaine buffer, pH 4.5, at a temperature of 37±0.5° C. as measured using an USP type I apparatus operated at a spindle rotation speed of about 100 rpm.

In some embodiments, about 20% to about 40% or about 25% to about 35% of dexpramipexole, or the pharmaceutically acceptable salt thereof, is released at about 4 hours of incubation of the tablet in about 900 mL of 0.1 N HCl at a temperature of 37±0.5° C. as measured using an USP type I apparatus operated at a spindle rotation speed of about 100 rpm. In some embodiments, about 40% to about 60% or about 45% to about 55% or about 55% to about 65% of dexpramipexole, or the pharmaceutically acceptable salt thereof, is released at about 8 hours of incubation of the tablet in about 900 mL of 0.1 N HCl at a temperature of 37±0.5° C. as measured using an USP type I apparatus operated at a spindle rotation speed of about 100 rpm. In some embodiments, about 60% to about 85%, or about 65% to about 75%, or about 75% to about 85% of dexpramipexole, or the pharmaceutically acceptable salt thereof, is released at about 12 hours of incubation of the tablet in about 900 mL of 0.1 N HCl at a temperature of 37±0.5° C. as measured using an USP type I apparatus operated at a spindle rotation speed of about 100 rpm. In some embodiments, about 75% to about 90%, or about 85% to about 90%, or about 75% to about 85% of dexpramipexole, or the pharmaceutically acceptable salt thereof, is released at about 16 hours of incubation of the tablet in about 900 mL of 0.1 N HCl at a temperature of 37±0.5° C. as measured using an USP type I apparatus operated at a spindle rotation speed of about 100 rpm. In some embodiments, about 80% to about 100%, or about 80% to about 90%, or about 90% to about 95% of dexpramipexole, or the pharmaceutically acceptable salt thereof, is released at about 20 hours of incubation of the tablet in about 900 mL of 0.1 N HCl at a temperature of 37±0.5° C. as measured using an USP type I apparatus operated at a spindle rotation speed of about 100 rpm. In some embodiments, about 80% to about 100%, or about 85% to about 95%, or about 90% to about 100%, or about 85% to about 90% of dexpramipexole, or the pharmaceutically acceptable salt thereof, is released at about 24 hours of incubation of the tablet in about 900 mL of 0.1 N HCl at a temperature of 37±0.5° C. as measured using an USP type I apparatus operated at a spindle rotation speed of about 100 rpm.

In some embodiments, at least about 65% but no more than about 75% of dexpramipexole, or the pharmaceutically acceptable salt thereof, is released at about 12 hours of incubation of the tablet in about 900 mL of 0.1 N HCl at a temperature of 37±0.5° C. as measured using an USP type I apparatus operated at a spindle rotation speed of about 100 rpm. In some embodiments, at least about 75% but no more than about 85% of dexpramipexole, or the pharmaceutically acceptable salt thereof, is released at about 12 hours of incubation of the tablet in about 900 mL of 0.1 N HCl at a temperature of 37±0.5° C. as measured using an USP type I apparatus operated at a spindle rotation speed of about 100 rpm. In some embodiments, at least about 75% but no more than about 85% of dexpramipexole, or the pharmaceutically acceptable salt thereof, is released at about 16 hours of incubation of the tablet in about 900 mL of 0.1 N HCl at a temperature of 37±0.5° C. as measured using an USP type I apparatus operated at a spindle rotation speed of about 100 rpm. In some embodiments, at least about 85% but no more than about 95% of dexpramipexole, or the pharmaceutically acceptable salt thereof, is released at about 16 hours of incubation of the tablet in about 900 mL of 0.1 N HCl at a temperature of 37±0.5° C. as measured using an USP type I apparatus operated at a spindle rotation speed of about 100 rpm. In some embodiments, at least about 85% but no more than about 95% of dexpramipexole, or the pharmaceutically acceptable salt thereof, is released at about 24 hours of incubation of the tablet in about 900 mL of 0.1 N HCl at a temperature of 37±0.5° C. as measured using an USP type I apparatus operated at a spindle rotation speed of about 100 rpm. In some embodiments, at least about 90% of dexpramipexole, or the pharmaceutically acceptable salt thereof, is released at about 24 hours of incubation of the tablet in about 900 mL of 0.1 N HCl at a temperature of 37±0.5° C. as measured using an USP type I apparatus operated at a spindle rotation speed of about 100 rpm.

In some embodiments, the pharmaceutical composition is capable of providing a sigmoidal, pseudo-zero order, or zero order release of dexpramipexole, or a pharmaceutically acceptable salt thereof. In some embodiments, the sigmoidal, pseudo-zero order, or zero order release is from the tablet core of the composition, once the immediate release drug coating is dissolved. In specific embodiments, the release is a zero order release.

In some embodiments, the pharmaceutical composition comprises 0.05% or less pramipexole, or a pharmaceutically acceptable salt thereof, by weight of the tablet. In some embodiments, the pharmaceutical composition comprises 0.04% or less pramipexole, or a pharmaceutically acceptable salt thereof, by weight of the tablet. In some embodiments, the pharmaceutical composition comprises 0.03% or less pramipexole, or a pharmaceutically acceptable salt thereof, by weight of the tablet. In some embodiments, the pharmaceutical composition comprises 0.02% or less pramipexole, or a pharmaceutically acceptable salt thereof, by weight of the tablet. In some embodiments, the pharmaceutical composition comprises 0.015% or less pramipexole, or a pharmaceutically acceptable salt thereof, by weight of the tablet. In some embodiments, the pharmaceutical composition comprises 0.014% or less pramipexole, or a pharmaceutically acceptable salt thereof, by weight of the tablet. In some embodiments, the pharmaceutical composition comprises 0.01% or less pramipexole, or a pharmaceutically acceptable salt thereof, by weight of the tablet.

In specific embodiments, the pharmaceutical composition comprises 0.015% or less pramipexole, or a pharmaceutically acceptable salt thereof, by weight of the tablet.

In some embodiments, the pharmaceutical composition does not comprise pramipexole, or a pharmaceutically acceptable salt thereof, at the detection limit when analyzed by high performance liquid chromatography (HPLC).

In some embodiments, dexpramipexole, or a pharmaceutically acceptable salt thereof, in the pharmaceutical composition has a chiral purity for dexpramipexole, or the pharmaceutically acceptable salt thereof, of 99.95% or more. “Having a chiral purity for dexpramipexole, or the pharmaceutically acceptable salt thereof, of 99.95% or more” means that the portion of pramipexole, or a pharmaceutically acceptable salt of pramipexole (see formula (II) above showing the chemical structure of pramipexole), respectively, in the dexpramipexole, or a pharmaceutically acceptable salt of dexpramipexole in the pharmaceutical composition is 0.05% or less. In some embodiments, dexpramipexole, or a pharmaceutically acceptable salt thereof, in the pharmaceutical composition has a chiral purity for dexpramipexole, or the pharmaceutically acceptable salt thereof, of 99.96% or more. In some embodiments, dexpramipexole, or a pharmaceutically acceptable salt thereof, in the pharmaceutical composition has a chiral purity for dexpramipexole, or the pharmaceutically acceptable salt thereof, of 99.97% or more. In some embodiments, dexpramipexole, or a pharmaceutically acceptable salt thereof, in the pharmaceutical composition has a chiral purity for dexpramipexole, or the pharmaceutically acceptable salt thereof, of 99.98% or more. In some embodiments, dexpramipexole, or a pharmaceutically acceptable salt thereof, in the pharmaceutical composition has a chiral purity for dexpramipexole, or the pharmaceutically acceptable salt thereof, of 99.99% or more. In some embodiments, dexpramipexole, or a pharmaceutically acceptable salt thereof, in the pharmaceutical composition has a chiral purity for dexpramipexole, or the pharmaceutically acceptable salt thereof, of 100%. The chiral purity might be determined by analyzing a sample of dexpramipexole, or a pharmaceutically acceptable salt thereof, by high performance liquid chromatography (HPLC).

In specific embodiments, dexpramipexole, or a pharmaceutically acceptable salt thereof, in the pharmaceutical composition has a chiral purity for dexpramipexole, or the pharmaceutically acceptable salt thereof, of 99.96% or more.

In specific embodiments of the present disclosure, the amount of dexpramipexole, or a pharmaceutically acceptable salt thereof, in the pharmaceutical composition is about 50 mg to about 400 mg (such as about 75 mg, about 150 mg, about 300 mg, or about 376 mg) dexpramipexole dihydrochloride equivalent, the inorganic osmotic agent constitutes about 15% to about 25% by weight of the tablet core, the plasticizer constitutes about 10% to about 40% by weight of the semipermeable membrane coating, and the weight ratio of the semipermeable membrane coating to the tablet core is about 0.04:1 to about 0.09:1. In these specific embodiments, the inorganic osmotic agent may be sodium chloride and/or the plasticizer may be polyethylene glycol. In the specific embodiments described in this paragraph, the pharmaceutical composition may comprise 0.015% or less pramipexole, or a pharmaceutically acceptable salt thereof, by weight of the tablet. The weight of the tablet may be about 1100 mg or less (such as about 800 mg to about 1050 mg).

In specific embodiments of the present disclosure, the amount of dexpramipexole, or a pharmaceutically acceptable salt thereof, in the pharmaceutical composition is about 50 mg to about 350 mg (such as about 75 mg, about 150 mg, or about 300 mg) dexpramipexole dihydrochloride equivalent, the inorganic osmotic agent constitutes about 15% to about 25% by weight of the tablet core, the plasticizer constitutes about 10% to about 20% by weight of the semipermeable membrane coating, and the weight ratio of the semipermeable membrane coating to the tablet core is about 0.06:1 to about 0.08:1. In these specific embodiments, the inorganic osmotic agent may be sodium chloride and/or the plasticizer may be polyethylene glycol. In the specific embodiments described in this paragraph, the pharmaceutical composition may comprise 0.015% or less pramipexole, or a pharmaceutically acceptable salt thereof, by weight of the tablet. The weight of the tablet may be about 1000 mg or less (such as about 800 mg to about 1000 mg).

In more specific embodiments of the present disclosure, the amount of dexpramipexole, or a pharmaceutically acceptable salt thereof, in the pharmaceutical composition is about 250 mg to about 350 mg dexpramipexole dihydrochloride equivalent, the inorganic osmotic agent constitutes about 15% to about 25% by weight of the tablet core, the plasticizer constitutes about 10% to about 20% by weight of the semipermeable membrane coating, and the weight ratio of the semipermeable membrane coating to the tablet core is about 0.06:1 to about 0.08:1. In these more specific embodiments, the inorganic osmotic agent may be sodium chloride and/or the plasticizer may be polyethylene glycol. In the more specific embodiments described in this paragraph, the pharmaceutical composition may comprise 0.015% or less pramipexole, or a pharmaceutically acceptable salt thereof, by weight of the tablet. The weight of the tablet may be about 1000 mg or less (such as about 800 mg to about 1000 mg).

In even more specific embodiments, the amount of dexpramipexole, or a pharmaceutically acceptable salt thereof, in the pharmaceutical composition is about 300 mg dexpramipexole dihydrochloride equivalent, the inorganic osmotic agent constitutes about 20% by weight of the tablet core, the plasticizer constitutes about 15% by weight of the semipermeable membrane coating, and the weight ratio of the semipermeable membrane coating to the tablet core is about 0.06:1 to about 0.08:1. In these even more specific embodiments, the inorganic osmotic agent may be sodium chloride and/or the plasticizer may be polyethylene glycol. In the even more specific embodiments described in this paragraph, the pharmaceutical composition may comprise 0.015% or less pramipexole, or a pharmaceutically acceptable salt thereof, by weight of the tablet. The weight of the tablet may be about 1000 mg or less (such as about 800 mg to about 1000 mg).

In even more specific embodiments, the amount of dexpramipexole, or a pharmaceutically acceptable salt thereof, in the pharmaceutical composition is about 376 mg dexpramipexole dihydrochloride equivalent, the inorganic osmotic agent constitutes about 20% by weight of the tablet core, the plasticizer constitutes about 15% by weight of the semipermeable membrane coating, and the weight ratio of the semipermeable membrane coating to the tablet core is about 0.04:1 to about 0.09:1. In these even more specific embodiments, the inorganic osmotic agent may be sodium chloride and/or the plasticizer may be polyethylene glycol. In the even more specific embodiments described in this paragraph, the pharmaceutical composition may comprise 0.015% or less pramipexole, or a pharmaceutically acceptable salt thereof, by weight of the tablet. The weight of the tablet may be about 1100 mg or less (such as about 800 mg to about 1050 mg).

In some embodiments, one or more of the ingredients of the pharmaceutical composition other than dexpramipexole, or the pharmaceutically acceptable salt thereof, such as one or more of an inorganic osmotic agent, a lubricant, a binder, and/or a diluent, are “generally recognized as safe” (in short: “GRAS”) by the United States Food and Drug Administration (FDA) and/or are recognized as safe by the provisions of the International Council for Harmonisation of Technical Requirements for Registration of Pharmaceuticals for Human Use (ICH). In some embodiments, one or more of the ingredients of the pharmaceutical composition other than dexpramipexole, or the pharmaceutically acceptable salt thereof, such as one or more of inorganic osmotic agent, a lubricant, a binder, and/or a diluent, are such ingredients that are listed in a Pharmacopoeia, such as the European and/or US Pharmacopoeia.

In some embodiments, the pharmaceutical composition of the present disclosure can be part of a kit comprising the pharmaceutical composition packaged into a container (which might be, for example, a blister package or a bottle), the container being accompanied by a package insert providing pertinent information such as, for example, dosage and administration information, contraindications, precautions, drug interactions and adverse reactions. In some aspects, the kit further comprises a desiccant. A desiccant is a hygroscopic substance that is used to induce or sustain a state of dryness (desiccation) in its vicinity. Examples of desiccants are silica gel, activated charcoal, calcium sulfate, calcium chloride, and molecular sieves (typically, zeolites).

### III—Methods of Treatment or Prevention

In certain aspects, the present disclosure further relates to methods of treating or preventing a disorder, disease, or condition in a human subject in need thereof, the methods comprising orally administering to the human subject the pharmaceutical composition of the present disclosure. Regarding the pharmaceutical composition as referred to in this chapter (“III—Methods of Treatment or Prevention”) below, reference is made to the pharmaceutical composition as described herein (in particular, as described in the chapter “II—Pharmaceutical Compositions” above) (briefly indicated below by reference to “the pharmaceutical composition described herein”). The term “orally administering” as used herein may include the act of self-administration by a human in need thereof or administration by another person such as a health care provider.

In some embodiments, the present disclosure is directed to a method of treating or preventing a respiratory disease in a human subject in need thereof, the method comprising orally administering to the human subject the pharmaceutical composition described herein.

In some embodiments, the present disclosure is directed to a method of treating or preventing asthma in a human subject in need thereof, the method comprising orally administering to the human subject the pharmaceutical composition described herein. Asthma is a heterogeneous disease, usually characterized by chronic airway inflammation. Asthma can be defined by the history of respiratory symptoms such as wheeze, shortness of breath, chest tightness, and cough that vary over time and in intensity, together with expiratory airflow limitation. Asthma is a condition in which the airways narrow due to airways smooth muscle constriction and mucosal swelling. The increase of mucus production in the airway lumen further contributes to obstructing airflow and worsening asthma symptoms. This can make breathing difficult and trigger coughing, wheezing and shortness of breath. Asthma may lead to life-threatening asthma attacks or exacerbations.

In some embodiments, treating asthma comprises a reduction in frequency of asthma exacerbations in the human subject.

In some embodiments, treating asthma comprises that the human subject shows improvement in a measurement selected from the group consisting of forced expiratory volume in 1 second (FEVI), forced vital capacity (FVC), annualized CompEx event rate, morning peak expiratory flow (PEF), score on Asthma Control Questionnaire (ACQ), score of Asthma Quality of Life Questionnaire (AQLQ), score on St. George's Respiratory Questionnaire, and any combinations thereof.

In some embodiments, the asthma is severe asthma. Severe asthma is defined as asthma that is uncontrolled on GINA Step 4 or 5 therapy. “GINA” refers to “Global Initiative For Asthma.”

In some embodiments, the asthma is eosinophilic asthma. Eosinophilic asthma (or “asthma of the eosinophilic phenotype”) is a form of asthma that involves abnormally high levels of eosinophils in the blood or in the lungs. A human subject having eosinophilic asthma has an eosinophil level of at least 150 cells per microliter in the peripheral blood. In some embodiments, the eosinophil level is at least 300 cells per microliter in the peripheral blood.

In some embodiments, the eosinophilic asthma is moderate eosinophilic asthma. In some embodiments, the eosinophilic asthma is severe eosinophilic asthma.

In some embodiments, the present disclosure is directed to a method of treating or preventing chronic obstructive pulmonary disease (COPD) in a human subject in need thereof, the method comprising orally administering to the human subject the pharmaceutical composition described herein. In some embodiments, the chronic obstructive pulmonary disease is emphysema or chronic bronchitis.

In some embodiments, the present disclosure is directed to a method of treating or preventing an eosinophilic disorder (i.e., disorders involving elevated levels of eosinophils) in a human subject in need thereof, the method comprising orally administering to the human subject the pharmaceutical composition described herein.

In some embodiments, the eosinophilic disorder is selected from the group consisting of hypereosinophilic syndrome, chronic rhinosinusitis with nasal polyps, nasal polyposis, atopic dermatitis, eosinophilic granulomatosis with polyangiitis, eosinophilic gastroenteritis, eosinophilic esophagitis, and any combination thereof.

In some embodiments, the present disclosure is directed to a method of treating or preventing eosinophilic granulomatosis with polyangiitis in a human subject in need thereof, the method comprising orally administering to the human subject the pharmaceutical composition described herein. “Eosinophilic granulomatosis with polyangiitis” is also known in the art as Churg-Strauss syndrome.

In some embodiments, the present disclosure is directed to a method of treating or preventing hypereosinophilic syndrome in a human subject in need thereof, the method comprising orally administering to the human subject the pharmaceutical composition described herein. In some embodiments, hypereosinophilic syndrome is characterized by levels of eosinophils at or above about 1,000 cells per microliter in the peripheral blood. In some embodiments, hypereosinophilic syndrome is characterized by levels of eosinophils at or above about 1,500 cells per microliter in the peripheral blood.

In some embodiments, the disclosure is directed to a method of treating or preventing an ear, nose, throat or eye disease in a human subject in need thereof, the method comprising orally administering to the human subject the pharmaceutical composition described herein.

In some embodiments, the disclosure is directed to a method of treating or preventing chronic rhinosinusitis with nasal polyps in a human subject in need thereof, the method comprising orally administering to the human subject the pharmaceutical composition described herein.

In some embodiments, the present disclosure as directed to a method of treating or preventing a gastrointestinal disease in a human subject in need thereof, the method comprising orally administering to the human subject the pharmaceutical composition described herein.

In some embodiments, the present disclosure as directed to a method of treating or preventing eosinophilic esophagitis in a human subject in need thereof, the method comprising orally administering to the human subject the pharmaceutical composition described herein.

In some embodiments, the present disclosure as directed to a method of treating or preventing eosinophilic gastroenteritis in a human subject in need thereof, the method comprising orally administering to the human subject the pharmaceutical composition described herein.

In specific embodiments, the pharmaceutical composition is administered once daily. Once daily is also referred to herein as quaque die (QD). “Once daily” administration as used herein refers to administration about every 24 hours.

In some embodiments, the pharmaceutical composition is administered about every 22 to 26 hours. In some embodiments, the pharmaceutical composition is administered about every 23 to 25 hours.

In some embodiments, the pharmaceutical composition is administered to the subject as maintenance therapy (e.g., administration might occur over a period of several years, or over the lifetime of the human subject).

In some embodiments, the pharmaceutical composition is administered about 2 hours after the human subject has consumed a meal. In some embodiments, the pharmaceutical composition is administered orally with about 200 mL to about 300 mL of water.

In some embodiments, the present disclosure is directed to the pharmaceutical composition described herein (in particular, described above in the chapter “II—Pharmaceutical Compositions”) for use as a medicament.

In some embodiments, the present disclosure is directed to the pharmaceutical composition described herein (in particular, described above in the chapter “II—Pharmaceutical Compositions”) for use in a method of treating or preventing as described herein (in particular, described above in this chapter (“III—Methods of Treatment or Prevention”)).

In some embodiments, the present disclosure is directed to the use of the pharmaceutical composition described herein (in particular, described above in the chapter “II—Pharmaceutical Compositions”) in the manufacture of a medicament for a method of treating or preventing as described herein (in particular, described above in this chapter (“III—Methods of Treatment or Prevention”)).

### IV—Methods of Manufacturing

Although the pharmaceutical composition as described herein can be prepared by any suitable method of manufacturing known to a person of ordinary skill in the art, the present disclosure further relates to a particular suitable method of manufacturing of the pharmaceutical composition as described below in this chapter.

In certain aspects, the present disclosure thus also relates to a method of manufacturing a pharmaceutical composition in the form of an orally deliverable tablet comprising dexpramipexole, or a pharmaceutically acceptable salt thereof, the method comprising:
preparing a pre-blend comprising dexpramipexole, or a pharmaceutically acceptable salt thereof, and an inorganic osmotic agent;
preparing a blend comprising the pre-blend and a lubricant;
compressing the blend to form a tablet core; and
coating the tablet core with a semipermeable membrane coating comprising a plasticizer; wherein dexpramipexole, or the pharmaceutically acceptable salt thereof, constitutes about 37% to about 47% of dexpramipexole dihydrochloride monohydrate equivalent by weight of the blend, wherein the inorganic osmotic agent constitutes about 10% to about 40% by weight of the blend, wherein the semipermeable membrane coating comprises about 5% to about 25% plasticizer by weight of the semipermeable membrane coating and wherein the weight ratio of the semipermeable membrane coating to the tablet core is about 0.03:1 to about 0.11:1. The semipermeable membrane coating surrounds the tablet core.

In some aspects, the pre-blend further comprises additional excipients. In some aspects, the blend further comprises additional excipients.

In some embodiments, the pre-blend comprises an antioxidant. In some embodiments, the antioxidant is capable of acting as a nitrite scavenger. Non-limiting examples of nitrite scavengers include, but are not limitd to, ascorbic acid, L-cysteine, caffeic acid, cysteine HCL, methionine, tartaric acid, gallic acid, uric acid, and sodium sulphite. In some embodiments, the pre-blend comprises one nitrite scavenger. In another embodiment, the pre-blend comprises multiple nitrite scavengers. In some embodiments, the nitrite scavengers constitute about 0.1% to about 1.5% by weight of the tablet core. In some embodiments, the nitrite scavenger constitutes about 0.1% to about 1.0% by weight of the tablet core. In some embodiments, the nitrite scavenger constitutes about 0.1% to about 0.5% by weight of the tablet core. In some embodiments, the nitrite scavenger constitutes about 0.5% to about 1.5% by weight of the tablet core. In some embodiments, the nitrite scavenger constitutes about 1% to about 1.5% by weight of the tablet core. In some embodiments, the nitrite scavenger constitutes about 0.1%, about 0.2%, about 0.3%, about 0.4%, about 0.5%, about 0.6%, about 0.7%, about 0.8%, about 0.9%, about 1%, about 1.1%, about 1.2%, about 1.3%, about 1.4%, or about 1.5% by weight of the tablet core.

In certain aspects, the present disclosure thus also relates to a method of manufacturing a pharmaceutical composition in the form of an orally deliverable tablet comprising dexpramipexole, or a pharmaceutically acceptable salt thereof, the method comprising:
preparing a pre-blend comprising dexpramipexole, or a pharmaceutically acceptable salt thereof, and an inorganic osmotic agent;
preparing a blend comprising the pre-blend and a lubricant;
compressing the blend to form a tablet core; and
coating the tablet core with a semipermeable membrane coating comprising a plasticizer; wherein dexpramipexole, or the pharmaceutically acceptable salt thereof, constitutes about 37% to about 47% of dexpramipexole dihydrochloride monohydrate equivalent by weight of the blend, wherein the inorganic osmotic agent constitutes about 10% to about 40% by weight of the blend, wherein the semipermeable membrane coating comprises about 5% to about 40% plasticizer by weight of the semipermeable membrane coating and wherein the weight ratio of the semipermeable membrane coating to the tablet core is about 0.03:1 to about 0.11:1. The semipermeable membrane coating surrounds the tablet core.

In some aspects, the pre-blend further comprises additional excipients. In some aspects, the blend further comprises additional excipients.

The expression “compressing the blend to form a tablet core” as used in the context of the method of manufacturing described herein does not necessarily imply that the tablet core comprises only the compressed blend. The tablet core may comprise one layer of the compressed blend and one or more additional layers. Consequently, the tablet core may be, for example, a single-layer or a bi-layer tablet core as described further below in this chapter.

In some embodiments, the press speed during the compression of the blend to form a tablet core is about 20 to about 30 rpm, such as about 23 rpm to about 27 rpm. In some embodiments, the press speed during the compression for the blend to form a tablet core is about 25 rpm. In some embodiments, the feeder speed during the compression of the blend to form a tablet core is about 30 to about 40 rpm, such as about 33 rpm to about 37 rpm. In some embodiments, the feeder speed during the compression for the blend to form a tablet core is about 35 rpm. In some embodiments, compression of the blend is performed using a Korsch XL-100 rotary tablet press.

Regarding the chiral purity of dexpramipexole, or a pharmaceutically acceptable salt thereof, applied in the method of manufacturing described herein, reference is generally made to Chapter II, Section 8 above (“Further embodiments of the pharmaceutical composition”). In specific embodiments, dexpramipexole, or the pharmaceutically acceptable salt thereof, applied in the method of manufacturing described herein has a chiral purity for dexpramipexole, or the pharmaceutically salt thereof, of 99.96% or more. In specific embodiments, the pharmaceutical composition comprises 0.015% or less pramipexole, or a pharmaceutically acceptable salt thereof, by weight of the tablet.

In some embodiments, dexpramipexole, or the pharmaceutically acceptable salt thereof, constitutes about 38% to about 46% of dexpramipexole dihydrochloride monohydrate equivalent by weight of the blend. In some embodiments, dexpramipexole, or the pharmaceutically acceptable salt thereof, constitutes about 39% to about 45% of dexpramipexole dihydrochloride monohydrate equivalent by weight of the blend. In some embodiments, dexpramipexole, or the pharmaceutically acceptable salt thereof, constitutes about 40% to about 44% of dexpramipexole dihydrochloride monohydrate equivalent by weight of the blend. In some embodiments, dexpramipexole, or the pharmaceutically acceptable salt thereof, constitutes about 41% to about 43% of dexpramipexole dihydrochloride monohydrate equivalent by weight of the blend. In specific embodiments, dexpramipexole, or the pharmaceutically acceptable salt thereof, constitutes about 42% of dexpramipexole dihydrochloride monohydrate equivalent by weight of the blend. In some embodiments, dexpramipexole, or the pharmaceutically acceptable salt thereof, constitutes about 37%, about 38%, about 39%, about 40%, about 41%, about 42%, about 43%, about 44%, about 45%, about 46%, or about 47% of dexpramipexole dihydrochloride monohydrate equivalent by weight of the blend.

In some embodiments, the inorganic osmotic agent constitutes about 11% to about 39%, about 12% to about 38%, about 13% to about 37%, or about 14% to about 36% by weight of the blend. In some embodiments, the inorganic osmotic agent constitutes about 15% to about 35%, about 16% to about 34%, about 17% to about 33%, about 18% to about 32%, or about 19% to about 31% by weight of the blend. In some embodiments, the inorganic osmotic agent constitutes about 20% to about 30% by weight of the blend. In specific embodiments, the inorganic osmotic agent constitutes about 20% by weight of the blend. In other specific embodiments, the inorganic osmotic agent constitutes about 30% by weight of the blend.

In some embodiments, the inorganic osmotic agent constitutes about 10% to about 30%, about 11% to about 29%, about 12% to about 28%, about 13% to about 27%, about 14% to about 26%, about 15% to about 25%, about 16% to about 24%, about 17% to about 23%, about 18% to about 22%, about 19% to about 21%, or about 20% by weight of the blend.

In some embodiments, the inorganic osmotic agent constitutes about 20% to about 30% by weight of the blend. In some embodiments, the inorganic osmotic agent constitutes about 15% to about 25% by weight of the blend. In some embodiments, the inorganic osmotic agent constitutes about 18% to about 22% by weight of the blend. In specific embodiments, the inorganic osmotic agent constitutes about 20% by weight of the blend.

In some embodiments, the inorganic osmotic agent constitutes about 10%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, about 30%, about 31%, about 32%, about 33%, about 34%, about 35%, about 36%, about 37%, about 38%, about 39%, or about 40% by weight of the blend.

As regards suitable inorganic osmotic agents for use in the method of manufacturing described herein, reference is generally made to Chapter II, Section 3 above (“Tablet core”). In some embodiments, the inorganic osmotic agent is sodium chloride, potassium chloride, magnesium chloride, sodium hydrogen phosphate, potassium hydrogen phosphate, or any combination thereof. In specific embodiments, the inorganic osmotic agent is sodium chloride.

In specific embodiments, the inorganic osmotic agent constitutes about 15% to about 25% (such as about 20%) by weight of the blend. The inorganic osmotic agent may be sodium chloride.

In specific embodiments, dexpramipexole, or the pharmaceutically acceptable salt thereof, constitutes about 40% to about 44% of dexpramipexole dihydrochloride monohydrate equivalent by weight of the blend and the inorganic osmotic agent constitutes about 18% to about 22% by weight of the blend. In more specific embodiments, dexpramipexole, or the pharmaceutically acceptable salt thereof, constitutes about 42% of dexpramipexole dihydrochloride monohydrate equivalent by weight of the blend and the inorganic osmotic agent constitutes about 20% by weight of the blend. The inorganic osmotic agent may be sodium chloride and/or the pharmaceutically acceptable salt of dexpramipexole may be dexpramipexole dihydrochloride monohydrate.

In some embodiments, the blend does not comprise polyethylene oxide. In some embodiments, the blend does not comprise a swellable polymeric osmotic agent.

In some embodiments, the pre-blend further comprises a diluent. In some embodiments, the diluent constitutes about 20% to about 40%, about 21% to about 39%, about 22% to about 38%, about 23% to about 37%, about 24% to about 36%, or about 25% to about 35% by weight of the blend. In some embodiments, the diluent constitutes about 26% to about 34% by weight of the blend. In some embodiments, the diluent constitutes about 27% to about 33% by weight of the blend. In some embodiments, the diluent constitutes about 28% to about 32% by weight of the blend. In some embodiments, the diluent constitutes about 29% to about 31% by weight of the blend. In some embodiments, the diluent constitutes about 29% to about 33% by weight of the blend. In some embodiments, the diluent constitutes about 30% to about 32% by weight of the blend. In some embodiments, the diluent constitutes about 31% by weight of the blend. In some embodiments, the diluent constitutes about 30% to about 31% by weight of the blend. In some embodiments, the diluent constitutes about 20%, about 21%, about 22%, about 23%, about 24%, about 25%, about 26%, about 27%, about 28%, about 29%, about 30%, about 30.5%, about 31%, about 32%, about 33%, about 34%, about 35%, about 36%, about 37%, about 38%, about 39%, or about 40% by weight of the blend. In specific embodiments, the diluent constitutes about 30.5% by weight of the blend. As regards suitable diluents for use in the method of manufacturing described herein, reference is generally made to Chapter II, Section 3 above (“Tablet core”). In specific embodiments, the diluent is microcrystalline cellulose.

In some embodiments, the pre-blend further comprises a binder. In some embodiments, the binder constitutes about 2% to about 12%, about 3% to about 11%, or about 4% to about 10% by weight of the blend. In some embodiments, the binder constitutes about 5% to about 9% by weight of the blend. In some embodiments, the binder constitutes about 6% to about 8% by weight of the blend. In some embodiments, the binder constitutes about 6.5% to about 7.5% by weight of the blend. In specific embodiments, the binder constitutes about 7% by weight of the blend. In some embodiments, the binder constitutes about 2%, about 3%, about 4%, about 5%, about 6%, about 6.5%, about 7%, about 7.5%, about 8%, about 9%, about 10%, about 11%, or about 12% by weight of the blend. As regards suitable binders for use in the method of manufacturing described herein, reference is generally made to Chapter II, Section 3 above (“Tablet core”). In specific embodiments, the binder is polyvinylpyrrolidone-vinyl acetate copolymer.

In specific embodiments, the pre-blend further comprises microcrystalline cellulose, polyvinylpyrrolidone-vinyl acetate copolymer, or both. In more specific embodiments, the pre-blend further comprises microcrystalline cellulose and polyvinylpyrrolidone-vinyl acetate copolymer.

In some embodiments, the lubricant constitutes about 0.1% to about 0.9%, about 0.15% to about 0.85%, about 0.2% to about 0.8%, or about 0.25% to about 0.75% by weight of the blend. In some embodiments, the lubricant constitutes about 0.3% to about 0.7% or about 0.35% to about 0.65% by weight of the blend. In some embodiments, the lubricant constitutes about 0.4% to about 0.6% by weight of the blend. In some embodiments, the lubricant constitutes about 0.45% to about 0.55% by weight of the blend. In specific embodiments, the lubricant constitutes about 0.5% by weight of the blend. In some embodiments, the lubricant constitutes about 0.1%, about 0.15%, about 0.2%, about 0.25%, about 0.3%, about 0.35%, about 0.4%, about 0.45%, about 0.5%, about 0.55%, about 0.6%, about 0.65%, about 0.7%, about 0.75%, about 0.8%, about 0.85%, or about 0.9% by weight of the blend. As regards suitable lubricants for use in the method of manufacturing described herein, reference is generally made to Chapter II, Section 3 above (“Tablet core”). In specific embodiments, the lubricant is magnesium stearate.

In some embodiments, the blend consists essentially of:
(a) about 40% to about 44% dexpramipexole dihydrochloride monohydrate by weight of the blend;
(b) about 28% to about 32% microcrystalline cellulose by weight of the blend;
(c) about 5% to about 9% polyvinylpyrrolidone-vinyl acetate copolymer by weight of the blend;
(d) about 18% to about 22% sodium chloride by weight of the blend; and
(e) about 0.25% to about 0.75% magnesium stearate by weight of the blend,
   wherein sodium chloride is the inorganic osmotic agent and magnesium stearate is the lubricant.

In some embodiments, the blend consists essentially of:
(a) about 42% dexpramipexole dihydrochloride monohydrate by weight of the blend;
(b) about 30.5% microcrystalline cellulose by weight of the blend;
(c) about 7% polyvinylpyrrolidone-vinyl acetate copolymer by weight of the blend;
(d) about 20% sodium chloride by weight of the blend; and
(e) about 0.5% magnesium stearate by weight of the blend,
   wherein sodium chloride is the inorganic osmotic agent and magnesium stearate is the lubricant.

In some embodiments, the tablet core is a single-layer tablet core. A “single-layer” tablet core as used herein in the context of the method of manufacturing of the present disclosure refers to a tablet core consisting of a single layer of the blend described above, which comprises the inorganic osmotic agent and dexpramipexole, or a pharmaceutically acceptable salt thereof, in the amounts specified herein. An example of an osmotic tablet with a single-layer tablet core is an elementary osmotic pump.

In other embodiments, the tablet core is a bi-layer tablet core. A “bi-layer” tablet core as used herein in the context of the method of manufacturing of the present disclosure refers to a tablet core consisting of a first and a second layer. More specifically, the “bi-layer” tablet core consists of a first layer of the blend described above, which comprises the inorganic osmotic agent and dexpramipexole, or the pharmaceutically acceptable salt thereof, in the amounts specified herein, and a second layer, the second layer comprising an additional osmotic agent, such as a swellable polymeric osmotic agent, but the second layer not comprising dexpramipexole, or a pharmaceutically acceptable salt thereof. An example of an osmotic tablet with a bi-layer tablet core is a push-pull osmotic pump.

In some embodiments, the plasticizer in the semipermeable membrane coating constitutes about 5% to about 40%, about 7% to about 38%, about 9% to about 36%, or about 11% to about 34% plasticizer by weight of the semipermeable membrane coating. In some embodiments, the plasticizer constitutes about 10% to about 35% by weight of the semipermeable membrane coating. In specific embodiments, the plasticizer constitutes about 15% by weight of the semipermeable membrane coating. The plasticizer may be polyethylene glycol. In specific embodiments, the plasticizer constitutes about 18% by weight of the semipermeable membrane coating. The plasticizer may be polyethylene glycol. In specific embodiments, the plasticizer constitutes about 25% by weight of the semipermeable membrane coating. The plasticizer may be polyethylene glycol. In specific embodiments, the plasticizer constitutes about 30% by weight of the semipermeable membrane coating. The plasticizer may be polyethylene glycol.

In some embodiments, the plasticizer in the semipermeable membrane coating constitutes about 6% to about 24%, about 7% to about 23%, about 8% to about 22%, or about 9% to about 21% plasticizer by weight of the semipermeable membrane coating. In some embodiments, the plasticizer constitutes about 10% to about 20% by weight of the semipermeable membrane coating. In some embodiments, the plasticizer constitutes about 11% to about 19%, about 12% to about 18%, or about 13% to about 17% by weight of the semipermeable membrane coating. In some embodiments, the plasticizer constitutes about 14% to about 16% by weight of the semipermeable membrane coating. In some embodiments, the plasticizer constitutes about 14.5% to about 15.5% by weight of the semipermeable membrane coating. In specific embodiments, the plasticizer constitutes about 15% by weight of the semipermeable membrane coating. The plasticizer may be polyethylene glycol.

In some embodiments, the plasticizer constitutes about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, about 30%, about 31%, about 32%, about 33%, about 34%, or about 35% by weight of the semipermeable membrane coating. The plasticizer may be polyethylene glycol.

In some embodiments, coating the tablet core with the semipermeable membrane coating comprises spraying a semipermeable membrane coating solution comprising the plasticizer on the tablet core, thereby providing the semipermeable membrane coating surrounding the tablet core. In some embodiments, the pan size used for semipermeable membrane coating is about 22 to about 26 in, such as about 24 in. In some embodiments, the nozzle port opening used for semipermeable membrane coating is about 1.0 to about 1.4 mm, such as about 1.2 mm. In some embodiments, the drum speed used for semipermeable membrane coating is about 6 to about 14 rpm, such as about 8 to about 12 rpm. In some embodiments, the inlet air volume used for semipermeable membrane coating is about 200 to about 350 cfm, such as about 250 to about 300 cfm. In some embodiments, the inlet temperature used for semipermeable membrane coating is about 25° C. to about 45° C., such as about 30° C. to about 40° C. In some embodiments, the product temperature used for semipermeable membrane coating is about 20° C. to about 30° C., such as about 24° C. to about 26° C. In some embodiments, the exhaust air temperature used for semipermeable membrane coating is about 20° C. to about 50° C., such as about 20° C. to about 30° C. or about 49° C. to about 50° C. In some embodiments, the spray rate used for semipermeable membrane coating is about 60 to about 110 g/min, such as about 80 to about 100 g/min or about 70 to about 100 g/min. In some embodiments, the pattern air pressure used for semipermeable membrane coating is about 15 to about 25 psi, such as about 20 psi. In some embodiments, the atomizing air pressure used for semipermeable membrane coating is about 10 to about 20 psi, such as about 15 psi. In some embodiments, semipermeable membrane coating is performed using an O'hara coating machine configured with a fully perforated pan.

In some embodiments, the method further comprises drying the tablet after spraying the semipermeable membrane coating solution on the tablet core.

In some embodiments, the semipermeable membrane coating (solution) in addition to the plasticizer further comprises a polymer that is substantially permeable to water but substantially impermeable to solutes including dexpramipexole, or a pharmaceutically acceptable salt thereof. As regards suitable polymers for use in the method of manufacturing described herein, reference is generally made to Chapter II, Section 4 above (“Semipermeable membrane coating”).

In some embodiments, the semipermeable membrane coating solution in addition to the plasticizer further comprises cellulose acetate. In some embodiments, the plasticizer and cellulose acetate constitute about 3% to about 7% by weight of the semipermeable membrane coating solution. In some embodiments, the plasticizer and cellulose acetate constitute about 4% to about 6% by weight of the semipermeable membrane coating solution. In some embodiments, the plasticizer and cellulose acetate constitute about 5% by weight of the semipermeable membrane coating solution. In some embodiments, the solvent of the semipermeable membrane coating solution is a mixture of acetone and water, such as purified water. In some embodiments, the solvent of the semipermeable membrane coating solution is a mixture of acetone:water (such as purified water) ranging from about 95:5 to about 90:10 (wherein the ratio is given as % values, e.g., 95% acetone and 5% water). In some embodiments, the solvent of the semipermeable membrane coating solution is a mixture of about 95:5, or about 94:6, or about 90:10 acetone:water, such as purified water.

In some embodiments, the semipermeable membrane coating in addition to the plasticizer further comprises cellulose acetate. In some embodiments, the semipermeable membrane coating comprises about 60% to about 95% cellulose acetate by weight of the semipermeable membrane coating. In some embodiments, the semipermeable membrane coating comprises about 62% to about 94%, about 64% to about 93%, about 66% to about 92%, or about 68% to about 91% cellulose acetate by weight of the semipermeable membrane coating. In some embodiments, the semipermeable membrane coating comprises about 65% to about 90% cellulose acetate by weight of the semipermeable membrane coating. In specific embodiments, the semipermeable membrane coating comprises about 70% cellulose acetate by weight of the semipermeable membrane coating. In specific embodiments, the semipermeable membrane coating comprises about 75% cellulose acetate by weight of the semipermeable membrane coating. In specific embodiments, the semipermeable membrane coating comprises about 83% cellulose acetate by weight of the semipermeable membrane coating. In specific embodiments, the semipermeable membrane coating comprises about 85% cellulose acetate by weight of the semipermeable membrane coating.

In some embodiments, the semipermeable membrane coating in addition to the plasticizer further comprises cellulose acetate. In some embodiments, the semipermeable membrane coating comprises about 75% to about 95% cellulose acetate by weight of the semipermeable membrane coating. In some embodiments, the semipermeable membrane coating comprises about 76% to about 94%, about 77% to about 93%, about 78% to about 92%, or about 79% to about 91% cellulose acetate by weight of the semipermeable membrane coating. In some embodiments, the semipermeable membrane coating comprises about 80% to about 90% cellulose acetate by weight of the semipermeable membrane coating. In some embodiments, the semipermeable membrane coating comprises about 81% to about 89% or about 82% to about 88% cellulose acetate by weight of the semipermeable membrane coating. In some embodiments, the semipermeable membrane coating comprises about 83% to about 87% cellulose acetate by weight of the semipermeable membrane coating. In some embodiments, the semipermeable membrane coating comprises about 84% to about 86% cellulose acetate by weight of the semipermeable membrane coating. In specific embodiments, the semipermeable membrane coating comprises about 85% cellulose acetate by weight of the semipermeable membrane coating.

In some embodiments, the semipermeable membrane coating comprises about 65%, about 66%, about 67%, about 68%, about 69%, about 70%, about 71%, about 72%, about 73%, about 74%, about 75%, about 76%, about 77%, about 78%, about 79%, about 80%, about 81%, about 82%, about 83%, about 84%, about 85%, about 86%, about 87%, about 88%, about 89%, about 90%, about 91%, about 92%, about 93%, about 94% or about 95% cellulose acetate by weight of the semipermeable membrane coating.

In some embodiments, the semipermeable membrane coating comprises about 65% to about 95% cellulose acetate by weight of the semipermeable membrane coating and about 35% to about 5% plasticizer by weight of the semipermeable membrane coating. In some embodiments, the semipermeable membrane coating comprises about 70% to about 95% cellulose acetate by weight of the semipermeable membrane coating and about 30% to about 5% plasticizer by weight of the semipermeable membrane coating. In some embodiments, the semipermeable membrane coating comprises about 75% to about 95% cellulose acetate by weight of the semipermeable membrane coating and about 25% to about 5% plasticizer by weight of the semipermeable membrane coating. In some embodiments, the semipermeable membrane coating comprises about 80% to about 90% cellulose acetate by weight of the semipermeable membrane coating and about 20% to about 10% plasticizer by weight of the semipermeable membrane coating. In some embodiments, the semipermeable membrane coating comprises about 83% to about 87% cellulose acetate by weight of the semipermeable membrane coating and about 17% to about 13% plasticizer by weight of the semipermeable membrane coating. In some embodiments, the semipermeable membrane coating comprises about 84% to about 86% cellulose acetate by weight of the semipermeable membrane coating and about 16% to about 14% plasticizer by weight of the semipermeable membrane coating. In specific embodiments, the semipermeable membrane coating comprises about 85% cellulose acetate by weight of the semipermeable membrane coating and about 15% plasticizer by weight of the semipermeable membrane coating. The plasticizer may be polyethylene glycol.

In some embodiments, the weight ratio of the semipermeable membrane coating to the tablet core is about 0.04:1 to about 0.1:1. In some embodiments, the weight ratio of the semipermeable membrane coating to the tablet core is about 0.05:1 to about 0.09:1. In some embodiments, the weight ratio of the semipermeable membrane coating to the tablet core is about 0.06:1 to about 0.08:1.

In some embodiments, the weight ratio of the semipermeable membrane coating to the tablet core is about 0.05:1 to about 0.07:1. In some embodiments, the weight ratio of semipermeable membrane coating to tablet core is about 0.055:1 to about 0.065:1. In some embodiments, the weight ratio of semipermeable membrane coating to tablet core is about 0.055:1. In specific embodiments, the weight ratio of the semipermeable membrane coating to the tablet core is about 0.06:1. In other specific embodiments, the weight ratio of the semipermeable membrane coating to the tablet core is about 0.062:1. In some embodiments, the weight ratio of semipermeable membrane coating to tablet core is about 0.07:1.

In some embodiments, the weight ratio of semipermeable membrane coating to tablet core is about 0.07:1 to about 0.09:1. In some embodiments, the weight ratio of semipermeable membrane coating to tablet core is about 0.075:1 to about 0.085:1. In specific embodiments, the weight ratio of semipermeable membrane coating to tablet core is about 0.08:1.

In specific embodiments, the weight ratio of the semipermeable membrane coating to the tablet core is about 0.06:1 to about 0.08:1.

In some embodiments, the weight ratio of the semipermeable membrane coating to the tablet core is about 0.04:1, about 0.045:1, about 0.05:1, about 0.055:1, about 0.06:1, about 0.062:1, about 0.065:1, about 0.07:1, about 0.075:1, about 0.08:1, about 0.085:1, about 0.09:1, about 0.095:1, or about 0.10:1.

In some embodiments, the semipermeable membrane coating thickness is about 100 μm to about 700 μm.

In some embodiments, the semipermeable membrane coating thickness is about 100 μm to about 300 μm. In some embodiments, the semipermeable membrane coating thickness is about 150 μm to about 250 μm or about 200 μm to about 220 μm. In some embodiments, the semipermeable membrane coating thickness is about 210 μm. In some embodiments, the semipermeable membrane coating thickness is about 100 μm, about 110 μm, about 120 μm, about 130 μm, about 140 μm, about 150 μm, about 160 μm, about 170 μm, about 180 μm, about 190 μm, about 200 μm, about 210 μm, about 220 μm, about 230 μm, about 240 μm, about 250 μm, about 260 μm, about 270 μm, about 280 μm, about 290 μm, or about 300 μm.

In some embodiments, the semipermeable membrane coating thickness is about 500 μm to about 650 μm or about 580 μm to about 600 μm. In some embodiments, the semipermeable membrane coating thickness is about 592 μm. In some embodiments, the semipermeable membrane coating thickness is about 500 μm, about 510 μm, about 520 μm, about 530 μm, about 540 μm, about 550 μm, about 560 μm, about 570 μm, about 580 μm, about 590 μm, or about 600 μm.

As regards suitable plasticizers for use in the method of manufacturing described herein, reference is generally made to Chapter II, Section 4 above (“Semipermeable membrane coating”). In specific embodiments, the plasticizer is polyethylene glycol.

In specific embodiments, the plasticizer constitutes about 10% to about 20% (such as about 15%) by weight of the semipermeable membrane coating and the weight ratio of the semipermeable membrane coating to the tablet core is about 0.06:1 to about 0.08:1. The plasticizer may be polyethylene glycol.

In some embodiments, cellulose acetate in the semipermeable membrane coating (solution) has an acetyl content of about 30% to about 50%. In some embodiments, the cellulose acetate in the semipermeable membrane coating (solution) has an acetyl content of about 31% to about 49%, about 32% to about 48%, about 33% to about 47%, or about 34% to about 46%. In some embodiments, cellulose acetate in the semipermeable membrane coating (solution) has an acetyl content of about 35% to about 45%. In some embodiments, cellulose acetate in the semipermeable membrane coating (solution) has an acetyl content of about 36% to about 44%, about 37% to about 41%, or about 38% to about 42%. In some embodiments, cellulose acetate in the semipermeable membrane coating (solution) has an acetyl content of about 39% to about 41%. In some embodiments, cellulose acetate in the semipermeable membrane coating (solution) has an acetyl content of about 40%. In some embodiments, the cellulose acetate in the semipermeable membrane coating (solution) has an acetyl content of about 30%, about 31%, about 32%, about 33%, about 34%, about 35%, about 36%, about 37%, about 38%, about 39%, about 40%, about 41%, about 42%, about 43%, about 44%, about 45%, about 46%, about 47%, about 48%, about 49%, or about 50%.

In some embodiments, the semipermeable membrane coating constitutes about 2% to about 10% by weight of the tablet. In some embodiments, the semipermeable membrane coating constitutes about 3% to about 9% by weight of the tablet. In some embodiments, the semipermeable membrane coating constitutes about 4% to about 8% by weight of the tablet. In some embodiments, the semipermeable membrane coating constitutes about 5% to about 7% by weight of the tablet. In some embodiments, the semipermeable membrane coating constitutes about 6% by weight of the tablet.

In some embodiments, the semipermeable membrane coating constitutes about 2%, about 3%, about 4%, about 5%, about 6%, about 7%, about 8%, about 9%, or about 10% by weight of the tablet.

In some embodiments, the method further comprises:
drilling one or more delivery orifices in the semipermeable membrane coating.

In some embodiments, the one or more delivery orifices are drilled in the semipermeable membrane coating by laser drilling. In some embodiments, the spindle speed used for laser drilling is about 700 to about 900 rpm, such as about 800 rpm. In some embodiments, the system speed used for laser drilling is about 400 to about 600 links/min, such as about 500 links/min or about 360 links/mm. In some embodiments, the pitch used for laser drilling is about 0.02 to about 0.08%, such as about 0.04% or about 0.06%. In some embodiments, the drill speed used for laser drilling is about 2300 to about 2700 mm/sec, such as about 2500 mm/sec, or about 800 to about 1200 mm/sec, such as about 1000 mm/sec. In some embodiments, the power level used for laser drilling is about 70 to about 110%, such as about 80% to about 100%, such as about 90%. In some embodiments, the PWM frequency used for laser drilling is about 20 to about 30 KHz, such as about 25 KHz. An example of a suitable laser drilling machine is an Ackley Laser drilling machine.

In some embodiments, the one or more delivery orifices are drilled in the semipermeable membrane coating by mechanical drilling.

As outlined above, the one or more delivery orifices can be drilled in the semipermeable membrane coating any time after the semipermeable membrane coating is applied. For example, drilling one or more delivery orifices in the semipermeable membrane coating can take place directly after applying the semipermeable membrane coating on the tablet core or only after applying a further coating surrounding the semipermeable membrane coating, such as a seal coating, an immediate release drug coating, and/or a film coating. If the drilling takes place after applying a further coating surrounding the semipermeable membrane coating, such as a seal coating, an immediate release drug coating, and/or a film coating, the one or more delivery orifices consequently will span also through such further coatings that surround the semipermeable membrane coating (such as a seal coating, an immediate release drug coating, and/or a film coating. In some embodiments, the one or more delivery orifices are drilled in the semipermeable membrane coating by laser drilling after a film coating is applied.

In some embodiments, the one or more delivery orifices have a diameter of about 0.35 mm to about 2.0 mm. In some embodiments, the one or more delivery orifices have a diameter of about 0.4 mm to about 1.0 mm, about 0.4 mm to about 0.9 mm, about 0.4 mm to about 0.8 mm, or about 0.4 mm to about 0.7 mm. In some embodiments, the one or more delivery orifices have a diameter of about 0.4 mm to about 0.6 mm. In some embodiments, the one or more delivery orifices have a diameter of about 0.45 mm to about 0.55 mm. In specific embodiments, the one or more delivery orifices have a diameter of about 0.5 mm.

In some embodiments, the one or more delivery orifices have a diameter of about 0.35 mm, about 0.4 mm, about 0.45 mm, about 0.5 mm, about 0.55 mm, about 0.6 mm, about 0.65 mm, about 0.7 mm, about 0.75 mm, about 0.8 mm, about 0.85 mm, about 0.9 mm, about 0.95 mm, about 1.0 mm, about 1.05 mm, about 1.1 mm, about 1.15 mm, about 1.2 mm, about 1.25 mm, about 1.3 mm, about 1.35 mm, about 1.4 mm, about 1.45 mm, about 1.5 mm, about 1.55 mm, about 1.6 mm, about 1.65 mm, about 1.7 mm, about 1.75 mm, about 1.8 mm, about 1.85 mm, about 1.9 mm, about 1.95 mm, or about 2.0 mm.

As outlined above, within the meaning of this disclosure, the “diameter” of a delivery orifice is referring to the diameter of the delivery orifice as measured at the surface of the semipermeable membrane coating, which is not in contact with, for example, a seal coating or a film coating, but instead is in contact with the tablet core.

In some embodiments, drilling one or more delivery orifices in the semipermeable membrane coating takes place after applying a further coating surrounding the semipermeable membrane coating, such as a seal coating, an immediate release drug coating, and/or a film coating (see also embodiments below that refer to application of such coatings).

In some embodiments, drilling one or more delivery orifices in the semipermeable membrane coating takes place after applying a further coating surrounding the semipermeable membrane coating, such as a seal coating, an immediate release drug coating, and/or a film coating, wherein the one or more delivery orifices are drilled in the semipermeable membrane coating (and consequently also in the seal coating, the immediate release drug coating, and/or the film coating) by laser drilling. As outlined above, while mechanical drilling usually results in a cylindrical delivery orifice with a (substantially) constant diameter throughout the entire orifice, laser drilling usually results in a conical delivery orifice, for which the diameter is getting narrower towards the tablet core. As also outlined above, a delivery orifice obtained by laser drilling thus usually provides the smallest diameter at the surface of the semipermeable membrane coating, which is in contact with the tablet core (said smallest diameter being referred to herein as “diameter”), and the largest diameter at the opposite site of the delivery orifice, such as at the outer surface of the tablet when drilling the delivery orifice takes place after an outer film coating is applied (said largest diameter being referred to herein as “outer diameter” of a delivery orifice obtained by laser drilling). In some of the embodiments described in this paragraph, the one or more delivery orifices obtained by laser drilling have a diameter of about 0.4 mm to about 0.6 mm and an outer diameter of about 0.8 mm to about 1.4 mm, such as about 1 mm to about 1.2 mm. In specific embodiments of the embodiments described in this paragraph, the one or more delivery orifices obtained by laser drilling have a diameter of about 0.5 mm and an outer diameter of about 1 mm to about 1.2 mm.

In some embodiments, the semipermeable membrane coating comprises one to ten delivery orifices. In some embodiments, the semipermeable membrane coating comprises one to four delivery orifices. In some embodiments, the semipermeable membrane coating comprises two delivery orifices. In specific embodiments, the semipermeable membrane coating comprises one delivery orifice.

In some embodiments, the method further comprises:
coating the semipermeable membrane coating with a seal coating. The seal coating surrounds the semipermeable membrane coating.

In some embodiments, coating the semipermeable membrane coating with a seal coating comprises spraying a seal coating solution on the semipermeable membrane coating, thereby providing the seal coating surrounding the semipermeable membrane coating. In some embodiments, the pan size used for seal coating is about 22 to about 26 in, such as about 24 in. In some embodiments, the nozzle port opening used for seal coating is about 1.0 to about 1.4 mm, such as about 1.2 mm. In some embodiments, the drum speed used for seal coating is about 10 to about 18 rpm, such as about 12 to about 16 rpm. In some embodiments, the inlet air volume used for seal coating is about 200 to about 500 cfm, such as about 250 to about 450 cfm. In some embodiments, the inlet temperature used for seal coating is about 50° C. to about 70° C., such as about 55° C. to about 65° C. In some embodiments, the exhaust air temperature used for seal coating is about 30° C. to about 60° C., such as about 40° C. to about 50° C. In some embodiments, the spray rate used for seal coating is about 20 to about 50 g/min, such as about 25 to about 45 g/min, such as about 35 g/min. In some embodiments, the pattern air pressure used for seal coating is about 20 to about 30 psi, such as about 25 psi. In some embodiments, the atomizing air pressure used for seal coating is about 30 to about 40 psi, such as about 35 psi. In some embodiments, seal coating is performed using an O'hara coating machine configured with a fully perforated pan.

In some embodiments, the method further comprises drying the tablet after spraying the seal coating solution on the semipermeable membrane coating.

As regards suitable seal coatings, reference is generally made to Chapter II, Section 6 above (“Seal coating”). In some embodiments, the seal coating (solution) comprises hydroxypropyl methylcellulose and/or polyethylene glycol. In some embodiments, the seal coating consists of hydroxypropyl methylcellulose and polyethylene glycol. In some embodiments, the weight ratio of the seal coating to the tablet core is about 0.01:1 to about 0.05:1. In some embodiments, the weight ratio of the seal coating to the tablet core is about 0.02:1 to about 0.04:1. In some embodiments, the weight ratio of the seal coating to the tablet core is about 0.03:1. In some embodiments, the weight ratio of the seal coating to the tablet core is about 0.032:1. In some embodiments, the weight ratio of the seal coating to the tablet core is about 0.01:1, about 0.02:1, about 0.03:1, about 0.04:1, or about 0.05:1. In some embodiments, the seal coating constitutes about 1% to about 4% by weight of the tablet. In some embodiments, the seal coating constitutes about 1.5% to about 3.5% or about 1.5% to about 3% or about 1.5% to about 2.5% or about 1.5% to about 2% by weight of the tablet. In some embodiments, the seal coating constitutes about 2% to about 3% or about 2% to about 2.5% by weight of the tablet. In some embodiments, the seal coating constitutes about 2% by weight of the tablet. In some embodiments, the seal coating constitutes about 2.2% by weight of the tablet. In some embodiments, the seal coating solution comprises hydroxypropyl methylcellulose and polyethylene glycol, which constitute about 5% to about 15% by weight of the seal coating solution. In some embodiments, the seal coating solution comprises hydroxypropyl methylcellulose and polyethylene glycol, which constitute about 10% by weight of the seal coating solution. In some embodiments, the solvent of the seal coating solution is purified water.

In some embodiments, the method further comprises:
coating the seal coating with an immediate release drug coating (briefly referred to herein also as “drug coating”) comprising dexpramipexole, or a pharmaceutically acceptable salt thereof. The drug coating surrounds the seal coating.

In some embodiments, coating the seal coating with the immediate release drug coating comprises spraying an immediate release drug coating solution comprising dexpramipexole, or a pharmaceutically acceptable salt thereof, (briefly referred to herein also as “drug coating solution”) on the seal coating, thereby providing the drug coating surrounding the seal coating. In some embodiments, the pan size used for drug coating is about 22 to about 26 in, such as about 24 in. In some embodiments, the nozzle port opening used for drug coating is about 1.0 to about 1.4 mm, such as about 1.2 mm. In some embodiments, the drum speed used for drug coating is about 10 to about 18 rpm, such as about 12 to about 16 rpm. In some embodiments, the inlet air volume used for drug coating is about 200 to about 500 cfm, such as about 250 to about 450 cfm. In some embodiments, the inlet temperature used for drug coating is about 60° C. to about 80° C., such as about 65° C. to about 75° C. In some embodiments, the exhaust air temperature used for drug coating is about 30° C. to about 60° C., such as about 40° C. to about 50° C. In some embodiments, the spray rate used for drug coating is about 20 to about 50 g/min, such as about 25 to about 45 g/min, such as about 35 g/min. In some embodiments, the pattern air pressure used for drug coating is about 20 to about 30 psi, such as about 25 psi. In some embodiments, the atomizing air pressure used for drug coating is about 30 to about 40 psi, such as about 35 psi. In some embodiments, drug coating is performed using an O'hara coating machine configured with a fully perforated pan.

In some embodiments, the method further comprises drying the tablet after spraying the drug coating solution onto the seal coating.

In other embodiments (wherein the tablet does not comprise a seal coating between the semipermeable membrane coating and the immediate release drug coating), the method further comprises:
coating the semipermeable membrane coating with an immediate release drug coating comprising dexpramipexole, or a pharmaceutically acceptable salt thereof. The drug coating surrounds the semipermeable membrane coating.

In some embodiments, coating the semipermeable membrane coating with the immediate release drug coating comprises spraying an immediate release drug coating solution comprising dexpramipexole, or a pharmaceutically acceptable salt thereof, on the semipermeable membrane coating, thereby providing the drug coating surrounding the semipermeable membrane coating. In some embodiments, the method further comprises drying the tablet after spraying the drug coating solution onto the semipermeable membrane coating.

As regards suitable drug coatings, reference is generally made to Chapter II, Section 5 above (“Immediate release drug coating”).

In some embodiments, the drug coating (solution) further comprises a binder. In some embodiments, the weight ratio of dexpramipexole dihydrochloride monohydrate equivalent to binder in the drug coating is about 1:1 to about 1:4. In some embodiments, the weight ratio of dexpramipexole dihydrochloride monohydrate equivalent to binder in the drug coating is about 1:2 to about 1:4. In some embodiments, the weight ratio of dexpramipexole dihydrochloride monohydrate equivalent to binder in the drug coating is about 1:2.5 to about 1:3.5. In specific embodiments, the weight ratio of dexpramipexole dihydrochloride monohydrate equivalent to binder in the drug coating is about 1:3. In even more specific embodiments, the weight ratio of dexpramipexole dihydrochloride monohydrate equivalent to binder in the drug coating is about 1:3 and the binder comprises hydroxypropyl methylcellulose and polyethylene glycol. In other specific embodiments, the weight ratio of dexpramipexole dihydrochloride monohydrate equivalent to binder in the drug coating is about 1:2. In some embodiments, the weight ratio of dexpramipexole dihydrochloride monohydrate equivalent to binder in the drug coating is about 1:1, about 1:1.5, about 1:2, about 1:2.5, about 1:3, about 1:3.5, or about 1:4.

In some embodiments, the binder and dexpramipexole, or a pharmaceutically acceptable salt thereof, constitute about 5% to about 15% by weight of the drug coating solution. In some embodiments, the binder and dexpramipexole, or a pharmaceutically acceptable salt thereof, constitute about 10% by weight of the drug coating solution. In some embodiments, the solvent of the drug coating solution is purified water.

In some embodiments, the binder in the drug coating (solution) comprises hydroxypropyl methylcellulose and polyethylene glycol. In other embodiments, the binder in the drug coating (solution) consists of hydroxypropyl methylcellulose and polyethylene glycol.

In some embodiments, the weight ratio of the drug coating to the tablet core is about 0.1:1 to about 0.5:1, about 0.11:1 to about 0.49:1, about 0.12:1 to about 0.48:1, about 0.13:1 to about 0.47:1, about 0.14:1 to about 0.46:1, about 0.15:1 to about 0.45:1, about 0.16:1 to about 0.44:1, about 0.17:1 to about 0.43:1, about 0.18:1 to about 0.42:1, about 0.19:1 to about 0.41:1, about 0.20:1 to about 0.40:1, about 0.21:1 to about 0.39:1, about 0.22:1 to about 0.38:1, about 0.23:1 to about 0.37:1, about 0.24:1 to about 0.36:1, about 0.25:1 to about 0.35:1, 0.26:1 to about 0.34:1, about 0.27:1 to about 0.33:1, or about 0.28:1 to about 0.32:1. In some embodiments, the weight ratio of the drug coating to the tablet core is about 0.26:1 to about 0.28:1. In some embodiments, the weight ratio of the drug coating to the tablet core is about 0.27:1. In some embodiments, the weight ratio of the drug coating to the tablet core is about 0.24:1 to about 0.3:1. In some embodiments, the weight ratio of the drug coating to the tablet core is about 0.25:1 to about 0.29:1. In some embodiments, the weight ratio of the drug coating to the tablet core is about 0.26:1 to about 0.28:1. In some embodiments, the weight ratio of the drug coating to the tablet core is about 0.27:1. In some embodiments, the weight ratio of the drug coating to the tablet core is about 0.29:1 to about 0.31:1. In some embodiments, the weight ratio of the drug coating to the tablet core is about 0.30:1. In some embodiments, the weight ratio of the drug coating to the tablet core is about 0.296:1. In some embodiments, the weight ratio of the drug coating to the tablet core is about 0.3:1. In some embodiments, the weight ratio of the drug coating to the tablet core is about 0.1:1, about 0.11:1, about 0.12:1, about 0.13:1, about 0.14:1, about 0.15:1, about 0.16:1, about 0.17:1, about 0.18:1, about 0.19:1, about 0.20:1, about 0.21:1, about 0.22:1, about 0.23:1, about 0.24:1, about 0.25:1, about 0.26:1, about 0.27:1, about 0.28:1, about 0.29:1, about 0.30:1, about 0.31:1, about 0.32:1, about 0.33:1, or about 0.34:1.

In some embodiments, the weight ratio of the drug coating to the tablet core is about 0.18:1 to about 0.26:1. In some embodiments, the weight ratio of the drug coating to the tablet core is about 0.19:1 to about 0.25:1. In some embodiments, the weight ratio of the drug coating to the tablet core is about 0.20:1 to about 0.24:1. In some embodiments, the weight ratio of the drug coating to the tablet core is about 0.21:1 to about 0.23:1. In some embodiments, the weight ratio of the drug coating to the tablet core is about 0.22:1. In some embodiments, the weight ratio of the drug coating to the tablet core is about 0.18:1, about 0.19:1, about 0.20:1, about 0.21:1, about 0.22:1, about 0.23:1, about 0.24:1, about 0.25:1, or about 0.26:1.

In some embodiments, the drug coating constitutes about 5% to about 35% by weight of the tablet. In some embodiments, the drug coating constitutes about 10% to about 30% by weight of the tablet. In some embodiments, the drug coating constitutes about 15% to about 25% by weight of the tablet. In some embodiments, the drug coating constitutes about 16% to about 24% or about 17% to about 23% by weight of the tablet. In some embodiments, the drug coating constitutes about 18% to about 22% by weight of the tablet. In some embodiments, the drug coating constitutes about 19% to about 21% by weight of the tablet. In some embodiments, the drug coating constitutes about 20% by weight of the tablet. In some embodiments, the drug coating constitutes about 10% to about 22% by weight of the tablet. In some embodiments, the drug coating constitutes about 12% to about 20% or about 13% to about 19% by weight of the tablet. In some embodiments, the drug coating constitutes about 14% to about 18% by weight of the tablet. In some embodiments, the drug coating constitutes about 15% to about 17% by weight of the tablet. In some embodiments, the drug coating constitutes about 16% by weight of the tablet. In some embodiments, the drug coating constitutes about about 5%, about 6%, about 7%, about 8%, about 9%, 10%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, or about 30% by weight of the tablet.

In the embodiments wherein no seal coating and no immediate release drug coating are applied, the method further comprising:
coating the semipermeable membrane coating with a film coating. The film coating surrounds the semipermeable membrane coating.

In some embodiments, coating the semipermeable membrane coating with a film coating comprises spraying a film coating solution on the semipermeable membrane coating, thereby providing a film coating surrounding the semipermeable membrane coating. In some embodiments, the pan size used for film coating is about 22 to about 26 in, such as about 24 in. In some embodiments, the nozzle port opening used for film coating is about 1.0 to about 1.4 mm, such as about 1.2 mm. In some embodiments, the drum speed used for film coating is about 10 to about 18 rpm, such as about 12 to about 16 rpm. In some embodiments, the inlet air volume used for film coating is about 200 to about 500 cfm, such as about 250 to about 450 cfm. In some embodiments, the inlet temperature used for film coating is about 50° C. to about 70° C., such as about 55° C. to about 65° C. In some embodiments, the inlet temperature used for film coating is about 4° C. to about 8° C., such as about 6° C. In some embodiments, the exhaust air temperature used for film coating is about 30° C. to about 60° C., such as about 40° C. to about 50° C. In some embodiments, the spray rate used for film coating is about 30 to about 60 g/min, such as about 40 to about 50 g/min, such as about 45 g/min. In some embodiments, the pattern air pressure used for film coating is about 20 to about 30 psi, such as about 25 psi. In some embodiments, the atomizing air pressure used for film coating is about 30 to about 40 psi, such as about 35 psi. In some embodiments, film coating is performed using an O'hara coating machine configured with a fully perforated pan.

In some embodiments, the method further comprises drying the tablet after spraying the film coating solution on the semipermeable membrane coating.

In the embodiments wherein an immediate release drug coating and optionally also a seal coating are applied, the method further comprising:
coating the drug coating with a film coating. The film coating surrounds the drug coating.

In some embodiments, coating the drug coating with a film coating comprises spraying a film coating solution on the drug coating, thereby providing a film coating surrounding the drug coating. In some embodiments, the method further comprises drying the tablet after spraying the film coating solution onto the drug coating.

As regards suitable film coatings, reference is generally made to Chapter II, Section 7 above (“Film coating”).

In some embodiments, the film coating (solution) comprises polyvinyl alcohol. In some embodiments, the film coating (solution) comprises polyvinyl alcohol, titanium dioxide, polyethylene glycol, and talc. In some embodiments, the film coating consists of polyvinyl alcohol, titanium dioxide, polyethylene glycol, and talc.

In some embodiments, the weight ratio of the film coating to the tablet core is about 0.01:1 to about 0.06:1. In some embodiments, the weight ratio of the film coating to the tablet core is about 0.02:1 to about 0.05:1. In some embodiments, the weight ratio of the film coating to the tablet core is about 0.03:1 to about 0.04:1. In some embodiments, the weight ratio of the film coating to the tablet core is about 0.03:1. In some embodiments, the weight ratio of the film coating to the tablet core is about 0.04:1. In some embodiments, the weight ratio of the film coating to the tablet core is about 0.032:1. In some embodiments, the weight ratio of the film coating to the tablet core is about 0.042:1. In some embodiments, the weight ratio of the film coating to the tablet core is about 0.01:1, about 0.015:1, about 0.02:1, about 0.025:1, about 0.03:1, about 0.035:1, about 0.04:1, about 0.045:1, about 0.05:1, about 0.055:1, or about 0.06:1.

In some embodiments, the film coating constitutes about 1% to about 5% by weight of the tablet. In some embodiments, the film coating constitutes about 2% to about 4% by weight of the tablet. In some embodiments, the film coating constitutes about 3% by weight of the tablet. In some embodiments, the film coating constitutes about 1%, about 1.5%, about 2%, about 2.5%, about 3%, about 3.5%, about 4%, about 4.5%, or about 5% by weight of the tablet.

In some embodiments, the film coating solution comprises polyvinyl alcohol, titanium dioxide, polyethylene glycol, and talc, which constitute about 15% to about 25% by weight of the film coating solution. In some embodiments, the film coating solution comprises polyvinyl alcohol, titanium dioxide, polyethylene glycol, and talc, which constitute about 20% by weight of the film coating solution. In some embodiments, the solvent of the film coating solution is purified water.

As regards suitable pharmaceutically acceptable salts for dexpramipexole in the pre-blend and drug coating, reference is generally made to Chapter II, Section 1 above (“Pharmaceutically acceptable salts of dexpramipexole”). In some embodiments, the pharmaceutically acceptable salt of dexpramipexole in the pre-blend is dexpramipexole dihydrochloride or a hydrate thereof. In some embodiments, the pharmaceutically acceptable salt of dexpramipexole in the immediate release coating is dexpramipexole dihydrochloride or a hydrate thereof. In some embodiments, the pharmaceutically acceptable salt of dexpramipexole in the pre-blend and in the immediate release coating is dexpramipexole dihydrochloride or a hydrate thereof. In some embodiments, the pharmaceutically acceptable salt of dexpramipexole in the pre-blend is dexpramipexole dihydrochloride monohydrate. In some embodiments, the pharmaceutically acceptable salt of dexpramipexole in the immediate release drug coating is dexpramipexole dihydrochloride monohydrate. In some embodiments, the pharmaceutically acceptable salt of dexpramipexole in the pre-blend and in the immediate release drug coating is dexpramipexole dihydrochloride monohydrate. In some embodiments, the pharmaceutically acceptable salt of dexpramipexole in the pre-blend is dexpramipexole dihydrochloride. In some embodiments, the pharmaceutically acceptable salt of dexpramipexole in the immediate release drug coating is dexpramipexole dihydrochloride. In some embodiments, the pharmaceutically acceptable salt of dexpramipexole in the pre-blend and in the immediate release drug coating is dexpramipexole dihydrochloride.

In addition to the embodiments expressly described in this chapter (“IV—Method of Manufacturing”) above, reference is generally also made to further embodiments regarding the pharmaceutical composition and its properties as described, in particular, in the chapter “II—Pharmaceutical Compositions” above.

In some aspects, the present disclosure is also directed to a pharmaceutical composition obtained by the method of manufacturing as described in this chapter (“IV—Method of Manufacturing”) above.

### EXAMPLES

The following examples are intended solely as an illustration and should not be regarding as restricting the disclosure in any way.

In the examples below, orally deliverable osmotic tablets comprising dexpramipexole dihydrochloride monohydrate have been manufactured. In vitro dissolution profiles of the manufactured tablets were compared to Mirapex® extended release (ER) tablets comprising 4.5 mg of pramipexole dihydrochloride monohydrate. Inactive ingredients of the pramipexole extended release tablets are, according to the manufacturer, hypromellose, cornstarch, carbomer homopolymer, colloidal silicon dioxide, and magnesium stearate.

Dissolution profiles were recorded using USP <711> Dissolution Apparatus I (Baskets, B). Dissolution was measured in 900 mL of solvent, equilibrated to 37.0±0.5° C., with agitation at 100 rpm for 24 hours. Samples were collected after 1, 2, 4, 6, 8, 10, 12, 16, 20 and 24 hours. The analytical finish method employed isocratic reversed phase HPLC with UV detection at 265 nm to resolve the active peak. Following confirmation of system suitability, quantitation of the percentage released was achieved by comparing the dexpramipexole peak area in the sample solution chromatograms to the mean dexpramipexole peak area in standard solution chromatograms, correcting for the quantity of drug substance removed during sampling at earlier time points. Different solvents were used for dissolution studies, including 0.1 N HCl, McIlvaine Buffer, pH 4.5, and 50 mM monobasic potassium phosphate buffer, pH 6.8. As known in the art, McIlvaine Buffer is made from citric acid and disodium hydrogen phosphate.

In Examples 1 to 5, the different tablet batches were manufactured each at a scale of ˜500 g blend. In Example 6, the manufacturing scale was 1 kg blend per batch unless otherwise indicated. The tablet cores were manufactured using direct compression process. Compression was performed using a Korsch XL-100 rotary tablet press (for all manufacturing scales). Coating was performed using a Vector LDCS-5 Pan Coater equipped with 6″ or 12″ partially perforated coating pans for the smaller scale batches (up to 1 kg blend). Larger scale batches (engineering batches and clinical trial batches) were coated using an O'hara coating machine configured with a fully perforated pan. The manufacturing process is described in further detail in Examples 1 and 7. Example 9 provides additional osmotic tablet formulations evaluated.

271 mg dexpramipexole dihydrochloride monohydrate is equivalent to 255 mg dexpramipexole dihydrochloride. 319 mg dexpramipexole dihydrochloride monohydrate is equivalent to 300 mg dexpramipexole dihydrochloride. 400 mg dexpramipexole dihydrochloride monohydrate is equivalent to 376 mg dexpramipexole dihydrochloride. Cellulose acetate CA 398 has an acetyl content of 39.8%.

In the Examples below, dexpramipexole dihydrochloride monohydrate comprising 0.04% or less of pramipexole dihydrochloride monohydrate was applied (i.e., the chiral purity for dexpramipexole dihydrochloride monohydrate is 99.96% or more). Stability data indicated that the chiral purity did not change over time during tablet manufacture and storage.

### Example 1—Inorganic Osmotic Agent

First, the effect of an inorganic osmotic agent in the tablet core mixed with dexpramipexole dihydrochloride monohydrate was evaluated. Sodium chloride was selected as an inorganic osmotic agent and three levels of sodium chloride were evaluated for impact on release rate. Table 1 describes the compositions that were prepared for studying the impact of sodium chloride on the in vitro dissolution profiles.

TABLE 1
Compositions used to study the impact of the presence of sodium chloride
in the tablet core on the release of dexpramipexole dihydrochloride
monohydrate. Microcrystalline cellulose = diluent, copovidone = binder;
sodium chloride = inorganic osmotic agent; magnesium stearate = lubricant.

| | Summary | | | | | | | |
|---|---|---|---|---|---|---|---|---|
| | Lot Number | | | | | | | |
| | Formulation component | | | | | | | |
| | AZF-1-FD-3-23B | | AZF-1-FD-3-15D | | AZF-1-FD-3-8A | | AZF-1-FD-3-15A | |
| | Sodium Chloride (Osmotic agent) amount | | | | | | | |
| | 0% w/w of core | | 20% w/w of core | | 20% w/w of core | | 30% w/w of core | |
| | PEG 3350 (Plasticizer) amount | | | | | | | |
| | 5.5% w/w | | 5.5% w/w | | 4% w/w | | 4% w/w | |
| Material | mg/tab | % w/w core | mg/tab | % w/w core | mg/tab | % w/w core | mg/tab | % w/w core |

Tablet Core

| | | | | | | | | |
|---|---|---|---|---|---|---|---|---|
| Dexpramipexole | 319.00** | 42.00 | 319.00* | 42.00 | 319.00** | 42.00 | 319.00** | 42.00 |
| Dihydrochloride | | | | | | | | |
| Monohydrate | | | | | | | | |
| Microcrystalline | 383.55 | 50.50 | 231.85 | 30.50 | 231.85 | 30.50 | 115.69 | 20.50 |
| cellulose | | | | | | | | |
| (Avicel PH 102) | | | | | | | | |
| Copovidone | 53.17 | 7.00 | 53.17 | 7.00 | 53.17 | 7.00 | 53.17 | 7.00 |
| (Kollidon VA | | | | | | | | |
| 64 fine) | | | | | | | | |
| Sodium Chloride | 0.00 | 0.00 | 151.90 | 20.00 | 151.90 | 20.00 | 227.86 | 30.00 |
| (Emprove ®) | | | | | | | | |
| Magnesium | 3.80 | 0.50 | 3.80 | 0.50 | 3.80 | 0.50 | 3.80 | 0.50 |
| Total | 759.52 | 100.00 | 759.52 | 100.00 | 759.52 | 100.00 | 759.52 | 100.00 |

Semipermeable Membrane Coating\hat{ }\hat{ }

| | | | | | | | | |
|---|---|---|---|---|---|---|---|---|
| Opadry CA | 41.77 | . . . | 41.77 | . . . | . . . | . . . | . . . | . . . |
| 500F190003 | | | | | | | | |
| clear (Cellulose | | | | | | | | |
| acetate | | | | | | | | |
| 398-10:PEG 3350 = | | | | | | | | |
| 85:15) | | | | | | | | |
| Opadry CA | . . . | . . . | . . . | . . . | 30.38 | . . . | 30.38 | . . . |
| 500F190011 clear | | | | | | | | |
| (Cellulose acetate | | | | | | | | |
| 398-10:PEG 3350 = | | | | | | | | |
| 95:5) | | | | | | | | |
| Acetone\hat{ } | q.s. | . . . | q.s. | . . . | q.s. | . . . | q.s. | . . . |
| Purified water\hat{ } | q.s. | . . . | q.s. | . . . | q.s. | . . . | q.s. | . . . |
| Total | 801.29 | . . . | 801.29 | . . . | 789.90 | . . . | 789.90 | . . . |

q.s.: Quantity sufficient
**Conversion factor for Dexpramipexole Dihydrochloride Monohydrate to Dexpramipexole Dihydrochloride = 0.9404
\hat{ }Evaporates during processing
\hat{ }\hat{ }Solid content of Semipermeable Membrane Coating Solution = 5% w/w, Acetone:Purified water = 95:5

The blend for the tablet core was prepared as follows:
(1) Co-sift dexpramipexole, microcrystalline cellulose (Avicel PH 102), copovidone and sodium chloride through #30 US std sieve;
(2) Blend co-sifted materials of Step (1) in V-blender at 25 rpm for 10 minutes;
(3) Screen magnesium stearate through #40 US std sieve;
(4) Lubricate blend of Step (2) using sifted magnesium stearate of Step (3) in v-blender at 25 rpm for 5 minutes.

Table 2 shows the in-process compression parameters for each lot in Table 1.

TABLE 2
In-process compression parameters for lots in Table 1.

| | Lot Number | | | |
|---|---|---|---|---|
| | AZF-1- | AZF-1- | AZF-1- | AZF-1- |
| Parameter | FD-3-23B | FD-3-15D | FD-3-8A | FD-3-15A |
| Tooling | 9.50 mm * | 9.50 mm * | 9.50 mm * | 9.50 mm * |
| | 17.12 mm | 17.12 mm | 17.12 mm | 17.12 mm |
| Average tablet | 758 | 758 | 758 | 758 |
| weight (mg) | | | | |
| Average Hardness (kP) | 18.3 | 20.2 | 20.2 | 17.2 |
| Thickness (mm) | 6.94 | 5.71 | 5.71 | 5.71 |
| Friability (%) | 0.10 | 0.12 | 0.08 | 0.19 |

The solution for semipermeable membrane coating was prepared as follows:
(1) Add purified water and acetone to a stainless-steel container;
(2) Put stainless-steel container on a magnetic stirrer and turn on the stier; adjust the speed of stirrer till vortex is formed;
(3) Slowly add Opadry CA to the vortex;
(4) After complete addition, mix the content until 60 minutes; at the end of60 minutes, record if a smooth homogeneous solution is obtained.

Table 3 shows the in-process coating parameters for each lot in Table 1.

TABLE 3
In-process coating parameters for lots in Table 1.

| | Lot Number | | | |
|---|---|---|---|---|
| | AZF-1- | AZF-1- | AZF-1- | AZF-1- |
| Parameter | FD-3-23B | FD-3-15D | FD-3-8A | FD-3-15A |
| Pan load (g) | 350 | 350 | 350 | 350 |
| Inlet temperature (° C.) | 36-39 | 36-39 | 33-38 | 35-38 |
| Product | 26-28 | 26-28 | 26-28 | 26-27 |
| temperature (° C.) | | | | |
| Spray rate (g/min) | 10 | 10 | 10 | 10 |
| Atomization (PSI) | 20 | 20 | 20 | 20 |
| Pan speed (rpm) | 20 | 20 | 20 | 20 |
| Drying time (min) | 5 | 5 | 5 | 5 |

The coated tablets of each lot were drilled with 0.5 mm delivery orifice using a mechanical drill press.

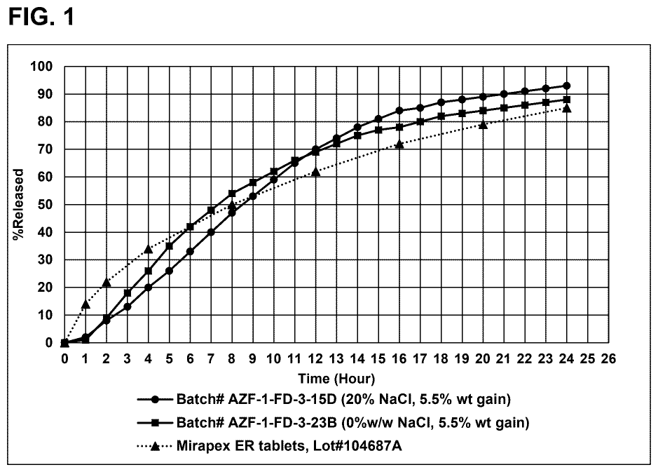

The dissolution results for Lot #AZF-1-FD-3-23B and Lot #AZF-1-FD-3-15D are provided in FIG. 1. FIG. 1 shows that pseudo-zero order profile could be obtained with Lot #AZF-1-FD-3-23B even without addition of any osmotic agent to the tablet core. However, terminal release at 24 hours was merely around 80%. To achieve zero-order release profile, the addition of an amount of inorganic osmotic agent (applied here was sodium chloride) was necessary. The formulation with sodium chloride showed zero-order release profile (Lot #AZF-1-FD-3-15D). Lot #AZF-1-FD-3-15D showed a constant release rate (3-5%/hour) between 2 to 16 hours. With addition of osmotic agent, terminal release at 24 hour could be increased up to 90%.

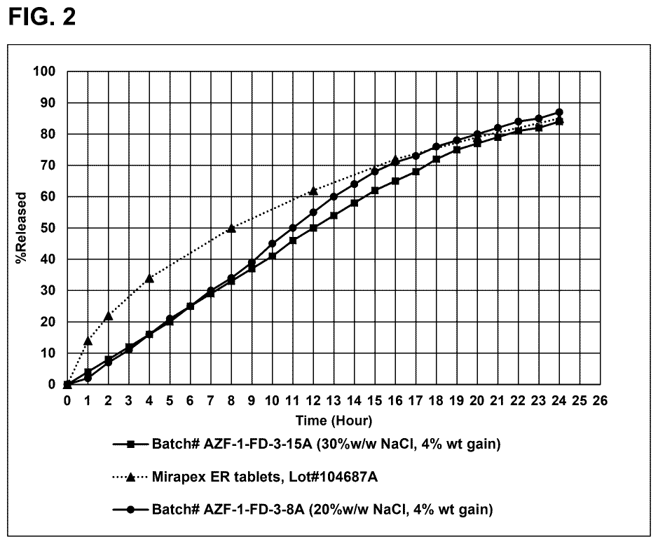

FIG. 2 shows the dissolution profiles for Lot #AZF-1-FD-3-8A and Lot #AZF-1-FD-3-15A. It can be taken from the profiles of FIG. 2 that increasing the amount of inorganic osmotic agent (applied here was sodium chloride) from 20% w/w (of the tablet core) to 30% w/w (of the tablet core) did not have significant impact on release rate or terminal release.

Thus, it was shown that the presence of an inorganic osmotic agent at specific amounts in the tablet core has beneficial impacts on the in vitro dissolution profiles of dexpramipexole dihydrochloride monohydrate.

### Example 2—Swellable Polymeric Osmotic Agent

Swellable polymeric osmotic agents can act synergistically with inorganic osmotic agents in a mixture with drug to provide 100% terminal release. Polyethylene oxide (Polyox) WSR N80 and Polyethylene oxide (Polyox) WSR 205 were selected as swellable polymeric osmotic agents to evaluate the impact on release rate. Viscosity (in mPa·S at 25° C.) of a 5% solution of Polyox WSR N80 is 55-90 and of Polyox WSR 205 is 4500-8800. Table 4 describes the compositions used to study the impact of swellable polymeric osmotic agent on release profiles.

TABLE 4
Compositions used to study the impact of polyethylene oxide in the tablet
core on the release of dexpramipexole dihydrochloride monohydrate.

| | |
|---|---|
| Formulation | Lot Number |

| | component | AZF-1-FD-3-29B | AZF-1-FD-3-23D |
|---|---|---|---|
| Summary | Sodium Chloride | 20% w/w of core | 20% w/w of core |
| | (Osmotic agent) | | |
| | amount | | |
| | Polyox amount | 20% w/w of core | 20% w/w of core |
| | Polyox grade | WSR N80 | WSR 205 |
| | PEG 3350 | 15% w/w of | 15% w/w of |
| | (Plasticizer) | coating solids | coating solids |
| | amount | | |
| | Coating Weight | 5.5% w/w | 5.5% w/w |
| | gain | | |

| | | | | |
|---|---|---|---|---|
| Material | mg/tab | % w/w core | mg/tab | % w/w core |

Tablet Core

| | | | | |
|---|---|---|---|---|
| Dexpramipexole Dihydrochloride Monohydrate | 319.00** | 42.00 | 319.00** | 42.00 |
| Microcrystalline cellulose (Avicel PH 102) | 79.75 | 10.50 | 79.75 | 10.50 |
| Copovidone (Kollidon VA 64 fine) | 53.17 | 7.00 | 53.17 | 7.00 |
| Sodium Chloride (Emprove ®) | 151.90 | 20.00 | 151.90 | 20.00 |
| Polyox WSR N80 | 151.90 | 20.00 | . . . | . . . |
| Polyox WSR 205 | . . . | . . . | 151.90 | 20.00 |
| Magnesium stearate (Vegetable source) | 3.80 | 0.50 | 3.80 | 0.50 |
| Total | 759.52 | 100.00 | 759.52 | 100.00 |

Semipermeable Membrane Coating\hat{ }\hat{ }

| | | | | |
|---|---|---|---|---|
| Opadry CA 500F190003 clear (Cellulose | 41.77 | . . . | 41.77 | . . . |
| acetate 398-10:PEG 3350 = 85:15) | | | | |
| Acetone\hat{ } | q.s. | . . . | q.s. | . . . |
| Purified water\hat{ } | q.s. | . . . | q.s. | . . . |
| Total | 801.29 | . . . | 801.29 | . . . |

Microcrystalline cellulose = diluent; copovidone = binder; sodium chloride = inorganic osmotic agent; magnesium stearate = lubricant; Polyox WSR N80 and Polyox WSR 205 = polymeric osmogen.
q.s.: Quantity sufficient
**Conversion factor for Dexpramipexole Dihydrochloride Monohydrate to Dexpramipexole Dihydrochloride = 0.9404
\hat{ }Evaporates during processing
\hat{ }\hat{ }Solid content of Coating Solution = 5% w/w, Acetone:Purified water = 95:5

The manufacturing process for preparing the blend and the procedure to prepare the semipermeable coating solution were the same as described above under Example 1. The in-process compression parameters and the in-process coating parameters for the lots in Table 4 are shown in Tables 5 and 6, respectively.

TABLE 5
In-process compression parameters for lots in Table 4.

| | Lot Number | |
|---|---|---|
| Parameter | AZF-1-FD-3-29B | AZF-1-FD-3-23D |
| Tooling | 9.50 mm * | 9.50 mm * |
| | 17.12 mm | 17.12 mm |
| Average tablet weight (mg) | 758 | 759 |
| Average Hardness (kP) | 17.4 | 16.9 |
| Thickness (mm) | 6.01 | 6.07 |
| Friability (%) | 0.11 | 0.14 |

TABLE 6
In-process coating parameters for lots in Table 4.

| | Lot Number | |
|---|---|---|
| Parameter | AZF-1-FD-3-29B | AZF-1-FD-3-23D |
| Pan load (g) | 350 | 350 |
| Inlet temperature (° C.) | 35-37 | 35-38 |
| Product temperature (° C.) | 27-28 | 26-27 |
| Spray rate (g/min) | 10 | 10 |
| Atomization (PSI) | 20 | 20 |
| Pan speed (rpm) | 22 | 20 |
| Drying time (min) | 5 | 5 |

The coated tablets of Table 4 were drilled with 0.5 mm delivery orifice using a mechanical drill press.

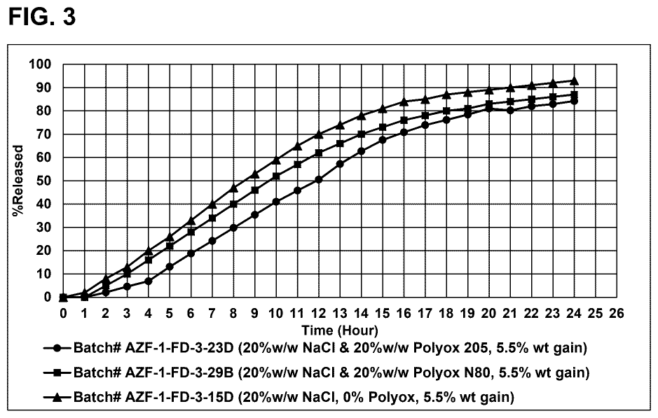

The dissolution results for Lot #AZF-1-FD-3-29B and Lot #AZ1F-171D-3-231D are provided in FIG. 3. They are compared with the dissolution profile of Lot #AZF-171D-3-15D of Example 1, which did not comprise a swellable polymeric osmotic agent.

The dissolution profiles in FIG. 3 show that swellable polymeric osmotic agent like polyethylene oxide was not acting synergistically with sodium chloride on the release of dexpramipexole dihydrochloride monohydrate. Although the formulations with swellable polymeric osmotic agent provide for suitable in vitro dissolution profiles (i.e., dissolution profiles that render the corresponding tablets suitable for once daily oral administration to a human), the release rate and terminal release were reduced in tablets comprising swellable polymeric osmotic agent.

### Example 3—Plasticizer Level

Further, different levels of plasticizer were evaluated for impact on release rate. Below, polyethylene glycol (PEG 3350) was selected as a plasticizer. The compositions used to study the impact of plasticizer amount are given in Table 7.

TABLE 7
Compositions used to study the impact of plasticizer amount
on the release of dexpramipexole dihydrochloride monohydrate.

| | |
|---|---|
| Formulation | Lot Number |

| | component | AZF-1-FD-3-8A | AZF-1-FD-3-15C |
|---|---|---|---|
| Summary | Sodium Chloride | 20% w/w of core | 20% w/w of core |
| | (Osmotic agent) | | |
| | amount | | |
| | PEG 3350 | 5% w/w of | 15% w/w of |
| | (Plasticizer) | coating solids | coating solids |
| | amount | | |
| | Coating Weight | 4% w/w | 4% w/w |
| | gain | | |

| | | | | |
|---|---|---|---|---|
| Material | mg/tab | % w/w core | mg/tab | % w/w core |

Tablet Core

| | | | | |
|---|---|---|---|---|
| Dexpramipexole Dihydrochloride Monohydrate | 319.00** | 42.00 | 319.00** | 42.00 |
| Microcrystalline cellulose (Avicel PH 102) | 231.85 | 30.50 | 231.85 | 30.50 |
| Copovidone (Kollidon VA 64 fine) | 53.17 | 7.00 | 53.17 | 7.00 |
| Sodium Chloride (Emprove ®) | 151.90 | 20.00 | 151.90 | 20.00 |
| Magnesium stearate (Vegetable source) | 3.80 | 0.50 | 3.80 | 0.50 |
| Total | 759.52 | 100.00 | 759.52 | 100.00 |

Semipermeable Membrane Coating\hat{ }\hat{ }

| | | | | |
|---|---|---|---|---|
| Opadry CA 500F190003 clear (Cellulose | . . . | . . . | 30.38 | . . . |
| acetate 398-10:PEG 3350 = 85:15) | | | | |
| Opadry CA 500F190011 clear (Cellulose | 30.38 | . . . | . . . | . . . |
| acetate 398-10:PEG 3350 = 95:5) | | | | |
| Acetone\hat{ } | q.s. | . . . | q.s. | . . . |
| Purified water\hat{ } | q.s. | . . . | q.s. | . . . |
| Total | 789.90 | . . . | 789.90 | . . . |

Microcrystalline cellulose = diluent; copovidone = binder; sodium chloride = inorganic osmotic agent; magnesium stearate = lubricant.
q.s.: Quantity sufficient
**Conversion factor for Dexpramipexole Dihydrochloride Monohydrate to Dexpramipexole Dihydrochloride = 0.9404
\hat{ }Evaporates during processing
\hat{ }\hat{ }Solid content of Coating Solution = 5% w/w, Acetone:Purified water = 95:5

The manufacturing process for preparing the blend and the procedure to prepare the coating solution were the same as described above under Example 1. The in-process compression parameters and the in-process coating parameters for the lots in Table 7 are shown in Tables 8 and 9, respectively.

TABLE 8
In-process compression parameters for lots in Table 7.

| | Lot Number | |
|---|---|---|
| Parameter | AZF-1-FD-3-8A | AZF-1-FD-3-15C |
| Tooling | 9.50 mm * | 9.50 mm * |
| | 17.12 mm | 17.12 mm |
| Average tablet weight (mg) | 758 | 758 |
| Average Hardness (kP) | 20.2 | 17.2 |
| Thickness (mm) | 5.71 | 5.71 |
| Friability (%) | 0.08 | 0.19 |

TABLE 9
In-process coating parameters for lots in Table 7.

| | Lot Number | |
|---|---|---|
| Parameter | AZF-1-FD-3-8A | AZF-1-FD-3-15C |
| Pan load (g) | 350 | 350 |
| Inlet temperature (° C.) | 33-38 | 35-37 |
| Product temperature (° C.) | 26-28 | 26-27 |
| Spray rate (g/min) | 10 | 10 |
| Atomization (PSI) | 20 | 20 |
| Pan speed (rpm) | 20 | 20 |
| Drying time (min) | 5 | 5 |

The coated tablets of Table 7 were drilled with 0.5 mm delivery orifice using a mechanical drill press.

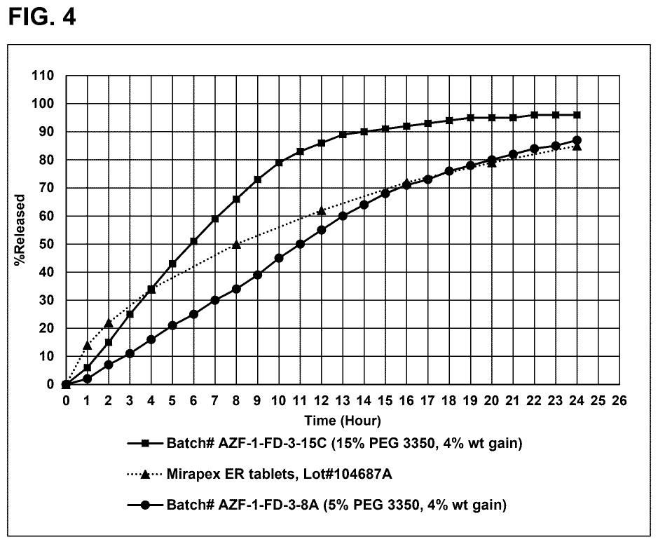

The dissolution results for Lot #AZF-1-FD-3-8A and Lot #AZF-1-FD-3-15C are provided in FIG. 4. The data show that release rate is increased upon increasing the plasticizer concentration from 5% w/w (of coatings solids) to 15% w/w (of coating solids). A higher plasticizer concentration (15% w/w of coating solids) requires higher coating weight gain (or membrane thickness) to achieve a beneficial in vitro dissolution profile, which is in turn favorable in terms of process efficiency for downstream coating process.

### Example 4—Coating Weight Gain/Membrane Thickness

Generally, a gain in coating weight results in an increase in semipermeable membrane coating thickness. In the following, three levels of coating weight gain were evaluated for impact on the release rate. Table 10 shows the compositions for evaluating the impact of coating weight gain.

TABLE 10
Compositions used to study the impact of membrane thickness/coating weight gain on
the release of dexpramipexole dihydrochloride monohydrate. Microcrystalline cellulose =
diluent; copovidone = binder; sodium chloride = inorganic osmotic agent;
magnesium stearate = lubricant.

| | Summary | | | | | |
|---|---|---|---|---|---|---|
| | Formulation component | | | | | |
| | Lot Number | | | | | |
| | AZF-1-FD-3-15C | | AZF-1-FD-3-15D | | AZF-1-FD-3-15E | |
| | Sodium Chloride (Osmotic agent) amount | | | | | |
| | 20% w/w of core | | 20% w/w of core | | 20% w/w of core | |
| | PEG 3350 (Plasticizer) amount | | | | | |
| | 15% w/w of | | 15% w/w of | | 15% w/w of | |
| | coating solids | | coating solids | | coating solids | |
| | Coating Weight gain | | | | | |
| | 4% w/w | | 5.5% w/w | | 7% w/w | |
| Material | mg/ tab | % w/w core | mg/ tab | % w/w core | mg/ tab | % w/w core |

Tablet Core

| | | | | | | |
|---|---|---|---|---|---|---|
| Dexpramipexole | 319.00** | 42.00 | 319.00** | 42.00 | 319.00** | 42.00 |
| Dihydrochloride | | | | | | |
| Monohydrate | | | | | | |
| Microsrystalline | 231.85 | 30.50 | 231.85 | 30.50 | 231.85 | 30.50 |
| cellulose | | | | | | |
| (Avicel PH 102) | | | | | | |
| Copovidone | 53.17 | 7.00 | 53.17 | 7.00 | 53.17 | 7.00 |
| (Kollidon VA | | | | | | |
| 64 fine) | | | | | | |
| Sodium Chloride | 151.90 | 20.00 | 151.90 | 20.00 | 151.90 | 20.00 |
| (Emprove ®) | | | | | | |
| Magnesium | 3.80 | 0.50 | 3.80 | 0.50 | 3.80 | 0.50 |
| stearate (Vegetable | | | | | | |
| source) | | | | | | |
| Total | 759.52 | 100.00 | 759.52 | 100.00 | 759.52 | 100.00 |

Semipermeable Membrane Coating\hat{ }\hat{ }

| | | | | | |
|---|---|---|---|---|---|
| Opadry CA | 30.38 | . . . | 41.77 | . . . | 53.17 |
| 500F190003 clear | | | | | |
| (Cellulose acetate | | | | | |
| 389-10:PEG 3350 = | | | | | |
| 85:15) | | | | | |
| Acetone\hat{ } | q.s. | . . . | q.s. | . . . | q.s. |
| Purified water\hat{ } | q.s. | . . . | q.s. | . . . | q.s. |
| Total | 789.90 | . . . | 801.29 | . . . | 812.69 |

q.s.: Quantity sufficient
**Conversion factor for Dexpramipexole Dihydrochloride Monoohydrate to Dexpramipexole Dihydrochloride = 0.9404
\hat{ }Evaporates during processing
\hat{ }\hat{ }Solid content of Coating Solution = 5% w/w, Acetone:Purified water = 95:5

The manufacturing process for preparing the blend and the procedure to prepare the coating solution were the same as described above under Example 1. The in-process compression parameters and the in-process coating parameters for the lots in Table 10 are shown in Tables 11 and 12, respectively.

TABLE 11
In-process compression parameters for lots in Table 10.

| | Lot Number |
|---|---|
| Parameter | AZF-1-FD-3-15C/15D/15E |
| Tooling | 9.50 mm * 17.12 mm |
| Average tablet weight (mg) | 758 |
| Average Hardness (kP) | 17.2 |
| Thickness (mm) | 5.71 |
| Friability (%) | 0.19 |

TABLE 12
In-process coating parameters for lots in Table 10.

| | Lot Number |
|---|---|
| Parameter | AZF-1-FD-3-15C/15D/15E |
| Pan load (g) | 350 |
| Inlet temperature (° C.) | 35-37 |
| Product temperature (° C.) | 26-27 |
| Spray rate (g/min) | 10 |
| Atomization (PSI) | 20 |
| Pan speed (rpm) | 20 |
| Drying time (min) | 5 |

The coated tablets of Table 10 were drilled with 0.5 mm delivery orifice using a mechanical drill press.

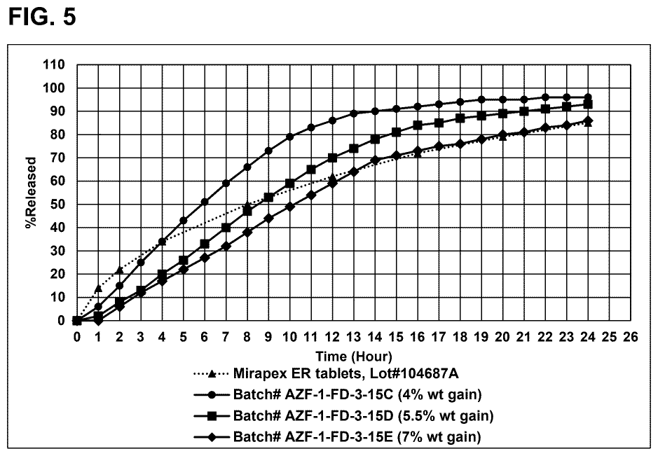

The dissolution results for Lot #AZF-1-FD-3-15C/15D/15E are provided in FIG. 5. The results show that release rate was reduced upon increasing the coating weight gain or membrane thickness. A weight gain of 5.5% w/w or more resulted in particularly favorable release profiles.

### Example 5—Number of Delivery Orifices

Next, the impact of number of delivery orifices on the release rate was evaluated. Table 13 shows the compositions tested to evaluate the impact of the number of delivery orifices.

TABLE 13
Compositions used to study the impact of the number of delivery orifices
on the release of dexpramipexole dihydrochloride monohydrate.

| | Formulation | Lot Number | |
|---|---|---|---|
| | component | AZF-1-FD-3-8B | AZF-1-FD-3-8C |
| Summary | Sodium Chloride | 20% w/w of core | 20% w/w of core |
| | (Osmotic agent) | | |
| | amount | | |
| | PEG 3350 | 5% w/w of | 5% w/w of |
| | (Plasticizer) | coating solids | coating solids |
| | amount | | |

| | | | | |
|---|---|---|---|---|
| Coating Weight | 10% | w/w | 10% | w/w |
| gain | | | | |
| Delivery Orifice | 0.5 | mm | 0.5 | mm |

| | | |
|---|---|---|
| Size | | |
| Number of | 1 | 4 |
| Delivery orifice | | |

| | | | | |
|---|---|---|---|---|
| Material | mg/tab | % w/w core | mg/tab | % w/w core |

Tablet Core

| | | | | |
|---|---|---|---|---|
| Dexpramipexole Dihydrochloride Monohydrate | 319.00** | 42.00 | 319.00** | 42.00 |
| Microcrystalline cellulose (Avicel PH 102) | 231.85 | 30.50 | 231.85 | 30.50 |
| Copovidone (Kollidon VA 64 fine) | 53.17 | 7.00 | 53.17 | 7.00 |
| Sodium Chloride (Emprove ®) | 151.90 | 20.00 | 151.90 | 20.00 |
| Magnesium stearate (Vegetable source) | 3.80 | 0.50 | 3.80 | 0.50 |
| Total | 759.52 | 100.00 | 759.52 | 100.00 |

Semipermeable Membrane Coating\hat{ }\hat{ }

| | | | | |
|---|---|---|---|---|
| Opadry CA 500F190011 clear (Cellulose | 75.92 | . . . | 75.92 | . . . |
| acetate 398-10:PEG 3350 = 95:5) | | | | |
| Acetone\hat{ } | q.s. | . . . | q.s. | . . . |
| Purified water\hat{ } | q.s. | . . . | q.s. | . . . |
| Total | 835.44 | . . . | 835.44 | . . . |

Microcrystalline cellulose = diluent; copovidone = binder, sodium chloride = inorganic osmotic agent; magnesium stearate = lubricant.
q.s.: Quantity sufficient
**Conversion factor for Dexpramipexole Dihydrochloride Monohydrate to Dexpramipexole Dihydrochloride = 0.9404
\hat{ }Evaporates during processing
\hat{ }\hat{ }Solid content of Coating Solution = 5% w/w, Acetone:Purified water = 95:5

The manufacturing process for preparing the blend and the procedure to prepare the coating solution were the same as described above under Example 1. The in-process compression parameters and the in-process coating parameters for the lots in Table 13 are shown in Tables 14 and 15, respectively.

TABLE 14
In-process compression parameters for lots in Table 13.

| | Lot Number |
|---|---|
| Parameter | AZF-1-FD-3-8B/8C |
| Tooling | 9.50 mm*17.12 mm |
| Average tablet weight (mg) | 758 |
| Average Hardness (kP) | 20.2 |
| Thickness (mm) | 5.71 |
| Friability (%) | 0.08 |

TABLE 15
In-process coating parameters for lots in Table 13.

| | Lot Number |
|---|---|
| Parameter | AZF-1-FD-3-8B/8C |
| Pan load (g) | 350 |
| Inlet temperature (° C.) | 33-38 |
| Product temperature (° C.) | 26-28 |
| Spray rate (g/min) | 10 |
| Atomization (PSI) | 20 |
| Pan speed (rpm) | 20 |
| Drying time (min) | 5 |

The coated tablets of Table 13 were drilled with 0.5 mm delivery orifice using a mechanical drill press. Coated tablets of Lot No. AZF-1-FD-3-8B were drilled with one delivery orifice and coated tablets of Lot No. AZF-1-FD-3-8C were drilled with four delivery orifices.

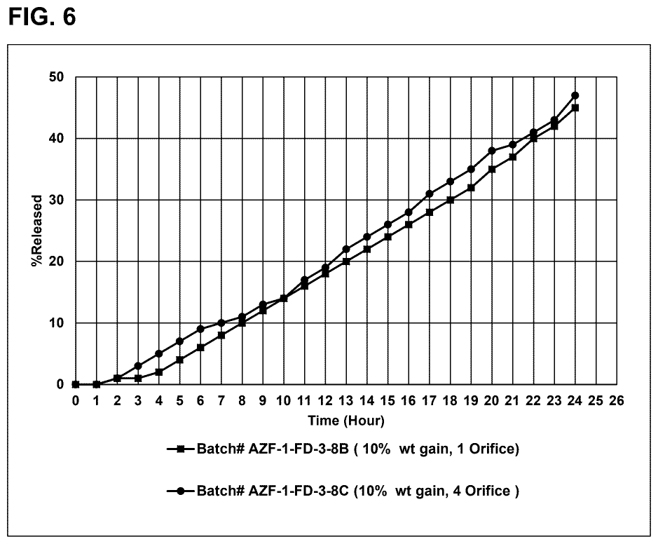

The dissolution results for Lot #AZF-1-FD-3-8B and Lot #AZF-1-FD-3-8C are provided in FIG. 6. In general, no significant impact on the release profile was observed when the number of delivery orifice was increased from one to four. Nevertheless, the in vitro dissolution profiles show that the slope of the zero order release profile was more constant for the formulation with one delivery orifice.

### Example 6—Immediate Release Coating

In addition to osmotic tablets comprising dexpramipexole dihydrochloride monohydrate solely in a sustained release form (such as the tablets prepared under Examples 1 to 5 above), tablets were prepared that in addition to a sustained release form of dexpramipexole dihydrochloride monohydrate also provide dexpramipexole dihydrochloride monohydrate in an immediate release form.

In the following, two different tablet formulations (Batches AZF-1-FD-4-11A and AZF-1-FD-4-11B) were manufactured. In the first formulation, 100% of the total dexpramipexole dihydrochloride monohydrate amount (corresponding to 300 mg dexpramipexole dihydrochloride equivalent) was incorporated into the tablet core. In the second formulation, 90% of the total dexpramipexole dihydrochloride monohydrate amount (corresponding to 270 mg dexpramipexole dihydrochloride equivalent) was incorporated into the tablet core and 10% of total amount (corresponding to 30 mg dexpramipexole dihydrochloride equivalent) was incorporated as an immediate release drug coating surrounding the semipermeable membrane-coated tablet core. An immediate release drug coating can reduce the lag time for drug release and result in a quick onset of action in patients. The tablet cores were prepared using a direct compression process.

Batches AZF-1-FD-4-11A and AZF-1-FD-4-11B (“confirmation batches”) were manufactured at a larger scale (1 kg blend). The formulation of the batches evaluated in this Example is given in Tables 16 and 17.

TABLE 16
Formulation of batch AZF-1-FD-4-11A
(without immediate release layer).

| | | Batch Number | |
|---|---|---|---|
| | Formulation component | AZF-1-FD-4-11A | |
| Summary | Tablet Core manufacturing Process | Direct Compression | |
| | Drug substance (Dexpramipexole | 300 | mg |
| | Dihydrochloride equivalent) in core | | |
| | Drug substance (Dexpramipexole | 0 | mg |
| | Dihydrochloride equivalent) in | | |
| | Immediate release coating | | |
| | Membrane Coating Weight gain | 6.2% | w/w |
| | Plasticizer (PEG 3350) level | 15% w/w of | |
| | | coating solids | |
| | Orifice Size | 0.5 | mm |

| | | |
|---|---|---|
| Material | mg/tab | % w/w core |

Tablet Core

| | | |
|---|---|---|
| Dexpramipexole Dihydrochloride Monohydrate | 319.00** | 42.00 |
| Microcrystalline cellulose (Avicel PH 102) | 231.85 | 30.50 |
| Copovidone (Kollidon VA 64 fine) | 53.17 | 7.00 |
| Sodium Chloride (Emprove ®) | 151.90 | 20.00 |
| Magnesium stearate (Vegetable source) | 3.80 | 0.50 |
| Total | 759.52 | 100.00 |

Semipermeable Membrane Coating\hat{ }\hat{ }

| | | |
|---|---|---|
| Opadry CA 500F190003 clear (Cellulose | 47.09 | . . . |
| acetate 398-10:PEG 3350 = 85:15) | | |
| Acetone\hat{ } | q.s. | . . . |
| Purified water\hat{ } | q.s. | . . . |
| Total | 806.61 | . . . |

Film Coating^{α}

| | | |
|---|---|---|
| Opadry II 85F18422 white | 24.20 | . . . |
| Purified water\hat{ } | q.s. | . . . |
| Total | 830.81 | . . . |

Microcrystalline cellulose = diluent; copovidone = binder, sodium chloride = inorganic osmotic agent; magnesium stearate = lubricant.
q.s.: Quantity sufficient
**Conversion factor for Dexpramipexole Dihydrochloride Monohydrate to Dexpramipexole Dihydrochloride = 0.9404
\hat{ }Evaporates during processing
\hat{ }\hat{ }Solid content of Membrane Coating Solution = 5% w/w, Acetone:Purified water = 95:5
^{α}Solid content of Film Coating dispersion = 12% w/w.

TABLE 17
Formulation of batch AZF-1-FD-4-
11B (with immediate release layer).

| | | Batch Number | |
|---|---|---|---|
| | Formulation component | AZF-1-FD-4-11B | |
| Summary | Tablet Core manufacturing Process | Direct Compression | |
| | Drug substance (Dexpramipexole | 270 | mg |
| | Dihydrochloride equivalent) in core | | |
| | Drug substance (Dexpramipexole | 30 | mg |
| | Dihydrochloride equivalent) in | | |
| | Immediate release coating | | |
| | Membrane Coating Weight gain | 7.3% | w/w |
| | Plasticizer (PEG 3350) level | 15% w/w of | |
| | | coating solids | |
| | Orifice Size | 0.5 | mm |

| | | |
|---|---|---|
| Material | mg/tab | % w/w core |

Tablet Core

| | | |
|---|---|---|
| Dexpramipexole Dihydrochloride Monohydrate | 287.10** | 42.00 |
| Microcrystalline cellulose (Avicel PH 102) | 208.49 | 30.50 |
| Copovidone (Kollidon VA 64 fine) | 47.85 | 7.00 |
| Sodium Chloride (Emprove ®) | 136.71 | 20.00 |
| Magnesium stearate (Vegetable source) | 3.42 | 0.50 |
| Total | 683.57 | 100.00 |

Semipermeable Membrane Coating\hat{ }\hat{ }

| | | |
|---|---|---|
| Opadry CA 500F190003 clear (Cellulose | 49.90 | . . . |
| acetate 398-10:PEG 3350 = 85:15) | | |
| Acetone\hat{ } | q.s. | . . . |
| Purified water\hat{ } | q.s. | . . . |
| Total | 733.47 | . . . |

Seal Coating^{β}

| | | |
|---|---|---|
| Opadry YS-1-19025-A clear | 22.00 | |
| Purified water\hat{ } | q.s. | . . . |
| Total | 755.47 | . . . |

Immediate Release Drug Coating^{€}

| | | |
|---|---|---|
| Dexpramipexole Dihydrochloride Monohydrate | 31.90** | . . . |
| Opadry YS-1-19025-A clear (Binder) | 31.90 | . . . |
| Purified water\hat{ } | q.s. | . . . |
| Total | 819.27 | . . . |

Film Coating^{α}

| | | |
|---|---|---|
| Opadry II 85F18422 white | . . . | . . . |
| Purified water\hat{ } | . . . | . . . |
| Total | 819.27 | . . . |

Microcrystalline cellulose = diluent; copovidone = binder, sodium chloride = inorganic osmotic agent; magnesium stearate = lubricant.
q.s.: Quantity sufficient
**Conversion factor for Dexpramipexole Dihydrochloride Monohydrate to Dexpramipexole Dihydrochloride = 0.9404
\hat{ }Evaporates during processing
\hat{ }\hat{ }Solid content of Membrane Coating Solution = 5% w/w, Acetone:Purified water = 95:5
^{β, €}Solid content of Seal/Drug Coating Solution = 10% w/w
^{α}Solid content of Film Coating dispersion = 12% w/w.

The final blend properties such as bulk density, tapped density, Carr's Index, and flow function coefficient (ffc) were measured for Batch No. AZF-1-FD-4-11A and the results are provided below in Table 18. As shown in Table 18, the blend has a good flow. Compression process parameters and in-process compression checks of Batch No. AZF-1-FD-4-11A and AZF-1-FD-4-11B are described in Example 7.1 below. The in-process parameters for the membrane coating of Batch No. AZF-1-FD-4-11A and AZF-1-FD-4-11B are given in Table 19 and the in-process parameters for the seal coating of Batch No. AZF-1-FD-4-11B are given in Table 20. The in-process parameters for the immediate release drug coating of Batch No. AZF-1-FD-4-11B are given in Table 21 and the in-process parameters for the film coating of Batch No. AZF-1-FD-4-11A are provided in Table 22.

TABLE 18
Final blend properties for Lot# AZF-1-FD-4-11A (Table 16).

| Property | Value |
|---|---|
| Bulk Density | 0.64 g/mL |
| Tapped Density | 0.77 g/mL |
| Carr's Index | 17.2 |
| Flow function coefficient (ffc)* | 16.2 |

*Measured using Shear cell FT4 powder rheometer at 3 MPa shear stress

TABLE 19
In-process coating parameters for membrane
coating for lots in Tables 16 and 17.

| | Batch Number | |
|---|---|---|
| Parameter | AZF-1-FD-4-11A | AZF-1-FD-4-11B |
| Pan load (g) | 836 | 836 |
| Inlet temperature (° C.) | 36-38 | 36-38 |
| Product temperature (° C.) | 26-27 | 26-27 |
| Spray rate (g/min) | 16 | 16 |
| Atomization (PSI) | 20 | 20 |
| Pan speed (rpm) | 17 | 17 |
| Drying time (min) | 5 | 5 |

TABLE 20
In-process coating parameters for seal coating
of Batch No. AZF-1-FD-4-11B (Table 17).

| | Batch Number |
|---|---|
| Parameter | AZF-1-FD-4-11B |
| Pan load (g) | 850 |
| Inlet temperature (° C.) | 60-62 |
| Product temperature (° C.) | 40-42 |
| Spray rate (g/min) | 8 |
| Atomization (PSI) | 15 |
| Pan speed (rpm) | 17 |
| Drying time (min) | 5 |

TABLE 21
In-process coating parameters for drug coating
of Batch No. AZF-1-FD-4-11B (Table 17).

| | Batch Number |
|---|---|
| Parameter | AZF-1-FD-4-11B |
| Pan load (g) | 823 |
| Inlet temperature (° C.) | 60-62 |
| Product temperature (° C.) | 39-42 |
| Spray rate (g/min) | 10 |
| Atomization (PSI) | 15 |
| Pan speed (rpm) | 17 |
| Drying time (min) | 5 |

TABLE 22
In-process coating parameters for the film coating
of Batch No. AZF-1-FD-4-11A (Table 16).

| | Batch Number |
|---|---|
| Parameter | AZF-1-FD-4-11B |
| Pan load (g) | 850 |
| Inlet temperature (° C.) | 61-64 |
| Product temperature (° C.) | 41-42 |
| Spray rate (g/min) | 8 |
| Atomization (PSI) | 15 |
| Pan speed (rpm) | 17 |
| Drying time (min) | 5 |

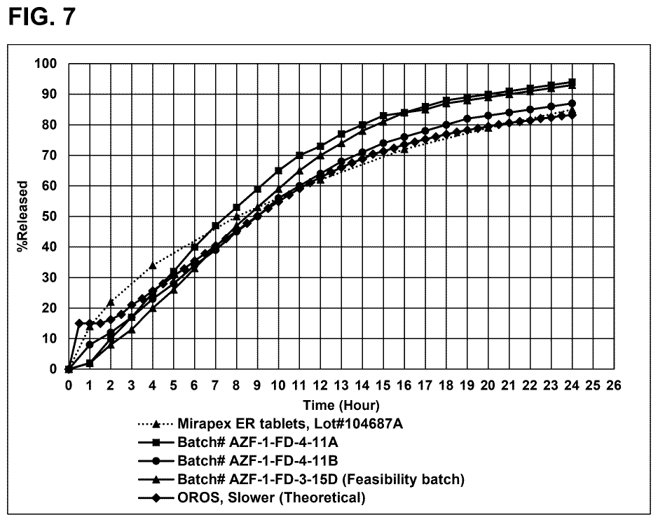

Dissolution results for the tablets in Tables 16 and 17 are shown in FIG. 7. Batch No. AZF-1-FD-3-15D (Example 4; prepared using ˜500 g blend) was also included for comparison. The data show that there was no significant impact of scale up process on the dissolution. Thus, the formulation is reproducible with respect to process. Furthermore, the results suggest there was no significant impact of the presence of a seal coating or film coating on the dissolution of the tablet cores. A theoretical release profile for a formulation having 255 mg drug substance (mg-amount refers to dexpramipexole dihydrochloride) in the tablet core (85% of the total dose) and 45 mg drug substance (mg-amount refers to dexpramipexole dihydrochloride) in the immediate release drug coating (15% of the total dose) was also generated based on release profile of Batch No. AZF-1-FD-4-111B.

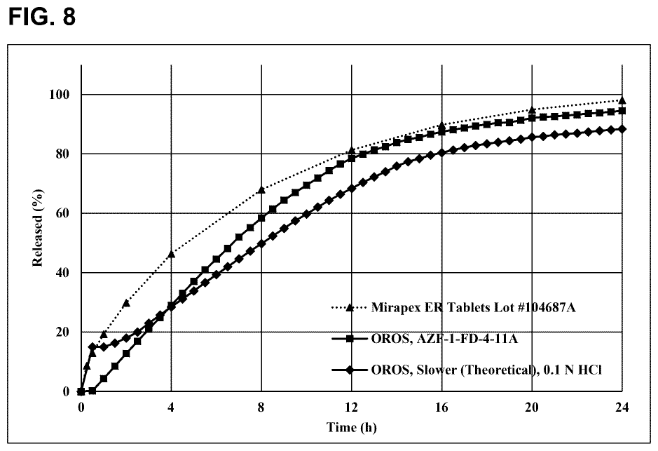

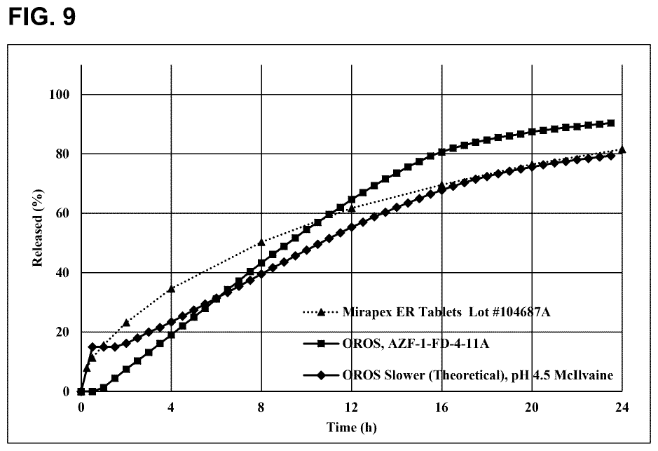

Dissolution was further evaluated in 0.1 N HCl and McIlvaine Buffer, pH 4.5 (FIGS. 8 and 9). A theoretical release profile for a formulation having 255 mg drug substance (mg-amount refers to dexpramipexole dihydrochloride) in the tablet core (85% of the total dose) and 45 mg drug substance (mg-amount refers to dexpramipexole dihydrochloride) in coating (15% of the total dose) was also generated based on release profile of Batch No. AZF-1-FD-4-111B. The results indicate that the dissolution behavior of the osmotic tablets was pH-independent.

Another tablet batch (AZF-1-FD-4-28D) with an immediate release drug coating was manufactured using direct compression at a scale of ˜500 g (see Table 23).

TABLE 23
Formulation of batch AZF-1-FD-4-
28D (with immediate release layer).

| | | Batch Number | |
|---|---|---|---|
| | Formulation component | AZF-1-FD-4-28D | |
| Summary | Tablet Core manufacturing Process | Direct Compression | |
| | Drug substance (Dexpramipexole | 255 | mg |
| | Dihydrochloride equivalent) in core | | |
| | Drug substance (Dexpramipexole | 45 | mg |
| | Dihydrochloride equivalent) in | | |
| | Immediate release coating | | |
| | Membrane Coating Weight gain | 8.0% | w/w |

| | |
|---|---|
| Plasticizer (PEG 3350) level | 15% w/w of |
| | coating solids |

| | | |
|---|---|---|
| Material | mg/tab | % w/w core |

Tablet Core

| | | |
|---|---|---|
| Dexpramipexole Dihydrochloride Monohydrate | 271.15** | 42.00 |
| Microcrystalline cellulose (Avicel PH 102) | 196.90 | 30.50 |
| Copovidone (Kollidon VA 64 fine) | 45.19 | 7.00 |
| Sodium Chloride (Emprove ®) | 129.12 | 20.00 |
| Magnesium stearate (Vegetable source) | 3.23 | 0.50 |
| Total | 645.59 | 100.00 |

Semipermeable Membrane Coating\hat{ }\hat{ }

| | | |
|---|---|---|
| Opadry CA 500F190003 clear (Cellulose | 51.65 | . . . |
| acetate 398-10:PEG 3350 = 85:15) | | |
| Acetone\hat{ } | q.s. | . . . |
| Purified water\hat{ } | q.s. | . . . |
| Total | 697.24 | . . . |

Seal Coating^{β}

| | | |
|---|---|---|
| Opadry YS-1-19025-A clear | 20.92 | |
| Purified water\hat{ } | q.s. | . . . |
| Total | 718.16 | . . . |

Immediate Release Drug Coating^{€}

| | | |
|---|---|---|
| Dexpramipexole Dihydrochloride Monohydrate | 47.85** | . . . |
| Opadry YS-1-19025-A clear (Binder) | 47.85 | . . . |
| Purified water\hat{ } | q.s. | . . . |
| Total | 813.86 | . . . |

Film Coating^{α}

| | | |
|---|---|---|
| Opadry II 85F18422 white | 24.41 | . . . |
| Purified water\hat{ } | q.s. | . . . |
| Total | 838.27 | . . . |

Microcrystalline cellulose = diluent; copovidone = binder, sodium chloride = inorganic osmotic agent; magnesium stearate = lubricant.
q.s.: Quantity sufficient
**Conversion factor for Dexpramipexole Dihydrochloride Monohydrate to Dexpramipexole Dihydrochloride = 0.9404
\hat{ }Evaporates during processing
\hat{ }\hat{ }Solid content of Membrane Coating Solution = 5% w/w, Acetone: Purified water = 95:5
^{β, €}Solid content of Seal/Drug Coating Solution = 10% w/w
^{α}Solid content of Film Coating dispersion = 12% w/w.

### Example 7—Process Development

In general, manufacturing the osmotic tablets without an immediate release coating was carried out by: preparing a pre-blend comprising the drug substance (dexpramipexole, or a pharmaceutically acceptable salt thereof) and one or more further ingredients such as the inorganic osmotic agent, preparing a blend from the pre-blend and a lubricant (such as magnesium stearate), compressing the blend to form a tablet core, coating the tablet core with a semipermeable membrane coating solution to provide a semipermeable membrane coating surrounding the tablet core, and optionally coating the semipermeable membrane coating with a film coating solution to provide a film coating surrounding the semipermeable membrane coating (more than one film coating may be applied). Further, drilling one or more delivery orifices in the semipermeable membrane coating (such as with mechanical or laser drilling) was carried out.

In general, manufacturing the osmotic tablets with an immediate release coating was carried out by: preparing a pre-blend comprising the drug substance (dexpramipexole, or a pharmaceutically acceptable salt thereof) and one or more further ingredients such as the inorganic osmotic agent, preparing a blend from the pre-blend and a lubricant (such as magnesium stearate), compressing the blend to form a tablet core, coating the tablet core with a semipermeable membrane coating solution to provide a semipermeable membrane coating surrounding the tablet core, coating the semipermeable membrane coating with a seal coating solution to provide a seal coating surrounding the semipermeable membrane coating, coating the seal coating with an immediate release drug coating solution to provide an immediate release drug coating surrounding the seal coating, and optionally further coating the drug coating with a film coating solution to provide a film coating surrounding the drug coating (more than one film coating may be applied). Further, drilling one or more delivery orifices in the semipermeable membrane coating (such as with mechanical or laser drilling) was carried out.

In general, during preparation of the pre-blend and the blend, no water was added or heat was applied. Further, during compression of the blend, no further excipients or water were introduced. The solvent of the semipermeable membrane coating solution was acetone: purified water at the indicated ratios. The drilling process did not introduce any new excipients or applied water. Purified water was the solvent for preparing the seal coating solution, the immediate release drug coating solution, and the film coating solution.

In Examples 7.1 and 7.2 below, compression of the blend and application of semipermeable membrane coating were evaluated further.

### Example 7.1—Compression Process Development

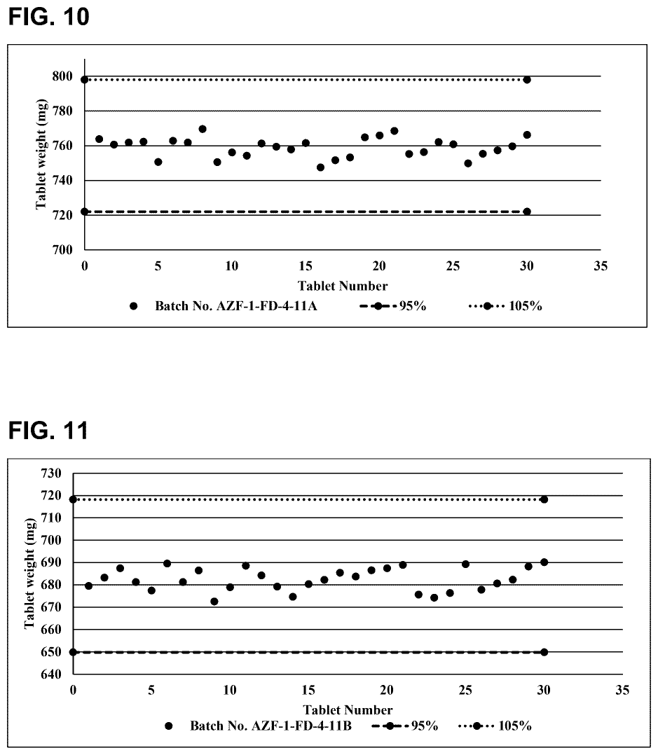

Compression was carried out using a Korsch XL 100 rotary tablet press. A summary of the process parameters used for batches AZF-1FD-4-11A and AZF-1-FD-4-11B (see Example 6 above) is given in Table 24. Beginning-, middle-, and end-of-batch samples were collected for weight, thickness, and hardness measurements. The results are summarized in Tables 25 and 26. Weight variation between the different tablets prepared per batch was low (FIGS. 10 and 11).

TABLE 24
Summary of compression process parameters
for batches in Tables 16 and 17.

| | Confirmation Batches | |
|---|---|---|
| Parameter | AZF-1-FD-4-11A | AZF-1-FD-4-11B |

Press Set-Up

| Tooling | 9.50 mm × 17.12 mm | | 9.50 mm × 17.12 mm | |
|---|---|---|---|---|
| Turret used | 10 station B | | 10 station B | |
| Number of punches used | 4 stations | | 4 stations | |
| Fill cam used | 8-14 | mm | 8-14 | mm |

Parameters

| | | | | |
|---|---|---|---|---|
| Pre-compression force | 0.5-0.7 | kN | 0.2-0.6 | kN |
| Main compression force | 35.2-42.7 | kN | 30.8-34.9 | kN |
| Feeder speed | 20 | rpm | 20 | rpm |
| Press speed | 20 | rpm | 20 | rpm |

TABLE 25
Summary of in-process compression checks
for batch AZF-1-FD-4-11A (Table 16).

| Tablet | Beginning | Middle | End |
|---|---|---|---|
| No. | Weight (mg) | Weight (mg) | Weight (mg) |
| 1 | 763.8 | 754.2 | 768.5 |
| 2 | 760.6 | 761.3 | 755.2 |
| 3 | 761.8 | 759.4 | 756.3 |
| 4 | 762.3 | 757.8 | 762.1 |
| 5 | 750.6 | 761.5 | 760.8 |
| 6 | 762.8 | 747.5 | 749.8 |
| 7 | 761.8 | 751.6 | 755.3 |
| 8 | 769.6 | 753.2 | 757.3 |
| 9 | 750.5 | 764.8 | 759.6 |
| 10 | 756.1 | 765.9 | 766.2 |
| Avg. | 760.0 | 757.9 | 759.1 |
| Min. | 750.5 | 747.5 | 749.3 |
| Max | 769.6 | 765.9 | 768.5 |
| Average | 17.5 | 18.8 | 19.8 |
| Hardness (kp) | | | |
| Average | 6.01 | 6.03 | 5.99 |
| Thickness mm) | | | |

TABLE 26
Summary of in-process compression checks
for batch AZF-1-FD-4-11B (Table 17).

| Tablet | Beginning | Middle | End |
|---|---|---|---|
| No. | Weight (mg) | Weight (mg) | Weight (mg) |
| 1 | 679.5 | 688.5 | 688.9 |
| 2 | 683.2 | 684.2 | 675.6 |
| 3 | 687.4 | 679.2 | 674.2 |
| 4 | 681.2 | 674.6 | 676.3 |
| 5 | 677.4 | 680.3 | 689.2 |
| 6 | 689.5 | 682.2 | 677.8 |
| 7 | 681.2 | 685.4 | 680.6 |
| 8 | 686.4 | 683.7 | 682.3 |
| 9 | 672.5 | 686.5 | 688.2 |
| 10 | 678.9 | 687.4 | 690.1 |
| Avg. | 681.7 | 683.2 | 682.3 |
| Min. | 672.5 | 674.6 | 675.6 |
| Max | 689.5 | 688.5 | 690.1 |
| Average | 18.5 | 19.3 | 19.2 |
| Hardness (kp) | | | |
| Average | 5.85 | 5.84 | 5.83 |
| Thickness (mm) | | | |

### Example 7.2—Membrane Coating Process Development

Smaller scale manufacturing was performed using a Vector LDCS-5 pan coater configured with partially perforated pan. Larger scale manufacturing was performed using an O'hara coating machine configured with fully perforated pan. A summary of the process parameters used for the development batches described in Examples 1 to 5 and for the two confirmation batches described in Example 6 (see Table 16 and Table 17) is provided in Table 27. The results show that process efficiency was close to 100% for all batches indicating the robustness of the membrane coating process.

TABLE 27
Summary of in-process coating parameters for membrane coating
of development batches and confirmation batches.

| | Development | Confirmation |
|---|---|---|
| Parameter | batches | batches |
| Pan size | 6 inches | 12 inches |
| Pan load (g) | 350 | 836 |
| Inlet temperature (° C.) | 33-38 | 36-38 |
| Product temperature (° C.) | 26-28 | 26-27 |
| Spray rate (g/min) | 10 | 16 |
| Atomization (PSI) | 20 | 20 |
| Pan speed (rpm) | 20 | 17 |
| Drying time (min) | 5 | 5 |
| Process Efficiency | 97% | 99.5% |

### Example 7.3—Engineering Batches

Engineering scale-up batches were manufactured at 10 kg scale. The formulations are provided in Table 28. Instead of mechanical drilling, laser drilling was performed using an Ackley Laser drilling machine. Laser drilling was performed after finishing film coating of the tablets.

TABLE 28
Formulation of engineering batches.

| | Batch Number | | | |
|---|---|---|---|---|
| | 5635331 | | 5635332 | |
| | Fast Prototype | | Slow Prototype | |
| | | % w/w | | % w/w |
| Material | mg/tab | core | mg/tab | core |

Core tablet

| | | | | |
|---|---|---|---|---|
| Dexpramipexole Dihydrochloride Monohydrate | 319.01** | 42.00 | 271.16** | 42.00 |
| Microcrystalline cellulose (Avicel PH 102) | 231.65 | 30.50 | 196.90 | 30.50 |
| Copovidone (Kollidon VA 64 fine) | 53.17 | 7.00 | 45.19 | 7.00 |
| Sodium Chloride (Emprove ®) | 151.90 | 20.00 | 129.12 | 20.00 |
| Magnesium stearate (Vegetable source) | 3.80 | 0.50 | 3.23 | 0.50 |
| Total | 759.53 | 100.00 | 645.60 | 100.00 |

Membrane Coating\hat{ }\hat{ }

| | | | | |
|---|---|---|---|---|
| Opadry CA 500F190003 clear (Cellulose | 47.09 | . . . | 51.65 | . . . |
| acetate 398-10:PEG 3350 = 85:15) | | | | |
| Acetone\hat{ } | 841.03\hat{ } | . . . | 922.47\hat{ } | . . . |
| Purified water\hat{ } | 53.68\hat{ } | . . . | 58.88\hat{ } | . . . |
| Total | 806.62 | . . . | 697.25 | . . . |

Seal Coating^{β}

| | | | | |
|---|---|---|---|---|
| Opadry YS-1-19025-A clear | . . . | . . . | 20.92 | . . . |
| Purified water\hat{ } | . . . | . . . | 188.28\hat{ } | . . . |
| Total | . . . | . . . | 718.17 | . . . |

Immediate Release Drug Coating^{€}

| | | | | |
|---|---|---|---|---|
| Dexpramipexole Dihydrochloride Monohydrate | . . . | . . . | 47.85** | . . . |
| Opadry YS-1-19025-A clear | . . . | . . . | 47.85 | . . . |
| Purified water\hat{ } | . . . | . . . | 861.30\hat{ } | . . . |
| Total | . . . | . . . | 813.87 | . . . |

Film Coating^{α#}

| | | | | |
|---|---|---|---|---|
| Opadry II 85F18422 white | 24.20 | . . . | 24.42 | . . . |
| Purified water\hat{ } | 96.80\hat{ } | . . . | 97.68\hat{ } | . . . |
| Total | 830.82 | . . . | 838.29 | . . . |

Microcrystalline cellulose = diluent; copovidone = binder, sodium chloride = inorganic osmotic agent; magnesium stearate = lubricant.
q.s.: Quantity sufficient,
**Conversion factor for Dexpramipexole Dihydrochloride Monohydrate to Dexpramipexole Dihydrochloride = 0.9404,
\hat{ }Evaporates during processing,
\hat{ }\hat{ }Solid content of Coating Solution = 5% w/w, Acetone:water = 94:6,
^{β, €}Solid content of Coating Solution = 10% w/w,
^{α}Solid content of Coating dispersion = 20% w/w,
^{#}Laser drilling done at the end of film coating

A summary of the scale-up process parameters is provided in Table 29.

TABLE 29
Summary of process parameters for engineering batches 5635331 and 5635332.

| Process Parameters | | 5635331 (Fast) | | 5635332 (Slow) | |
|---|---|---|---|---|---|
| Compression | Target Weight (mg) | 760 | (722-798) | 646 | (614-678) |
| | Target Composite (mg) (10 tabs) | 7600 | (7370-7830) | 6460 | (6270-6650) |
| | Target Thickness (mm) | Report Results | | Report Results | |
| | Target Hardness (kP) | 20 | (15-25) | 15 | (10-20) |
| | Press Speed (rpm) | 25 | | 25 | |
| | Feeder Speed (rpm) | 35 | | 35 | |
| Membrane | Pan size (in) | 24 | | 24 | |
| Coating | Nozzle port opening (mm) | 1.2 | | 1.2 | |
| | Drum Speed (rpm) | 8-12 | | 8-12 | |
| | Inlet Air Volume (cfm) | 250-275 | | 250-275 | |
| | Inlet Air Temperature (° C.) | 30-40 | | 30-40 | |
| | Product Temperature (° C.) | 25-27 | | 24-26 | |
| | Exhaust Air Temperature (° C.) | 20-30 | | 49-50 | |
| | Target Weight Gain | 6.2% | (6.0%-6.4%) | 8.0% | (7.8%-8.2%) |
| | Spray Rate (g/min) | 80-100 | | 70-100 | |
| | Pattern Air Pressure (psi) | 20 | | 20 | |
| | Atomizing Air Pressure (psi) | 15 | | 15 | |
| Seal Coating | Pan size (in) | . . . | | 24 | |
| | Nozzle port opening (mm) | | | 1.2 | |
| | Drum Speed (rpm) | | | 12-16 | |
| | Inlet Air Volume (cfm) | | | 250-300 | |
| | Inlet Air Temperature (° C.) | | | 55-65 | |
| | Product Temperature (° C.) | | | Not recorded | |
| | Exhaust Air Temperature (° C.) | | | 40-50 | |
| | Target Weight Gain | | | 3.0% | (2.5%-3.5%) |
| | Spray Rate (g/min) | | | 35 | |
| | Pattern Air Pressure (psi) | | | 25 | |
| | Atomizing Air Pressure (psi) | | | 35 | |
| Immediate | Pan size (in) | . . . | | 24 | |
| Release Drug | Nozzle port opening (mm) | | | 1.2 | |
| Coating | Drum Speed (rpm) | | | 12-16 | |
| | Inlet Air Volume (cfm) | | | 250-300 | |
| | Inlet Air Temperature (° C.) | | | 55-65 | |
| | Product Temperature (° C.) | | | Not recorded | |
| | Exhaust Air Temperature (° C.) | | | 40-50 | |
| | Target Weight Gain | | | 13.33% | (12.83%-13.83%) |
| | Spray Rate (g/min) | | | 35 | |
| | Pattern Air Pressure (psi) | | | 25 | |
| | Atomizing Air Pressure (psi) | | | 35 | |
| Film | Pan size (in) | 24 | | 24 | |
| Coating | Nozzle port opening (mm) | 1.2 | | 1.2 | |
| | Drum Speed (rpm) | 12-16 | | 12-16 | |
| | Inlet Air Volume (cfm) | 250-300 | | 250-300 | |
| | Inlet Air Temperature (° C.) | 55-65 | | 55-65 | |
| | Product Temperature (° C.) | Not recorded | | Not recorded | |
| | Exhaust Air Temperature (° C.) | 40-50 | | 40-50 | |
| | Target Weight Gain | 3.0% | (2.5%-3.5%) | 3.0% | (2.5%-3.5%) |

| | | | |
|---|---|---|---|
| | Spray Rate (g/min) | 45 | 35 |
| | Pattern Air Pressure (psi) | 25 | 25 |
| | Atomizing Air Pressure (psi) | 35 | 35 |
| Laser | Spindle speed (rpm) | 800 | 800 |
| Drilling | System speed (links/min) | 500 | 500 |
| | Pitch (%) | 0.040 | 0.040 |
| | Drill speed (mm/sec) | 2500 | 2500 |
| | Power level (%) | 90 | 90 |
| | PWM Frequency (KHz) | 25 | 25 |
| | Hole count per tablet | 1 | 1 |
| | Hole Diameter (mm) | 0.5 | 0.5 |

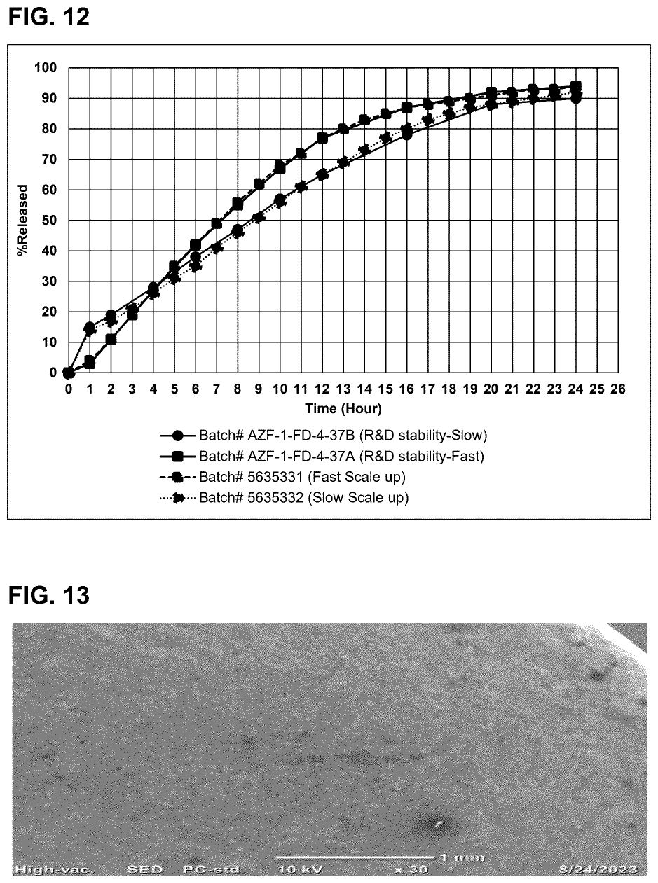

FIG. 12 provides a comparison of the engineering batches with corresponding smaller-scale laboratory batches. The formulation of batch AZF-1-FD-4-37A is identical to the formulation of batch 5635331. The formulation of batch AZ1-FD-4-37B is identical to the formulation of batch 5635332. The results indicate good reproducibility.

### Example 7.4—Variation of Drug to Binder Ratio in Immediate Release Drug Coating

Another tablet formulation was prepared in the following that comprises an immediate release drug coating with a ratio of drug to binder of 1:3 (Table 30).

TABLE 30
Formulation of batch AZF-1-FD-5-2B with 1:3 drug to
binder ratio in immediate release drug coating.

| | | Lot No. AZF-1-FD-5-2B | |
|---|---|---|---|
| Material | Function | mg/tab | % w/w core |

Core tablet

| | | | |
|---|---|---|---|
| Dexpramipexole Dihydrochloride Monohydrate | Drug substance | 271.16** | 42.00 |
| Microcrystalline cellulose (Avicel PH 102) | Diluent | 196.90 | 30.50 |
| Copovidone (Kollidon VA 64 fine) | Binder | 45.19 | 7.00 |
| Sodium Chloride (Emprove ®) | Inorganic | 129.12 | 20.00 |
| | osmogene | | |
| Magnesium stearate (Vegetable source) | Lubricant | 3.23 | 0.50 |

| | | |
|---|---|---|
| Total | 645.60 | 100.00 |

Membrane Coating\hat{ }\hat{ }

| | | | |
|---|---|---|---|
| Opadry CA 500F190003 clear (Cellulose | Semipermeable | 51.65 | . . . |
| acetate 398-10:PEG 3350 = 85:15) | coat | | |
| Acetone\hat{ } | Solvent | 922.47\hat{ } | . . . |
| Purified water\hat{ } | Solvent | 58.88\hat{ } | . . . |

| | | |
|---|---|---|
| Total | 697.25 | . . . |

Seal Coating^{β}

| | | | |
|---|---|---|---|
| Opadry YS-1-19025-A clear | Seal coat | 20.92 | . . . |
| Purified water\hat{ } | Solvent | 188.28\hat{ } | . . . |

| | | |
|---|---|---|
| Total | 718.17 | . . . |

Immediate Release Drug Coating^{€}

| | | | |
|---|---|---|---|
| Dexpramipexole Dihydrochloride Monohydrate | Drug substance | 47.85** | . . . |
| Opadry YS-1-19025-A clear | Binder | 143.55 | . . . |
| Purified water\hat{ } | Solvent | 1722.60\hat{ } | . . . |

| | | |
|---|---|---|
| Total | 909.57 | . . . |

q.s.: Quantity sufficient,
**Conversion factor for Dexpramipexole Dihydrochloride Monohydrate to Dexpramipexole Dihydrochloride = 0.9404,
\hat{ }Evaporates during processing,
\hat{ }\hat{ }Solid content of Coating Solution = 5% w/w, Acetone:water = 94:6,
^{β, €}Solid content of Coating Solution = 10% w/w.

Scanning electron microscopy images of immediate release drug coated tablets of Lot No. AZF-1-FD-5-2B are presented in FIG. 13 and reveal that the tablet surface was smooth.

### Example 7.5—Clinical Trial Batches

For evaluation in clinical trials, two osmotic tablet formulations (“slow” and “fast”) were prepared. The “fast” formulation contained drug solely in the tablet core (300 mg dexpramipexole dihydrochloride equivalent) and the semipermeable membrane coating weight gain was 6.2% w/w. The “slow” formulation comprised 255 mg dexpramipexole dihydrochloride equivalent in the tablet core (85% of the total dose) and 45 mg dexpramipexole dihydrochloride equivalent in an immediate release form as drug coating (15% of the total dose). The semipermeable membrane coating weight gain was 8.0% w/w.

Phase 1 clinical trial material batches were manufactured at a scale of 12 kg. The formulations are provided in Table 31. A summary of process parameters is given in Table 32. A re-laser drilling step followed the laser-drilling step for Batch Number 5715488 to drill one more hole.

TABLE 31
Dexpramipexole dihydrochloride monohydrate osmotic tablet formulations
(“slow” and “fast”) prepared for evaluation in clinical
trials. An amount of 319.01 mg dexpramipexole dihydrochloride monohydrate
is equivalent to 300.00 mg dexpramipexole dihydrochloride.

| | Batch Number | | | |
|---|---|---|---|---|
| | 5670621 | | 5715488 | |
| | Fast Prototype | | Slow Prototype | |
| | | % w/w | | % w/w |
| Material | mg/tab | core | mg/tab | core |

Core tablet

| | | | | |
|---|---|---|---|---|
| Dexpramipexole Dihydrochloride Monohydrate | 319.01** | 42.00 | 271.16** | 42.00 |
| Microcrystalline cellulose (Avicel PH 102) | 231.66 | 30.50 | 196.91 | 30.50 |
| Copovidone (Kollidon VA 64 fine) | 53.17 | 7.00 | 45.19 | 7.00 |
| Sodium Chloride (Emprove ®) | 151.91 | 20.00 | 129.12 | 20.00 |
| Magnesium stearate (Vegetable source) | 3.80 | 0.50 | 3.23 | 0.50 |
| Total | 759.55 | 100.00 | 645.61 | 100.00 |

Semipermeable membrane coating\hat{ }\hat{ }

| | | | | |
|---|---|---|---|---|
| Opadry CA 500F190003 clear (Cellulose | 47.09 | . . . | 51.65 | . . . |
| acetate 398-10:PEG 3350 = 85:15) | | | | |
| Acetone\hat{ } | 841.03\hat{ } | . . . | 922.43\hat{ } | . . . |
| Purified water\hat{ } | 53.68\hat{ } | . . . | 58.88\hat{ } | . . . |
| Total | 806.64 | . . . | 697.26 | . . . |

Seal coating^{β}

| | | | | |
|---|---|---|---|---|
| Opadry YS-1-19025-A clear (hydroxypropyl | . . . | . . . | 20.92 | . . . |
| methylcellulose, polyethylene glycol) | | | | |
| Purified water\hat{ } | . . . | . . . | 188.28\hat{ } | . . . |
| Total | . . . | . . . | 718.18 | . . . |

Immediate release drug coating^{€}

| | | | | |
|---|---|---|---|---|
| Dexpramipexole Dihydrochloride Monohydrate | . . . | . . . | 47.85** | . . . |
| Opadry YS-1-19025-A clear (hydroxypropyl | . . . | . . . | 143.55 | . . . |
| methylcellulose, polyethylene glycol) | . . . | . . . | | . . . |
| Purified water\hat{ } | . . . | . . . | 1722.60\hat{ } | . . . |
| Total | . . . | . . . | 909.58 | . . . |

Film coating^{α#}

| | | | | |
|---|---|---|---|---|
| Opadry II 85F18422 white (polyvinyl alcohol, | 24.20 | . . . | 27.29 | . . . |
| titanium dioxide, polyethylene glycol, talc) | | | | |
| Purified water\hat{ } | 96.82\hat{ } | . . . | 109.16\hat{ } | . . . |
| Total | 830.84 | . . . | 936.87 | . . . |

q.s.: Quantity sufficient
**Conversion factor for Dexpramipexole Dihydrochloride Monohydrate to Dexpramipexole Dihydrochloride = 0.9404 (271.16 mg of Dexpramipexole Dihydrochloride monohydrate is equivalent to 255.00 mg of Dexpramipexole Dihydrochloride; 319.00 mg of Dexpramipexole Dihydrochloride monohydrate is equivalent to 300.00 mg of Dexpramipexole Dihydrochloride; 47.85 mg of Dexpramipexole Dihydrochloride monohydrate is equivalent to 45.00 mg of Dexpramipexole Dihydrochloride)
\hat{ }Evaporates during processing
\hat{ }\hat{ }Solid content of Coating Solution = 5% w/w, Acetone:purified water = 94:6
^{β, €}Solid content of Coating Solution = 10% w/w, drug to binder ratio in drug coating = 1:3
^{α}Solid content of Coating dispersion = 20% w/w
^{#}Laser drilling done at the end of film coating.

TABLE 32
Summary of process parameters for clinical trial batches 5670621 and 5715488.

| Process Parameters | | 5670621 (Fast) | | 5715488 (Slow) | |
|---|---|---|---|---|---|
| Compression | Target Weight (mg) | 760 | (722-798) | 646 | (614-678) |
| | Target Composite (mg) (10 tabs) | 7600 | (7370-7830) | 6460 | (6270-6650) |
| | Target Thickness (mm) | Report Results | | Report Results | |
| | Target Hardness (kP) | 20 | (15-25) | 15 | (10-20) |
| | Press Speed (rpm) | 25 | | 25 | |
| | Feeder Speed (rpm) | 35 | | 35 | |
| Membrane | Pan size (in) | 24 | | 24 | |
| Coating | Nozzle port opening (mm) | 1.2 | | 1.2 | |
| | Drum Speed (rpm) | 8-12 | | 8-12 | |
| | Inlet Air Volume (cfm) | 250-300 | | 250-300 | |
| | Inlet Air Temperature (° C.) | 30-40 | | 30-40 | |
| | Product Temperature (° C.) | Not recorded | | 24-26 | |
| | Exhaust Air Temperature (° C.) | 20-30 | | 49-50 | |
| | Target Weight Gain | 6.2% | (5.9%-6.5%) | 8.0% | (7.8%-8.2%) |
| | Spray Rate (g/min) | 80-100 | | 70-100 | |
| | Pattern Air Pressure (psi) | 20 | | 20 | |
| | Atomizing Air Pressure (psi) | 15 | | 15 | |
| Seal | Pan size (in) | . . . | | 24 | |
| Coating | Nozzle port opening (mm) | | | 1.2 | |
| | Drum Speed (rpm) | | | 12-16 | |
| | Inlet Air Volume (cfm) | | | 250-450 | |
| | Inlet Air Temperature (° C.) | | | 55-65 | |
| | Product Temperature (° C.) | | | Not recorded | |
| | Exhaust Air Temperature (° C.) | | | 40-50 | |
| | Target Weight Gain | | | 3.0% | (2.5%-3.5%) |
| | Spray Rate (g/min) | | | 35 | |
| | Pattern Air Pressure (psi) | | | 25 | |
| | Atomizing Air Pressure (psi) | | | 35 | |
| Immediate | Pan size (in) | . . . | | 24 | |
| Release | Nozzle port opening (mm) | | | 1.2 | |
| Drug | Drum Speed (rpm) | | | 12-16 | |
| Coating | Inlet Air Volume (cfm) | | | 250-450 | |
| | Inlet Air Temperature (° C.) | | | 65-75 | |
| | Product Temperature (° C.) | | | Not recorded | |
| | Exhaust Air Temperature (° C.) | | | 40-50 | |
| | Target Weight Gain | | | 26.7% | (26.2%-27.2%) |
| | Spray Rate (g/min) | | | 35 | |
| | Pattern Air Pressure (psi) | | | 25 | |
| | Atomizing Air Pressure (psi) | | | 35 | |
| Film Coating | Pan size (in) | 24 | | 24 | |
| | Nozzle port opening (mm) | 1.2 | | 1.2 | |
| | Drum Speed (rpm) | 12-16 | | 12-16 | |
| | Inlet Air Volume (cfm) | 250-450 | | 250-450 | |
| | Inlet Air Temperature (° C.) | 55-65 | | 55-65 | |
| | Product Temperature (° C.) | Not recorded | | Not recorded | |
| | Exhaust Air Temperature (° C.) | 40-50 | | 40-50 | |
| | Target Weight Gain | 3.0% | (2.5%-3.5%) | 3.0% | (2.5%-3.5%) |

| | | | |
|---|---|---|---|
| | Spray Rate (g/min) | 45 | 45 |
| | Pattern Air Pressure (psi) | 25 | 25 |
| | Atomizing Air Pressure (psi) | 35 | 35 |
| Laser | Spindle speed (rpm) | 800 | 800 |
| Drilling | System speed (links/min) | 500 | 500 |
| | Pitch (%) | 0.040 | 0.040 |
| | Drill speed (mm/sec) | 2500 | 2500 |
| | Power level (%) | 90 | 90 |
| | PWM Frequency (KHz) | 25 | 25 |
| | Hole count per tablet | 1 | 1 |
| | Hole Diameter (mm) | 0.5 | 0.5 |
| Re-Laser | Spindle speed (rpm) | . . . | 800 |
| Drilling | System speed (links/min) | | 360 |
| | Pitch (%) | | 0.062 |
| | Drill speed (mm/sec) | | 1000 |
| | Power level (%) | | 90 |
| | PWM Frequency (KHz) | | 25 |
| | Hole count per tablet | | 2 |
| | Hole Diameter (mm) | | 0.5 |

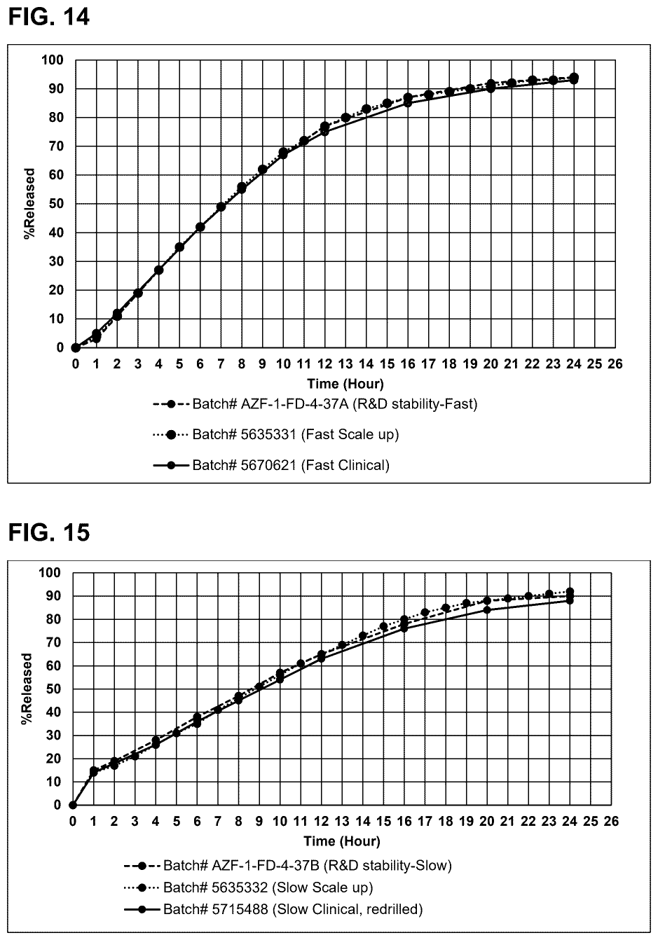

FIGS. 14 and 15 provide a comparison of the clinical trial batches with the engineering batches and corresponding smaller-scale laboratory batches. The results indicate very good reproducibility.

### Example 8—Phase I Clinical Trial in Healthy Participants (EXHALE-6)

Osmotic tablets formulated as shown in Table 31 are given to healthy human volunteers once a day (QD; quaque die) to assess bioavailability of dexpramipexole. The clinical study is designed to compare four ER tablet formulations (QD tablet formulations), including the two osmotic tablet formulations of Table 31, to an immediate release (IR) tablet formulation (bis in die (BID) tablet formulation). The ER tablet formulations provide a dose of 300 mg dexpramipexole dihydrochloride equivalent (which corresponds to a daily dose of 300 mg dexpramipexole dihydrochloride equivalent, as the ER tablet formulations are given once a day). The JR tablet formulation provides a dose of 150 mg dexpramipexole dihydrochloride equivalent (which also corresponds to a daily dose of 300 mg dexpramipexole dihydrochloride equivalent, as the JR tablet formulation is given twice a day). The composition of the JR tablet formulation is given in Table 33.

TABLE 33
Composition of BID IR tablet formulation.

| Component | Function | Quantity per Tablet | |
|---|---|---|---|
| Dexpramipexole | Drug Substance | 159.50 | mg* |
| dihydrochloride monohydrate | | | |
| Microcrystalline cellulose | Diluent | 132.19 | mg |
| Mannitol | Diluent | 65.50 | mg |
| Crospovidone | Disintegrant | 15.00 | mg |
| Magnesium stearate | Glidant | 2.81 | mg |

| | | |
|---|---|---|
| Core Total | 375.0 | mg |

Tablet Coat

| | | | |
|---|---|---|---|
| Opadry II white | Film former | 11.25 | mg |

| | | |
|---|---|---|
| Coated tablet Total | 386.25 | mg |

*159.50 mg of dexpramipexole dihydrochloride monohydrate is equivalent to 150.0 mg dexpramipexole dihydrochloride based on molecular weight ratio of monohydrate to anhydrous API.

### Study Design

A Phase I, open-label, single-center, five-period, randomized, crossover study in 15 healthy adult male and female participants is conducted to evaluate the relative bioavailability of dexpramipexole extended release (ER) formulations to an immediate release (IR) tablet formulation. Participants provide informed consent and complete screening procedures within 21 days prior to Day −1. All participants eligible for enrollment must meet all inclusion criteria and none of the exclusion criteria. Participants are randomly assigned to one of five sequences (i.e., study arms or study groups) and receive single oral doses of dexpramipexole ER formulations on four dosing days and two oral doses of dexpramipexole IR formulation given every 12 hours (q12h) on one dosing day for a total of five dosing days/periods. Each participant receives a different formulation in each period. Participants are receiving dexpramipexole ER and IR formulations on Days 1, 4, 8, 11, and 15, according to random assignment. Drug plasma exposure is expected to occur within the first 24 hours following administration. The washout period before the next treatment period is expected to be sufficient given the study design and drug properties. Participants are required to be domiciled at the study site for the duration of the study (Day −1 to Day 17) and return for a follow-up visit on Day 18. The entire duration of the study, including Screening, is approximately 6 weeks.

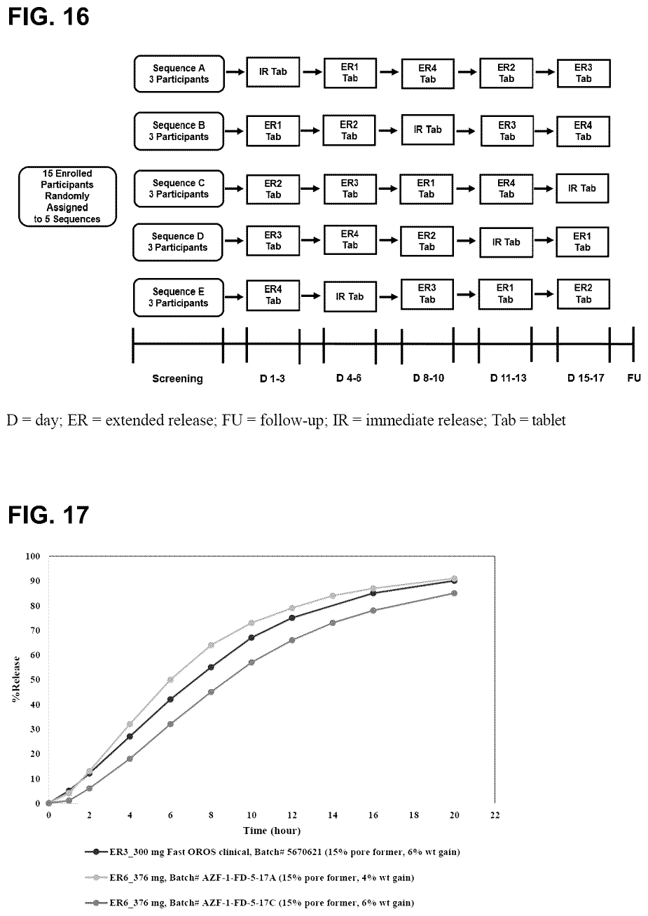

A schematic outline of the clinical study is given in FIG. 16 (wherein ER3 and ER4 correspond to the two osmotic tablet formulations of Table 31).

### Study Assessments—Pharmacokinetics (PK)

Blood samples (three mL each; up to 22 samples per period) for the preparation of plasma are collected at specified time points for 48 hours after oral administration of dexpramipexole (relative to first dose for IR formulation) for the determination of dexpramipexole concentrations in plasma. Thus, blood samples are collected for plasma dexpramipexole concentration at predose (0), 0.5, 1, 1.5, 2, 2.5, 3, 4, 5, 6, 8, 10, 12 (predose for second dose of IR formulation), 12.5, 13, 14, 15, 16, 20, 24, 36, and 48 hours postdose (relative to first dose for IR formulation). When a PK sample collection time point corresponds with study drug dosing, the PK sample is collected before study drug dosing. Table 34 provides the schedule for pharmacokinetic blood sample collection.

TABLE 34
Pharmacokinetic blood sample collection schedule.

| | | | | PK Blood Sample |
|---|---|---|---|---|
| Days | Time (h) | Collection Window Relative to Time | | Collection |
| Days 1, 4, 8, | 0 | Up to 60 min predose | | X |
| 11, and 15 | 0.5 | ±5 | min | X |
| | 1 | ±5 | min | X |
| | 1.5 | ±5 | min | X |
| | 2 | ±5 | min | X |
| | 2.5 | ±5 | min | X |
| | 3 | ±5 | min | X |
| | 4 | ±5 | min | X |
| | 5 | ±5 | min | X |
| | 6 | ±5 | min | X |
| | 8 | ±5 | min | X |
| | 10 | ±5 | min | X |
| | 12 | ±5 | min^{a} | X |
| | 12.5 | ±5 | min | X |
| | 13 | ±5 | min | X |
| | 14 | ±5 | min | X |
| | 15 | ±5 | min | X |
| | 16 | ±5 | min | X |
| | 20 | ±15 | min | X |
| Days 2, 5, 9, | 24 | ±15 | min | X |
| 12, and 16 | 36 | ±15 | min | X |
| Days 3, 6, 10, | 48 | ±15 | min | X^{b} |
| 13, and 17 | | | | |

Abbreviation: CRU = clinical research unit; h = hour; min = minute; PK = pharmacokinetic.
^{a}Must be collected before the second IR dose.
^{b}Participants will be discharged from the CRU after the 48-hour PK blood sample collection on Day 17 (Period 5).

Plasma dexpramipexole concentrations are analyzed using noncompartmental methods for the determination of the following PK parameters:
maximum observed plasma concentration (Cₘₐₓ; for IR tablet, calculated after each dose [Cₘₐₓ over the 0- to 12-hour interval, C_{max,0-12}; and Cₘₐₓ over the 12- to 24-hour interval, C_{max,12-24}] and overall),
time to the maximum observed plasma concentration (tₘₐₓ; for IR tablet, calculated after each dose [tₘₐₓ over the 0- to 12-hour interval, t_{max,0-12}; and tₘₐₓ over the 12- to 24-hour interval, t_{max,12-24}] and overall),
area under the concentration-time curve (AUC) from time 0 to the time of 24 hours postdose (AUC₀₋₂₄),
AUC from time 0 to the time of the last measurable concentration (AUC₀₋ₜ),
AUC from time 0 extrapolated to infinity (AUC_{0-inf}; for ER formulations only),
time prior to the first measurable (non-zero) concentration (t_{lag}; for IR tablet, calculated for the first dose only),
apparent terminal elimination rate constant (λ_{z}; for IR tablet, calculated after the second dose only), and
apparent terminal half-life (t_{1/2}; for IR tablet, calculated after the second dose only).

Additional PK parameters that are determined to fully characterize PK of dexpramipexole are described in the statistical analysis plan (SAP).

### Study Assessments—Safety

Safety and tolerability are monitored throughout the study by observed and reported adverse events (AEs; serious and non-serious AEs), clinical laboratory assessments (including serum chemistry, hematology, and urinalysis; see Table 35 showing clinical laboratory assessment tests that are performed), vital signs (blood pressure, heart rate, respiratory rate, and oral body temperature), electrocardiogram (ECG) measurements, and physical examinations. If an ECG is scheduled at the same time point as blood sample collection, the ECG is performed first.

TABLE 35
Clinical Laboratory Tests

| Hematology | Serum Chemistry | Urinalysis |
|---|---|---|
| Complete Blood Count (CBC) | Alanine aminotransferase (ALT) | Appearance |
| Hematocrit | Albumin | Bilirubin |
| Hemoglobin | Alkaline phosphatase | Blood |
| Platelet count | Aspartate aminotransferase (AST) | Color |
| Red blood cell count | Bicarbonate | Glucose |
| White blood cell count | Blood urea nitrogen (BUN) | Ketones |
| (with automated | Calcium | Leukocyte esterase |
| differential) | Chloride | Nitrite |
| | Creatinine | pH |
| | Direct bilirubin | Protein |
| | Indirect bilirubin | Specific gravity |
| | Gamma-glutamyl transferase | Urobilinogen |
| | (GGT) | Microscopic examination: |
| | Glucose | red blood cells; white |
| | Estimated glomerular filtration | blood cells; epithelial |
| | rate (eGFR)^{a} | cells; bacteria, crystals, |
| | Lactic dehydrogenase (LDH) | casts, etc. (if present) |
| | Magnesium | |
| | Phosphorus | |
| | Potassium | |
| | Sodium | |
| | Total bilirubin | |
| | Total protein | |
| | Uric acid | |

Diagnostic Screening Tests:

| Blood | Urine Drug Screen and Cotinine |
|---|---|
| Serology: hepatitis panel (hepatitis C antibody, | Urine drug screen: amphetamines, barbiturates, |
| hepatitis B surface antigen, hepatitis B core | benzodiazepines, cannabinoids, cocaine, alcohol, |
| antibody), and HIV antibody (Screening) | and opiates (Screening and Check-in [Day −1]). |
| Serum pregnancy test (all female participants at | Cotinine: |
| Screening, Check-in [Day −1], and before | (Screening and Check-in [Day −1]). |
| Discharge/Early Termination) | Additional urine may be collected as needed per |
| FSH (female participants not surgically sterile | the schedule of assessments. |
| who have had spontaneous amenorrhea for at least | |
| 1 year) at Screening | |

^{a}eGFR is calculated using the Chronic Kidney Disease Epidemiology Collaboration formula at Screening.

An AL is any untoward medical occurrence associated with the use of a drug in humans and which does not necessarily have to have a causal relationship with this treatment. An AL can be any unfavorable and unintended sign, symptom, or disease temporally associated with the use of a medicinal product, whether or not considered related to the medicinal product [ICH E2A Guideline, 1994].

A serious adverse event (SAL) is any untoward medical occurrence that at any dose:
Results in death
Is life threatening, i.e., the participant was, in the opinion of the investigator, at immediate risk of death from the event as it occurred (it does not include an event that, had it occurred in a more severe form, might have caused death)
Results in a significant, persistent, or permanent change, impairment, damage or disruption in the participant's body function/structure, physical activities and/or quality of life
Requires in-participant hospitalization or prolongs hospitalization
Is a congenital anomaly/birth defect, or
Is an important medical event.

It is determined whether there is a reasonable possibility or not that the investigational product caused an AE, based on whether there is evidence that suggests a causal relationship between the investigational product and the AE.

Each AE is rated for severity (intensity) according to the following definitions:
Mild: No interference with daily activities
Moderate: Some interference with activity not requiring medical intervention
Severe: Prevents daily activity and requires medical intervention
Potentially Life Threatening: ER visit or hospitalization

Adverse events of any intensity are reported as SAEs if at least 1 criteria established above is met. The occurrence of a life-threatening adverse event results in the individual being discontinued from the study drug. The event is followed until it resolves or stabilizes.

The schedule of assessments for Periods 1, 2, 3, 4, and 5 is given in Table 36 below.

TABLE 36

| | | | | | | | | | | | | | | | | | | | | | | | | Days | |
|---|---|---|---|---|---|---|---|---|---|---|---|---|---|---|---|---|---|---|---|---|---|---|---|---|---|
| | | | | | | | | | | | | | | | | | | | | | | Days 2, | | 3, 6, | |
| | | | | | | | | | | | | | | | | | | | | | | 5, 9, 12, | | 10, 13, | |
| | | | Day 1, 4, 8, 11, and 15 | | | | | | | | | | | | | | | | | | | and 16 | | and 17 | |
| | Day −21 | Day −1 | 0 | 0.5 | 1 | 1.5 | 2 | 2.5 | 3 | 4 | 5 | 6 | 8 | 10 | 12 | 12.5 | 13 | 14 | 15 | 16 | 20 | 24 | 36 | 48 | |
| Event | Screening^{b} | Check-In | h | h | h | h | h | h | h | h | h | h | h | h | h | h | h | h | h | h | h | h | h | h | 18^{a} |
| Informed consent | x | | | | | | | | | | | | | | | | | | | | | | | | |
| Medical history | x | | | | | | | | | | | | | | | | | | | | | | | | |
| Physical examination | x | | | | | | | | | | | | | | | | | | | | | | | x | x |
| (including weight | | | | | | | | | | | | | | | | | | | | | | | | | |
| and height)^{c} | | | | | | | | | | | | | | | | | | | | | | | | | |
| BMI | x | | | | | | | | | | | | | | | | | | | | | | | | |
| Vital signsd | x | x | x | | | | x | | | x | | | x | | | | | | | | | x | | x | x |
| ECG (12-lead) | x | x | | | | | | | | | | | | | | | | | | | | | | x | x |
| Hematology | x | x | | | | | | | | | | | | | | | | | | | | | | x | x |
| Clinical chemistry | x | x | | | | | | | | | | | | | | | | | | | | | | x | x |
| Urinalysis | x | x | | | | | | | | | | | | | | | | | | | | | | x | x |
| HIV antibody | x | | | | | | | | | | | | | | | | | | | | | | | | |
| Hepatitis serology^{e} | x | | | | | | | | | | | | | | | | | | | | | | | | |
| Serum pregnancy | x | x^{g} | | | | | | | | | | | | | | | | | | | | | | | x |
| test^{f} | | | | | | | | | | | | | | | | | | | | | | | | | |
| Urine cotinine screen | x | x^{g} | | | | | | | | | | | | | | | | | | | | | | | |
| Urine drug screen^{h} | x | x^{g} | | | | | | | | | | | | | | | | | | | | | | | |
| PK sampling | | | x | x | x | x | x | x | x | x | x | x | x | x | x | x | x | x | x | x | x | x | x | x | xⁱ |
| ER tablet study | | | x | | | | | | | | | | | | | | | | | | | | | | |
| drug administration^{j} | | | | | | | | | | | | | | | | | | | | | | | | | |
| IR tablet study drug | | | x | | | | | | | | | | | | x | | | | | | | | | | |
| administration^{j} | | | | | | | | | | | | | | | | | | | | | | | | | |

| | | | |
|---|---|---|---|
| Concomitant | x | x | [symbol] |
| Medications | | | |
| Adverse Events | x | x | [symbol] |

| | | |
|---|---|---|
| Clinic Visit | x | [symbol] |

ECG = electrocardiogram;
ER = extended release;
FU = follow-up;
IR = immediate release;
PK = pharmacokinetic
^{a}Day 18 procedures should be conducted within 72-96 hrs after last dose. Day 18 study evaluations are also to be preformed as early termination procedures.
^{b}All screening procedures are to be completed within 21 days prior to Day −1.
^{c}Height will be measured only at screening.
^{d}Vital signs will be assessed within 1 hour of dosing.
^{e}Hepatitis C antibody, hepatitis B surface antigen, hepatitis B core antibody.
^{f}Serum pregnancy testing for all female participants at screening. check-in [Day −1], and before discharge/early termination. Follicle-strimulating hormone testing at screening only for female participants not surgically sterile and who have had spontaneous amenorrhea for a least 12 months.
^{g}Negative pregnancy test, drug screen and continine results were to be obtained prior to first dose.
^{h}Amphetamines, benzodiazepines, cocaine, barbiturates, cannabis, opiates, and alcohol.
ⁱPK samples to be collected only as a part of early termination procedures.
^{j}Participant will be administered dose based on sequence assignment.
^{k}Participants will be confined on Day −1 until completion of the 48-hour postdose evaluations on Day 17.

### Study Objectives

Primary Objective: To compare the relative bioavailability of a single dose of each ER formulation to two doses of the IR formulation given every 12 hours (q12h).

Secondary Objectives: To evaluate the safety and tolerability of each formulation and to describe the pharmacokinetics (PK) of the IR dosage formulation and each ER dosage formulation.

### Study Endpoints

Primary Endpoints: Cₘₐₓ and AUC₀₋₂₄

Key Secondary Endpoints: Safety endpoints: AEs, vital signs (systolic and diastolic blood pressure, respiratory rate, pulse, and temperature), clinical laboratory assessments, ECGs, and physical examinations

Other Secondary Endpoints: AUC₀₋ₜ, AUC_{0-inf}, tₘₐₓ, t_{lag}, and other parameters, as appropriate

### Inclusion Criteria

A participant may be included in the study only if all of the following inclusion criteria are met:
1. Is a healthy male or female at least 18 and not greater than 55 years old at screening
2. Is a non-smoker defined by a history of non-smoking or smoking cessation and tobacco (including nicotine replacement) abstinence for over 1 year from screening
3. Has a body mass index (BMI) of ≥18 and ≤32 kg/m²: (BMI=weight (kg)÷[height (m)]²) at screening
4. Has normal findings upon physical examination (i.e., no clinically significant abnormal physical findings)
5. Has normal or not clinically significant 12-lead electrocardiogram (ECG) findings at screening and Day −1
6. Has a normal systemic blood pressure defined as a systolic pressure of 90-140 mmHg and a diastolic pressure of 50-90 mmHg, may repeat at discretion of the Investigator at screening.
7. Has a normal resting heart rate between 50 and 100 beats per minute (bpm) on vitals, may repeat at discretion of Investigator at screening.
8. Is willing and able to communicate with the study team and abide by all study requirements and restrictions.
9. Is willing to provide signed, written informed consent.

### Exclusion Criteria

A participant meeting any of the following criteria is not eligible to participate in the study:
1. Has a history or current evidence of any significant acute or chronic medical illness.
2. Has a history of any gastrointestinal issues or surgery (except simple appendectomy, cholecystectomy, or hernia repair) that could possibly impact drug absorption.
3. Has any major surgery within 12 weeks prior to (Day −1).
4. Has a history of recent (within 6 months) drug or alcohol abuse.
5. Has evidence of organ dysfunction or any clinically significant deviation from normal in the medical history, physical examination, vital signs, ECG, and/or clinical laboratory determinations, may repeat at discretion of Investigator.
6. Women of childbearing potential (WOCBP; after menarche) must use either of the following methods of birth control, from Screening through the End of Study Visit:
   a. A highly effective form of birth control (confirmed by the investigator). Highly effective forms of birth control include: true sexual abstinence, a vasectomized sexual partner, Implanon, female sterilization by tubal occlusion, any effective Intrauterine device (IUD), IUD/intrauterine system (IUS), Levonorgestrel Intrauterine system, or combined (estrogen and progestogen-containing or progestogen-only) hormonal contraceptive associated with inhibition of ovulation.
   OR
   b. Two protocol acceptable methods of contraception (i.e., progestogen-only oral hormonal conception, where inhibition of ovulation is not the primary mode of action; male or female condom with or without spermicide; cap, diaphragm or sponge with spermicide) in tandem.
Women not of childbearing potential are defined as women who are either permanently sterilized (hysterectomy, bilateral oophorectomy, or bilateral salpingectomy), or who are postmenopausal. Women will be considered postmenopausal if they have been amenorrheic for 12 months prior to the planned date of the Screening Visit without an alternative medical cause. The following age specific requirements apply:
   c. women <50 years old will be considered postmenopausal if they have been amenorrheic for 12 months or more following cessation of exogenous hormonal treatment and follicle stimulating hormone (FSH) levels are in the postmenopausal range.
   d. Women ≥50 years old will be considered postmenopausal if they have been amenorrheic for 12 months or more following cessation of all exogenous hormonal treatment.
7. Has any other clinically significant medical, psychiatric and/or social reason for being ineligible as determined by the Principal Investigator.
8. Has a prior history or propensity for orthostatic hypotension, history of repeated fainting or hemodynamic instability.
9. Has a screening 12-lead ECG demonstrating QTcF >470 msec for women or >450 msec for men, and/or history or evidence of long QT syndrome.
10. Donated blood or has a significant loss of blood (≥480 mL) within 4 weeks prior to screening.
11. Donated plasma within 2 weeks prior to screening.
12. If female, has a positive serum pregnancy test at screening visit or at study check in.
13. Has a positive urine screen for drugs of abuse (including alcohol) during screening visit or at study check in.
14. Has a positive urine screen for cotinine during screening visit or at study check in.
15. Has a positive blood screen for hepatitis C antibody, hepatitis B surface antigen, hepatitis B core antibody, or HIV.
16. Has a history of significant drug allergy.
17. Has a prior exposure to dexpramipexole.
18. Has prior exposure to any investigational drug or placebo within 4 weeks or 5 half lives of the drug (whichever is longer) prior to Check-In (Day −1).
19. Has used any over the counter acid controllers/blockers within 4 weeks prior to Check-In (Day −1) or has achlorhydria.
20. Has a history of dopamine agonist use (e.g., Mirapex®, Mirapex-ER®, Requip®, bromocriptine, Sinemet®).
21. Has received any prescription (excluding acceptable birth control or hormone replacement therapy), non-prescription, nutritional supplements, or herbal medication 14 days or 5 half-lives (whichever is longer) prior to Check-In (Day −1).
22. Is unable or has difficulty swallowing or tolerating oral medication.
23. Poor venous access.
24. Pregnant women or women breastfeeding.
25. Males who are unwilling to use an acceptable method of birth control during the entire study period (i.e., condom with spermicide).

### Study Period

Five study periods of 72 hours.

### Study Duration

The entire duration of the study, including Screening, is approximately 6 weeks.
Twenty-one day screening period
Day −1—admission/check-in
Five 3-day treatment periods
Follow-up is completed 72-96 hours after last dose

### Investigational Product Administration

Participants are consuming a standard meal 2 hours before the dose. Water may be ingested ad libitum up to 1-hour predose, and after 2 hours postdose. Dexpramipexole tablets are administered orally with 240 mL of room temperature tap water. Participants may receive standardized meals and snacks after their 4-hour postdose blood draw.

### Statistical Methods—Pharmacokinetics

Plasma dexpramipexole concentrations and the PK parameters are summarized using the appropriate descriptive statistics.

To determine the relative bioavailability of the test (ER tablets) to the reference (IR tablet) formulations, an analysis of variance (ANOVA) is performed on the natural logarithm (ln)-transformed PK parameters Cₘₐₓ and AUC₀₋₂₄. The ANOVA model includes treatment, sequence, and period as fixed effects and participant nested within sequence as a random effect. Treatment differences as measured by least squares (LS) mean differences and the 90% CIs are constructed for the In-scale values of each parameter, back-transformed, and expressed as the ratio of geometric means. The ratios (and 90% CIs) for Cₘₐₓ and AUC₀₋₂₄ estimates are expressed as a percentage relative to the reference formulation. The In-transformed PK parameter AUC₀₋ₜ is compared using the same model, with the results presented as supportive information.

The comparisons of interest for relative bioavailability assessments are as follows:
ER tablet 1 versus IR tablet given g12h
ER tablet 2 versus IR tablet given g12h
ER tablet 3 versus IR tablet given g12h
ER tablet 4 versus IR tablet given g12h

### Statistical Methods—Safety

The safety summary includes the analysis of treatment-emergent adverse events (TEAE; i.e. AEs that begin after the first dose of study drug), including serious adverse events (SAEs) and the adverse events leading to study discontinuation, by treatment and overall.

Clinical laboratory results, vital signs, and ECG measurements are summarized using descriptive statistics and the changes from baseline by treatment and time point of collection. A shift table is provided for selected laboratory parameters, as appropriate.

### Analysis Population

Pharmacokinetics: The PK population includes all participants who have a calculable PK parameter for the reference treatment, and at least one test treatment in order to have a comparison of interest for the primary objective. Data from participants in this population is used for all PK summaries.

Safety: The safety population includes all participants who receive at least 1 dose of study drug. Data from participants in this population are used for demographic and safety summaries.

### Sample Size Determination

Fifteen participants are enrolled in the study, with three participants in each sequence. A sample size of 12 participants is typical for pilot relative bioavailability studies (per Food and Drug Administration [FDA] guidance) and deemed adequate for the assessment of relative bioavailability; 3 additional participants are enrolled to allow for dropouts (20% dropout rate).

### Example 9—Osmotic Tablet Formulations

Five additional osmotic tablet formulations were prepared. The “fast” formulation contained drug solely in the tablet core (300 mg dexpramipexole dihydrochloride equivalent), and the semipermeable membrane coating weight gain was 6.2% w/w. The “slow” formulation comprised 255 mg dexpramipexole dihydrochloride equivalent in the tablet core (85% of the total dose) and 48 mg dexpramipexole dihydrochloride equivalent in an immediate release form as drug coating (15% of the total dose). The semipermeable membrane coating weight gain was 8.0% w/w. The “ER6” formulation contained drug solely in the tablet core (376 mg dexpramipexole dihydrochloride equivalent) and the semipermeable membrane coating weight gain was 6.7% w/w. The “ER8” formulation contained drug solely in the tablet core (300 mg dexpramipexole dihydrochloride equivalent), and the semipermeable membrane coating weight gain was 4.5% w/w. The “ER8b” formulation contained drug solely in the tablet core (300 mg dexpramipexole dihydrochloride equivalent), and the semipermeable membrane coating weight gain was 7.0% w/w. The formulations are provided in Table 37.

TABLE 37
Dexpramipexole dihydrochloride monohydrate osmotic tablet formulations were prepared for evaluation.

| | Tablet Formulation | | | | | | | | | |
|---|---|---|---|---|---|---|---|---|---|---|
| | OROS Fast (ER3) | | OROS Slow (ER4) | | ER6 | | ER8 | | ER8b | |
| Material | mg/tab | % w/w core | mg/tab | % w/w core | mg/tab | % w/w core | mg/tab | % w/w core | mg/tab | % w/w core |

Core tablet

| | | | | | | | | | | |
|---|---|---|---|---|---|---|---|---|---|---|
| Dexpramipexole | 319.01 | 42.00 | 271.15 | 42.00 | 399.82 | 42.00 | 319.01 | 42.00 | 319.01 | 42.00 |
| Dihydrochloride | | | | | | | | | | |
| Monohydrate | | | | | | | | | | |
| Microcrystalline | 231.66 | 30.50 | 196.91 | 30.50 | n/a | n/a | N/A | N/A | N/A | N/A |
| cellulose | | | | | | | | | | |
| (Avicel PH 102) | | | | | | | | | | |
| Silicified | n/a | n/a | n/a | n/a | 290.35 | 30.50 | 231.66 | 30.50 | 231.66 | 30.50 |
| Microcrystalline | | | | | | | | | | |
| Cellulose | | | | | | | | | | |
| (Prosolv SMCC | | | | | | | | | | |
| HD90) | | | | | | | | | | |
| Copovidone | 53.17 | 7.00 | 45.19 | 7.00 | 66.64 | 7.00 | 53.17 | 7.00 | 53.17 | 7.00 |
| (Kollidon VA | | | | | | | | | | |
| 64 fine) | | | | | | | | | | |
| Sodium Chloride | 151.91 | 20.00 | 129.12 | 20.00 | 190.39 | 20.00 | 151.91 | 20.00 | 151.91 | 20.00 |
| (Emprove ®) | | | | | | | | | | |
| Magnesium | 3.80 | 0.50 | 3.23 | 0.50 | 4.76 | 0.50 | 3.80 | 0.50 | 3.80 | 0.50 |
| stearate | | | | | | | | | | |
| (Vegetable source) | | | | | | | | | | |
| Total | 759.55 | 100.00 | 645.61 | 100.00 | 951.96 | 100.00 | 759.55 | 100.00 | 759.55 | 100.00 |

Semipermeable membrane coating\hat{ }\hat{ }

| | | | | | | | | | | |
|---|---|---|---|---|---|---|---|---|---|---|
| Opadry CA | 47.09 | n/a | 51.65 | n/a | n/a | n/a | n/a | n/a | n/a | n/a |
| 500F190003 clear | | | | | | | | | | |
| (Cellulose acetate | | | | | | | | | | |
| 398-10:PEG 3350 = | | | | | | | | | | |
| 85:15) | | | | | | | | | | |
| Opadry CA | n/a | n/a | n/a | n/a | n/a | n/a | 34.18 | n/a | n/a | n/a |
| 500F190015 clear | | | | | | | | | | |
| (CA:PEG 3350 = | | | | | | | | | | |
| 75:25) | | | | | | | | | | |
| Opadry CA | n/a | n/a | n/a | n/a | n/a | n/a | n/a | n/a | 53.17 | n/a |
| 500F190006 clear | | | | | | | | | | |
| (CA:PEG 3350 = | | | | | | | | | | |
| 70:30) | | | | | | | | | | |
| Opadry CA | n/a | n/a | n/a | n/a | 63.78 | n/a | n/a | n/a | n/a | n/a |
| 500F190033 clear | | | | | | | | | | |
| (CA:PEG 3350 = | | | | | | | | | | |
| 82.5:17.5) | | | | | | | | | | |
| Acetone\hat{ } | 841.03\hat{ } | n/a | 922.43\hat{ } | n/a | 1139.13 | n/a | 584.47 | n/a | 909.18 | n/a |
| Purified water\hat{ } | 53.68\hat{ } | n/a | 58.88\hat{ } | n/a | 72.17 | n/a | 64.94 | n/a | 101.02 | n/a |
| Total | 806.64 | n/a | 697.26 | n/a | 1015.74 | n/a | 793.73 | n/a | 812.72 | n/a |

Seal coating^{β}

| | | | | | | | | | | |
|---|---|---|---|---|---|---|---|---|---|---|
| Opadry | n/a | n/a | 20.92 | n/a | n/a | n/a | n/a | n/a | n/a | n/a |
| YS-1-19025-A | | | | | | | | | | |
| clear (hydroxypropyl | | | | | | | | | | |
| methylcellulose, | | | | | | | | | | |
| polyethylene | | | | | | | | | | |
| glycol) | | | | | | | | | | |
| Purified water\hat{ } | n/a | n/a | 188.28\hat{ } | n/a | n/a | n/a | n/a | n/a | n/a | n/a |
| Total | n/a | n/a | 718.18 | n/a | n/a | n/a | n/a | n/a | n/a | n/a |

Immediate release drug coating^{€}

| | | | | | | | | | | |
|---|---|---|---|---|---|---|---|---|---|---|
| Dexpramipexole | n/a | n/a | 47.85 | n/a | n/a | n/a | n/a | n/a | n/a | n/a |
| Dihydrochloride | | | | | | | | | | |
| Monohydrate | | | | | | | | | | |
| Opadry | n/a | n/a | 47.85 | n/a | n/a | n/a | n/a | n/a | n/a | n/a |
| YS-1-19025-A | | | | | | | | | | |
| clear (hydroxypropyl | | | | | | | | | | |
| methylcellulose, | | | | | | | | | | |
| polyethylene | | | | | | | | | | |
| glycol) | | | | | | | | | | |
| Purified water\hat{ } | n/a | n/a | 861.30\hat{ } | n/a | n/a | n/a | n/a | n/a | n/a | n/a |
| Total | n/a | n/a | 813.88 | n/a | n/a | n/a | n/a | n/a | n/a | n/a |

Film coating^{α#}

| | | | | | | | | | | |
|---|---|---|---|---|---|---|---|---|---|---|
| Opadry II 85F18422 | 24.20 | n/a | 24.42 | n/a | n/a | n/a | n/a | n/a | n/a | n/a |
| white (polyvinyl | | | | | | | | | | |
| alcohol, titanium | | | | | | | | | | |
| dioxide, polyethylene | | | | | | | | | | |
| glycol, talc) | | | | | | | | | | |
| Opadry II 85F130089 | n/a | n/a | n/a | n/a | 30.47 | n/a | 23.81 | n/a | 24.38 | n/a |
| Pink (Polyvinyl | | | | | | | | | | |
| Alcohol, Titanium | | | | | | | | | | |
| Dioxide, | | | | | | | | | | |
| Polyethylene | | | | | | | | | | |
| Glycol, Talc, Ferric | | | | | | | | | | |
| Oxide Red) | | | | | | | | | | |
| Purified water\hat{ } | 96.82\hat{ } | n/a | 97.68\hat{ } | n/a | 121.88 | n/a | 95.24 | n/a | 97.52 | n/a |
| Total | 830.84 | n/a | 838.30 | n/a | 1046.21 | n/a | 817.54 | n/a | 837.10 | n/a |

Conversion factor for Dexpramipexole Dihydrochloride Monohydrate to Dexpramipexole Dihydrochloride Dihydrochloride = 0.9404 (271.15 mg of Dexpramipexole Dihydrochloride monohydrate is equivalent to 255.00 mg of Dexpramipexole Dihydrochloride; 319.01 mg of Dexpramipexole Dihydrochloride monohydrate is equivalent to 300.00 mg of Dexpramipexole Dihydrochloride; 399.82 mg of Dexpramipexole Dihydrochloride monohydrate is equivalent to 376.00 mg of Dexpramipexole Dihydrochloride; and 47.85 mg of Dexpramipexole Dihydrochloride monohydrate is equivalent to 45.00 mg of Dexpramipexole Dihydrochloride)
\hat{ }Evaporates during processing
\hat{ }\hat{ }Solid content of Coating Solution = 5% w/w, Acetone:purified water = 94:6
^{β,€}Solid content of Coating Solution = 10% w/w, drug to binder ratio in drug coating = 94:6
^{α}Solid content of Coating dispersion = 20% w/w
^{#}Laser drilling done at the end of film coating.

A quality target product profile (QTPP) was defined for Dexpramipexole Controlled-Release Tablets and is given in Table 38.

TABLE 38
Quality target product profile (QTPP) for Dexpramipexole
Dihydrocholoride Controlled-Release Tablets.

| QTPP Element | | | Target |
|---|---|---|---|
| Dosage Form and Design | | | Osmotic Tablet, Extended Release |
| Route of Administration | | | Oral |
| Dosage Strength | | | ER6: 280 mg Dexpramipexole (equivalent to 376 |
| | | | mg Dexpramipexole Dihydrocholoride) |
| | | | ER8 and ER9: 223 mg Dexpramipexole |
| | | | (equivalent to 300 mg of Dexpramipexole |
| | | | Dihydrocholoride) |
| Drug Product Quality | Physical | Appearance | No visual tablet defects observed. |
| Attributes | Attributes | Shape and | Oval or capsule shaped due to large tablet size |
| | | Size | |
| | | Friability | NMT 1.0% |
| | | Coating | Film coating |

| Assay | 90.0-110.0% |
|---|---|
| Content Uniformity | Conforms to USP <905> requirements (by weight |
| | variation) |
| Dissolution | ER6: release profile matching ER3 |
| | ER8, and ER9: release profile within the range of |
| | IVIVC model prediction |
| Degradation Products | Meets ICH requirements (0.2% Identification |

Threshold)

In general, manufacturing the osmotic tablets without an immediate release coating was carried out by: preparing a pre-blend comprising the drug substance (dexpramipexole, or a pharmaceutically acceptable salt thereof) and one or more further ingredients such as the inorganic osmotic agent, preparing a blend from the pre-blend and a lubricant (such as magnesium stearate), compressing the blend to form a tablet core, coating the tablet core with a semipermeable membrane coating solution to provide a semipermeable membrane coating surrounding the tablet core, and optionally coating the semipermeable membrane coating with a film coating solution to provide a film coating surrounding the semipermeable membrane coating (more than one film coating may be applied). Further, drilling one or more delivery orifices in the semipermeable membrane coating (such as with mechanical or laser drilling) was carried out.

In general, manufacturing the osmotic tablets with an immediate release coating was carried out by: preparing a pre-blend comprising the drug substance (dexpramipexole, or a pharmaceutically acceptable salt thereof) and one or more further ingredients such as the inorganic osmotic agent, preparing a blend from the pre-blend and a lubricant (such as magnesium stearate), compressing the blend to form a tablet core, coating the tablet core with a semipermeable membrane coating solution to provide a semipermeable membrane coating surrounding the tablet core, coating the semipermeable membrane coating with a seal coating solution to provide a seal coating surrounding the semipermeable membrane coating, coating the seal coating with an immediate release drug coating solution to provide an immediate release drug coating surrounding the seal coating, and optionally further coating the drug coating with a film coating solution to provide a film coating surrounding the drug coating (more than one film coating may be applied). Further, drilling one or more delivery orifices in the semipermeable membrane coating (such as with mechanical or laser drilling) was carried out.

In general, during preparation of the pre-blend and the blend, no water was added or heat was applied. Further, during compression of the blend, no further excipients or water were introduced. The solvent of the semipermeable membrane coating solution was acetone: purified water at the indicated ratios. The drilling process did not introduce any new excipients or applied water. Purified water was the solvent for preparing the seal coating solution, the immediate release drug coating solution, and the film coating solution.

### Example 9.1—ER6 Formulation Development

The objective of formulation ER6 was to increase the strength to 280 mg Dexpramipexole (equivalent to 376 mg Dexpramipexole diHCl) and achieve the same release profile as ER3 in 0.1N HCl media (ER3 correspond to the two osmotic tablet formulations of Table 31). The approach that was adopted was to dose proportionally scale up the core ER3 formulation from 223 mg Dexpramipexole strength to 280 mg strength and adjust the coating weight gain and/or plasticizer (or pore former) level since the specific surface area will reduce as the tablet gets bigger. Two levels of pore former were studied during this phase i.e. 15% of coating and 20% w/w of coating. Samples were tested at different weight gains. The formulations used to study impact of the polymer level are summarized in Table 39.

TABLE 39
Formulation compositions used to study the impact of coating weight
gain and pore former level on the CQA dissolution

| | | Summary | | | | | | | |
|---|---|---|---|---|---|---|---|---|---|
| | | Formulation component | | | | | | | |
| | | Lot Number | | | | | | | |
| | | AZF-1-FD-5-17A | | AZF-1-FD-5-17C | | AZF-1-FD-5-23C | | AZF-1-FD-5-23F | |
| | | PEG 3350 (Plasticizer) amount | | | | | | | |
| | | 15% w/w of | | 15% w/w of | | 15% w/w of | | 15% w/w of | |
| | | coating | | coating | | coating | | coating | |
| | | Coating Weight gain | | | | | | | |
| | | 4% w/w | | 6% w/w | | 4% w/w | | 7% w/w | |
| | | | % w/w | | % w/w | | % w/w | | % w/w |
| Material | Function | mg/tab | core | mg/tab | core | mg/tab | core | mg/tab | core |

Core Tablet

| | | | | | | | | | |
|---|---|---|---|---|---|---|---|---|---|
| Dexpramipexole | Drug | 399.82 | 42.00 | 399.82 | 42.00 | 399.82 | 42.00 | 399.82 | 42.00 |
| Dihydrochloride | Substance | | | | | | | | |
| Monohydrate* | | | | | | | | | |
| Silicified | Diluent | 290.35 | 30.50 | 290.35 | 30.50 | 290.35 | 30.50 | 290.35 | 30.50 |
| Microcrystalline | | | | | | | | | |
| Cellulose | | | | | | | | | |
| (Prosolv SMCC | | | | | | | | | |
| HD90) | | | | | | | | | |
| Copovidone | Binder | 66.64 | 7.00 | 66.64 | 7.00 | 66.64 | 7.00 | 66.64 | 7.00 |
| (Kollidon VA | | | | | | | | | |
| 64 fine) | | | | | | | | | |
| Sodium Chloride | Inorganic | 190.39 | 20.00 | 190.39 | 20.00 | 190.39 | 20.00 | 190.39 | 20.00 |
| (Emprove ®) | Osmogen | | | | | | | | |
| Magnesium | Lubricant | 4.76 | 0.50 | 4.76 | 0.50 | 4.76 | 0.50 | 4.76 | 0.50 |
| stearate | | | | | | | | | |
| (Vegetable | | | | | | | | | |
| source) | | | | | | | | | |

| | | | | | | | | |
|---|---|---|---|---|---|---|---|---|
| Total | 951.96 | 100.00 | 951.96 | 100.00 | 951.96 | 100.00 | 951.96 | 100.00 |

Coating Membrane\hat{ }\hat{ }

| | | | | | | | | | |
|---|---|---|---|---|---|---|---|---|---|
| Opadry CA | CA: | 38.08 | n/a | 57.12 | n/a | n/a | n/a | n/a | n/a |
| 500F190003 | Film | | | | | | | | |
| clear (CA | former & | | | | | | | | |
| 398-10:PEG | PEG 3350: | | | | | | | | |
| 3350 = 85:15) | Pore former | | | | | | | | |
| Opadry CA | | n/a | n/a | n/a | n/a | 38.08 | n/a | 66.64 | n/a |
| 500F190012 | | | | | | | | | |
| clear (CA | | | | | | | | | |
| 398-10:PEG | | | | | | | | | |
| 3350 = 80:20) | | | | | | | | | |
| Acetone\hat{ } | Solvent | q.s. | n/a | q.s. | n/a | q.s. | n/a | q.s. | n/a |
| Purified water\hat{ } | Solvent | q.s. | n/a | q.s. | n/a | q.s. | n/a | q.s. | n/a |
| Total | | 989.68 | n/a | 1009.08 | n/a | 989.68 | n/a | 1018.60 | n/a |

q.s.: Quantity sufficient
*Conversion fator for Dexpramipexole Dihydrochloride Monohydrate to Dexpramipexole Dihydrochloride = 0.9404
\hat{ }Evaporates during processing
\hat{ }\hat{ }Solid content of Coating Solution = 5% w/w, Acetone:water = 94:6

Based on the high-risk formulation variables outlined above, multiple batches were manufactured at a scale of about 500 g with the sole objective being dissolution optimization. Laboratory scale compression was performed using a Korsch XL-100 rotary tablet press. Laboratory scale coating was performed using a Vector LDCS-5 Pan Coater equipped with 6″ partially perforated coating pans.

The following is the process used to prepare blend for core tablets:
Co-sift Dexpramipexole, Prosolv SMCC HD90, Co-povidone and Sodium chloride through Quadro Co-mill 197 with 024R screen at −1700 rpm.
Blend co-sifted materials of step-1 in V-blender at 25 rpm for 10 minutes.
Screen Magnesium stearate through #40 US std sieve. Lubricate blend of step-2 using sifted Magnesium stearate of step-3 in v-blender at 25 rpm for 5 minutes.

Table 40 details the in-process compression parameters for each lot. The parameters were intentionally kept constant to evaluate impact of formulation variables.

TABLE 40
In-process compression parameters for trials of ER6 Formulation Development

| | Lot Number | | | |
|---|---|---|---|---|
| | AZF-1-FD-5- | AZF-1-FD-5- | AZF-1-FD-5- | AZF-1-FD-5- |
| Parameter | 17A | 17C | 23C | 23F |
| Tooling | 8.51 mm* 19.00 | 8.51 mm* 19.00 | 8.51 mm* 19.00 | 8.51 mm* 19.00 |
| | mm capsule | mm capsule | mm capsule | mm capsule |
| | shaped | shaped | shaped | shaped |
| Average tablet weight | 950 | 950 | 950 | 950 |
| (mg) (10 tablets) | | | | |
| Average Hardness (kP) | 26.1 | 26.1 | 26.1 | 26.1 |
| Thickness (mm) | 6.1 | 6.1 | 6.1 | 6.1 |
| Friability (%) | 0.05 | 0.05 | 0.05 | 0.05 |

The following are the steps to prepare solution for membrane coating:
Add purified water and Acetone to a stainless-steel container and record the starting temperature using Infra-red (IR) gun.
Place acetone/water solution container into a second larger stainless-steel container (aka: water-bath) containing about 1/10ₜₕ of warm (˜70° C.) purified water. When solution reaches Not less than (NLT) 30° C., record time and continue with Opadry addition.
Using an overhead mixer, create a vortex.
Slowly add Opadry CA to the vortex.
After complete addition, mix the content NLT 15 minutes. At the end of 15 minutes, record if a homogenous solution is obtained.

Table 41 details the in-process coating parameters for each lot. The parameters were selected based on recommendations of vendor of Opadry CA, and they were intentionally kept constant to evaluate impact of formulation variables.

TABLE 41
In-process coating parameters for trials of ER6 Formulation Development

| | Lot Number | | | |
|---|---|---|---|---|
| | AZF-1-FD-5- | AZF-1-FD-5- | AZF-1-FD-5- | AZF-1-FD-5- |
| Parameter | 17A | 17C | 23C | 23F |
| Pan load (g) | 300 | 300 | 300 | 300 |
| Inlet temperature (° C.) | 32-35 | 32-35 | 32-33 | 32-33 |
| Product temperature (° C.) | 27-28 | 27-28 | 25-26 | 25-26 |
| Spray rate (g/min) | 12 | 12 | 12 | 12 |
| Atomization (PSI) | 20 | 20 | 20 | 20 |
| Pan speed (rpm) | 24 | 24 | 24 | 24 |

Coated tablets of each lot were drilled with 0.5 mm Delivery orifice using a mechanical drill press. The dissolution results for Lot #AZF-1-FD-5-17A and Lot #AZF-1-FD-5-17C are provided in FIG. 17.

These results confirmed that after dose proportionally scaling up from 223 mg to 280 mg, the release became slower at similar weight gain due to the reduced tablet specific surface area (ER3 clinical batch vs. AZF-1-FD-5-17C). Only 4.5-5% weight gain is needed to match the dissolution profile with ER3. However, it is advisable to avoid target weight gains of less than 6% to have reproducible scale up process and dissolution profiles.

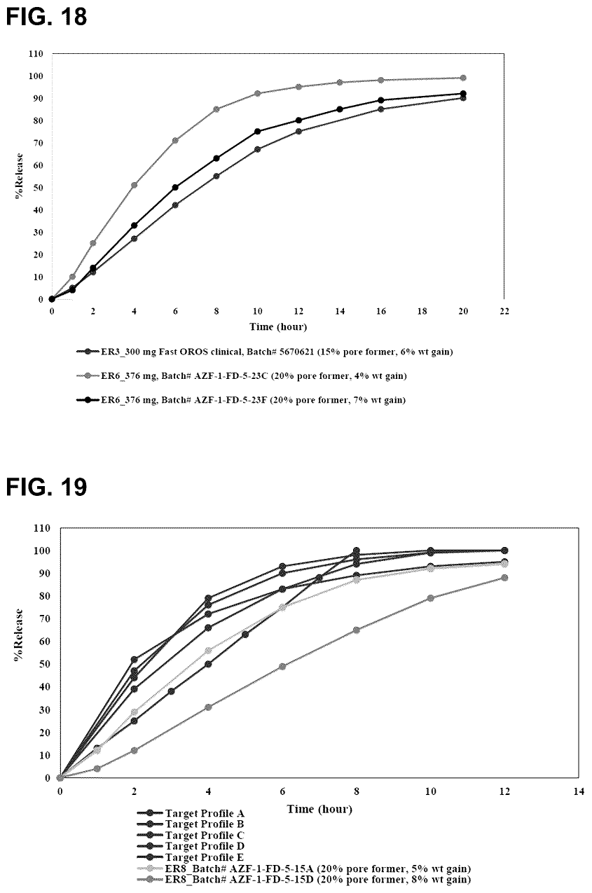

The dissolution results for Lot #AZF-1-FD-5-23C and Lot #AZF-1-FD-5-23F are provided in FIG. 18.

These results confirmed that when pore former level was increased to 20% (of dry polymer) to 15%, release rate is significantly increased. Even at 7% weight gain, release rate is faster (ER3 clinical batch vs. AZF-1-FD-5-23F). To have a target weight gain level between 6% w/w to 7% w/w, it was decided to manufacture confirmation batch with intermediate pore former level of 17.5%.

### Example 9.2—ER8 Formulation Development

The objective of ER8 development was to use higher level of pore former and fall in the target dissolution range. Two levels of pore former were studied during this phase i.e. 20% of coating and 25% w/w of coating. Samples were tested at different weight gains. The formulations used to study impact of the polymer level are summarized in Table 42.

TABLE 42
Formulation compositions used to study the impact of coating weight
gain and pore former level on the CQA dissolution

| | | Summary | | | | | | | |
|---|---|---|---|---|---|---|---|---|---|
| | | Formulation component | | | | | | | |
| | | Lot Number | | | | | | | |
| | | AZF-1-FD-5-15A | | AZF-1-FD-5-15D | | AZF-1-FD-5-16A | | AZF-1-FD-5-16D | |
| | | PEG 3350 (Plasticizer) amount | | | | | | | |
| | | 20% w/w of | | 20% w/w of | | 25% w/w of | | 25% w/w of | |
| | | coating | | coating | | coating | | coating | |
| | | Coating Weight gain | | | | | | | |
| | | 5% w/w | | 8% w/w | | 5% w/w | | 8% w/w | |
| | | | % w/w | | % w/w | | % w/w | | % w/w |
| Material | Function | mg/tab | core | mg/tab | core | mg/tab | core | mg/tab | core |

Core Tablet

| | | | | | | | | | |
|---|---|---|---|---|---|---|---|---|---|
| Dexpramipexole | Drug | 319.01 | 42.00 | 319.01 | 42.00 | 319.01 | 42.00 | 319.07 | 42.00 |
| Dihydrochloride | Substance | | | | | | | | |
| Monohydrate* | | | | | | | | | |
| Silicified | Diluent | 231.66 | 30.50 | 231.66 | 30.50 | 231.66 | 30.50 | 231.66 | 30.50 |
| Microcrystalline | | | | | | | | | |
| Cellulose | | | | | | | | | |
| (Prosolv SMCC | | | | | | | | | |
| HD90) | | | | | | | | | |
| Copovidone | Binder | 53.17 | 7.00 | 53.17 | 7.00 | 53.17 | 7.00 | 53.17 | 7.00 |
| (Kollidon VA | | | | | | | | | |
| 64 fine) | | | | | | | | | |
| Sodium Chloride | Inorganic | 151.91 | 20.00 | 151.91 | 20.00 | 151.91 | 20.00 | 151.91 | 20.00 |
| (Emprove ®) | Osmogen | | | | | | | | |
| Magnesium | Lubricant | 3.80 | 0.50 | 3.80 | 0.50 | 3.80 | 0.50 | 3.80 | 0.50 |
| stearate | | | | | | | | | |
| (Vegetable | | | | | | | | | |
| source) | | | | | | | | | |

| | | | | | | | | |
|---|---|---|---|---|---|---|---|---|
| Total | 759.55 | 100.00 | 759.55 | 100.00 | 759.55 | 100.00 | 759.55 | 100.00 |

Coating Membrane\hat{ }\hat{ }

| | | | | | | | | | |
|---|---|---|---|---|---|---|---|---|---|
| Opadry CA | CA: | 37.98 | n/a | 60.76 | n/a | n/a | n/a | n/a | n/a |
| 500F190003 | Film | | | | | | | | |
| clear (CA | former & | | | | | | | | |
| 398-10:PEG | PEG 3350: | | | | | | | | |
| 3350 = 85:15) | Pore former | | | | | | | | |
| Opadry CA | | n/a | n/a | n/a | n/a | 37.98 | n/a | 60.76 | n/a |
| 500F190012 | | | | | | | | | |
| clear (CA | | | | | | | | | |
| 398-10:PEG | | | | | | | | | |
| 3350 = 80:20) | | | | | | | | | |
| Acetone\hat{ } | Solvent | q.s. | n/a | q.s. | n/a | q.s. | n/a | q.s. | n/a |
| Purified water\hat{ } | Solvent | q.s. | n/a | q.s. | n/a | q.s. | n/a | q.s. | n/a |
| Total | | 797.53 | n/a | 820.31 | n/a | 797.31 | n/a | 820.31 | n/a |

q.s.: Quantity sufficient
*Conversion fator for Dexpramipexole Dihydrochloride Monohydrate to Dexpramipexole Dihydrochloride = 0.9404
\hat{ }Evaporates during processing
\hat{ }\hat{ }Solid content of Coating Solution = 5% w/w, Acetone:water = 94:6

Based on the high-risk formulation variables outlined above, multiple batches were manufactured at a scale of about 500 g with the sole objective being dissolution optimization. Laboratory scale compression was performed using a Korsch XL-100 rotary tablet press. Laboratory scale coating was performed using a Vector LDCS-5 Pan Coater equipped with 6″ partially perforated coating pans.

The following is the process used to prepare blend for core tablets:
Co-sift Dexpramipexole, Prosolv SMCC HD90, Co-povidone and Sodium chloride through Quadro Co-mill 197 with 024R screen at ˜1700 rpm.
Blend co-sifted materials of step-1 in V-blender at 25 rpm for 10 minutes.
Screen Magnesium stearate through #40 US std sieve. Lubricate blend of step-2 using sifted Magnesium stearate of step-3 in v-blender at 25 rpm for 5 minutes.

Table 43 details the in-process compression parameters for each lot. The parameters were intentionally kept constant to evaluate impact of formulation variables.

TABLE 43
In-process compression parameters for trials of ER8 Formulation Development

| | Lot Number | | | |
|---|---|---|---|---|
| | AZF-1-FD-5- | AZF-1-FD-5- | AZF-1-FD-5- | AZF-1-FD-5- |
| Parameter | 15A | 15D | 16A | 16D |
| Tooling | 7.7 mm* 17.02 | 7.7 mm* 17.02 | 7.7 mm* 17.02 | 7.7 mm* 17.02 |
| | mm capsule | mm capsule | mm capsule | mm capsule |
| | shaped | shaped | shaped | shaped |
| Average tablet weight | 760 | 760 | 760 | 760 |
| (mg) (10 tablets) | | | | |
| Average Hardness (kP) | 23.1 | 23.1 | 23.1 | 23.1 |
| Thickness (mm) | 5.94 | 5.94 | 5.94 | 5.94 |
| Friability (%) | 0.11 | 0.11 | 0.11 | 0.11 |

The following are the steps to prepare solution for membrane coating:
Add purified water and Acetone to a stainless-steel container and record the starting temperature using Infra-red (IR) gun.
Place acetone/water solution container into a second larger stainless-steel container (aka: water-bath) containing about 1/10ₜₕ of warm (˜70° C.) purified water. When solution reaches Not less than (NLT) 30° C., record time and continue with Opadry addition.
Using an overhead mixer, create a vortex.
Slowly add Opadry CA to the vortex.
After complete addition, mix the content NLT 15 minutes. At the end of 15 minutes, record if a homogenous solution is obtained.

Table 44 details the in-process coating parameters for each lot. The parameters were selected based on recommendations of vendor of Opadry CA, and they were intentionally kept constant to evaluate impact of formulation variables.

TABLE 44
In-process coating parameters for trials of ER8 Formulation Development

| | Lot Number | | | |
|---|---|---|---|---|
| | AZF-1-FD-5- | AZF-1-FD-5- | AZF-1-FD-5- | AZF-1-FD-5- |
| Parameter | 15A | 15D | 16A | 16D |
| Pan load (g) | 300 | 300 | 300 | 300 |
| Inlet temperature (° C.) | 34-37 | 34-37 | 34-37 | 34-37 |
| Product temperature (° C.) | 27-28 | 27-28 | 27-28 | 27-28 |
| Spray rate (g/min) | 12 | 12 | 12 | 12 |
| Atomization (PSI) | 20 | 20 | 20 | 20 |
| Pan speed (rpm) | 24 | 24 | 24 | 24 |
| Drying Time (min) | 5 | 5 | 5 | 5 |

Coated tablets of each lot were drilled with 0.5 mm Delivery orifice using a mechanical drill press. The dissolution results for Lot #AZF-1-FD-5-15A and Lot #AZF-1-FD-5-15D are provided in FIG. 19.

These results show that release profile of 5% weight gain (Batch No. AZF-1-FD-1-5-15A) is closer to one of the target profiles, and release profile of 8% weight gain (Batch No. AZF-1-FD-1-5-15D) is significantly lower than any of the target profiles. However, it is advisable to avoid target weight gains of less than 6% to have reproducible scale up process and dissolution profiles.

The dissolution results for Lot #AZF-1-FD-5-16A and Lot #AZF-1-FD-5-16D are provided in FIG. 20.

These results show that release profile of 5% weight gain (Batch No. AZF-1-FD-1-5-16A) falls within the range of target release profiles. However, since the batch size very small and 5% w/w was one of the samples collected during the manufacturing of batch, it is possible that practical weight gain achieved for that sample may not be accurately 5% w/w.

### Example 9.3—ER9 Formulation Development

Dexpramipexole Dihydrochloride Monohydrate is a water-soluble salt and results in some osmotic pressure across the membrane. The objective of ER9 development was to reduce the amount (% w/w) of sodium chloride, compensate it with increased amount (% w/w) of drug substance and fall in the target dissolution range. The formulations used to study impact of the reduced Sodium chloride level are summarized in Table 45.

TABLE 45
Formulation compositions used to study the impact of coating weight
gain and pore former level on the CQA dissolution

| | | Summary | | | | | |
|---|---|---|---|---|---|---|---|
| | | Formulation component | | | | | |
| | | Lot Number | | | | | |
| | | AZF-1-FD-5-18A | | AZF-1-FD-5-18C | | AZF-1-FD-5-18D | |
| | | Sodium chloride level (% w/w of core) | | | | | |
| | | 15% w/w of | | 15% w/w of | | 15% w/w of | |
| | | coating | | coating | | coating | |
| | | Coating Weight gain | | | | | |
| | | 5% w/w | | 8% w/w | | 5% w/w | |
| | | | % w/w | | % w/w | | % w/w |
| Material | Function | mg/tab | core | mg/tab | core | mg/tab | core |

Core Tablet

| | | | | | | | |
|---|---|---|---|---|---|---|---|
| Dexpramipexole | Drug | 319.01 | 52.50 | 319.01 | 52.50 | 319.01 | 52.50 |
| Dihydrochloride | Substance | | | | | | |
| Monohydrate* | | | | | | | |
| Silicified | Diluent | 185.32 | 30.50 | 185.32 | 30.50 | 185.32 | 30.50 |
| Microcrystalline | | | | | | | |
| Cellulose | | | | | | | |
| (Prosolv SMCC | | | | | | | |
| HD90) | | | | | | | |
| Copovidone | Binder | 42.54 | 7.00 | 42.54 | 7.00 | 42.54 | 7.00 |
| (Kollidon VA | | | | | | | |
| 64 fine) | | | | | | | |
| Sodium Chloride | Inorganic | 57.7 | 10.00 | 57.7 | 10.00 | 57.7 | 10.00 |
| (Emprove ®) | Osmogen | | | | | | |
| Magnesium | Lubricant | 3.01 | 0.50 | 3.01 | 0.50 | 3.01 | 0.50 |
| stearate | | | | | | | |
| (Vegetable | | | | | | | |
| source) | | | | | | | |

| | | | | | | |
|---|---|---|---|---|---|---|
| Total | 607.62 | 100.00 | 607.62 | 100.00 | 607.62 | 100.00 |

Coating Membrane

| | | | | | | | |
|---|---|---|---|---|---|---|---|
| Opadry CA | CA: | 30.38 | n/a | 42.53 | n/a | 48.61 | n/a |
| 500F190003 | Film | | | | | | |
| clear (CA | former & | | | | | | |
| 398-10:PEG | PEG 3350: | | | | | | |
| 3350 = 85:15) | Pore former | | | | | | |
| Acetone\hat{ } | Solvent | q.s. | n/a | q.s. | n/a | q.s. | n/a |
| Purified water\hat{ } | Solvent | q.s. | n/a | q.s. | n/a | q.s. | n/a |
| Total | | 638.00 | n/a | 650.15 | n/a | 656.23 | n/a |

q.s.: Quantity sufficient
*Conversion fator for Dexpramipexole Dihydrochloride Monohydrate to Dexpramipexole Dihydrochloride = 0.9404
\hat{ }Evaporates during processing
\hat{ }\hat{ }Solid content of Coating Solution = 5% w/w, Acetone:water = 94:6

Based on the high-risk formulation variables outlined above, multiple batches were manufactured at a scale of about 500 g with the sole objective being dissolution optimization. Laboratory scale compression was performed using a Korsch XL-100 rotary tablet press. Laboratory scale coating was performed using a Vector LDCS-5 Pan Coater equipped with 6″ partially perforated coating pan.

The following is the process used to prepare blend for core tablets.
Co-sift Dexpramipexole, Prosolv SMCC HD90, Co-povidone and Sodium chloride through Quadro Co-mill 197 with 024R screen at ˜1700 rpm.
Blend co-sifted materials of step-1 in V-blender at 25 rpm for 10 minutes.
Screen Magnesium stearate through #40 US std sieve. Lubricate blend of step-2 using sifted Magnesium stearate of step-3 in v-blender at 25 rpm for 5 minutes.

Table 46 details the in-process compression parameters for each lot. The parameters were intentionally kept constant to evaluate impact of formulation variables.

TABLE 46
In-process compression parameters for trials of ER9 Formulation Development

| | Lot Number | | |
|---|---|---|---|
| Parameter | AZF-1-FD-5-18A | AZF-1-FD-5-18C | AZF-1-FD-5-18D |
| Tooling | 8.00 mm* 16.50 | 8.00 mm* 16.50 mm | 8.00 mm* 16.50 mm |
| | mm Oval shaped | Oval shaped | Oval shaped |
| Average tablet weight | 610 | 610 | 610 |
| (mg) (10 tablets) | | | |
| Average Hardness (kP) | 16.5 | 16.5 | 16.5 |
| Thickness (mm) | 5.7 | 5.7 | 5.7 |
| Friability (%) | 0.15 | 0.15 | 0.15 |

The following are the steps to prepare solution for membrane coating.
Add purified water and Acetone to a stainless-steel container and record the starting temperature using Infra-red (IR) gun.
Place acetone/water solution container into a second larger stainless-steel container (aka: water-bath) containing about 1/10th of warm (˜70° C.) purified water. When solution reaches Not less than (NLT) 30° C., record time and continue with Opadry addition.
Using an overhead mixer, create a vortex.
Slowly add Opadry CA to the vortex.
After complete addition, mix the content NLT 15 minutes. At the end of 15 minutes, record if a homogenous solution is obtained.

Table 47 details the in-process coating parameters for each lot. The parameters were selected based on recommendations of vendor of Opadry CA, and they were intentionally kept constant to evaluate impact of formulation variables.

TABLE 47
In-process coating parameters for trials of ER9 Formulation Development

| | Lot Number | | |
|---|---|---|---|
| Parameter | AZF-1-FD-5-18A | AZF-1-FD-5-18C | AZF-1-FD-5-18D |
| Pan load (g) | 300 | 300 | 300 |
| Inlet temperature (° C.) | 34-36 | 34-36 | 34-36 |
| Product temperature (° C.) | 26-27 | 26-27 | 26-27 |
| Spray rate (g/min) | 12 | 12 | 12 |
| Atomization (PSI) | 20 | 20 | 20 |
| Pan speed (rpm) | 26 | 26 | 26 |
| Drying Time (min) | 5 | 5 | 5 |

Coated tablets of each lot were drilled with 0.5 mm Delivery orifice using a mechanical drill press. The dissolution results for Lot #AZF-1-FD-5-18A, Lot #AZF-1-FD-5-18C and Lot #AZF-1-FD-5-18D are provided in FIG. 21.

These results show that release profiles of 5%, 7%, and 8% weight gain (Batch Nos. AZF-1-FD-1-5-15A/15C/15D) are significantly slower than any of the target profiles. Due to reduction of sodium chloride, slope of the profiles is also reduced, and profiles shift towards pseudo zero-order or first order from zero-order. Despite being a salt, the drug substance does not have similar osmogenic property as Sodium chloride.

### Example 9.4—Confirmation Batches Round 1

To manufacture the first round of confirmation batches of ER6 and ER8 formulations, 2506.45 g of common blend was manufactured and then divided into two parts to compress the tablets of 376 mg strength (ER6) and 300 mg strength (ER8). The formulation composition of common blend is summarized in Table 48.

TABLE 48
Formulation of common blend of Dexpramipexole CR Osmotic
tablets Confirmation batch lot AZF-1-FD-5-25

| | | AZF-1-FD-5-25 | | | |
|---|---|---|---|---|---|
| | | mg/unit | mg/unit | | |
| | | (376 mg | (300 mg | | Qty/batch |
| Material | Function | strength) | strength) | % w/w | (g) |
| Dexpramipexole | Drug | 399.82 | 319.01 | 42.00 | 1052.73 |
| Dihydrochloride Monohydrate* | Substance | | | | |
| Silicified Microcrystalline | Diluent | 332.50 | 231.66 | 30.50 | 764.45 |
| Cellulose (Prosolv SMCC | | | | | |
| HD90) | | | | | |
| Copovidone (Kollidon VA 64 | Binder | 142.50 | 53.17 | 7.00 | 175.46 |
| fine) | | | | | |
| Sodium Chloride (Emprove ®) | Inorganic | 236.11 | 151.91 | 20.00 | 501.27 |
| | Osmogen | | | | |
| Magnesium stearate (Vegetable | Lubricant | 2.81 | 3.80 | 0.50 | 12.54 |
| source) | | | | | |

| | | | | |
|---|---|---|---|---|
| Total | 951.96 | 759.55 | 100.00 | 2506.45 |

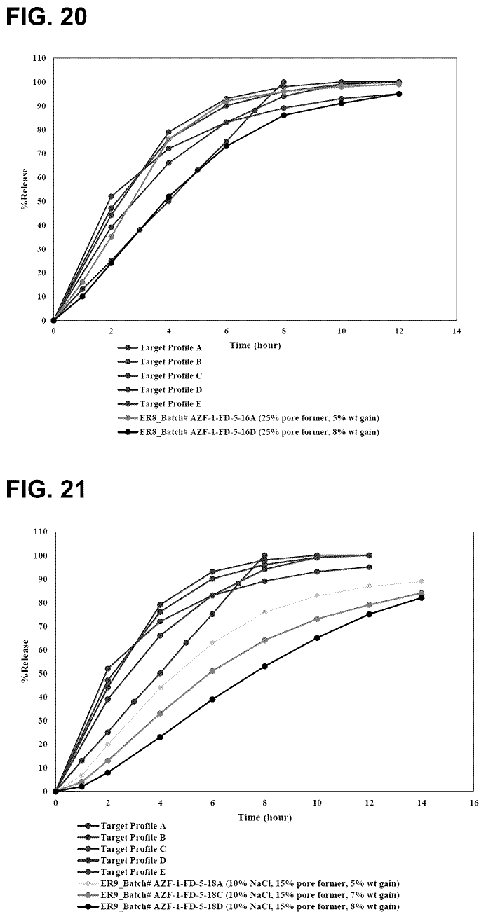

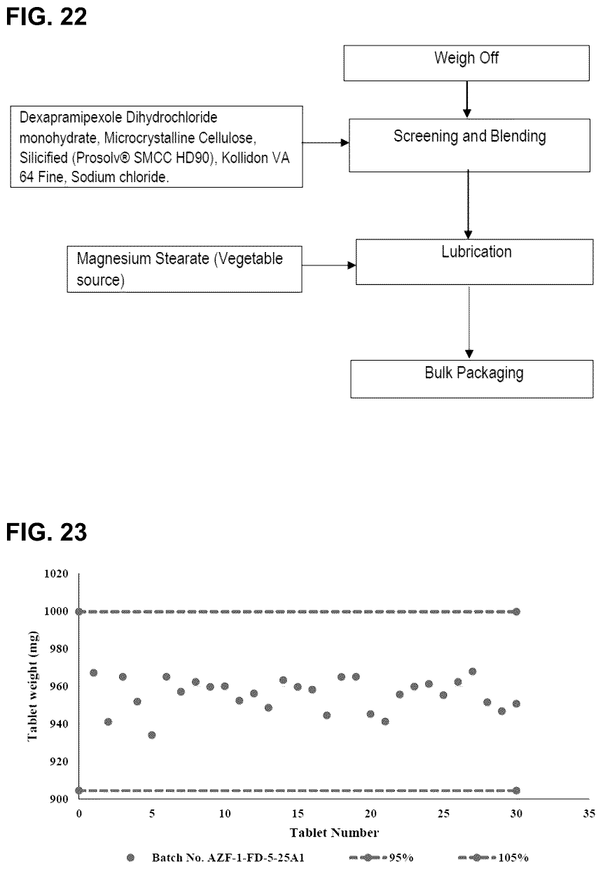

A process schematic is shown in FIG. 22.

The process parameters for common blending are summarized in Table 49.

TABLE 49
Process parameters of common blend of
Dexpramipexole ER Osmotic tablets
Confirmation batch lot AZF-1-FD-5-25.
Process Parameters

| Equipment | Diffusion blender |
|---|---|
| Pre-Mix (revolutions) | 250 |
| Lubrication (revolutions) | 125 |

### Example 9.4.1—ER6 First Confirmation Batch

Laboratory scale compression was performed using a Korsch XL-100 rotary tablet press. About 1 kg tablets were compressed using common blend of Lot No. AZF-1-FD-5-25. Laboratory scale coating was performed using a Vector LDCS-5 Pan Coater equipped with 12″ partially perforated coating pan. For semipermeable membrane coating, plasticizer (pore former) level was kept at 17.5% of coating and samples were collected at 6.2% weight gain (Lot No. AZF-1-FD-5-25A1) and 7.20% w/w (Lot No. AZF-1-FD-5-25A2). The formulation composition of first ER6 confirmation batch is in Table 50.

TABLE 50
Formulation compositions of first ER6 confirmation batch.

| | Summary | | | | |
|---|---|---|---|---|---|
| | Formulation | Lot Number | | | |
| | component | AZF-1-FD-5-25A1 | | AZF-1-FD-5-25A2 | |
| | PEG 3350 (Plasticizer) | 17.5% w/w | | 17.5% w/w | |
| | amount | of coating | | of coating | |
| | Coating Weight gain | 6.2% w/w | | 7.2% w/w | |
| Material | Function | mg/tab | % w/w core | mg/tab | % w/w core |

Core tablet

| | | | | | |
|---|---|---|---|---|---|
| Dexpramipexole Dihydrochloride Monohydrate* | Drug Substance | 399.82 | 42.00 | 399.82 | 42.00 |
| Silicified Microcrystalline Cellulose (Prosolv SMCC | Diluent | 332.50 | 30.50 | 332.50 | 30.50 |
| HD90) | | | | | |
| Copovidone (Kollidon VA 64 fine) | Binder | 142.50 | 7.00 | 142.50 | 7.00 |
| Sodium Chloride (Emprove ®) | Inorganic Osmogen | 236.11 | 20.00 | 236.11 | 20.00 |
| Magnesium stearate (Vegetable source) | Lubricant | 2.81 | 0.50 | 2.81 | 0.50 |

| | | | | |
|---|---|---|---|---|
| Total | 951.96 | 100.00 | 951.96 | 100.00 |

Coating Membrane\hat{ }\hat{ }

| | | | | | |
|---|---|---|---|---|---|
| Opadry CA 500F190003 clear (CA 398-10:PEG | CA: Film former & | 59.02 | n/a | 68.54 | n/a |
| 3350 = 85:15) | PEG 3350: Pore former | | | | |
| Acetone\hat{ } | Solvent | q.s. | n/a | q.s. | n/a |
| Purified water\hat{ } | Solvent | q.s. | n/a | q.s. | n/a |

| | | | | |
|---|---|---|---|---|
| Total | 1010.98 | n/a | 1020.50 | n/a |

Film Coating\hat{ }\hat{ }\hat{ }

| | | | | |
|---|---|---|---|---|
| Opadry II 85F130089 Pink | n/a | n/a | 30.62 | n/a |
| Purified water\hat{ } | n/a | n/a | q.s. | n/a |

| | | | | |
|---|---|---|---|---|
| Total | n/a | n/a | 1051.12 | n/a |

q.s.: Quantity sufficient
*Conversion factor for Dexpramipexole Dihydrochloride Monohydrate to Dexpramipexole Dihydrochloride = 0.9404
\hat{ }Evaporates during processing
\hat{ }\hat{ }Solid content of Coating Solution = 5% w/w, Acetone: water = 94:6
\hat{ }\hat{ }\hat{ }Solid content of Coating dispersion = 15% w/w

The compression parameters used were based on standard operational parameters for Korsch XL 100 compression. The main compression forces applied were to achieve the target hardness of 23-28 kp. Beginning, middle, and end of batch samples were collected for weight, thickness, hardness, and tensile strength measurements. The results are summarized in Tables 51 and 52.

TABLE 51
Summary of compression process parameters studied
during first Confirmatory batch of ER6.

| | |
|---|---|
| | Confirmation Batch |
| Parameter | AZF-1-FD-5-25A |

Press Set-Up

| Tooling | 8.50 mm × 19.0 mm capsule shaped | |
|---|---|---|
| Turret | 8 station D | |
| Number of Punches | 2 stations | |
| Fill Cam | 8-14 | mm |

Parameters

| Main Compression Force | 25-35 | kN |
|---|---|---|
| Ejection Force | 160 | N |
| Feeder Speed | Gravity feeder used | |
| Press Speed | 20 | rpm |

TABLE 52
Summary of in-process compression checks for laboratory
first confirmation batch of ER6 (Lot No. AZF-1-FD-5-25A.

| | Beginning | Middle | End |
|---|---|---|---|
| Tablet No. | Weight (mg) | Weight (mg) | Weight (mg) |
| 1 | 967.1 | 952.3 | 941.2 |
| 2 | 941.0 | 956.1 | 955.6 |
| 3 | 965.0 | 948.5 | 959.8 |
| 4 | 951.8 | 963.2 | 961.2 |
| 5 | 934.0 | 959.6 | 955.2 |
| 6 | 965.0 | 958.1 | 962.3 |
| 7 | 957.0 | 944.5 | 967.8 |
| 8 | 962.3 | 964.9 | 951.4 |
| 9 | 959.6 | 965.0 | 946.7 |
| 10 | 960.0 | 945.2 | 950.6 |
| Avg. | 956.0 | 955.7 | 955.2 |
| Min. | 934.0 | 944.2 | 941.2 |
| Max. | 967.1 | 965.0 | 967.8 |
| Avg. Hardness (kp) | 23.6 | 24.1 | 23.4 |
| Avg. Thickness (mm) | 3.12 | 6.15 | 6.11 |
| Tensile Strength (MPa) | 2.43 | 2.48 | 2.41 |

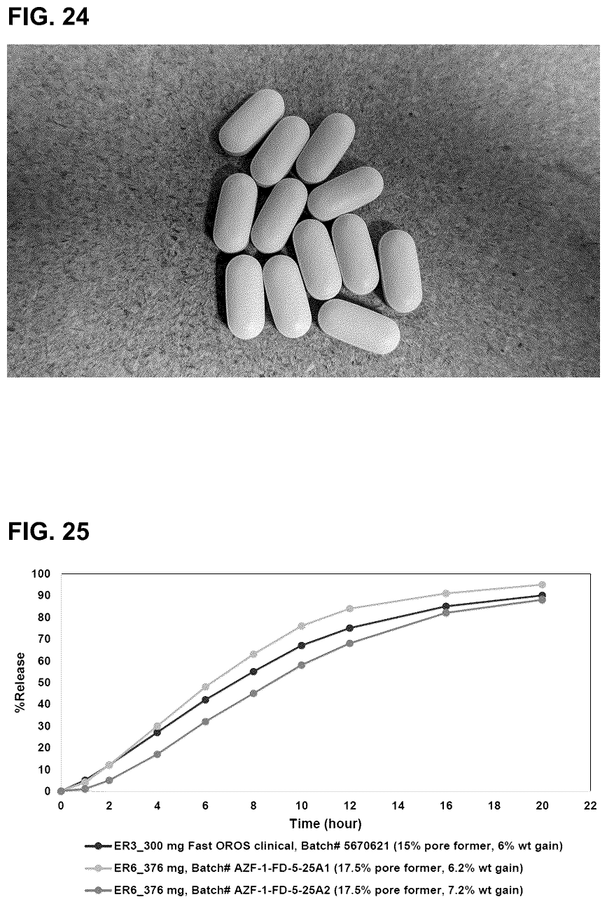

Weight variation was evaluated for all 30 tablets collected during compression (see FIG. 23). Process parameters for semipermeable membrane coating and film coating are described in Table 53. The appearance of coated tablets from Lot No. AZF-1-FD-5-25A is shown in FIG. 24.

TABLE 53
Process parameters of fist confirmation batch of ER6,
Lot No. AZF-1-FD-5-25A

| | Lot No. AZF-1-FD-5-25A | |
|---|---|---|
| | Semipermeable | |
| Parameters | membrane coating | Film Coating |
| Equipment | LDCS 5 | LDCS 5 |
| Pan load (g) | 900 | 900 |
| Pan Size (inch) | 12 | 12 |
| Nozzle Size (mm) | 0.5 | 0.5 |
| Atomization Pressure (psi) | 21 | 20 |
| Drum Speed (rpm) | 17 | 18 |
| Target Product Temperature (° C.) | 25 | 42 |
| Spray Rate (g/min) | 18 | 7 |

To test dissolution, film coated tablets were drilled with 0.5 mm Delivery orifice using a mechanical drill press. The dissolution results for Lot #AZF-1-FD-5-25A1 (6.2% weight gain), and Lot #AZF-1-FD-5-25A2 (7.2% weight gain) are provided in FIG. 25.

These results show that release profile of 6.2% weight gain sample is faster than target profile of ER3, and 7% and release profile of 7.200 weight gain sample is slower than target profile of ER3. Hence, 6.7% weight gain can be finalized as a target weight gain for future work.

### Example 9.4.2 ER8 First Confirmation Batch

Laboratory scale compression was performed using a Korsch XL-100 rotary tablet press. About 1 kg tablets were compressed using common blend of Lot No. AZF-1-FD-5-25. Laboratory scale coating was performed using a Vector LDCS-5 Pan Coater equipped with 12″ partially perforated coating pan. For semipermeable membrane coating, plasticizer (pore former) level was kept at 25% of coating and coating was done up to 50% weight gain. The formulation composition of first ER8 confirmation batch is in Table 54.

TABLE 54
Formulation compositions of first ER8 confirmation batch.

| Summary | Formulation component | Lot Number | |
|---|---|---|---|
| | | AZF-1-FD-5-25B | |
| | PEG 3350 (Plasticizer) | 25% w/w | |
| | amount | of coating | |
| | Coating Weight gain | 5.0% w/w | |
| Material | Function | mg/tab | % w/w core |

Core Tablet

| | | | |
|---|---|---|---|
| Dexpramipexole Dihydrochloride | Drug Substance | 319.01 | 42.00 |
| Monohydrate* | | | |
| Silicified Microcrystalline Cellulose | Diluent | 231.66 | 30.50 |
| (Prosolv SMCC HD90) | | | |
| Copovidone (Kollidon VA 64 fine) | Binder | 53.17 | 7.00 |
| Sodium Chloride (Emprove ®) | Inorganic Osmogen | 151.91 | 20.00 |
| Magnesium stearate (Vegetable | Lubricant | 3.80 | 0.50 |
| source) | | | |

| | | |
|---|---|---|
| Total | 759.55 | 100.00 |

Coating Membrane\hat{ }\hat{ }

| | | | |
|---|---|---|---|
| Opadry CA 500F190003 clear (CA | CA: Film former & | 37.98 | n/a |
| 398-10:PEG 3350 = 85:15) | PEG 3350: Pore former | | |
| Acetone\hat{ } | Solvent | q.s. | n/a |
| Purified water\hat{ } | Solvent | q.s. | n/a |

| | | |
|---|---|---|
| Total | 797.53 | n/a |

Film Coating

| | | |
|---|---|---|
| Opadry II 85F130089 Pink | 23.93 | n/a |
| Purified water\hat{ } | n/a | n/a |

| | | |
|---|---|---|
| Total | 821.46 | n/a |

q.s.: Quantity sufficient
*Conversion factor for Dexpramipexole Dihydrochloride Monohydrate to Dexpramipexole Dihydrochloride = 0.9404
\hat{ }Evaporates during processing
\hat{ }\hat{ }Solid content of Coating Solution = 5% w/w, Acetone:water = 94:6
\hat{ }\hat{ }\hat{ }Solid content of Coating dispersion = 15% w/w

Compression parameters were established during set-up ofthe Korsch XL 100 to achieve the target tablet weight, thickness, hardness, and friability. The main compression forces applied was to achieve the target hardness of 20-25 kp. Beginning, middle, and end of batch samples were collected for weight, thickness, hardness, and tensile strength measurements, the results are summarized in Tables 55 and 56.

TABLE 55
Summary of compression process parameters studied
during first Confirmatory batch of ER8.

| | |
|---|---|
| | Confirmation Batch |
| Parameter | AZF-1-FD-5-25B |

Press Set-Up

| Tooling | 7.75 mm × 17.0 mm capsule shaped | |
|---|---|---|
| Turret | 10 station B | |
| Number of Punches | 2 stations | |
| Fill Cam | 8-14 | mm |

Parameters

| Main Compression Force | 18-25 | KN |
|---|---|---|
| Feeder Speed | Gravity feeder used | |
| Press Speed | 20 | rpm |

TABLE 56
Summary of in-process compression checks for laboratory
first confirmation batch of ER8 (Lot No. AZF-1-FD-5-25B).

| | Beginning | Middle | End |
|---|---|---|---|
| Tablet No. | Weight (mg) | Weight (mg) | Weight (mg) |
| 1 | 740 | 750 | 762 |
| 2 | 750 | 752 | 752 |
| 3 | 748 | 755 | 756 |
| 4 | 750 | 759 | 759 |
| 5 | 742 | 760 | 754 |
| 6 | 746 | 751 | 766 |
| 7 | 740 | 763 | 755 |
| 8 | 743 | 752 | 766 |
| 9 | 742 | 758 | 757 |
| 10 | 748 | 766 | 750 |
| Avg. | 745 | 757 | 758 |
| Min. | 740 | 751 | 752 |
| Max | 750 | 766 | 766 |
| Average Hardness (kp) | 19.1 | 20.1 | 18.2 |
| Average Thickness mm) | 5.93 | 6.01 | 5.99 |
| Tensile strength (MPa) | 2.25 | 2.32 | 2.11 |

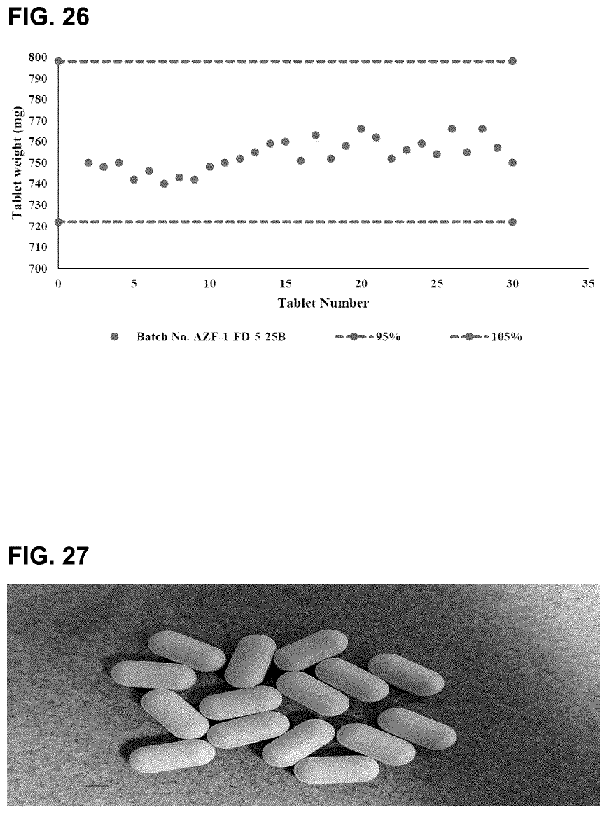

Weight variation was evaluated for all 30 tablets collected during compression (see FIG. 26). Process parameters for semipermeable membrane coating and film coating are described in Table 57. The appearance of coated tablets from Lot No. AZF-1-FD-5-25B is shown in FIG. 27.

TABLE 57
Process parameters of fist confirmation batch of ER8,
Lot No. AZF-1-FD-5-25B

| | Lot No. AZF-1-FD-5-25B | |
|---|---|---|
| | Semipermeable | |
| Parameters | membrane coating | Film Coating |
| Equipment | LDCS 5 | LDCS 5 |
| Pan load (g) | 900 | 900 |
| Pan Size (inch) | 12 | 12 |
| Nozzle Size (mm) | 0.5 | 0.5 |
| Atomization Pressure (psi) | 21 | 19 |
| Drum Speed (rpm) | 16 | 18 |
| Target Product Temperature (° C.) | 24 | 42 |
| Spray Rate (g/min) | 18 | 7 |

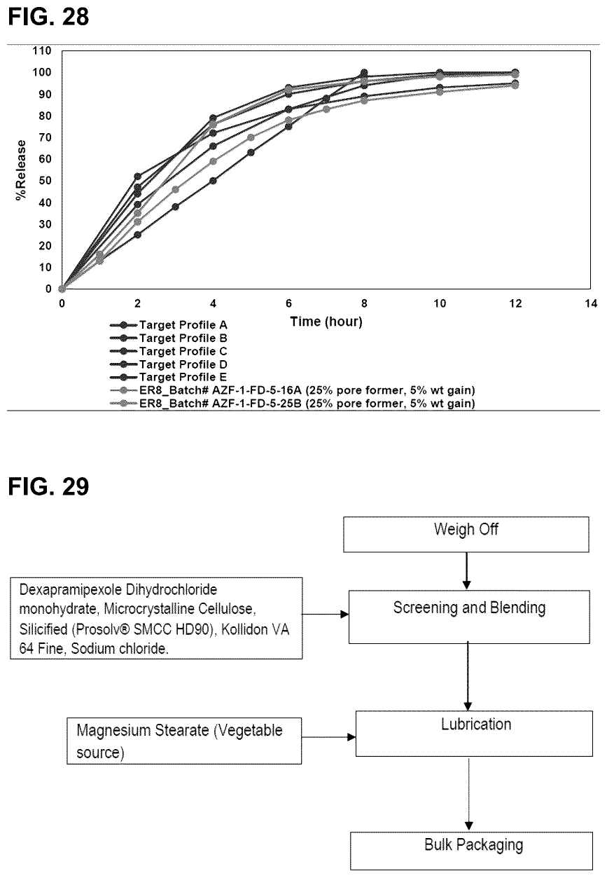

To test dissolution, film coated tablets were drilled with 0.5 mm Delivery orifice using a mechanical drill press. The dissolution results for Lot #AZF-1-FD-5-25B (5.0% weight gain) are provided in FIG. 28.

These results show that release profile of first confirmatory batch No. AZF-1-FD-5-25B is slower than release profile of development batch No. AZF-1-FD-5-16A. This discrepancy can be because of lower membrane weight gain achieved during the development batch (as mentioned above). Hence, 40% or 4.500 weight gain can be finalized as a target weight gain for future work.

### Example 9.5 Confirmation Batches Round 2

To manufacture the second round of confirmation batches of ER6 and ER8 formulations, 4557.18 g of common blend was manufactured and then divided into two parts to compress the tablets of 376 mg strength (ER6) and 300 mg strength (ER8). The formulation composition of common blend is summarized in Table 58.

TABLE 58
Formulation of common blend of Dexpramipexole ER Osmotic tablets
Confirmation batch lot AZF-1-FD-7-4

| | | AZF-1-FD-7-4 | | | |
|---|---|---|---|---|---|
| | | mg/unit | mg/unit | | |
| | | (376 mg | (300 mg | | Qty/batch |
| Material | Function | strength) | strength) | % w/w | (g) |
| Dexpramipexole Dihydrochloride | Drug | 399.82 | 319.01 | 42.00 | 1914.06 |
| Monohydrate* | Substance | | | | |
| Silicified Microcrystalline | Diluent | 332.50 | 231.66 | 30.50 | 1389.90 |
| Cellulose (Prosolv SMCC HD90) | | | | | |
| Copovidone (Kollidon VA 64 fine) | Binder | 142.50 | 53.17 | 7.00 | 319.02 |
| Sodium Chloride (Emprove ®) | Inorganic | 236.11 | 151.91 | 20.00 | 911.40 |
| | Osmogen | | | | |
| Magnesium stearate (Vegetable | Lubricant | 2.81 | 3.80 | 0.50 | 22.80 |
| source) | | | | | |

| | | | | |
|---|---|---|---|---|
| Total | 951.96 | 759.55 | 100.00 | 4557.18 |

*Conversion factor for Dexpramipexole Dihydrochloride Monohydrate to Dexpramipexole Dihydrochloride = 0.9404

A process schematic is shown in FIG. 29. The process parameters for common blending are summarized in Table 59.

TABLE 59
Process parameters of common blend of Dexpramipexole ER
Osmotic tablets Confirmation batch lot AZF-1-FD-7-4
Process Parameters

| Equipment | Diffusion blender |
|---|---|
| Pre-Mix (revolutions) | 250 |
| Lubrication (revolutions) | 125 |

### Example 9.5.1—ER6 Second Confirmation Batch

Laboratory scale compression was performed using a Korsch XL-100 rotary tablet press. About 1 kg tablets were compressed using common blend of Lot No. AZF-1-FD-7-4. Laboratory scale coating was performed using a Vector LDCS-5 Pan Coater equipped with 12″ partially perforated coating pan. For semipermeable membrane coating, plasticizer (pore former) level was kept at 17.500 of coating and tablets were coated up to 6.70% weight gain. The formulation composition of second ER6 confirmation batch is in Table 60.

TABLE 60
Formulation compositions of first ER6 confirmation batch.

| Summary | Formulation component | Lot Number | |
|---|---|---|---|
| | | AZF-1-FD-7-4C | |
| | PEG 3350 (Plasticizer) amount | 17.5% w/w of coating | |
| | Coating Weight gain | 6.7% w/w | |
| | | | % w/w |
| Material | Function | mg/tab | core |

Core Tablet

| | | | |
|---|---|---|---|
| Dexpramipexole Dihydrochloride | Drug Substance | 399.82 | 42.00 |
| Monohydrate* | | | |
| Silicified Microcrystalline Cellulose | Diluent | 332.50 | 30.50 |
| (Prosolv SMCC HD90) | | | |
| Copovidone (Kollidon VA 64 fine) | Binder | 142.50 | 7.00 |
| Sodium Chloride (Emprove ®) | Inorganic Osmogen | 236.11 | 20.00 |
| Magnesium stearate (Vegetable source) | Lubricant | 2.81 | 0.50 |

| | | |
|---|---|---|
| Total | 951.96 | 100.00 |

Coating Membrane\hat{ }\hat{ }

| | | | |
|---|---|---|---|
| Opadry CA 500F190003 clear | CA: Film former & | 63.78 | n/a |
| (CA 398-10:PEG 3350 = 85:15) | PEG 3350: Pore former | | |
| Acetone\hat{ } | Solvent | q.s. | n/a |
| Purified water\hat{ } | Solvent | q.s. | n/a |

| | | |
|---|---|---|
| Total | 1015.74 | n/a |

q.s.: Quantity sufficient
*Conversion factor for Dexpramipexole Dihydrochloride Monohydrate to Dexpramipexole Dihydrochloride = 0.9404
\hat{ }Evaporates during processing
\hat{ }\hat{ }?Solid content of Coating Solution = 5% w/w, Acetone:water = 94:6

The compression parameters used were based on standard operational parameters for Korsch XL 100 compression. The main compression forces applied was to achieve the target hardness of 23-28 kp. The process parameters for compression are summarized in Table 61.

TABLE 61
Process parameters of Dexpramipexole ER Osmotic 280 mg tablets
(376 mg diHCl) [ER6] second confirmation batch,
Lot No. AZF-1-FD-7-4C
Process Parameters

| | |
|---|---|
| Equipment | Korsch XL100 |
| Tooling | 8.5* 19.0 mm capsule shaped |
| Target Tablet weight (mg) | 952 |
| Target Hardness (kp) | 25 |
| Turret Speed (rpm) | 20 |
| Feeder Speed (rpm) | Gravity feeder used |
| Main compression force (kN) | 30-40 kN |

Process parameters for semipermeable membrane coating are described in Table 62.

TABLE 62
Process parameters of fist confirmation batch of ER6,
Lot No. AZF-1-FD-7-4C

| | Lot No. AZF-1-FD-7-4C |
|---|---|
| Parameters | Semipermeable membrane coating |
| Equipment | LDCS 5 |
| Pan load (g) | 900 |
| Pan Size (inch) | 12 |
| Nozzle Size (mm) | 0.5 |
| Atomization Pressure (psi) | 21 |
| Drum Speed (rpm) | 18 |
| Target Product Temperature (° C.) | 24 |
| Spray Rate (g/min) | 18 |

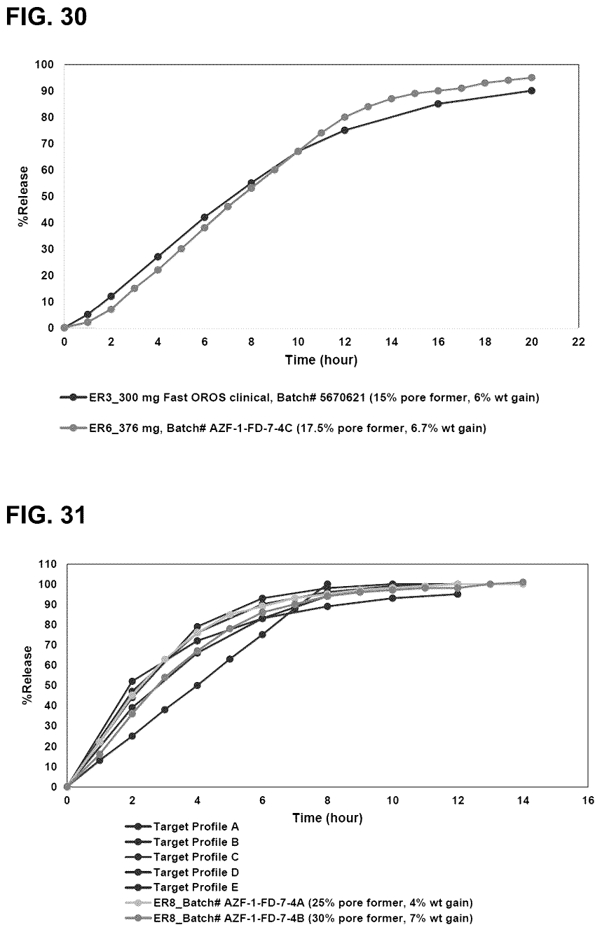

To test dissolution, film coated tablets were drilled with 0.5 mm Delivery orifice using a mechanical drill press. The dissolution results for Lot #AZF-1-FD-7-4C (6.7% weight gain) are provided in FIG. 30.

These results show that release profile of 6.7% weight gain sample closely matches with target profile of ER3.

### Example 9.5.2—ER8 Second Confirmation Batch

Laboratory scale compression was performed using a Korsch XL-100 rotary tablet press. About 2 kg tablets were compressed using common blend of Lot No. AZF-1-FD-7-4. Laboratory scale coating was performed in two lots using a Vector LDCS-5 Pan Coater equipped with 12″ partially perforated coating pan. For first lot (Lot No. AZF-1-FD-7-4A), semipermeable membrane coating, plasticizer (pore former) level was kept at 25% of coating and coating was done up to 4% weight gain. For second lot (Lot No. AZF-1-FD-7-4B), semipermeable membrane coating, plasticizer (pore former) level was kept at 30% of coating and coating was done up to 7% weight gain. The formulation compositions of both lots of ER8 confirmation batch is in Table 63.

TABLE 63
Formulation compositions of second ER8 confirmation batch.

| Summary | Formulation component | Lot Number | | | |
|---|---|---|---|---|---|
| | | AZF-1-FD-7-4A | | AZF-1-FD-7-4B | |
| | PEG 3350 (Plasticizer) amount | 25% w/w of coating | | 30% w/w of coating | |
| | Coating Weight gain | 4.0% w/w | | 7.0% w/w | |
| | | | % w/w | | % w/w |
| Material | Function | mg/tab | core | mg/tab | core |

Core Tablet

| | | | | | |
|---|---|---|---|---|---|
| Dexpramipexole Dihydrochloride | Drug Substance | 319.01 | 42.00 | 319.01 | 42.00 |
| Monohydrate* | | | | | |
| Silicified Microcrystalline Cellulose | Diluent | 231.66 | 30.50 | 231.66 | 30.50 |
| (Prosolv SMCC HD90) | | | | | |
| Copovidone (Kollidon VA 64 fine) | Binder | 53.17 | 7.00 | 53.17 | 7.00 |
| Sodium Chloride (Emprove ®) | Inorganic Osmogen | 151.91 | 20.00 | 151.91 | 20.00 |
| Magnesium stearate (Vegetable | Lubricant | 3.80 | 0.50 | 3.80 | 0.50 |
| source) | | | | | |

| | | | | |
|---|---|---|---|---|
| Total | 759.55 | 100.00 | 759.55 | 100.00 |

Coating Membrane\hat{ }\hat{ }

| | | | | | |
|---|---|---|---|---|---|
| Opadry CA 500F190003 clear (CA | CA: Film former & | 30.38 | n/a | n/a | n/a |
| 398-10:PEG 3350 = 85:15) | PEG 3350: Pore former | | | | |
| Opadry CA 500F190006 clear | | n/a | n/a | 53.17 | n/a |
| (Cellulose acetate 398-10:PEG | | | | | |
| 3350 = 70:30) | | | | | |
| Acetone\hat{ } | Solvent | q.s. | n/a | q.s. | n/a |
| Purified water\hat{ } | Solvent | q.s. | n/a | q.s. | n/a |

| | | | | |
|---|---|---|---|---|
| Total | 789.93 | n/a | 812.72 | n/a |

q.s.: Quantity sufficient
*Conversion factor for Dexpramipexole Dihydrochloride Monohydrate to Dexpramipexole Dihydrochloride = 0.9404
\hat{ }Evaporates during processing
\hat{ }\hat{ }Solid content of Coating Solution = 5% w/w, Acetone:water = 94:6

The compression parameters used determined by tablet size and shape. The main compression forces applied was to achieve the target hardness of 20-25 kp. The process parameters for compression are summarized in Table 64.

TABLE 64
Process parameters of Dexpramipexole ER Osmotic 223 mg tablets
(300 mg diHCl) second confirmation batch, Lot No. AZF-1-FD-7-4A/4B
Process Parameters

| | |
|---|---|
| Equipment | Korsch XL100 |
| Tooling | 7.75* 17.0 mm capsule shaped |
| Target Tablet weight (mg) | 760 |
| Target Hardness (kp) | 20 |
| Turret Speed (rpm) | 20 |
| Feeder Speed (rpm) | Gravity feeder used |
| Main compression force (kN) | 25-35 kN |

Process parameters for semipermeable membrane coating are described in Table 65.

TABLE 65
Process parameters of fist confirmation batch of ER6,
Lot No. AZF-1-FD-7-4A/4B

| | Lot No. | Lot No. |
|---|---|---|
| | AZF-1-FD-7-4A | AZF-1-FD-7-4B |
| | Semipermeable | Semipermeable |
| | membrane | membrane |
| Parameters | coating | coating |
| Equipment | LDCS 5 | LDCS 5 |
| Pan load (g) | 900 | 900 |
| Pan Size (inch) | 12 | 12 |
| Nozzle Size (mm) | 0.5 | 0.5 |
| Atomization Pressure (psi) | 20 | 20 |
| Drum Speed (rpm) | 18 | 18 |
| Target Product Temperature | 24 | 24 |
| (° C.) | | |
| Spray Rate (g/min) | 18 | 18 |

To test dissolution, film coated tablets were drilled with 0.5 mm Delivery orifice using a mechanical drill press. The dissolution results for Lot #AZF-1-FD-7-4A (4.0% weight gain) and Lot #AZF-1-FD-7-4B (7.0% weight gain) are provided in FIG. 31.

These results show that release profiles of both Lot No. AZF-1-FD-7-4A (25% pore former/4% weight gain) and Lot No. AZF-1-FD-7-4B (30% pore former/7% weight gain) closely match with target profiles for ER8.

### Example 9.6—Clinical Batches

Based on the results of the confirmatory batches, phase 1 clinical trial material batches were manufactured for both ER6 and ER8, at a slightly higher scale of 12 kg. The formulations are provided in Table 66. During the laser drilling, the process parameters were adjusted to achieve the targets. However, the priority was given to the cut penetration to assure that it passes through the semi-permeable membrane. The laser drilled holes are conical where a deeper cut penetration results in a larger hole diameter. An average hole diameter of 0.80-1.00 mm was observed with a cut penetration of ˜0.75-0.90 mm. Summary of process parameters is presented in Table 67.

TABLE 66
Clinical Trial batches of Dexpramipexole Dihydrochloride CR Osmotic Tablets, 300 mg.

| | | Batch Number | | | |
|---|---|---|---|---|---|
| | | ER6 (6018408) | | ER8 (6024503) | |
| | | | % w/w | | % w/w |
| Material | Function | mg/tab | core | mg/tab | core |

Core tablet

| | | | | | |
|---|---|---|---|---|---|
| Dexpramipexole Dihydrochloride Monohydrate* | Drug Substance | 399.82 | 42.00 | 319.01 | 42.00 |
| Silicified Microcrystalline Cellulose (Prosolv SMCC HD90) | Diluent | 290.35 | 30.50 | 231.66 | 30.50 |
| Copovidone (Kollidon VA 64 fine) | Binder | 66.64 | 7.00 | 53.17 | 7.00 |
| Sodium Chloride (Emprove ®) | Inorganic Osmogen | 190.34 | 20.00 | 151.91 | 20.00 |
| Magnesium stearate (Vegetable source) | Lubricant | 4.76 | 0.50 | 3.80 | 0.50 |

| | | | | |
|---|---|---|---|---|
| Total | 951.96 | 100.00 | 759.55 | 100.00 |

Coating Membrane\hat{ }\hat{ }, $

| | | | | | |
|---|---|---|---|---|---|
| Opadry CA 500F190033 clear (Cellulose acetate | CA: Film former & | 63.78 | n/a | n/a | n/a |
| 398-10:PEG 3350 = 82.5:17.5) | PEG 3350: Pore former | | | | |
| Opadry CA 500F190015 clear (Cellulose acetate | | n/a | n/a | 34.18 | n/a |
| 398-10:PEG 3350 = 75:25 | | | | | |
| Acetone\hat{ } | Solvent | q.s. | n/a | 584.47 | n/a |
| Purified water\hat{ } | Solvent | q.s. | n/a | 64.94 | n/a |

| | | | | |
|---|---|---|---|---|
| Total | 1015.74 | n/a | 793.73 | n/a |

Film Coating\hat{ }\hat{ }\hat{ }

| | | | | |
|---|---|---|---|---|
| Opadry II 85F130089 Pink | 30.47 | n/a | 23.81 | n/a |
| Purified water\hat{ } | 121.88 | n/a | 95.24 | n/a |

| | | | | |
|---|---|---|---|---|
| Total | 1046.21 | n/a | 817.54 | n/a |

q.s.: Quantity sufficient
*Conversion factor for Dexpramipexole Dihydrochloride Monohydrate to Dexpramipexole Dihydrochloride = 0.9404
\hat{ }Evaporates during processing
\hat{ }\hat{ }Solid content of Coating Solution = 5% w/w, Acetone:water = 94:6
$ Laser drilling done at the end of membrane coating
\hat{ }\hat{ }\hat{ }Solid content of Coating dispersion = 20% w/w

TABLE 67
Summary of process parameters for
Clinical trial batches 6018408 (ER6) and 6024503 (ER8)

| Process Parameters | | 6018408 (ER6) | | 6024503 (ER8) | |
|---|---|---|---|---|---|
| Compression | Target Weight (mg) | 952 | (904-1000) | 760 | (722-798) |
| | Target Composite | 9520 | (9234-9806) | 7600 | (7370-7830) |
| | (mg) (10 tabs) | | | | |
| | Target Thickness | Report Results | | Report Results | |
| | (mm) | | | | |
| | Target Hardness (kP) | 20 | (15-25) | 17 | (10-24) |
| | Press Speed (rpm) | 20 | | 20 | |
| | Feeder Speed (rpm) | 20 | | 20 | |
| Membrane Coating | Pan size (in) | 24 | | 24 | |
| | Nozzle port opening | 1.2 | | 1.2 | |
| | (mm) | | | | |
| | Drum Speed (rpm) | 10-12 | | 10 | |
| | Inlet Air Volume | 250-300 | | 250-300 | |
| | (cfm) | | | | |
| | Inlet Air Temperature | 30-40 | | 30-40 | |
| | (° C.) | | | | |
| | Product Temperature | Not recorded | | 24-27 | |
| | (° C.) | | | | |
| | Exhaust Air | 24-27 | | 24-27 | |
| | Temperature (° C.) | | | | |
| | Target Weight Gain | 6.7% | (6.4%-7.0%) | 4.5% | (4.2%-4.8%) |

| | Spray Rate (g/min) | 80-100 | | 80-100 |
|---|---|---|---|---|
| | Pattern Air Pressure | 20 | | 20 |
| | (psi) | | | |
| | Atomizing Air | 15 | | 15 |
| | Pressure (psi) | | | |
| Laser Drilling | Spindle speed (rpm) | 800 | | 800 |
| | System speed | 350 | | 300 |
| | (links/min) | | | |
| | Pitch (%) | 0.200 | | 0.040 |
| | Drill speed (mm/sec) | 500 | | 2500 |
| | Power level (%) | 90 | | 90 |
| | PWM Frequency | 25 | | 25 |
| | (KHz) | | | |
| | Hole count per tablet | 1 | | 1 |
| | Hole Diameter (mm) | 0.8 | | 0.8 |
| | Hole count per tablet | 1 | | 0.88 |
| | Hole aspect ratio | 1.0 | | 1.1 |
| | Cut penetration (mm) | 0.83 | | 0.88 |
| | (For non-centered | | | |
| | hole) | | | |
| Film Coating | Pan Size (in) | 24 | | Not performed |
| | Nozzle port opening | 1.2 | | |
| | (mm) | | | |
| | Drum Speed (rpm) | 12-15 | | |
| | Inlet Air Volume | 250-450 | | |
| | (cfm) | | | |
| | Inlet Air Temperature | 60-65 | | |
| | (° C.) | | | |
| | Product Temperature | Not recorded | | |
| | (° C.) | | | |
| | Exhaust Air | 40-55 | | |
| | Temperature (° C.) | | | |
| | Target Weight Gain | 3.0% | (2.5%-3.5%) | |

| | |
|---|---|
| Spray Rate (g/min) | 45 |
| Pattern Air Pressure | 25 |
| (psi) | |
| Atomizing Air | 35 |
| Pressure (psi) | |

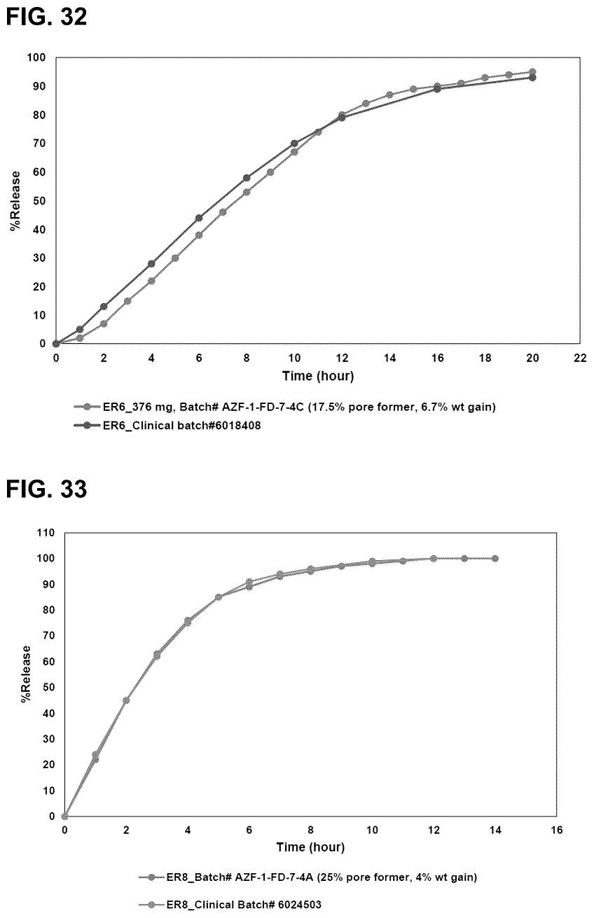

The reproducibility of clinical batch of ER6 compared to lab scale confirmatory batch No. AZF-11D-7-4C is presented in FIG. 32. ER6 clinical batch indicates very good reproducibility.

The reproducibility of clinical batch of ER8 as compared to lab scale confirmatory batch No. AZF-1-FD-7-4A is presented in FIG. 33. ER8 clinical batch indicates very good reproducibility.

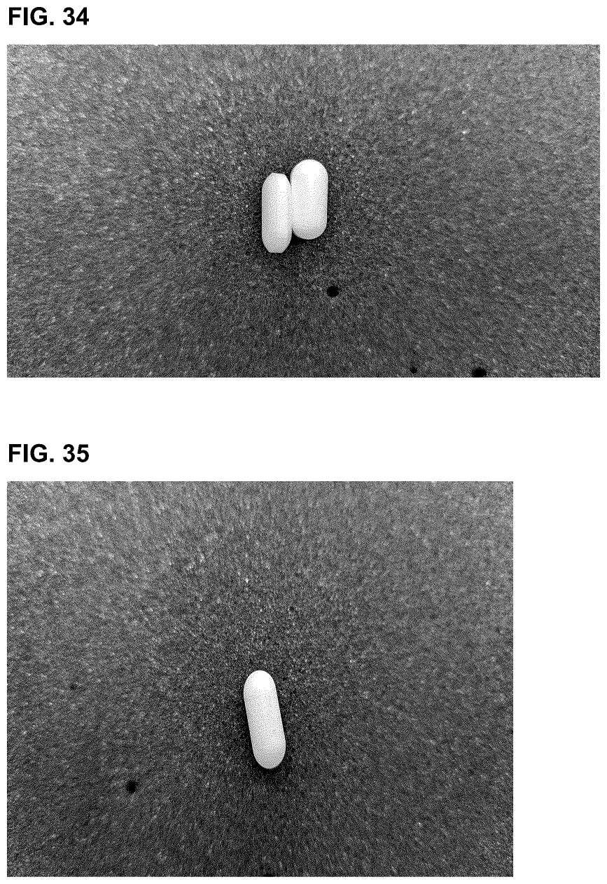

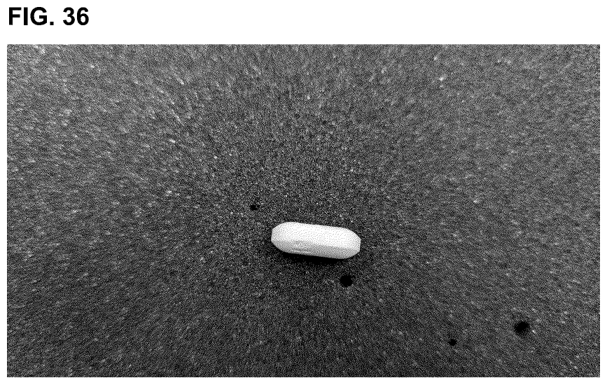

Twinning (tablet sticking) and peeling of the functional coated layer was observed during discharge of the completed membrane coated batch of ER8 (Batch No. 6045203) from the coating pan. A preliminary course visual inspection identified that the twinning and peeling was a result of the speed of the pan which remained at 10 rpm throughout the run (per batch record) and may have led to over wetting of the cores which caused the sticking and lead to peeling. An initial review identified approximately 2-2.5% of the batch had twinning and were segregated out of the bulk prior to Acceptable Quality level (AQL). As peeling of the functional coat could cause a potential risk to core exposure (critical defect), it was decided to perform 100% visual inspection of the batch. FIGS. 34-36 depict the tablets with twinning and peeling defects.

### Example 9 Conclusions

In this development, three OROS formulations were developed for Dexpramipexole controlled-release tablets. The highlights of the formulations are:
Formulation ER6 consists of target membrane weight gain of 6.7%, and Cellulose acetate and PEG 3350 in the ratio of 82.5:17.5 with 280 mg Dexpramipexole (equivalent to 376 mg Dexpramipexole diHCl).
Formulation ER8 consists of target membrane weight gain of 4.5%, and Cellulose acetate and PEG 3350 in the ratio of 75:25 with 223 mg Dexpramipexole (equivalent to 300 mg of Dexpramipexole diHCl).

All formulations met the requirements of targeted dissolution profile:
ER6 is comparable to ER3 clinical dissolution (see FIG. 32)
ER8 is within the dissolution target range predicted by IVIVC studies (see FIG. 33).

During the development, it was observed that, ER6 and ER8 were stable under the accelerated conditions and controlled room temperature and meet the dissolution criteria for clinical dosing.

Additionally, the osmotic tablet formulations shown in Table 37 are given to healthy human volunteers once a day (QD; quaque die) to assess bioavailability of dexpramipexole, as described in Example 8, above. The relative bioavailabilities of ER and ER8 formulations are shown in Table 68.

TABLE 68
Pharmacokinetics Relative Bioavailability Assessment (IR vs. ER)

| | | | Test | Reference | Geometric | | Intra- |
|---|---|---|---|---|---|---|---|
| Parameter | | | Geometric | Geometric | Mean Ratio | | Subject |
| (unit) | Ref. | Test | LSMs (n) | LSMs (n) | (%) | 90% CI | CV (%) |
| AUC₀₋₂₄ | IR | ER6 | 6091 (18) | 6047 (18) | 100.73 | 94.38-107.51 | 11.8 |
| (h*ng/mL) | | ER8 | 6462 (17) | 6047 (18) | 106.87 | 100.01-114.20 | |
| AUC₀₋₄₈ | IR | ER6 | 7946 (18) | 7302 (18) | 108.81 | 100.43-117.89 | 14.5 |
| (h*ng/mL) | | ER8 | 7389 (17) | 7302 (18) | 101.19 | 93.25-109.80 | |
| AUC₀₋₉₆ | IR | ER6 | 8351 (18) | 7549 (18) | 110.63 | 101.71-120.33 | 15.2 |
| (h*ng/mL) | | ER8 | 7613 (17) | 7549 (18) | 100.85 | 92.58-109.87 | |
| Cₘₐₓ | IR | ER6 | 400.3 (18) | 495.4 (18) | 80.82 | 75.27-86.78 | 12.9 |
| (ng/mL) | | ER8 | 505.6 (17) | 495.4 (18) | 102.07 | 94.93-109.74 | |

Acceptance Criteria: all AUC 90% CIs within 80-125%. AUC 0-48 & 0-96 most important for multiple dosing.
The ANOVA model includes treatment, sequence, and period as fixed effects and participant nested within sequence as a random effect . . . Intra-Sunject CV % = 100 × (square root (exp[MSE] − 1)), where MSE = residual variance from ANOVA;
CI = Confidence Interval;
CV = Coefficienct of Variation;
ER = Extended Release;
IR = Immediate Release;
LSM = Least-Squares Mean;
n = Number of Subjects.
Subject 2012 excluded from ER7 formulations due to early termination in Period 5.

### Conclusion on Examples

As outlined above, sustained release formulations in the form of an orally deliverable tablet cannot exceed a certain maximum weight (typically about 1500 mg), as the tablet must remain swallowable for a human. The comparably high amount of dexpramipexole that is typically used per day (generally, about 50 mg or more dexpramipexole dihydrochloride equivalent, such as about 75 mg, about 150 mg, or about 300 mg dexpramipexole dihydrochloride equivalent, per day) thus restricts the possibilities to formulate a sustained release composition that is suitable for once daily oral administration to a human. This was considered as problematic as a higher amount of drug usually also requires for a higher amount of additional ingredients such as diluents and/or binders in the sustained release formulation to provide for a composition that can be administered once daily. Thus, it could not have been reasonably expected that formulating the drug as a composition that is suitable for once daily oral administration to a human is possible. However, herein, osmotic tablet formulations that are suitable for once daily oral administration to a human were developed.

A tablet core providing for a homogeneous mixture of inorganic osmotic agent at amounts specified herein (for example, at amounts constituting about 20% to about 30% by weight of the tablet core) and dexpramipexole, or a pharmaceutically acceptable salt thereof (for example, at an amount of about 300 mg dexpramipexole dihydrochloride equivalent) was shown to be particularly beneficial for the drug release. For example, terminal release at 24 hours as measured by in vitro dissolution was increased in such formulations. It was also shown that such beneficial drug release profiles can be obtained with varying amounts of inorganic osmotic agent (for example, amounts constituting about 20% to about 30% by weight of the tablet core). See in particular Example 1.

Further, although swellable polymeric osmotic agents often act synergistically with inorganic osmotic agents in osmotic tablet formulations, release rate (in particular, terminal release rate) was reduced upon addition of swellable polymeric osmotic agent, in particular, polyethylene oxide, to the homogeneous mixture of inorganic osmotic agent and drug in the tablet core. See Example 2.

In addition, it was shown that the presence of plasticizer in the semipermeable membrane coating at amounts specified herein (for example, at amounts constituting about 10% to about 20% by weight of the semipermeable membrane coating) and a certain weight gain upon semipermeable membrane coating (for example, a weight ratio of the semipermeable membrane coating to the tablet core of about 0.06:1 to about 0.08:1) further exerted beneficial effects on the in vitro dissolution profiles. See in particular Examples 3 and 4.

It was further demonstrated that the specific number of delivery orifices (such as one or four) in the semipermeable membrane coating of the osmotic tablets did not significantly influence the dissolution profiles. See Example 5.

Herein, osmotic tablets comprising the entire amount of drug in a sustained release form as well as osmotic tablets comprising additionally a certain percentage (such as about 15%) of the drug amount in an immediate release form were prepared. See Examples 6 and 7. In these Examples, the immediate release form of dexpramipexole, or a pharmaceutically acceptable salt thereof, was provided in form of an immediate release drug coating surrounding the tablet core and the semipermeable membrane coating.

It was also demonstrated that the dissolution profiles of the osmotic tablets were reproducible with respect to manufacturing scale. See Examples 6 and 7. A particular suitable manufacturing process for the osmotic tablets described herein was also developed. See in particular Example 7.

Two osmotic tablet formulations with and without immediate release drug coating, respectively (“slow” and “fast”), were prepared for evaluation in clinical trials. See Examples 7.5 and 8.

Finally, five osmotic tablet formulations with and without immediate release drug coating, were prepared for evaluation. See Example 9. The osmotic tablet formulations described in Example 9 were also prepared and evaluated in clinical trials.

1. A pharmaceutical composition in the form of an orally deliverable tablet comprising a tablet core, a semipermeable membrane coating surrounding the tablet core, and dexpramipexole, or a pharmaceutically acceptable salt thereof, in an amount of about 50 mg to about 400 mg of dexpramipexole dihydrochloride equivalent, wherein
at least about 70% of the amount of dexpramipexole, or a pharmaceutically acceptable salt thereof, is in the tablet core;
the tablet core comprises a homogeneous mixture of an inorganic osmotic agent and the amount of dexpramipexole, or a pharmaceutically acceptable salt thereof, that is in the tablet core, wherein the inorganic osmotic agent constitutes about 10% to about 40% by weight of the tablet core;
the semipermeable membrane coating comprises about 5% to about 40% plasticizer by weight of the semipermeable membrane coating;
the weight ratio of the semipermeable membrane coating to the tablet core is about 0.03:1 to about 0.11:1; and
the weight of the tablet is about 1500 mg or less.

2-3. (canceled)

4. The pharmaceutical composition of claim 1, wherein the inorganic osmotic agent constitutes about 18% to about 22% by weight of the tablet core.

5-6. (canceled)

7. The pharmaceutical composition of claim 1, wherein the inorganic osmotic agent is sodium chloride.

8. The pharmaceutical composition of claim 1, wherein the homogeneous mixture in the tablet core does not comprise polyethylene oxide.

9. (canceled)

10. The pharmaceutical composition of claim 1, wherein the semipermeable membrane coating further comprises cellulose acetate.

11. (canceled)

12. The pharmaceutical composition of claim 10, wherein cellulose acetate has an acetyl content of about 38% to about 42%.

13-14. (canceled)

15. The pharmaceutical composition of claim 10, wherein the semipermeable membrane coating comprises about 70% to about 90% cellulose acetate by weight of the semipermeable membrane coating and about 30% to about 10% plasticizer by weight of the semipermeable membrane coating.

16. The pharmaceutical composition of claim 15, wherein the semipermeable membrane coating comprises about 70% cellulose acetate by weight of the semipermeable membrane coating and about 30% plasticizer by weight of the semipermeable membrane coating.

17-29. (canceled)

30. The pharmaceutical composition of claim 21, wherein the plasticizer is polyethylene glycol.

31. The pharmaceutical composition of claim 1, wherein the weight ratio of the semipermeable membrane coating to the tablet core is about 0.04:1 to about 0.1:1.

32-37. (canceled)

38. The pharmaceutical composition of claim 1, wherein the homogeneous mixture in the tablet core further comprises microcrystalline cellulose, polyvinylpyrrolidone-vinyl acetate copolymer, magnesium stearate, or any combination thereof.

39. (canceled)

40. The pharmaceutical composition of claim 38, wherein microcrystalline cellulose constitutes about 28% to about 32% by weight of the tablet core.

41. The pharmaceutical composition of any one of claim 38, wherein polyvinylpyrrolidone-vinyl acetate copolymer constitutes about 5% to about 9% by weight of the tablet core.

42. The pharmaceutical composition of any one of claim 38, wherein magnesium stearate constitutes about 0.25% to about 0.75% by weight of the tablet core.

43-49. (canceled)

50. The pharmaceutical composition of claim 1, wherein the pharmaceutically acceptable salt of dexpramipexole is dexpramipexole dihydrochloride or a hydrate thereof, such as dexpramipexole dihydrochloride monohydrate.

51. (canceled)

52. The pharmaceutical composition of claim 1, wherein the weight of the tablet is about 900 mg to about 1300 mg or about 1000 mg to about 1200 mg.

53-57. (canceled)

58. The pharmaceutical composition of claim 1, wherein at least 99% or 100% of the amount of dexpramipexole, or a pharmaceutically acceptable salt thereof, is in the tablet core and the homogeneous mixture in the tablet core consists essentially of:
(a) about 315 mg to about 325 mg dexpramipexole dihydrochloride monohydrate;
(b) about 227 mg to about 237 mg microcrystalline cellulose;
(c) about 48 mg to about 58 mg polyvinylpyrrolidone-vinyl acetate copolymer;
(d) about 147 mg to about 157 mg sodium chloride; and
(e) about 2 mg to about 6 mg magnesium stearate;
wherein the tablet comprises about 42 mg to about 52 mg semipermeable membrane coating, and wherein the semipermeable membrane coating comprises about 83% to about 87% cellulose acetate by weight of the semipermeable membrane coating and about 17% to about 13% plasticizer by weight of the semipermeable membrane coating.

59. The pharmaceutical composition of claim 58, wherein the homogeneous mixture in the tablet core consists essentially of:
(a) about 319 mg dexpramipexole dihydrochloride monohydrate;
(b) about 232 mg microcrystalline cellulose;
(c) about 53 mg polyvinylpyrrolidone-vinyl acetate copolymer;
(d) about 152 mg sodium chloride; and
(e) about 4 mg magnesium stearate;
wherein the tablet comprises about 47 mg semipermeable membrane coating, and wherein the semipermeable membrane coating comprises about 85% cellulose acetate by weight of the semipermeable membrane coating and about 15% plasticizer by weight of the semipermeable membrane coating.

60-63. (canceled)

64. The pharmaceutical composition of claim 58, further comprising a film coating surrounding the semipermeable membrane coating.

65-66. (canceled)

67. The pharmaceutical composition of claim 1, wherein
about 80% to about 95% of the amount of dexpramipexole, or a pharmaceutically acceptable salt thereof, is in the tablet core;
the tablet further comprises an immediate release drug coating surrounding the semipermeable membrane coating; and
about 20% to about 5% of the amount of dexpramipexole, or a pharmaceutically acceptable salt thereof, is in the drug coating.

68-76. (canceled)

77. The pharmaceutical composition of claim 1, wherein the homogeneous mixture in the tablet core consists essentially of:
(a) about 265 mg to about 275 mg dexpramipexole dihydrochloride monohydrate;
(b) about 192 mg to about 202 mg microcrystalline cellulose;
(c) about 40 mg to about 50 mg polyvinylpyrrolidone-vinyl acetate copolymer;
(d) about 125 mg to about 135 mg sodium chloride; and
(e) about 1 mg to about 5 mg magnesium stearate;
wherein the tablet comprises about 47 mg to about 57 mg semipermeable membrane coating, wherein the semipermeable membrane coating comprises about 83% to about 87% cellulose acetate by weight of the semipermeable membrane coating and about 17% to about 13% plasticizer by weight of the semipermeable membrane coating;
wherein the tablet further comprises about 15 mg to about 25 mg of a seal coating surrounding the semipermeable membrane coating, wherein the seal coating comprises hydroxypropyl methylcellulose and polyethylene glycol; and
wherein the tablet further comprises about 86 mg to about 106 mg of an immediate release drug coating surrounding the seal coating, wherein the drug coating comprises about 45 mg to about 50 mg dexpramipexole dihydrochloride monohydrate, and about 40 mg to about 50 mg of a binder, wherein the binder comprises hydroxypropyl methylcellulose and polyethylene glycol.

78. The pharmaceutical composition of claim 77, wherein the homogeneous mixture in the tablet core consists essentially of:
(a) about 271 mg dexpramipexole dihydrochloride monohydrate;
(b) about 197 mg microcrystalline cellulose;
(c) about 45 mg polyvinylpyrrolidone-vinyl acetate copolymer;
(d) about 129 mg sodium chloride; and
(e) about 3 mg magnesium stearate;
wherein the tablet comprises about 52 mg semipermeable membrane coating, wherein the semipermeable membrane coating comprises about 85% cellulose acetate by weight of the semipermeable membrane coating and about 15% plasticizer by weight of the semipermeable membrane coating;
wherein the tablet comprises about 21 mg seal coating; and
wherein the tablet comprises about 96 mg drug coating, wherein the drug coating comprises about 47.85 mg dexpramipexole dihydrochloride monohydrate, and about 47.85 mg of a binder, wherein the binder comprises hydroxypropyl methylcellulose and polyethylene glycol.

79-85. (canceled)

86. The pharmaceutical composition of claim 1, wherein at least 99% or 100% of the amount of dexpramipexole, or a pharmaceutically acceptable salt thereof, is in the tablet core and the homogeneous mixture in the tablet core consists essentially of:
a. about 390 mg to about 410 mg dexpramipexole dihydrochloride monohydrate;
b. about 280 mg to about 300 mg microcrystalline cellulose;
c. about 57 mg to about 77 mg polyvinylpyrrolidone-vinyl acetate copolymer;
d. about 180 mg to about 200 mg sodium chloride; and
e. about 3 mg to about 7 mg magnesium stearate;
wherein the tablet comprises about 54 mg to about 74 mg semipermeable membrane coating, and wherein the semipermeable membrane coating comprises about 81% to about 85% cellulose acetate by weight of the semipermeable membrane coating and about 15% to about 19% plasticizer by weight of the semipermeable membrane coating.

87. The pharmaceutical composition of claim 86, wherein the homogeneous mixture in the tablet core consists essentially of:
a. about 400 mg dexpramipexole dihydrochloride monohydrate;
b. about 290 mg microcrystalline cellulose;
c. about 67 mg polyvinylpyrrolidone-vinyl acetate copolymer;
d. about 190 mg sodium chloride; and
e. about 5 mg magnesium stearate;
wherein the tablet comprises about 64 mg semipermeable membrane coating, and wherein the semipermeable membrane coating comprises about 83% cellulose acetate by weight of the semipermeable membrane coating and about 17% plasticizer by weight of the semipermeable membrane coating.

88-95. (canceled)

96. The pharmaceutical composition of claim 1, wherein at least 99% or 100% of the amount of dexpramipexole, or a pharmaceutically acceptable salt thereof, is in the tablet core and the homogeneous mixture in the tablet core consists essentially of:
a. about 310 mg to about 330 mg dexpramipexole dihydrochloride monohydrate;
b. about 222 mg to about 242 mg microcrystalline cellulose;
c. about 43 mg to about 63 mg polyvinylpyrrolidone-vinyl acetate copolymer;
d. about 142 mg to about 162 mg sodium chloride; and
e. about 2 mg to about 6 mg magnesium stearate;
wherein the tablet comprises about 24 mg to about 44 mg semipermeable membrane coating, and wherein the semipermeable membrane coating comprises about 73% to about 77% cellulose acetate by weight of the semipermeable membrane coating and about 23% to about 27% plasticizer by weight of the semipermeable membrane coating.

97. The pharmaceutical composition of claim 96, wherein the homogeneous mixture in the tablet core consists essentially of:
a. about 319 mg dexpramipexole dihydrochloride monohydrate;
b. about 232 mg microcrystalline cellulose;
c. about 53 mg polyvinylpyrrolidone-vinyl acetate copolymer;
d. about 152 mg sodium chloride; and
e. about 4 mg magnesium stearate;
wherein the tablet comprises about 34 mg semipermeable membrane coating, and wherein the semipermeable membrane coating comprises about 75% cellulose acetate by weight of the semipermeable membrane coating and about 25% plasticizer by weight of the semipermeable membrane coating.

98-105. (canceled)

106. The pharmaceutical composition of claim 1, wherein at least 99% or 100% of the amount of dexpramipexole, or a pharmaceutically acceptable salt thereof, is in the tablet core and the homogeneous mixture in the tablet core consists essentially of:
a. about 310 mg to about 330 mg dexpramipexole dihydrochloride monohydrate;
b. about 222 mg to about 242 mg microcrystalline cellulose;
c. about 43 mg to about 63 mg polyvinylpyrrolidone-vinyl acetate copolymer;
d. about 142 mg to about 162 mg sodium chloride; and
e. about 2 mg to about 6 mg magnesium stearate;
wherein the tablet comprises about 43 mg to about 63 mg semipermeable membrane coating, and wherein the semipermeable membrane coating comprises about 68% to about 72% cellulose acetate by weight of the semipermeable membrane coating and about 28% to about 32% plasticizer by weight of the semipermeable membrane coating.

107. The pharmaceutical composition of claim 96, wherein the homogeneous mixture in the tablet core consists essentially of:
a. about 319 mg dexpramipexole dihydrochloride monohydrate;
b. about 232 mg microcrystalline cellulose;
c. about 53 mg polyvinylpyrrolidone-vinyl acetate copolymer;
d. about 152 mg sodium chloride; and
e. about 4 mg magnesium stearate;
wherein the tablet comprises about 53 mg semipermeable membrane coating, and wherein the semipermeable membrane coating comprises about 70% cellulose acetate by weight of the semipermeable membrane coating and about 30% plasticizer by weight of the semipermeable membrane coating.

108-123. (canceled)

124. The pharmaceutical composition of claim 1, wherein about 55% to about 85% of dexpramipexole, or the pharmaceutically acceptable salt thereof, is released at about 12 hours of incubation of the tablet in about 900 mL of 50 mM monobasic potassium phosphate buffer, pH 6.8, at a temperature of 37±0.5° C. as measured using an USP type I apparatus operated at a spindle rotation speed of about 100 rpm.

125-128. (canceled)

129. A method of treating or preventing asthma in a human subject in need thereof, the method comprising orally administering to the human subject the pharmaceutical composition of claim 1.

130. The method of claim 129, wherein the asthma is eosinophilic asthma.

131. A method of treating or preventing chronic obstructive pulmonary disease in a human subject in need thereof, the method comprising orally administering to the human subject the pharmaceutical composition of claim 1.

132. A method of treating or preventing an eosinophilic disorder in a human subject in need thereof, the method comprising orally administering to the human subject the pharmaceutical composition of claim 1.

133. The method of claim 132, wherein the eosinophilic disorder is selected from the group consisting of hypereosinophilic syndrome, chronic rhinosinusitis with nasal polyps, nasal polyposis, atopic dermatitis, eosinophilic granulomatosis with polyangiitis, eosinophilic gastroenteritis, eosinophilic esophagitis, and any combination thereof.

134-137. (canceled)

138. A method of manufacturing a pharmaceutical composition in the form of an orally deliverable tablet comprising dexpramipexole, or a pharmaceutically acceptable salt thereof, the method comprising:
preparing a pre-blend comprising dexpramipexole, or a pharmaceutically acceptable salt thereof, and an inorganic osmotic agent;
preparing a blend comprising the pre-blend and a lubricant;
compressing the blend to form a tablet core; and
coating the tablet core with a semipermeable membrane coating comprising a plasticizer;
wherein dexpramipexole, or the pharmaceutically acceptable salt thereof, constitutes about 37% to about 47% of dexpramipexole dihydrochloride monohydrate equivalent by weight of the blend, wherein the inorganic osmotic agent constitutes about 10% to about 40% by weight of the blend, wherein the semipermeable membrane coating comprises about 5% to about 40% plasticizer by weight of the semipermeable membrane coating, and wherein the weight ratio of the semipermeable membrane coating to the tablet core is about 0.03:1 to about 0.11:1.

139-146. (canceled)

147. The method of claim 138, wherein the pre-blend further comprises microcrystalline cellulose, polyvinylpyrrolidone-vinyl acetate copolymer, or both.

148. (canceled)

149. The method of claim 147, wherein microcrystalline cellulose constitutes about 25% to about 35% by weight of the blend.

150. The method of claim 147, wherein polyvinylpyrrolidone-vinyl acetate copolymer constitutes about 5% to about 9% by weight of the blend.

151. The method of claim 138, wherein the lubricant is magnesium stearate.

152. The method of claim 151, wherein the lubricant constitutes about 0.25% to about 0.75% by weight of the blend.

153-155. (canceled)

156. The method of claim 138, wherein the blend consists essentially of:
(a) about 40% to 44% dexpramipexole dihydrochloride monohydrate by weight of the blend;
(b) about 28% to about 32% microcrystalline cellulose by weight of the blend;
(c) about 5% to about 9% polyvinylpyrrolidone-vinyl acetate copolymer by weight of the blend;
(d) about 18% to about 22% sodium chloride by weight of the blend; and
(e) about 0.25% to about 0.75% magnesium stearate by weight of the blend, wherein sodium chloride is the inorganic osmotic agent and magnesium stearate is the lubricant.

157-205. (canceled)

206. The method of claim 138, further comprising:
coating the semipermeable membrane coating with an immediate release drug coating comprising dexpramipexole, or a pharmaceutically acceptable salt thereof.

207. The method of claim 206, wherein the drug coating further comprises a binder.

208-214. (canceled)
